(19) United States
(12) Patent Application Publication
(10) Pub. No.: US 2002/0120116 A1
(43) Pub. Date: Aug. 29, 2002

(54) ENTEROCOCCUS FAECALIS POLYNUCLEOTIDES AND POLYPEPTIDES

(76) Inventors: CHARLES A. KUNSCH, ATLANTA, GA (US); PATRICK J. DILLON, CARLSBAD, CA (US); STEVEN BARASH, ROCKVILLE, MD (US)

Correspondence Address: HUMAN GENOME SCIENCES INC, 9410 KEY WEST AVENUE, ROCKVILLE, MD, 20850

(21) Appl. No.: 09/070,927

(22) Filed: May 4, 1998

(51) Int. Cl. C07K 16/00

(52) U.S. Cl. 536/23.2; 435/69.1; 435/70.1; 435/71.1; 435/252.3; 435/320.1; 530/350; 530/387.9; 800/13

(57) ABSTRACT

The present invention provides polynucleotide sequences of the genome of Enterococcus faecalis, polypeptide sequences encoded by the polynucleotide sequences, corresponding polynucleotides and polypeptides, vectors and hosts comprising the polynucleotides, and assays and other uses thereof. The present invention further provides polynucleotide and polypeptide sequence information stored on computer readable media, and computer-based systems and methods which facilitate its use.

This application claims benefit of 35 U.S.C. section 119(e) based on copending U.S. Provisional Application Serial No. 60/046,655, filed May 16, 1997; 60/044,031, filed May 6, 1997; and 60/066,099, filed Nov. 14, 1997. Provisional Application Serial No. 60/066,099, filed Nov. 14, 1997 is herein incorporated by reference in its entirety.

### FIELD OF THE INVENTION

The present invention relates to the field of molecular biology. In particular, it relates to, among other things, nucleotide sequences of Enterococcus faecalis, contigs, ORFs, fragments, probes, primers and related polynucleotides thereof, peptides and polypeptides encoded by the sequences, and uses of the polynucleotides and sequences thereof, such as in fermentation, polypeptide production, assays and pharmaceutical development, among others.

### BACKGROUND OF THE INVENTION

Enterococci have been recognized as being pathogenic for humans since the turn of the century when they were first described by Thiercelin in 1988 as microscopic organisms. The genus Enterococcus includes the species Enterococcus faecalis or E. faecalis which is the most common pathogen in the group, accounting for 80-90 percent of all enterococcal infections. See Lewis et al. (1990) Eur J. Clin Microbiol Infect Dis. 9:111-117.

The incidence of enterococcal infections has increased in recent years and enterococci are now the second most frequently reported nosocomial pathogens. Enterococcal infection is of particular concern because of its resistance to antibiotics. Recent attention has focused on enterococci not only because of their increasing role in nosocomial infections, but also because of their remarkable and increasing resistance to antimicrobial agents. These factors are mutually reinforcing since resistance allows enterococci to survive in an environment in which antimicrobial agents are heavily used; the hospital setting provides the antibiotics which eliminate or suppress susceptible bacteria, thereby providing a selective advantage for resistant organisms, and the hospital also provides the potential for dissemination of resistant enterococci via the usual routes of hand and environmental contamination.

Antimicrobial resistance can be divided into two general types, inherent or intrinsic property and that which is acquired. The genes for intrinsic resistance, like other other species characteristics, appear to reside on the chromosome. Acquired resistance results from either a mutation in the existing DNA or acquisition of new DNA. The various inherent traits expressed by enterococci include resistance to semisynthetic penicillinase-resistant penicillins, cephalosporins, low levels of aminoglycosides, and low levels of clindamycin. Examples of acquired resistance include resistance to chloramphenicol, erythromycin, high levels of clindamycin, tetracycline, high levels of aminoglycosides, penicillin by means of penicillinase, fluoroquinolones, and vancomycin. Resistance to high levels of penicillin without penicillinase and resistance to fluoroquinolones are not known to be plasmid or transposon mediated and presumably are due to mutation(s).

Although the main reservoir for enterococci in humans is the gastrointestinal tract, the bacteria can also reside in the gallbladder, urethra and vagina.

E. faecalis has emerged as an important pathogen in endocarditis, bacteremia, urinary tract infections (UTIs), intraabdominal infections, soft tissue infections, and neonatal sepsis (Lewis 1990, supra). In the 1970s and 1980s enterococci became firmly established as major nosocomial pathogens. They are now the fourth leading cause of hospital-acquired infection and the third leading cause of bacteremia in the United States. Fatality ratios for enterococcal bactermia range from 12% to 68%, with death due to enterococcal sepsis in 4 to 50% of these cases. See Emori, T. G. (1993) Clin. Microbiol. Rev. 6:428-442.

The ability of enterococci to colonize the gastrointestinal tract, plus the many intrinsic and acquired resistance traits, means that these organisms, which usually seem to have relatively low intrinsic virulence, are given an excellent opportunity to become secondary invaders. Since nosocomial isolates of enterococci have displayed resistance to essentially every useful antimicrobial agent, it will likely become increasingly difficult to successfully treat and control enterococcal infections. Particularly when the various resistance genes come together in a single strain, an event almost certain to occur at some time in the future.

The etiology of diseases mediated or exacerbated by Enterococcus faecalis, involves the programmed expression of E. faecalis genes, and that characterizing these genes and their patterns of expression would dramatically add to our understanding of the organism and its host interactions. Knowledge of the E. faecalis gene and genomic organization would improve our understanding of disease etiology and lead to improved and new ways of preventing, treating and diagnosing diseases. Thus, there is a need to characterize the genome of E. faecalis and for polynucleotides of this organism.

### SUMMARY OF THE INVENTION

The present invention is based on the sequencing of fragments of the Enterococcus faecalis genome. The primary nucleotide sequences which were generated are provided in SEQ ID NOS: 1-982.

The present invention provides the nucleotide sequence of hundreds of contigs of the Enterococcus faecalis genome, which are listed in tables below and set out in the Sequence Listing submitted herewith, and representative fragments thereof, in a form which can be readily used, analyzed, and interpreted by a skilled artisan. In one embodiment, the present invention is provided as contiguous strings of primary sequence information corresponding to the nucleotide sequences depicted in SEQ ID NOS:1-982.

The present invention further provides nucleotide sequences which are at least 95%, 96%, 97%, 98%, and 99%, identical to the nucleotide sequences of SEQ ID NOS:1-982.

The nucleotide sequence of SEQ ID NOS:1-982, a representative fragment thereof, or a nucleotide sequence which is at least 95% identical to the nucleotide sequence of SEQ ID NOS:1-982 may be provided in a variety of mediums to facilitate its use. In one application of this embodiment, the sequences of the present invention are recorded on computer readable media. Such media includes, but is not limited to: magnetic storage media, such as floppy discs, hard disc storage medium, and magnetic tape; optical storage media such as CD-ROM; electrical storage media such as RAM and ROM; and hybrids of these categories such as magnetic/optical storage media.

The present invention further provides systems, particularly computer-based systems which contain the sequence information herein described stored in a data storage means. Such systems are designed to identify commercially important fragments of the Enterococcus faecalis genome.

Another embodiment of the present invention is directed to fragments of the Enterococcus faecalis genome having particular structural or functional attributes. Such fragments of the Enterococcus faecalis genome of the present invention include, but are not limited to, fragments which encode peptides, hereinafter referred to as open reading frames or ORFs, fragments which modulate the expression of an operably linked ORF, hereinafter referred to as expression modulating fragments or EMFs, and fragments which can be used to diagnose the presence of Enterococcus faecalis in a sample, hereinafter referred to as diagnostic fragments or DFs.

Each of the ORFs in fragments of the Enterococcus faecalis genome disclosed in Tables 1-3, and the EMFs found 5′ prime of the initiation codon, can be used in numerous ways as polynucleotide reagents. For instance, the sequences can be used as diagnostic probes or amplification primers for detecting or determining the presence of a specific microbe in a sample, to selectively control gene expression in a host and in the production of polypeptides, such as polypeptides encoded by ORFs of the present invention, particular those polypeptides that have a pharmacological activity.

The present invention further includes recombinant constructs comprising one or more fragments of the Enterococcus faecalis genome of the present invention. The recombinant constructs of the present invention comprise vectors, such as a plasmid or viral vector, into which a fragment of the Enterococcus faecalis has been inserted.

The present invention further provides host cells containing any of the isolated fragments of the Enterococcus faecalis genome of the present invention. The host cells can be a higher eukaryotic host cell, such as a mammalian cell, a lower eukaryotic cell, such as a yeast cell, or a procaryotic cell such as a bacterial cell.

The present invention is further directed to isolated polypeptides and proteins encoded by ORFs of the present invention. A variety of methods, well known to those of skill in the art, routinely may be utilized to obtain any of the polypeptides and proteins of the present invention. For instance, polypeptides and proteins of the present invention having relatively short, simple amino acid sequences readily can be synthesized using commercially available automated peptide synthesizers. Polypeptides and proteins of the present invention also may be purified from bacterial cells which naturally produce the protein. Yet another alternative is to purify polypeptide and proteins of the present invention from cells which have been altered to express them.

The invention further provides methods of obtaining homologs of the fragments of the Enterococcus faecalis genome of the present invention and homologs of the proteins encoded by the ORFs of the present invention. Specifically, by using the nucleotide and amino acid sequences disclosed herein as a probe or as primers, and techniques such as PCR cloning and colony/plaque hybridization, one skilled in the art can obtain homologs.

The invention further provides antibodies which selectively bind polypeptides and proteins of the present invention. Such antibodies include both monoclonal and polyclonal antibodies.

The invention further provides hybridomas which produce the above-described antibodies. A hybridoma is an immortalized cell line which is capable of secreting a specific monoclonal antibody.

The present invention further provides methods of identifying test samples derived from cells which express one of the ORFs of the present invention, or a homolog thereof. Such methods comprise incubating a test sample with one or more of the antibodies of the present invention, or one or more of the DFs of the present invention, under conditions which allow a skilled artisan to determine if the sample contains the ORF or product produced therefrom.

In another embodiment of the present invention, kits are provided which contain the necessary reagents to carry out the above-described assays.

Specifically, the invention provides a compartmentalized kit to receive, in close confinement, one or more containers which comprises: (a) a first container comprising one of the antibodies, or one of the DFs of the present invention; and (b) one or more other containers comprising one or more of the following: wash reagents, reagents capable of detecting presence of bound antibodies or hybridized DFs.

Using the isolated proteins of the present invention, the present invention further provides methods of obtaining and identifying agents capable of binding to a polypeptide or protein encoded by one of the ORFs of the present invention. Specifically, such agents include, as further described below, antibodies, peptides, carbohydrates, pharmaceutical agents and the like. Such methods comprise steps of: (a)contacting an agent with an isolated protein encoded by one of the ORFs of the present invention; and (b)determining whether the agent binds to said protein.

The present genomic sequences of Enterococcus faecalis will be of great value to all laboratories working with this organism and for a variety of commercial purposes. Many fragments of the Enterococcus faecalis genome will be immediately identified by similarity searches against GenBank or protein databases and will be of immediate value to Enterococcus faecalis researchers and for immediate commercial value for the production of proteins or to control gene expression.

The methodology and technology for elucidating extensive genomic sequences of bacterial and other genomes has and will greatly enhance the ability to analyze and understand chromosomal organization. In particular, sequenced contigs and genomes will provide the models for developing tools for the analysis of chromosome structure and function, including the ability to identify genes within large segments of genomic DNA, the structure, position, and spacing of regulatory elements, the identification of genes with potential industrial applications, and the ability to do comparative genomic and molecular phylogeny.

### DESCRIPTION OF THE FIGURES

FIG. 1 is a block diagram of a computer system (102) that can be used to implement computer-based systems of the present invention.

FIG. 2 is a schematic diagram depicting the data flow and computer programs used to collect, assemble, edit and annotate the contigs of the Enterococcus faecalis genome of the present invention. Both Macintosh and Unix platforms are used to handle the AB 373 and 377 sequence data files, largely as described in Kerlavage et al., Proceedings of the Twenty-Sixth Annual Hawaii International Conference on System Sciences, 585, IEEE Computer Society Press, Washington D.C. (1993). Factura (AB) is a Macintosh program designed for automatic vector sequence removal and end-trimming of sequence files. The program Sequis runs on a Macintosh platform and parses the feature data extracted from the sequence files by Factura to the Unix based Enterococcus faecalis relational database. Assembly of contigs (and whole genome sequences) is accomplished by retrieving a specific set of sequence files and their associated features using Extrseq, a Unix utility for retrieving sequences from an SQL database. The resulting sequence file is processed by seq_filter to trim portions of the sequences with more than 1% ambiguous nucleotides. The sequence files were assembled using TIGR Assembler, an assembly engine designed at The Institute for Genomic Research (TIGR) for rapid and accurate assembly of thousands of sequence fragments. The collection of contigs generated by the assembly step is loaded into the database with the lassie program. Identification of open reading frames (ORFs) is accomplished by processing contigs with GeneMark, described in Borodovsky, M. and McIninch, J. D. (1993) Comput. Chem., 17:123 133. The ORFs are searched against E. faecalis sequences from GenBank and against all protein sequences using the BLASTN and BLASTP programs, described in Altschul et al., J. Mol. Biol. 215: 403-410 (1990)). Results of the ORF determination and similarity searching steps were loaded into the database. As described below, some results of the determination and the searches are set out in Tables 1-3.

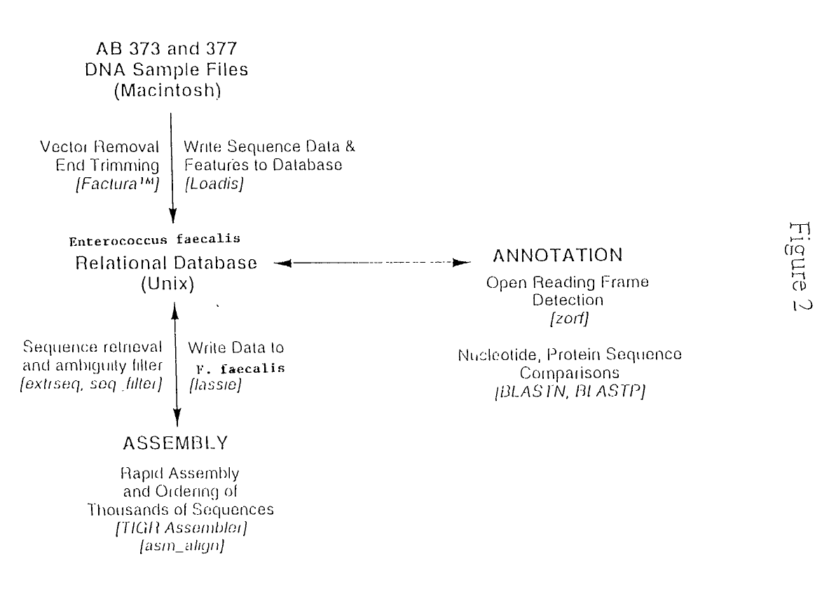

### DETAILED DESCRIPTION OF ILLUSTRATIVE EMBODIMENTS

The present invention is based on the sequencing of fragments of the Enterococcus faecalis genome and analysis of the sequences. The primary nucleotide sequences generated by sequencing the fragments are provided in SEQ ID NOS: 1-982. (As used herein, the “primary sequence” refers to the nucleotide sequence represented by the IUPAC nomenclature system.)

In addition to the aforementioned Enterococcus faecalis polynucleotide and polynucleotide sequences, the present invention provides the nucleotide sequences of SEQ ID NOS: 1-982, or representative fragments thereof, in a form which can be readily used, analyzed, and interpreted by a skilled artisan.

As used herein, a “representative fragment of the nucleotide sequence depicted in SEQ ID NOS:1-982” refers to any portion of the SEQ ID NOS: 1-982 which is not presently represented within a publicly available database. Preferred representative fragments of the present invention are Enterococcus faecalis open reading frames (ORFs), expression modulating fragment (EMFs) and fragments which can be used to diagnose the presence of Enterococcus faecalis in a sample (DFs). A non-limiting identification of preferred representative fragments is provided in Tables 1-3. As discussed in detail below, the information provided in SEQ ID NOS:1-982 and in Tables 1-3 together with routine cloning, synthesis, sequencing and assay methods will enable those skilled in the art to clone and sequence all “representative fragments” of interest, including open reading frames encoding a large variety of Enterococcus faecalis proteins.

The present invention is further directed to nucleic acid molecules encoding portions or fragments of the nucleotide sequences described herein. Fragments include portions of the nucleotide sequences of Table 1-3 and SEQ ID NOS:1-982, at least 10 contiguous nucleotides in length selected from any two integers, one of which representing a 5′ nucleotide position and a second of which representing a 3′ nucleotide position, where the first nucleotide for each nucleotide sequence in SEQ ID NOS:1-982 is position 1. That is, every combination of a 5′ and 3′ nucleotide position that a fragment at least 10 contiguous nucleotides in length could occupy is included in the invention. At least means a fragment may be 10 contiguous nucleotide bases in length or any integer between 10 and the length of an entire nucleotide sequence of SEQ ID NOS:1-982 minus 1. Therefore, included in the invention are contiguous fragments specified by any 5′ and 3′ nucleotide base positions of a nucleotide sequences of SEQ ID NOS:1-982 wherein the contiguous fragment is any integer between 10 and the length of an entire nucleotide sequence minus 1.

Further, the invention includes polynucleotides comprising fragments specified by size, in nucleotides, rather than by nucleotide positions. The invention includes any fragment size, in contiguous nucleotides, selected from integers between 10 and the length of an entire nucleotide sequence minus 1. Preferred sizes of contiguous nucleotide fragments include 20 nucleotides, 30 nucleotides, 40 nucleotides, 50 nucleotides. Other preferred sizes of contiguous nucleotide fragments, which may be useful as diagnostic probes and primers, include fragments 50-300 nucleotides in length which include, as discussed above, fragment sizes representing each integer between 50-300. Larger fragments are also useful according to the present invention corresponding to most, if not all, of the nucleotide sequences shown in SEQ ID NOS:1-982. The preferred sizes are, of course, meant to exemplify not limit the present invention as all size fragments, representing any integer between 10 and the length of an entire nucleotide sequence minus 1, of each SEQ ID NO:, are included in the invention.

The present invention also provides for the exclusion of any fragment, specified by 5′ and 3′ base positions or by size in nucleotide bases as described above for any nucleotide sequence of SEQ ID NOS:1-982. Any number of fragments of nucleotide sequences in SEQ ID NOS:1-982, specified by 5′ and 3′ base positions or by size in nucleotides, as described above, may be excluded from the present invention.

While the presently disclosed sequences of SEQ ID NOS:1-982 are highly accurate, sequencing techniques are not perfect and, in relatively rare instances, further investigation of a fragment or sequence of the invention may reveal a nucleotide sequence error present in a nucleotide sequence disclosed in SEQ ID NOS:1-982. However, once the present invention is made available (i.e., once the information in SEQ ID NOS:1-982 and Tables 1-3 has been made available), resolving a rare sequencing error in SEQ ID NOS: 1-982 will be well within the skill of the art. The present disclosure makes available sufficient sequence information to allow any of the described contigs or portions thereof to be obtained readily by straightforward application of routine techniques. Further sequencing of such polynucleotides may proceed in like manner using manual and automated sequencing methods which are employed ubiquitous in the art. Nucleotide sequence editing software is publicly available. For example, Applied Biosystem's (AB) AutoAssembler can be used as an aid during visual inspection of nucleotide sequences. By employing such routine techniques potential errors readily may be identified and the correct sequence then may be ascertained by targeting further sequencing effort, also of a routine nature, to the region containing the potential error.

Even if all of the very rare sequencing errors in SEQ ID NOS: 1-982 were corrected, the resulting nucleotide sequences would still be at least 95% identical, nearly all would be at least 99% identical, and the great majority would be at least 99.9% identical to the nucleotide sequences of SEQ ID NOS:1-982.

As discussed elsewhere herein, polynucleotides of the present invention readily may be obtained by routine application of well known and standard procedures for cloning and sequencing DNA. Detailed methods for obtaining libraries and for sequencing are provided below, for instance. A wide variety of Enterococcus faecalis strains that can be used to prepare E. faecalis genomic DNA for cloning and for obtaining polynucleotides of the present invention are available to the public from recognized depository institutions, such as the American Type Culture Collection (ATCC). While the present invention is enabled by the sequences and other information herein disclosed, the E. faecalis strain that provided the DNA of the present Sequence Listing, Strain V586, kindly provided by Dr. Michael Gilmore, University of Oklahoma, has been deposited in the ATCC, as a convenience to those of skill in the art. The E. faecalis strain V586 was deposited May 2, 1997 at the ATCC, 10801 University Blvd. Manassas, Va. 20110-2209, and given accession number 55969. The provision of the deposits is not a waiver of any rights of the inventors or their assignees in the present subject matter.

The nucleotide sequences of the genomes from different strains of Enterococcus faecalis differ somewhat. However, the nucleotide sequences of the genomes of all Enterococcus faecalis strains will be at least 95% identical, in corresponding part, to the nucleotide sequences provided in SEQ ID NOS: 1-982. Nearly all will be at least 99% identical and the great majority will be 99.9% identical.

The present application is further directed to nucleic acid molecules at least 90%, 95%, 96%, 97%, 98% or 99% identical to a nucleic acid sequence shown in SEQ ID NOS: 1-982. The above nucleic acid sequences are included irrespective of whether they encode a polypeptide having E. faecalis activity. This is because even where a particular nucleic acid molecule does not encode a polypeptide having E. faecalis activity, one of skill in the art would still know how to use the nucleic acid molecule, for instance, as a hybridization probe. Uses of the nucleic acid molecules of the present invention that do not encode a polypeptide having E. faecalis activity include, inter alia, isolating an E. faecalis gene or allelic variants thereof from a DNA library, and detecting E. faecalis mRNA expression samples, environmental samples, suspected of containing E. faecalis by Northern Blot analysis.

Preferred, are nucleic acid molecules having sequences at least 90%, 95%, 96%, 97%, 98% or 99% identical to the nucleic acid sequence shown in SEQ ID NOS: 1-982, which do, in fact, encode a polypeptide having E. faecalis protein activity By “a polypeptide having E. faecalis activity” is intended polypeptides exhibiting activity similar, but not necessarily identical, to an activity of the E. faecalis protein of the invention, as measured in a particular biological assay suitable for measuring activity of the specified protein.

Due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequences shown in SEQ ID NOS: 1-982 will encode a polypeptide having E. faecalis protein activity. In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having E. faecalis protein activity. This is because the skilled artisan is fully aware of amino acid substitutions that are either less likely or not likely to significantly effect protein function (e.g., replacing one aliphatic amino acid with a second aliphatic amino acid), as further described below.

The biological activity or function of the polypeptides of the present invention are expected to be similar or identical to polypeptides from other bacteria that share a high degree of structural identity/similarity. Tables 1 and 2 lists accession numbers and descriptions for the closest matching sequences of polypeptides available through Genbank. It is therefore expected that the biological activity or function of the polypeptides of the present invention will be similar or identical to those polypeptides from other bacterial genuses, species, or strains listed in Tables 1 and 2.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the E. faecalis polypeptide. In other words, to obtain a polynucleotide having a nucleotide sequence at least 95% identical to a reference nucleotide sequence, up to 5% of the nucleotides in the reference sequence may be deleted, inserted, or substituted with another nucleotide. The query sequence may be an entire sequence shown in SEQ ID NOS: 1-982, the ORF (open reading frame), or any fragment specified as described herein.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. See Brutlag et al. (1990) Comp. App. Biosci. 6:237-245. In a sequence alignment the query and subject sequences are both DNA sequences. An RNA sequence can be compared by first converting U's to T's. The result of said global sequence alignment is in percent identity. Preferred parameters used in a FASTDB alignment of DNA sequences to calculate percent identity arc: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only nucleotides outside the 5′ and 3′ nucleotides of the subject sequence, as displayed by the FASTDB alignment, which are not matched/aligned with the query sequence, are calculated for the purposes of manually adjusting the percent identity score.

For example, a 90 nucleotide subject sequence is aligned to a 100 nucleotide query sequence to determine percent identity. The deletions occur at the 5′ end of the subject sequence and therefore, the FASTDB alignment does not show a matched/alignment of the first 10 nucleotides at 5′ end. The 10 unpaired nucleotides represent 10% of the sequence (number of nucleotides at the 5′ and 3′ ends not matched/total number of nucleotides in the query sequence) so 10% is subtracted from the percent identity score calculated by the FASTDB program. If the remaining 90 nucleotides were perfectly matched the final percent identity would be 90%. In another example, a 90 nucleotide subject sequence is compared with a 100 nucleotide query sequence. This time the deletions are internal deletions so that there are no nucleotides on the 5′ or 3′ of the subject sequence which are not matched/aligned with the query. In this case the percent identity calculated by FASTDB is not manually corrected. Once again, only nucleotides 5′ and 3′ of the subject sequence which are not matched/aligned with the query sequence are manually corrected for. No other manual corrections are to made for the purposes of the present invention.

Computer Related Embodiments

The nucleotide sequences provided in SEQ ID) NOS: 1-982, a representative fragment thereof, or a nucleotide sequence at least 95%, preferably at least 99% and most preferably at least 99.9% identical to a polynucleotide sequence of SEQ ID NOS:1-982 may be “provided” in a variety of mediums to facilitate use thereof. As used herein, provided refers to a manufacture, other than an isolated nucleic acid molecule, which contains a nucleotide sequence of the present invention; i.e., a nucleotide sequence provided in SEQ ID NOS:1-982, a representative fragment thereof, or a nucleotide sequence at least 95%, preferably at least 99% and most preferably at least 99.9% identical to a polynucleotide of SEQ ID NOS:1-982. Such a manufacture provides a large portion of the Enterococcus faecalis genome and parts thereof (e.g., a Enterococcus faecalis open reading frame (ORF)) in a form which allows a skilled artisan to examine the manufacture using means not directly applicable to examining the Enterococcus faecalis genome or a subset thereof as it exists in nature or in purified form.

In one application of this embodiment, a nucleotide sequence of the present invention can be recorded on computer readable media. As used herein, “computer readable media” refers to any medium which can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories, such as magnetic/optical storage media. A skilled artisan can readily appreciate how any of the presently known computer readable mediums can be used to create a manufacture comprising computer readable medium having recorded thereon a nucleotide sequence of the present invention. Likewise, it will be clear to those of skill how additional computer readable media that may be developed also can be used to create analogous manufactures having recorded thereon a nucleotide sequence of the present invention.

As used herein, “recorded” refers to a process for storing information on computer readable medium. A skilled artisan can readily adopt any of the presently know methods for recording information on computer readable medium to generate manufactures comprising the nucleotide sequence information of the present invention. A variety of data storage structures are available to a skilled artisan for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and MicroSoft Word, or represented in the form of an ASCII file, stored in a database application, such as DB2, Sybase, Oracle, or the like. A skilled artisan can readily adapt any number of data-processor structuring formats (e.g., text file or database) in order to obtain computer readable medium having recorded thereon the nucleotide sequence information of the present invention.

Computer software is publicly available which allows a skilled artisan to access sequence information provided in a computer readable medium. Thus, by providing in computer readable form the nucleotide sequences of SEQ ID NOS:1-982, a representative fragment thereof, or a nucleotide sequence at least 95%, preferably at least 99% and most preferably at least 99.9% identical to a sequence of SEQ ID NOS: 1-982 the present invention enables the skilled artisan routinely to access the provided sequence information for a wide variety of purposes.

The examples which follow demonstrate how software which implements the BLAST (Altschul et al., J. Mol. Biol. 215:403-410 (1990)) and BLAZE (Brutlag et al., Comp. Chem. 17:203-207 (1993)) search algorithms on a Sybase system was used to identify open reading frames (ORFs) within the Enterococcus faecalis genome which contain homology to ORFs or proteins from both Enterococcus faecalis and from other organisms. Among the ORFs discussed herein are protein encoding fragments of the Enterococcus faecalis genome useful in producing commercially important proteins, such as enzymes used in fermentation reactions and in the production of commercially useful metabolites, proteins to be used as vaccines or in the generation of immuno-therapeutic reagents, or as drug screening targets.

The present invention further provides systems, particularly computer-based systems, which contain the sequence information described herein. Such systems are designed to identify, among other things, commercially important fragments of the Enterococcus faecalis genome.

As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based system are suitable for use in the present invention.

As stated above, the computer-based systems of the present invention comprise a data storage means having stored therein a nucleotide sequence of the present invention and the necessary hardware means and software means for supporting and implementing a search means.

As used herein, “data storage means” refers to memory which can store nucleotide sequence information of the present invention, or a memory access means which can access manufactures having recorded thereon the nucleotide sequence information of the present invention.

As used herein, “search means” refers to one or more programs which are implemented on the computer-based system to compare a target sequence or target structural motif with the sequence information stored within the data storage means. Search means are used to identify fragments or regions of the present genomic sequences which match a particular target sequence or target motif. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTX (NCBI). A skilled artisan can readily recognize that any one of the available algorithms or implementing software packages for conducting homology searches can be adapted for use in the present computer-based systems.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that searches for commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There arc a variety of target motifs known in the art. Protein target motifs include, but are not limited to, enzymic active sites and signal sequences. Nucleic acid target motifs include, but are not limited to, promoter sequences, hairpin structures and inducible expression elements (protein binding sequences).

A variety of structural formats for the input and output means can be used to input and output the information in the computer-based systems of the present invention. A preferred format for an output means ranks fragments of the Enterococcus faecalis genomic sequences possessing varying degrees of homology to the target sequence or target motif. Such presentation provides a skilled artisan with a ranking of sequences which contain various amounts of the target sequence or target motif and identifies the degree of homology contained in the identified fragment.

A variety of comparing means can be used to compare a target sequence or target motif with the data storage means to identify sequence fragments of the Enterococcus faecalis genome. In the present examples, implementing software which implement the BLAST algorithm, described in Altschul et al. (1990) J. Mol. Biol. 215: 403-410, is used to identify open reading frames within the Enterococcus faecalis genome. A skilled artisan can readily recognize that any one of the publicly available homology search programs can be used as the search means for the computer-based systems of the present invention. Of course, suitable proprietary systems that may be known to those of skill also may be employed in this regard.

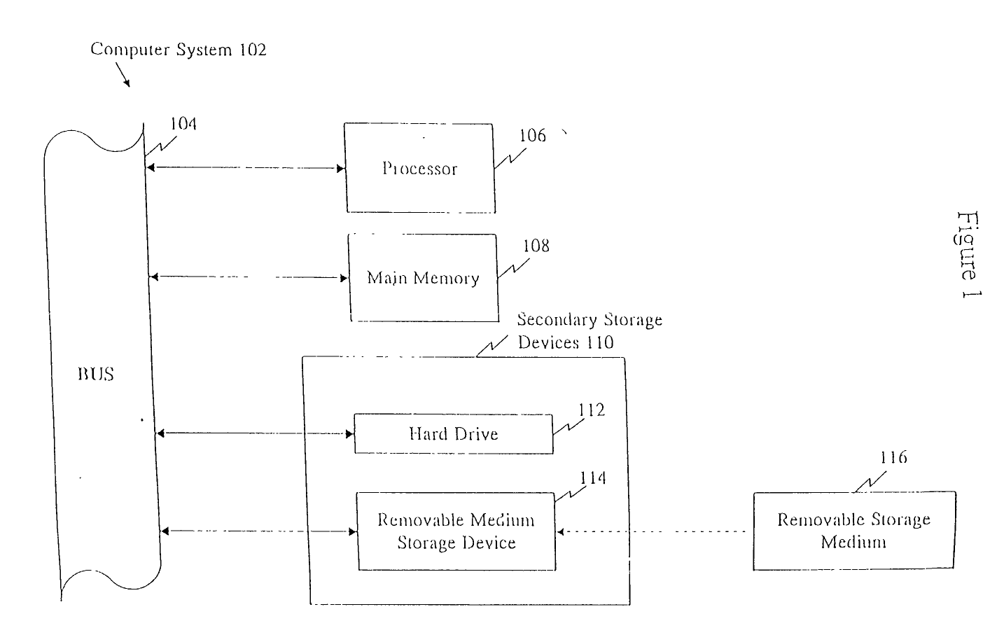

FIG. 1 provides a block diagram of a computer system illustrative of embodiments of this aspect of present invention. The computer system 102 includes a processor 106 connected to a bus 104. Also connected to the bus 104 are a main memory 108 (preferably implemented as random access memory, RAM) and a variety of secondary storage devices 110, such as a hard drive 112 and a removable medium storage device 114. The removable medium storage device 114 may represent, for example, a floppy disk drive, a CD-ROM drive, a magnetic tape drive, etc. A removable storage medium 116 (such as a floppy disk, a compact disk, a magnetic tape, etc.) containing control logic and/or data recorded therein may be inserted into the removable medium storage device 114. The computer system 102 includes appropriate software for reading the control logic and/or the data from the removable medium storage device 114, once it is inserted into the removable medium storage device 114.

A nucleotide sequence of the present invention may be stored in a well known manner in the main memory 108, any of the secondary storage devices 110, and/or a removable storage medium 116. During execution, software for accessing and processing the genomic sequence (such as search tools, comparing tools, etc.) reside in main memory 108, in accordance with the requirements and operating parameters of the operating system, the hardware system and the software program or programs.

Biochemical Embodiments

Other embodiments of the present invention are directed to isolated fragments of the Enterococcus faecalis genome. The fragments of the Enterococcus faecalis genome of the present invention include, but are not limited to fragments which encode peptides, hereinafter open reading frames (ORFs), fragments which modulate the expression of an operably linked ORF, hereinafter expression modulating fragments (EMFs) and fragments which can be used to diagnose the presence of Enterococcus faecalis in a sample, hereinafter diagnostic fragments (DFs).

As used herein, an “isolated nucleic acid molecule” or an “isolated fragment of the Enterococcus faecalis genome” refers to a nucleic acid molecule possessing a specific nucleotide sequence which has been subjected to purification means to reduce, from the composition, the number of compounds which are normally associated with the composition. Particularly, the term refers to the nucleic acid molecules having the sequences set out in SEQ ID NOS:1-982, to representative fragments thereof as described above, to polynucleotides at least 95%, preferably at least 99% and especially preferably at least 99.9% identical in sequence thereto, also as set out above.

A variety of purification means can be used to generate the isolated fragments of the present invention. These include, but are not limited to methods which separate constituents of a solution based on charge, solubility, or size.

In one embodiment, Enterococcus faecalis DNA can be enzymatically sheared to produce fragments of 15-20 kb in length. These fragments can then be used to generate a Enterococcus faecalis library by inserting them into lambda clones as described in the Examples below. Primers flanking, for example, an ORF, such as those enumerated in Tables 1-3 can then be generated using nucleotide sequence information provided in SEQ ID NOS:1-982. Well known and routine techniques of PCR cloning then can be used to isolate the ORF from the lambda DNA library or Enterococcus faecalis genomic DNA. Thus, given the availability of SEQ ID NOS:1-982, the information in Tables 1, 2 and 3, and the information that may be obtained readily by analysis of the sequences of SEQ ID NOS:1-982 using methods set out above, those of skill will be enabled by the present disclosure to isolate any ORF-containing or other nucleic acid fragment of the present invention.

The isolated nucleic acid molecules of the present invention include, but are not limited to single stranded and double stranded DNA, and single stranded RNA. As used herein, an “open reading frame,” ORF, means a series; of triplets coding for amino acids without any termination codons and is a sequence translatable into protein. Each sequence of SEQ ID NOS:1-982, however, begins and ends with a termination codon. For purposes of numbering and reference to polynucleotide and polypeptide sequences the entire sequence of each sequence of SEQ ID NOS:1-982 is included with the first nucleotide being position 1. Therefore, for reference purposes the numbering used in the present invention is that provided in the sequence listing for SEQ ID NOS:1-982.

Tables 1, 2, and 3 list ORFs in the Enterococcus faecalis genomic contigs of the present invention that were identified as putative coding regions by the GeneMark software using organism-specific second-order Markov probability transition matrices. It will be appreciated that other criteria can be used, in accordance with well known analytical methods, such as those discussed herein, to generate more inclusive, more restrictive, or more selective lists.

Table 1 sets out ORFs in the Enterococcus faecalis contigs of the present invention that over a continuous region of at least 50 bases are 95% or more identical (by BLAST analysis) to a nucleotide sequence available through GenBank in March, 1997.

Table 2 sets out ORFs in the Enterococcus faecalis contigs of the present invention that are not in Table 1 and match, with a BLASTP probability score of 0.01 or less, a polypeptide sequence available through GenBank in March, 1997.

Table 3 sets out ORFs in the Enterococcus faecalis contigs of the present invention that do not match significantly, by BLASTP analysis, a polypeptide sequence available through GenBank in March, 1997.

In each table, the first and second columns identify the ORF by, respectively, contig number and ORF number within the contig; the third column indicates the coordinate of the first nucleotide of the ORF, counting from the 5′ end of the contig strand; the fourth column indicates the coordinate of the final nucleotide of the ORF, counting from the 5′ end of the contig strand.

In Tables 1 and 2, column five lists the Reference for the closest matching sequence available through GenBank. These reference numbers are the database entry numbers commonly used by those of skill in the art, who will be familiar with their denominators. Descriptions of the nomenclature are available from the National Center for Biotechnology Information. Column six in Tables 1 and 2 provides the gene name of the matching sequence.

In Table 1, column seven provides the nucleotide BLAST percent identity score from the comparison of the ORF and the GenBank sequence, column eight indicates the length in nucleotides of the highest scoring segment pair identified by the BLAST identity analysis, and column nine provides the total length of the ORF in nucleotides.

In Table 2, column seven provides the protein BLAST percent similarity of the highest scoring segment pair identified, column eight provides the percent identity of the highest scoring segment pair, and column nine provides the total length of the ORF in nucleotides.

The concepts of percent identity and percent similarity of two polypeptide sequences is well understood in the art. For example, two polypeptides 10 amino acids in length which differ at three amino acid positions (e.g., at positions 1, 3 and 5) are said to have a percent identity of 70%. However, the same two polypeptides would be deemed to have a percent similarity of 80% if, for example at position 5, the amino acids moieties, although not identical, were “similar” (i.e., possessed similar biochemical characteristics). Many programs for analysis of nucleotide or amino acid sequence similarity, such as fasta and BLAST specifically list percent identity of a matching region as an output parameter. Thus, for instance, Tables 1 and 2 herein enumerate the percent identity of the highest scoring segment pair in each ORF and its listed relative. Further details concerning the algorithms and criteria used for homology searches are provided below and are described in the pertinent literature highlighted by the citations provided below.

It will be appreciated that other criteria can be used to generate more inclusive and more exclusive listings of the types set out in the tables. As those of skill will appreciate, narrow and broad searches both are useful. Thus, a skilled artisan can readily identify ORFs in contigs of the Enterococcus faecalis genome other than those listed in Tables 1-3, such as ORFs which are overlapping or encoded by the opposite strand of an identified ORF in addition to those ascertainable using the computer-based systems of the present invention.

As used herein, an “expression modulating fragment,” EMF, means a series of nucleotide molecules which modulates the expression of an operably linked ORF or EMF.

As used herein, a sequence is said to “modulate the expression of an operably linked sequence” when the expression of the sequence is altered by the presence of the EMF. EMFs include, but are not limited to, promoters, and promoter modulating sequences (inducible elements). One class of EMFs are fragments which induce the expression or an operably linked ORF in response to a specific regulatory factor or physiological event.

EMF sequences can be identified within the contigs of the Enterococcus faecalis genome by their proximity to the ORFs provided in Tables 1-3. An intergenic segment, or a fragment of the intergenic segment, from about 10 to 200 nucleotides in length, taken from any one of the ORFs of Tables 1-3 will modulate the expression of an operably linked ORF in a fashion similar to that found with the naturally linked ORF sequence. As used herein, an “intergenic segment” refers to fragments of the Enterococcus faecalis genome which are between two ORF(s) herein described. EMFs also can be identified using known EMFs as a target sequence or target motif in the computer-based systems of the present invention. Further, the two methods can be combined and used together.

The presence and activity of an EMF can be confirmed using an EMF trap vector. An EMF trap vector contains a cloning site linked to a marker sequence. A marker sequence encodes an identifiable phenotype, such as antibiotic resistance or a complementing nutrition auxotrophic factor, which can be identified or assayed when the EMF trap vector is placed within an appropriate host under appropriate conditions. As described above, a EMF will modulate the expression of an operably linked marker sequence. A more detailed discussion of various marker sequences is provided below.

A sequence which is suspected as being an EMF is cloned in all three reading frames in one or more restriction sites upstream from the marker sequence in the EMF trap vector. The vector is then transformed into an appropriate host using known procedures and the phenotype of the transformed host in examined under appropriate conditions. As described above, an EMF will modulate the expression of an operably linked marker sequence.

As used herein, a “diagnostic fragment,” DF, means a series of nucleotide molecules which selectively hybridize to Enterococcus faecalis sequences. DFs can be readily identified by identifying unique sequences within contigs of the Enterococcus faecalis genome, such as by using well-known computer analysis software, and by generating and testing probes or amplification primers consisting of the DF sequence in an appropriate diagnostic format which determines amplification or hybridization selectivity.

The sequences falling within the scope of the present invention are not limited to the specific sequences herein described, but also include allelic and species variations thereof. Allelic and species variations can be routinely determined by comparing the sequences provided in SEQ ID NOS:1-982, a representative fragment thereof, or a nucleotide sequence at least 99% and preferably 99.9% identical to SEQ ID NOS:1-982, with a sequence from another isolate of the same species. Furthermore, to accommodate codon variability, the invention includes nucleic acid molecules coding for the same amino acid sequences as do the specific ORFs disclosed herein. In other words, in the coding region of an ORF, substitution of one codon for another which encodes the same amino acid is expressly contemplated.

Any specific sequence disclosed herein can be readily screened for errors by resequencing a particular fragment, such as an ORF, in both directions (i.e., sequence both strands). Alternatively, error screening can be performed by sequencing corresponding polynucleotides of Enterococcus faecalis origin isolated by using part or all of the fragments in question as a probe or primer.

Each of the ORFs of the Enterococcus faecalis genome disclosed in Tables 1, 2 and 3, and the EMFs found 5 to the ORFs, can be used as polynucleotide reagents in numerous ways. For example, the sequences can be used as diagnostic probes or diagnostic amplification primers to detect the presence of a specific microbe in a sample, particularly Enterococcus faecalis. Especially preferred in this regard are ORFs such as those of Table 3, which do not match previously characterized sequences from other organisms and thus are most likely to be highly selective for Enterococcus faecalis. Also particularly preferred are ORFs that can be used to distinguish between strains of Enterococcus faecalis, particularly those that distinguish medically important strain, such as drug-resistant strains.

In addition, the fragments of the present invention, as broadly described, can be used to control gene expression through triple helix formation or antisense DNA or RNA, both of which methods are based on the binding of a polynucleotide sequence to DNA or RNA. Triple helix-formation optimally results in a shut-off of RNA transcription from DNA, while antisense RNA hybridization blocks translation of an mRNA molecule into polypeptide. Information from the sequences of the present invention can be used to design antisense and triple helix-forming oligonucleotides. Polynucleotides suitable for use in these methods are usually 20 to 40 bases in length and are designed to be complementary to a region of the gene involved in transcription, for triple-helix formation, or to the mRNA itself, for antisense inhibition. Both techniques have been demonstrated to be effective in model systems, and the requisite techniques are well known and involve routine procedures. Triple helix techniques are discussed in, for example, Lee et al., Nucl. Acids Res. 6:3073 (1979); Cooney et al., Science 241:456 (1988); and Dervan et al., Science 251:1360 (1991). Antisense techniques in general are discussed in, for instance, Okano, J. Neurochem. 56:560 (1991) and Oligodeoxynucleotides as Antisense Inhibitors of Gene Expression, CRC Press, Boca Raton, Fla. (1988)).

The present invention further provides recombinant constructs comprising one or more fragments of the Enterococcus faecalis genomic fragments and contigs of the present invention. Certain preferred recombinant constructs of the present invention comprise a vector, such as a plasmid or viral vector, into which a fragment of the Enterococcus faecalis genome has been inserted, in a forward or reverse orientation. In the case of a vector comprising one of the ORFs of the present invention, the vector may further comprise regulatory sequences, including for example, a promoter, operably linked to the ORF. For vectors comprising the EMFs of the present invention, the vector may further comprise a marker sequence or heterologous ORF operably linked to the EMF.

Large numbers of suitable vectors and promoters are known to those of skill in the art and are commercially available for generating the recombinant constructs of the present invention. The following vectors are provided by way of example. Useful bacterial vectors include phagescript, PsiX174, pBS SK (+ or −), pBS KS (+ or −), pNH8a, pNH16a, pNH18a, pNH46a (available from Stratagene); pTrc99A, pKK223-3, pKK233-3, pDR540, pRIT5 (available from Pharmacia). Useful eukaryotic vectors include pWLneo, pSV2cat, pOG44, pXT1, pSG (available from Stratagene) pSVK3, pBPV, pMSG, pSVL (available from Pharmacia).

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda PR, and trc. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

The present invention further provides host cells containing any one of the isolated fragments of the Enterococcus faecalis genomic fragments and contigs of the present invention, wherein the fragment has been introduced into the host cell using known methods. The host cell can be a higher eukaryotic host cell, such as a mammalian cell, a lower eukaryotic host cell, such as a yeast cell, or a procaryotic cell, such as a bacterial cell.

A polynucleotide of the present invention, such as a recombinant construct comprising an ORF of the present invention, may be introduced into the host by a variety of well established techniques that are standard in the art, such as calcium phosphate transfection, DEAE, dextran mediated transfection and electroporation, which are described in, for instance, Davis, L. et al., BASIC METHODS IN MOLECULAR BIOLOGY (1986).

A host cell containing one of the fragments of the Enterococcus faecalis genomic fragments and contigs of the present invention, can be used in conventional manners to produce the gene product encoded by the isolated fragment (in the case of an ORF) or can be used to produce a heterologous protein under the control of the EMF. The present invention further provides isolated polypeptides encoded by the nucleic acid fragments of the present invention or by degenerate variants of the nucleic acid fragments of the present invention. By “degenerate variant” is intended nucleotide fragments which differ from a nucleic acid fragment of the present invention (e.g., an ORF) by nucleotide sequence but, due to the degeneracy of the Genetic Code, encode an identical polypeptide sequence.

Preferred nucleic acid fragments of the present invention are the ORFs depicted in Tables 2 and 3 which encode proteins.

A variety of methodologies known in the art can be utilized to obtain any one of the isolated polypeptides or proteins of the present invention. At the simplest level, the amino acid sequence can be synthesized using commercially available peptide synthesizers. This is particularly useful in producing small peptides and fragments of larger polypeptides. Such short fragments as may be obtained most readily by synthesis are useful, for example, in generating antibodies against the native polypeptide, as discussed further below.

In an alternative method, the polypeptide or protein is purified from bacterial cells which naturally produce the polypeptide or protein. One skilled in the art can readily employ well-known methods for isolating polypeptides and proteins to isolate and purify polypeptides or proteins of the present invention produced naturally by a bacterial strain, or by other methods. Methods for isolation and purification that can be employed in this regard include, but are not limited to, immunochromatography, HPLC, size-exclusion chromatography, ion-exchange chromatography, and immuno-affinity chromatography.

The polypeptides and proteins of the present invention also can be purified from cells which have been altered to express the desired polypeptide or protein. Preferred polypeptides and proteins of the present invention are polypeptides and proteins coded for by the polynucleotides of SEQ ID NOS:1-982, wherein the polypeptides and proteins are coded in the same frame as the termination codon at the end of each sequence of SEQ ID NOS:1-982. As used herein, a cell is said to be altered to express a desired polypeptide or protein when the cell, through genetic manipulation, is made to produce a polypeptide or protein which it normally does not produce or which the cell normally produces at a lower level. Those skilled in the art can readily adapt procedures for introducing and expressing either recombinant or synthetic sequences into eukaryotic or prokaryotic cells in order to generate a cell which produces one of the polypeptides or proteins of the present invention.

The polypeptides of the present invention arc preferably provided in an isolated form, and preferably are substantially purified. A recombinantly produced version of the E. faecalis polypeptide can be substantially purified by the one-step method described by Smith et al. (1988) Gene 67:31-40. Polypeptides of the invention also can be purified from natural or recombinant sources using antibodies directed against the polypeptides of the invention in methods which are well known in the art of protein purification.

The invention further provides for isolated E. faecalis polypeptides comprising an amino acid sequence selected from the group including: (a) the amino acid sequence of a full-length E. faecalis polypeptide having the complete amino acid sequence from the first methionine codon to the termination codon of each sequence listed in SEQ ID NOS:1-982, wherein said termination codon is at the end of each SEQ ID NO: and said first methionine is the first methionine in frame with said termination codon; and (b) the amino acid sequence of a full-length E. faecalis polypeptide having the complete amino acid sequence in (a) excepting the N-terminal methionine.

The polypeptides of the present invention also include polypeptides having an amino acid sequence at least 80% identical, more preferably at least 90% identical, and still more preferably 95%, 96%, 97%, 98% or 99% identical to those described in (a) and (b) above.

The present invention is further directed to polynucleotide encoding portions or fragments of the amino acid sequences described herein as well as to portions or fragments of the isolated amino acid sequences described herein. Fragments include portions of the amino acid sequences described herein, are at least 5 contiguous amino acid in length, are selected from any two integers, one of which representing a N-terminal position. The initiation codon of the polypeptides of the present inventions position 1. The initiation codon (position 1) for purposes of the present invention is the first methionine codon of each sequence of SEQ ID NOS:1-982 which is in frame with the termination codon at the end of each said sequence. Every combination of a N-terminal and C-terminal position that a fragment at least 5 contiguous amino acid residues in length could occupy, on any given amino acid sequence encoded by a sequence of SEQ ID NOS:1-982 is included in the invention, i.e., from initiation codon up to the termination codon. At least means a fragment may be 5 contiguous amino acid residues in length or any integer between 5 and the number of residues in a full length amino acid sequence minus 1. Therefore, included in the invention are contiguous fragments specified by any N-terminal and C-terminal positions of amino acid sequence set forth in SEQ ID NOS:1-982 wherein the contiguous fragment is any integer between 5 and the number of residues in a full length sequence minus 1.

Further, the invention includes polypeptides comprising fragments specified by size, in amino acid residues, rather than by N-terminal and C-terminal positions. The invention includes any fragment size, in contiguous amino acid residues, selected from integers between 5 and the number of residues in a full length sequence minus 1. Preferred sizes of contiguous polypeptide fragments include about 5 amino acid residues, about 10 amino acid residues, about 20 amino acid residues, about 30 amino acid residues, about 40 amino acid residues, about 50 amino acid residues, about 100 amino acid residues, about 200 amino acid residues, about 300 amino acid residues, and about 400 amino acid residues. The preferred sizes are, of course, meant to exemplify, not limit, the present invention as all size fragments representing any integer between 5 and the number of residues in a full length sequence minus I are included in the invention. The present invention also provides for the exclusion of any fragments specified by N-terminal and C-terminal positions or by size in amino acid residues as described above. Any number of fragments specified by N-terminal and C-terminal positions or by size in amino acid residues as described above may be excluded.

The above fragments need not be active since they would be useful, for example, in immunoassays, in epitope mapping, epitope tagging, to generate antibodies to a particular portion of the protein, as vaccines, and as molecular weight markers.

Further polypeptides of the present invention include polypeptides which have at least 90% similarity, more preferably at least 95% similarity, and still more preferably at least 96%, 97%, 98% or 99% similarity to those described above.

A further embodiment of the invention relates to a polypeptide which comprises the amino acid sequence of a E. faecalis polypeptide having an amino acid sequence which contains at least one conservative amino acid substitution, but not more than 50 conservative amino acid substitutions, not more than 40 conservative amino acid substitutions, not more than 30 conservative amino acid substitutions, and not more than 20 conservative amino acid substitutions. Also provided are polypeptides which comprise the amino acid sequence of a E. faecalis polypeptide, having at least one, but not more than 10, 9, 8, 7, 6, 5, 4, 3, 2 or 1 conservative amino acid substitutions.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to the amino acid sequences encoded by the sequences of SEQ ID NOS:1-982, as described herein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., (1990) Comp. App. Biosci. 6:237-245. In a sequence alignment the query and subject sequences are both amino acid sequences. The result of said global sequence alignment is in percent identity. Preferred parameters used in a FASTDB amino acid alignment are: Matrix=PAM 0, k-tuple=2, Mismatch Penalty=1, Joining Penalty-20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, the results, in percent identity, must be manually corrected. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-terminal of the subject sequence, which are not matched/aligned with the query sequence, are considered for the purposes of manually adjusting the percent identity score. That is, only query amino acid residues outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not match/align with the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected. No other manual corrections are to made for the purposes of the present invention.

The above polypeptide sequences are included irrespective of whether they have their normal biological activity. This is because even where a particular polypeptide molecule does not have biological activity, one of skill in the art would still know how to use the polypeptide, for instance, as a vaccine or to generate antibodies. Other uses of the polypeptides of the present invention that do not have E. faecalis activity include, inter alia, as epitope tags, in epitope mapping, and as molecular weight markers on SDS-PAGE gels or on molecular sieve gel filtration columns using methods known to those of skill in the art.

As described below, the polypeptides of the present invention can also be used to raise polyclonal and monoclonal antibodies, which are useful in assays for detecting E. faecalis protein expression or as agonists and antagonists capable of enhancing or inhibiting E. faecalis protein function. Further, such polypeptides can be used in the yeast two-hybrid system to “capture” E. faecalis protein binding proteins which are also candidate agonists and antagonists according to the present invention. See, e.g., Fields et al. (1989) Nature 340:245-246.

Any host/vector system can be used to express one or more of the ORFs of the present invention. These include, but are not limited to, eukaryotic hosts such as HeLa cells, CV-1 cell, COS cells, and Sf9 cells, as well as prokaryotic host such as E. coli and B. subtilis. The most preferred cells are those which do not normally express the particular polypeptide or protein or which expresses the polypeptide or protein at low natural level.

“Recombinant,” as used herein, means that a polypeptide or protein is derived from recombinant (e.g., microbial or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern different from that expressed in mammalian cells.

“Nucleotide sequence” refers to a heteropolymer of deoxyribonucleotides. Generally, DNA segments encoding the polypeptides and proteins provided by this invention are assembled from fragments of the Enterococcus faecalis genome and short oligonucleotide linkers, or from a series of oligonucleotides, to provide a synthetic gene which is capable of being expressed in a recombinant transcriptional unit comprising regulatory elements derived from a microbial or viral operon.

Recombinant expression vehicle or “vector” refers to a plasmid or phage or virus or vector, for expressing a polypeptide from a DNA (RNA) sequence. The expression vehicle can comprise a transcriptional unit comprising an assembly of (1) a genetic regulatory elements necessary for gene expression in the host, including elements required to initiate and maintain transcription at a level sufficient for suitable expression of the desired polypeptide, including, for example, promoters and, where necessary, an enhancer and a polyadenylation signal; (2) a structural or coding sequence which is transcribed into mRNA and translated into protein, and (3) appropriate signals to initiate translation at the beginning of the desired coding region and terminate translation at its end. Structural units intended for use in yeast or eukaryotic expression systems preferably include a leader sequence enabling extracellular secretion of translated protein by a host cell. Alternatively, where recombinant protein is expressed without a leader or transport sequence, it may include an N-terminal methionine residue. This residue may or may not be subsequently cleaved from the expressed recombinant protein to provide a final product.

“Recombinant expression system” means host cells which have stably integrated a recombinant transcriptional unit into chromosomal DNA or carry the recombinant transcriptional unit extra chromosomally. The cells can be prokaryotic or eukaryotic. Recombinant expression systems as defined herein will express heterologous polypeptides or proteins upon induction of the regulatory elements linked to the DNA segment or synthetic gene to be expressed.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described in Sambrook et al., Molecular Cloning: A Laboratory Manual, 2nd Edition, Cold Spring Harbor Laboratory Press, Cold Spring Harbor, N.Y. (1989), the disclosure of which is hereby incorporated by reference in its entirety.

Generally, recombinant expression vectors will include origins of replication and selectable markers permitting transformation of the host cell, e.g., the ampicillin resistance gene of E. coli and S. cerevisiae TRP1 gene, and a promoter derived from a highly expressed gene to direct transcription of a downstream structural sequence. Such promoters can be derived from operons encoding glycolytic enzymes such as 3-phosphoglycerate kinase (PGK), alpha-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and, when desirable, provide amplification within the host.

Suitable prokaryotic hosts for transformation include strains of E. coli, B. subtilis, Salmonella typhimurium and various species within the genera Pseudomonas and Streptomyces. Others may, also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (available form Pharmacia Fine Chemicals, Uppsala, Sweden) and GEM 1 (available from Promega Biotec, Madison, Wis., USA). These pBR322 “backbone” sections are combined with an appropriate promoter and the structural sequence to be expressed.

Following transformation of a suitable host strain and growth of the host strain to an appropriate cell density, the selected promoter, where it is inducible, is derepressed or induced by appropriate means (e.g., temperature shift or chemical induction) and cells are cultured for an additional period to provide for expression of the induced gene product. Thereafter cells are typically harvested, generally by centrifugation, disrupted to release expressed protein, generally by physical or chemical means, and the resulting crude extract is retained for further purification.

Various mammalian cell culture systems can also be employed to express recombinant protein. Examples of mammalian expression systems include the COS-7 lines of monkey kidney fibroblasts, described in Gluzman, Cell 23:175 (1981), and other cell lines capable of expressing a compatible vector, for example, the C127, 3T3, CHO, HeLa and BHK cell lines.

Mammalian expression vectors will comprise an origin of replication, a suitable promoter and enhancer, and also any necessary ribosome binding sites, polyadenylation site, splice donor and acceptor sites, transcriptional termination sequences, and 5 flanking nontranscribed sequences. DNA sequences derived from the SV40 viral genome, for example, SV40 origin, early promoter, enhancer, splice, and polyadenylation sites may be used to provide the required nontranscribed genetic elements.

Recombinant polypeptides and proteins produced in bacterial culture is usually isolated by initial extraction from cell pellets, followed by one or more salting-out, aqueous ion exchange or size exclusion chromatography steps. Microbial cells employed in expression of proteins can be disrupted by any convenient method, including freeze-thaw cycling, sonication, mechanical disruption, or use of cell lysing agents. Protein refolding steps can be used, as necessary, in completing configuration of the mature protein. Finally, high performance liquid chromatography (HPLC) can be employed for final purification steps.

The present invention further includes isolated polypeptides, proteins and nucleic acid molecules which are substantially equivalent to those herein described. As used herein, substantially equivalent can refer both to nucleic acid and amino acid sequences, for example a mutant sequence, that varies from a reference sequence by one or more substitutions, deletions, or additions, the net effect of which does not result in an adverse functional dissimilarity between reference and subject sequences. For purposes of the present invention, sequences having equivalent biological activity, and equivalent expression characteristics are considered substantially equivalent. For purposes of determining equivalence, truncation of the mature sequence should be disregarded.

The invention further provides methods of obtaining homologs from other strains of Enterococcus faecalis, of the fragments of the Enterococcus faecalis genome of the present invention and homologs of the proteins encoded by the ORFs of the present invention. As used herein, a sequence or protein of Enterococcus faecalis is defined as a homolog of a fragment of the Enterococcus faecalis fragments or contigs or a protein encoded by one of the ORFs of the present invention,, if it shares significant homology to one of the fragments of the Enterococcus faecalis genome of the present invention or a protein encoded by one of the ORFs of the present invention. Specifically, by using the sequence disclosed herein as a probe or as primers, and techniques such as PCR cloning and colony/plaque hybridization, one skilled in the art can obtain homologs.

As used herein, two nucleic acid molecules or proteins are said to “share significant homology” if the two contain regions which possess greater than 85% sequence (amino acid or nucleic acid) homology. Preferred homologs in this regard are those with more than 90% homology. Especially preferred are those with 93% or more homology. Among especially preferred homologs those with 95% or more homology are particularly preferred. Very particularly preferred among these are those with 97% and even more particularly preferred among those are homologs with 99% or more homology. The most preferred homologs among these are those with 99.9% homology or more. It will be understood that, among measures of homology, identity is particularly preferred in this regard.

Region specific primers or probes derived from the nucleotide sequence provided in SEQ ID NOS:1-982 or from a nucleotide sequence at least 95%, particularly at least 99%, especially at least 99.5% identical to a sequence of SEQ ID NOS:1-982 can be used to prime DNA synthesis and PCR amplification, as well as to identify colonies containing cloned DNA encoding a homolog. Methods suitable to this aspect of the present invention are well known and have been described in great detail in many publications such as, for example, Innis et al., PCR Protocols, Academic Press, San Diego, Calif. (1990)).

When using primers derived from SEQ ID NOS:1-982 or from a nucleotide sequence having an aforementioned identity to a sequence of SEQ ID NOS: 1-982, one skilled in the art will recognize that by employing high stringency conditions (e.g., annealing at 50-60° C. in 6×SSPC and 50% formamicle, and washing at 50-65° C. in 0.5×SSPC) only sequences which are greater than 75% homologous to the primer will be amplified. By employing lower stringency conditions (e.g., hybridizing at 35-37° C. in 5×SSPC and 40-45% formamide, and washing at 42° C. in 0.5×SSPC), sequences which are greater than 40-50% homologous to the primer will also be amplified.

When using DNA probes derived from SEQ ID NOS:1-982, or from a nucleotide sequence having an aforementioned identity to a sequence of SEQ ID NOS:1-982, for colony/plaque hybridization, one skilled in the art will recognize that by employing high stringency conditions (e.g., hybridizing at 50-65° C. in 5×SSPC and 50% formamide, and washing at 50-65° C. in 0.5×SSPC), sequences having regions which are greater than 90% homologous to the probe can be obtained, and that by employing lower stringency conditions (e.g., hybridizing at 35-37° C. in 5×SSPC and 40-45% formamide, and washing at 42° C. in 0.5×SSPC), sequences having regions which are greater than 35-45% homologous to the probe will be obtained.

Any organism can be used as the source for homologs of the present invention so long as the organism naturally expresses such a protein or contains genes encoding the same. The most preferred organism for isolating homologs are bacteria which are closely related to Enterococcus faecalis.

Illustrative Uses of Compositions of the Invention

Each ORF provided in Tables 1 and 2 is identified with a function by homology to a known gene or polypeptide. As a result, one skilled in the art can use the polypeptides of the present invention for commercial, therapeutic and industrial purposes consistent with the type of putative identification of the polypeptide. Such identifications permit one skilled in the art to use the Enterococcus faecalis ORFs in a manner similar to the known type of sequences for which the identification is made; for example, to ferment a particular sugar source or to produce a particular metabolite. A variety of reviews illustrative of this aspect of the invention are available, including the following reviews on the industrial use of enzymes, for example, BIOCHEMICAL ENGINEERING AND BIOTECHNOLOGY HANDBOOK, 2nd Ed., MacMillan Publications, Ltd. NY (1991) and BIOCATALYSTS IN ORGANIC SYNTHESES, Tramper et al., Eds., Elsevier Science Publishers, Amsterdam, The Netherlands (1985). A variety of exemplary uses that illustrate this and similar aspects of the present invention are discussed below.

1. Biosynthetic Enzymes

Open reading frames encoding proteins involved in mediating the catalytic reactions involved in intermediary and macromolecular metabolism, the biosynthesis of small molecules, cellular processes and other functions includes enzymes involved in the degradation of the intermediary products of metabolism, enzymes involved in central intermediary metabolism, enzymes involved in respiration, both aerobic and anaerobic, enzymes involved in fermentation, enzymes involved in ATP proton motor force conversion, enzymes involved in broad regulatory function, enzymes involved in amino acid synthesis, enzymes involved in nucleotide synthesis, enzymes involved in cofactor and vitamin synthesis, can be used for industrial biosynthesis.

The various metabolic pathways present in Enterococcus faecalis can be identified based on absolute nutritional requirements as well as by examining the various enzymes identified in Table 1-3 and SEQ ID NOS:1-982.

Of particular interest are polypeptides involved in the degradation of intermediary metabolites as well as non-macromolecular metabolism. Such enzymes include amylases, glucose oxidases, and catalase.

Proteolytic enzymes are another class of commercially important enzymes. Proteolytic enzymes find use in a number of industrial processes including the processing of flax and other vegetable fibers, in the extraction, clarification and depectinization of fruit juices, in the extraction of vegetables' oil and in the maceration of fruits and vegetables to give unicellular fruits. A detailed review of the proteolytic enzymes used in the food industry is provided in Rombouts et al., Symbiosis 21:79 (1986) and Voragen et al. in Biocatalysts In Agricultural Biotechnology, Whitaker et al., Eds., American Chemical Society Symposium Series 389:93 (1989).

The metabolism of sugars is an important aspect of the primary metabolism of Enterococcus faecalis. Enzymes involved in the degradation of sugars, such as, particularly, glucose, galactose, fructose and xylose, can be used in industrial fermentation. Some of the important sugar transforming enzymes, from a commercial viewpoint, include sugar isomerases such as glucose isomerase. Other metabolic enzymes have found commercial use such as glucose oxidases which produces ketogulonic acid (KGA). KGA is an intermediate in the commercial production of ascorbic acid using the Reichstein's procedure, as described in Krueger et al., Biotechnology 6(A), Rhine et al., Eds., Verlag Press, Weinheim, Germany (1984).

Glucose oxidase (GOD) is commercially available and has been used in purified form as well as in an immobilized form for the deoxygenation of beer. See, for instance, Hartmeir et al., Biotechnology Letters 1:21 (1979). The most important application of GOD is the industrial scale fermentation of gluconic acid. Market for gluconic acids which are used in the detergent, textile, leather, photographic, pharmaceutical, food, feed and concrete industry, as described, for example, in Bigelis et al., beginning on page 357 in GENE MANIPULATIONS AND FUNGI; Benett et al., Eds., Academic Press, New York (1985). In addition to industrial applications, GOD has found applications in medicine for quantitative determination of glucose in body fluids recently in biotechnology for analyzing syrups from starch and cellulose hydrosylates. This application is described in Owusu et al., Biochem. et Biophysica. Acta. 872:83 (1986), for instance.

The main sweetener used in the world today is sugar which comes from sugar beets and sugar cane. In the field of industrial enzymes, the glucose isomerase process shows the largest expansion in the market today. Initially, soluble enzymes were used and later immobilized enzymes were developed (Krueger et al., Biotechnology, The Textbook of Industrial Microbiology, Sinauer Associated Incorporated, Sunderland, Mass. (1990)). Today, the use of glucose-produced high fructose syrups is by far the largest industrial business using immobilized enzymes. A review of the industrial use of these enzymes is provided by Jorgensen, Starch 40:307 (1988).

Proteinases, such as alkaline serine proteinases, are used as detergent additives and thus represent one of the largest volumes of microbial enzymes used in the industrial sector. Because of their industrial importance, there is a large body of published and unpublished information regarding the use of these enzymes in industrial processes. (See Faultman et al., Acid Proteases Structure Function and Biology, Tang, J., ed., Plenum Press, New York (1977) and Godfrey et al., Industrial Enzymes, MacMillan Publishers, Surrey, UK (1983) and Hepner et al., Report Industrial Enzymes by 1990, Hel Hepner & Associates, London (1986)).

Another class of commercially usable proteins of the present invention are the microbial lipases, described by, for instance, Macrae et al., Philosophical Transactions of the Chiral Society of London 310:227 (1985) and Poserke, Journal of the American Oil Chemist Society 61:1758 (1984). A major use of lipases is in the fat and oil industry for the production of neutral glycerides using lipase catalyzed inter-esterification of readily available triglycerides. Application of lipases include the use as a detergent additive to facilitate the removal of fats from fabrics in the course of the washing procedures.

The use of enzymes, and in particular microbial enzymes, as catalyst for key steps in the synthesis of complex organic molecules is gaining popularity at a great rate. One area of great interest is the preparation of chiral intermediates. Preparation of chiral intermediates is of interest to a wide range of synthetic chemists particularly those scientists involved with the preparation of new pharmaceuticals, agrochemicals, fragrances and flavors. (See Davies et al., Recent Advances in the Generation of Chiral Intermediates Using Enzymes, CRC Press, Boca Raton, Fla. (1990)). The following reactions catalyzed by enzymes are of interest to organic chemists: hydrolysis of carboxylic acid esters, phosphate esters, amides and nitrites, esterification reactions, trans-esterification reactions, synthesis of amides, reduction of alkanones and oxoalkanates, oxidation of alcohols to carbonyl compounds, oxidation of sulfides to sulfoxides, and carbon bond forming reactions such as the aldol reaction.

When considering the use of an enzyme encoded by one of the ORFs of the present invention for biotransformation and organic synthesis it is sometimes necessary to consider the respective advantages and disadvantages of using a microorganism as opposed to an isolated enzyme. Pros and cons of using a whole cell system on the one hand or an isolated partially purified enzyme on the other hand, has been described in detail by Bud et al., Chemistry in Britain (1987), p. 127.

Amino transferases, enzymes involved in the biosynthesis and metabolism of amino acids, are useful in the catalytic production of amino acids. The advantages of using microbial based enzyme systems is that the amino transferase enzymes catalyze the stereo-selective synthesis of only L-amino acids and generally possess uniformly high catalytic rates. A description of the use of amino transferases for amino acid production is provided by Roselle-David, Methods of Enzymology 136:479 (1987).

Another category of useful proteins encoded by the ORFs of the present invention include enzymes involved in nucleic acid synthesis, repair, and recombination.

2. Generation of Antibodies

As described here, the proteins of the present invention, as well as homologs thereof, can be used in a variety of procedures and methods known in the art which are currently applied to other proteins. The proteins of the present invention can further be used to generate an antibody which selectively binds the protein.

E. faecalis protein-specific antibodies for use in the present invention can be raised against the intact E. faecalis protein or an antigenic polypeptide fragment thereof, which may be presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Mab) is meant to include intact molecules, single chain whole antibodies, and antibody fragments. Antibody fragments of the present invention include Fab and F(ab′)2 and other fragments including single-chain Fvs (scFv) and disulfide-linked Fvs (sdFv). Also included in the present invention are chimeric and humanized monoclonal antibodies and polyclonal antibodies specific for the polypeptides of the present invention. The antibodies of the present invention may be prepared by any of a variety of methods. For example, cells expressing a polypeptide of the present invention or an antigenic fragment thereof can be administered to an animal in order to induce the production of sera containing polyclonal antibodies. For example, a preparation of E. faecalis polypeptide or fragment thereof is prepared and purified to render it substantially free of natural contaminants. Such a preparation is then introduced into an animal in order to produce polyclonal antisera of greater specific activity.

In a preferred method, the antibodies of the present invention are monoclonal antibodies or binding fragments thereof. Such monoclonal antibodies can be prepared using hybridoma technology. See, e.g., Harlow et al., ANTIBODIES: A LABORATORY MANUAL, (Cold Spring Harbor Laboratory Press, 2nd ed. 1988); Hammerling, et al., in: MONOCLONAL ANTIBODIES AND T-CELL HYBRIDOMAS 563-681 (Elsevier, N.Y., 1981). Fab and F(ab′)2 fragments may be produced by proteolytic cleavage, using enzymes such as papain (to produce Fab fragments) or pepsin (to produce F(ab′)2 fragments). Alternatively, E. faecalis polypeptide-binding fragments, chimeric, and humanized antibodies can be produced through the application of recombinant DNA technology or through synthetic chemistry using methods known in the art.

Alternatively, additional antibodies capable of binding to the polypeptide antigen of the present invention may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, E. faecalis polypeptide-specific antibodies arc used to immunize an animal, preferably a mouse. The splenocytes of such an animal are then used to produce hybridoma cells, and the hybridoma cells are screened to identify clones which produce an antibody whose ability to bind to the E. faecalis polypeptide-specific antibody can be blocked by the E. faecalis polypeptide antigen. Such antibodies comprise anti-idiotypic antibodies to the E. faecalis polypeptide-specific antibody and can be used to immunize an animal to induce formation of further E. faecalis polypeptide-specific antibodies.

Antibodies and fragements thereof of the present invention may be described by the portion of a polypeptide of the present invention recognized or specifically bound by the antibody. Antibody binding fragements of a polypeptide of the present invention may be described or specified in the same manner as for polypeptide fragements discussed above., i.e., by N-terminal and C-terminal positions or by size in contiguous amino acid residues. Any number of antibody binding fragments, of a polypeptide of the present invention, specified by N-terminal and C-terminal positions or by size in amino acid residues, as described above, may also be excluded from the present invention. Therefore, the present invention includes antibodies the specifically bind a particularly described fragement of a polypeptide of the present invention and allows for the exclusion of the same.

Antibodies and fragements thereof of the present invention may also be described or specified in terms of their cross-reactivity. Antibodies and fragements that do not bind polypeptides of any other species of Enterococcus other than E. faecalis are included in the present invention. Likewise, antibodies and fragements that bind only species of Enterococcus, i.e. antibodies and fragements that do not bind bacteria from any genus other than Enterococcus, are included in the present invention.

3. Diagnostic and Detection Assays and Kits

The present invention further relates to methods for assaying enterococcal infection in an animal by detecting the expression of genes encoding enterococcal polypeptides of the present invention. The methods comprise analyzing tissue or body fluid from the animal for Enterococcus-specific antibodies, nucleic acids, or proteins. Analysis of nucleic acid specific to Enterococcus is assayed by PCR or hybridization techniques using nucleic acid sequences of the present invention as either hybridization probes or primers. See, e.g., Sambrook et al. Molecular cloning: A Laboratory Manual (Cold Spring Harbor Laboratory Press, 2nd ed., 1983, page 54 reference); Eremeeva et al. (1994) J. Clin. Microbiol. 32:803-810 (describing differentiation among spotted fever group Rickettsiae species by analysis of restriction fragment length polymorphism of PCR-amplified DNA) and Chen et al. 1994 J. Clin. Microbiol. 32:589-595 (detecting B. burgdorferi nucleic acids via PCR).

Where diagnosis of a disease state related to infection with Enterococcus has already been made, the present invention is useful for monitoring progression or regression of the disease state whereby patients exhibiting enhanced Enterococcus gene expression will experience a worse clinical outcome relative to patients expressing these gene(s) at a lower level.

By “biological sample” is intended any biological sample obtained from an animal, cell line, tissue culture, or other source which contains Enterococcus polypeptide, mRNA, or DNA. Biological samples include body fluids (such as saliva, blood, plasma, urine, mucus, synovial fluid, etc.) tissues (such as muscle, skin, and cartilage) and any other biological source suspected of containing Enterococcus polypeptides or nucleic acids. Methods for obtaining biological samples such as tissue are well known in the art.

The present invention is useful for detecting diseases related to Enterococcus infections in animals. Preferred animals include monkeys, apes, cats, dogs, birds, cows, pigs, mice, horses, rabbits and humans. Particularly preferred are humans.

Total RNA can be isolated from a biological sample using any suitable technique such as the single-step guanidinium-thiocyanate-phenol-chloroform method described in Chomczynski et al. (1987) Anal. Biochem. 162:156-159. mRNA encoding Enterococcus polypeptides having sufficient homology to the nucleic acid sequences identified in SEQ ID NOS:1-982 to allow for hybridization between complementary sequences are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

Northern blot analysis can be performed as described in Harada et al. (1990) Cell 63:303-312. Briefly, total RNA is prepared from a biological sample as described above. For the Northern blot, the RNA is denatured in an appropriate buffer (such as glyoxal/dimethyl sulfoxide/sodium phosphate buffer), subjected to agarose gel electrophoresis, and transferred onto a nitrocellulose filter. After the RNAs have been linked to the filter by a UV linker, the filter is prehybridized in a solution containing formamide, SSC, Denhardt's solution, denatured salmon sperm, SDS, and sodium phosphate buffer. A E. faecalis polynucleotide sequence shown in SEQ ID NOS:1-982 labeled according to any appropriate method (such as the ³²P-multiprimed DNA labeling system (Amersham)) is used as probe. After hybridization overnight, the filter is washed and exposed to x-ray film. DNA for use as probe according to the present invention is described in the sections above and will preferably at least 15 nucleotides in length.

S1 mapping can be performed as described in Fujita et al. (1987) Cell 49:357-367. To prepare probe DNA for use in S1 mapping, the sense strand of an above-described E. faecalis DNA sequence of the present invention is used as a template to synthesize labeled antisense DNA. The antisense DNA can then be digested using an appropriate restriction endonuclease to generate further DNA probes of a desired length. Such antisense probes are useful for visualizing protected bands corresponding to the target mRNA (i.e., mRNA encoding Enterococcus polypeptides).

Levels of mRNA encoding Enterococcus polypeptides are assayed, for e.g., using the RT-PCR method described in Makino et al. (1990) Technique 2:295-301. By this method, the radioactivities of the “amplicons” in the polyacrylamide gel bands are linearly related to the initial concentration of the target mRNA. Briefly, this method involves adding total RNA isolated from a biological sample in a reaction mixture containing a RT primer and appropriate buffer. After incubating for primer annealing, the mixture can be supplemented with a RT buffer, cNTPs, DTT, RNase inhibitor and reverse transcriptase. After incubation to achieve reverse transcription of the RNA, the RT products are then subject to PCR using labeled primers. Alternatively, rather than labeling the primers, a labeled dNTP can be included in the PCR reaction mixture. PCR amplification can be performed in a DNA thermal cycler according to conventional techniques. After a suitable number of rounds to achieve amplification, the PCR reaction mixture is electrophoresed on a polyacrylamide gel. After drying the gel, the radioactivity of the appropriate bands (corresponding to the mRNA encoding the Enterococcus polypeptides of the present invention) are quantified using an imaging analyzer. RT and PCR reaction ingredients and conditions, reagent and gel concentrations, and labeling methods are well known in the art. Variations on the RT-PCR method will be apparent to the skilled artisan. Other PCR methods that can detect the nucleic acid of the present invention can be found in PCR PRIMER: A LABORATORY MANUAL (C. W. Dieffenbach et al. eds., Cold Spring Harbor Lab Press, 1995).

The polynucleotides of the present invention., including both DNA and RNA, may be used to detect polynucleotides of the present invention or Enterococcal species including E. faecalis using bio chip technology. The present invention includes both high density chip arrays (>1000 oligonucleotides per cm²) and low density chip arrays (<1000 oligonucleotides per cm²). Bio chips comprising arrays of polynucleotides of the present invention may be used to detect Enterococcal species, including E. faecalis, in biological and environmental samples and to diagnose an animal, including humans, with an E. faecalis or other Enterococcal infection. The bio chips of the present invention may comprise polynucleotide sequences of other pathogens including bacteria, viral, parasitic, and fungal polynucleotide sequences, in addition to the polynucleotide sequences of the present invention, for use in rapid differential pathogenic detection and diagnosis. The bio chips can also be used to monitor an E. faecalis or other Enterococcal infections and to monitor the genetic changes (deletions, insertions, mismatches, etc.) in response to drug therapy in the clinic and drug development in the laboratory. The bio chip technology comprising arrays of polynucleotides of the present invention may also be used to simultaneously monitor the expression of a multiplicity of genes, including those of the present invention. The polynucleotides used to comprise a selected array may be specified in the same manner as for the fragements, i.e., by their 5′ and 3′ positions or length in contigious base pairs and include from. Methods and particular uses of the polynucleotides of the present invention to detect Enterococcal species, including E. faecalis, using bio chip technology include those known in the art and those of: U.S. Pat. Nos. 5,510,270, 5,545,531, 5,445,934, 5,677,195, 5,532,128, 5,556,752, 5,527,681, 5,451,683, 5,424,186, 5,607,646, 5,658,732 and World Patent Nos. WO/9710365, WO/9511995, WO/9743447, WO/9535505, each incorporated herein in their entireties.

Biosensors using the polynucleotides of the present invention may also be used to detect, diagnose, and monitor E. faecalis or other Enterococcal species and infections thereof. Biosensors using the polynucleotides of the present invention may also be used to detect particular polynucleotides of the present invention. Biosensors using the polynucleotides of the present invention may also be used to monitor the genetic changes (deletions, insertions, mismatches, etc.) in response to drug therapy in the clinic and drug development in the laboratory. Methods and particular uses of the polynucleotides of the present invention to detect Enterococcal species, including E. faecalis, using biosenors include those known in the art and those of: U.S. Pat. Nos. 5,721,102, 5,658,732, 5,631,170, and World Patent Nos. WO97/35011, WO/97/20203, each incorporated herein in their entireties.

Thus, the present invention includes both bio chips and biosensors comprising polynucleotides of the present invention and methods of their use.

Assaying Enterococcus polypeptide levels in a biological sample can occur using any art-known method, such as antibody-based techniques. For example, Enterococcus polypeptide expression in tissues can be studied with classical immunohistological methods. In these, the specific recognition is provided by the primary antibody (polyclonal or monoclonal) but the secondary detection system can utilize fluorescent, enzyme, or other conjugated secondary antibodies. As a result, an immunohistological staining of tissue section for pathological examination is obtained. Tissues can also be extracted, e.g., with urea and neutral detergent, for the liberation of Enterococcus polypeptides for Western-blot or dot/slot assay. See, e.g., Jalkanen, M. et al. (1985) J. Cell. Biol. 101:976-985; Jalkanen, M. et al. (1987) J. Cell . Biol. 105:3087-3096. In this technique, which is based on the use of cationic solid phases, quantitation of a Enterococcus polypeptide can be accomplished using an isolated Enterococcus polypeptide as a standard. This technique can also be applied to body fluids.

Other antibody-based methods useful for detecting Enterococcus polypeptide gene expression include immunoassays, such as the ELISA and the radioimmunoassay (RIA). For example, a Enterococcus polypeptide-specific monoclonal antibodies can be used both as an immunoabsorbent and as an enzyme-labeled probe to detect and quantify a Enterococcus polypeptide. The amount of a Enterococcus polypeptide present in the sample can be calculated by reference to the amount present in a standard preparation using a linear regression computer algorithm. Such an ELISA is described in Iacobelli et al. (1988) Breast Cancer Research and Treatment 11:19-30. In another ELISA assay, two distinct specific monoclonal antibodies can be used to detect Enterococcus polypeptides in a body fluid. In this assay, one of the antibodies is used as the immunoabsorbent and the other as the enzyme-labeled probe.

The above techniques may be conducted essentially as a “one-step” or “two-step” assay. The “one-step” assay involves contacting the Enterococcus polypeptide with immobilized antibody and, without washing, contacting the mixture with the labeled antibody. The “two-step” assay involves washing before contacting the mixture with the labeled antibody. Other conventional methods may also be employed as suitable. It is usually desirable to immobilize one component of the assay system on a support, thereby allowing other components of the system to be brought into contact with the component and readily removed from the sample. Variations of the above and other immunological methods included in the present invention can also be found in Harlow et al., ANTIBODIES: A LABORATORY MANUAL, (Cold Spring Harbor Laboratory Press, 2nd ed. 1988).

Suitable enzyme labels include, for example, those from the oxidase group, which catalyze the production of hydrogen peroxide by reacting with substrate. Glucose oxidase is particularly preferred as it has good stability and its substrate (glucose) is readily available. Activity of an oxidase label may be assayed by measuring the concentration of hydrogen peroxide formed by the enzyme-labeled antibody/substrate reaction. Besides enzymes, other suitable labels include radioisotopes, such as iodine (¹²⁵I, ¹²¹I), carbon (¹⁴C), sulphur (³⁵S), tritium (³H), indium (¹¹²In), and technetium (^{99m}Tc), and fluorescent labels, such as fluorescein and rhodamine, and biotin.

Further suitable labels for the Enterococcus polypeptide-specific antibodies of the present invention are provided below. Examples of suitable enzyme labels include malate dehydrogenase, Enterococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹ Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is a preferred isotope where in vivo imaging is used since its avoids the problem of dehalogenation of the ¹²⁵I or ¹³¹I-labeled monoclonal antibody by the liver. In addition, this radionucleotide has a more favorable gamma emission energy for imaging. See, e.g., Perkins et al. (1985) Eur. J. Nucl. Med. 10:296-301; Carasquillo et al. (1987) J. Nucl. Med. 28:281-287. For example, ¹¹¹In coupled to monoclonal antibodies with 1-(P-isothiocyanatobenzyl)-DPTA has shown little uptake in non-tumors tissues, particularly the liver, and therefore enhances specificity of tumor localization. See, Esteban et al. (1987) J. Nucl. Med. 28:861-870.

Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, an o-phthaldehyde label, and a fluorescamine label.

Examples of suitable toxin labels include, Pseudomonas toxin, diphtheria toxin, ricin, and cholera toxin.

Examples of chemiluminescent labels include a luminal label, an isoluminal label, an aromatic acridinium ester label, an imidazole label, an acridinium salt label, an oxalate ester label, a luciferin label, a luciferase label, and an aequorin label.

Examples of nuclear magnetic resonance contrasting agents include heavy metal nuclei such as Gd, Mn, and iron.

Typical techniques for binding the above-described labels to antibodies are provided by Kennedy et al. (1976) Clin. Chim. Acta 70:1-31, and Schurs et al. (1977) Clin. Chim. Acta 81:1-40. Coupling techniques mentioned in the latter are the glutaraldehyde method, the periodate method, the dimaleimide method, the m-maleimidobenzyl-N-hydroxy-succinimide ester method, all of which methods are incorporated by reference herein.

In a related aspect, the invention includes a diagnostic kit for use in screening serum containing antibodies specific against E. faecalis infection. Such a kit may include an isolated E. faecalis antigen comprising an epitope which is specifically immunoreactive with at least one anti-E. faecalis antibody. Such a kit also includes means for detecting the binding of said antibody to the antigen. In specific embodiments, the kit may include a recombinantly produced or chemically synthesized peptide or polypeptide antigen. The peptide or polypeptide antigen may be attached to a solid support.

In a more specific embodiment, the detecting means of the above-described kit includes a solid support to which said peptide or polypeptide antigen is attached. Such a kit may also include a non-attached reporter-labeled anti-human antibody. In this embodiment, binding of the antibody to the E. faecalis antigen can be detected by binding of the reporter labeled antibody to the anti-E. faecalis polypeptide antibody.

In a related aspect, the invention includes a method of detecting E. faecalis infection in a subject. This detection method includes reacting a body fluid, preferably serum, from the subject with an isolated E. faecalis antigen, and examining the antigen for the presence of bound antibody. In a specific embodiment, the method includes a polypeptide antigen attached to a solid support, and serum is reacted with the support. Subsequently, the support is reacted with a reporter-labeled anti-human antibody. The support is then examined for the presence of reporter-labeled antibody.

The solid surface reagent employed in the above assays and kits is prepared by known techniques for attaching protein material to solid support material, such as polymeric beads, dip sticks, 96-well plates or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

The polypeptides and antibodies of the present invention, including fragments thereof, may be used to detect Enterococcal species including E. faecalis using bio chip and biosensor technology. Bio chip and biosensors of the present invention may comprise the polypeptides of the present invention to detect antibodies, which specifically recognize Enterococcal species, including E. faecalis. Bio chip and biosensors of the present invention may also comprise antibodies which specifically recognize the polypeptides of the present invention to detect Enterococcal species, including E. faecalis or specific polypeptides of the present invention. Bio chips or biosensors comprising polypeptides or antibodies of the present invention may be used to detect Enterococcal species, including E. faecalis, in biological and environmental samples and to diagnose an animal, including humans, with an E. faecalis or other Enterococcal infection. Thus, the present invention includes both bio chips and biosensors comprising polypeptides or antibodies of the present invention and methods of their use. The bio chips of the present invention may further comprise polypeptide sequences of other pathogens including bacteria, viral, parasitic, and fungal polypeptide sequences, in addition to the polypeptide sequences of the present invention, for use in rapid differential pathogenic detection and diagnosis. The bio chips of the present invention may further comprise antibodies or fragements thereof specific for other pathogens including bacteria, viral, parasitic, and fungal polypeptide sequences, in addition to the antibodies or fragements thereof of the present invention, for use in rapid differential pathogenic detection and diagnosis. The bio chips and biosensors of the present invention may also be used to monitor an E. faecalis or other Enterococcal infection and to monitor the genetic changes (amio acid deletions, insertions, substitutions, etc.) in response to drug therapy in the clinic and drug development in the laboratory. The bio chip and biosensors comprising polypeptides or antibodies of the present invention may also be used to simultaneously monitor the expression of a multiplicity of polypeptides, including those of the present invention. The polypeptides used to comprise a bio chip or biosensor of the present invention may be specified in the same manner as for the fragements, i.e., by their N-terminal and C-terminal positions or length in contigious amino acid residue. Methods and particular uses of the polypeptides and antibodies of the present invention to detect Enterococcal species, including E. faecalis, or specific polypeptides using bio chip and biosensor technology include those known in the art, those of the U.S. patent Nos. and World Patent Nos. listed above for bio chips and biosensors using polynucleotides of the present invention, and those of: U.S. Pat. Nos. 5,658,732, 5,135,852, 5,567,301, 5,677,196, 5,690,894 and World Patent Nos. WO9729366, WO9612957, each incorporated herein in their entireties.

4. Screening Assay for Binding Agents

Using the isolated proteins of the present invention, the present invention further provides methods of obtaining and identifying agents which bind to a protein encoded by one of the ORFs of the present invention or to one of the fragments and the Enterococcus faecalis fragment and contigs herein described.

In general, such methods comprise steps of:

(a) contacting an agent with an isolated protein encoded by one of the ORFs of the present invention, or an isolated fragment of the Enterococcus faecalis genome; and

(b) determining whether the agent binds to said protein or said fragment.

The agents screened in the above assay can be, but are not limited to, peptides, carbohydrates, vitamin derivatives, or other pharmaceutical agents. The agents can be selected and screened at random or rationally selected or designed using protein modeling techniques.

For random screening, agents such as peptides, carbohydrates, pharmaceutical agents and the like are selected at random and are assayed for their ability to bind to the protein encoded by the ORF of the present invention.

Alternatively, agents may be rationally selected or designed. As used herein, an agent is said to be “rationally selected or designed” when the agent is chosen based on the configuration of the particular protein. For example, one skilled in the art can readily adapt currently available procedures to generate peptides, pharmaceutical agents and the like capable of binding to a specific peptide sequence in order to generate rationally designed antipeptide peptides, for example see Hurby et al., “Application of Synthetic Peptides: Antisense Peptides,” in Synthetic Peptides, A User's Guide, W. H. Freeman, NY (1992), pp. 289-307, and Kaspczak et al., Biochemistry 28:9230-8 (1989), or pharmaceutical agents, or the like.

In addition to the foregoing, one class of agents of the present invention, as broadly described, can be used to control gene expression through binding to one of the ORFs or EMFs of the present invention. As described above, such agents can be randomly screened or rationally designed/selected. Targeting the ORF or EMF allows a skilled artisan to design sequence specific or element specific agents, modulating the expression of either a single ORF or multiple ORFs which rely on the same EMF for expression control.

One class of DNA binding agents are agents which contain base residues which hybridize or form a triple helix by binding to DNA or RNA. Such agents can be based on the classic phosphodiester, ribonucleic acid backbone, or can be a variety of sulfhydryl or polymeric derivatives which have base attachment capacity.

Agents suitable for use in these methods usually contain 20 to 40 bases and are designed to be complementary to a region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense—Okano, J. Neurochem. 56:560 (1991); Oligodeoxynucleotides as Antisense Inhibitors of Gene Expression, CRC Press, Boca Raton, Fla. (1988)). Triple helix-formation optimally results in a shut-off of RNA transcription from DNA, while antisense RNA hybridization blocks translation of an mRNA molecule into polypeptide. Both techniques have been demonstrated to be effective in model systems. Information contained in the sequences of the present invention can be used to design antisense and triple helix-forming oligonucleotides, and other DNA binding agents.

5. Pharmaceutical Compositions and Vaccines

The present invention further provides pharmaceutical agents which can be used to modulate the growth or pathogenicity of Enterococcus faecalis, or another related organism, in vivo or in vitro. As used herein, a “pharmaceutical agent” is defined as a composition of matter which can be formulated using known techniques to provide a pharmaceutical compositions. As used herein, the “pharmaceutical agents of the present invention” refers the pharmaceutical agents which are derived from the proteins encoded by the ORFs of the present invention or are agents which are identified using the herein described assays.

As used herein, a pharmaceutical agent is said to “modulate the growth and/or pathogenicity of Enterococcus faecalis or a related organism, in vivo or in vitro,” when the agent reduces the rate of growth, rate of division, or viability of the organism in question. The pharmaceutical agents of the present invention can modulate the growth or pathogenicity of an organism in many fashions, although an understanding of the underlying mechanism of action is not needed to practice the use of the pharmaceutical agents of the present invention. Some agents will modulate the growth by binding to an important protein thus blocking the biological activity of the protein, while other agents may bind to a component of the outer surface of the organism blocking attachment or rendering the organism more prone to act the bodies nature immune system. Alternatively, the agent may comprise a protein encoded by one of the ORFs of the present invention and serve as a vaccine. The development and use of a vaccine based on outer membrane components are well known in the art.

As used herein, a “related organism” is a broad term which refers to any organism whose growth can be modulated by one of the pharmaceutical agents of the present invention. In general, such an organism will contain a homolog of the protein which is the target of the pharmaceutical agent or the protein used as a vaccine. As such, related organisms do not need to be bacterial but may be fungal or viral pathogens.

The pharmaceutical agents and compositions of the present invention may be administered in a convenient manner, such as by the oral, topical, intravenous, intraperitoneal, intramuscular, subcutaneous, intranasal or intradermal routes. The pharmaceutical compositions are administered in an amount which is effective for treating and/or prophylaxis of the specific indication. In general, they are administered in an amount of at least about 1 mg/kg body weight and in most cases they will be administered in an amount not in excess of about 1 g/kg body weight per day. In most cases, the dosage is from about 0.1 mg/kg to about 10 g/kg body weight daily, taking into account the routes of administration, symptoms, etc.

The agents of the present invention can be used in native form or can be modified to form a chemical derivative. As used herein, a molecule is said to be a “chemical derivative” of another molecule when it contains additional chemical moieties not normally a part of the molecule. Such moieties may improve the molecule's solubility, absorption, biological half life, etc. The moieties may alternatively decrease the toxicity of the molecule, eliminate or attenuate any undesirable side effect of the molecule, etc. Moieties capable of mediating such effects are disclosed in, among other sources, REMINGTON'S PHARMACEUTICAL SCIENCES (1980) cited elsewhere herein.

For example, such moieties may change an immunological character of the functional derivative, such as affinity for a given antibody. Such changes in immunomodulation activity are measured by the appropriate assay, such as a competitive type immunoassay. Modifications of such protein properties as redox or thermal stability, biological half-life, hydrophobicity, susceptibility to proteolytic degradation or the tendency to aggregate with carriers or into multimers also may be effected in this way and can be assayed by methods well known to the skilled artisan.

The therapeutic effects of the agents of the present invention may be obtained by providing the agent to a patient by any suitable means (e.g., inhalation, intravenously, intramuscularly, subcutaneously, enterally, or parenterally). It is preferred to administer the agent of the present invention so as to achieve in effective concentration within the blood or tissue in which the growth of the organism is to be controlled. To achieve an effective blood concentration, the preferred method is to administer the agent by injection. The administration may be by continuous infusion, or by single or multiple injections.

In providing a patient with one of the agents of the present invention, the dosage of the administered agent will vary depending upon such factors as the patient's age, weight, height, sex, general medical condition, previous medical history, etc. In general, it is desirable to provide the recipient with a dosage of agent which is in the range of from about 1 pg/kg to 10 mg/kg (body weight of patient), although a lower or higher dosage may be administered. The therapeutically effective dose can be lowered by using combinations of the agents of the present invention or another agent.

As used herein, two or more compounds or agents are said to be administered “in combination” with each other when either (1) the physiological effects of each compound, or (2) the serum concentrations of each compound can be measured at the same time. The composition of the present invention can be administered concurrently with, prior to, or following the administration of the other agent.

The agents of the present invention are intended to be provided to recipient subjects in an amount sufficient to decrease the rate of growth (as defined above) of the target organism.

The administration of the agent(s) of the invention may be for either a “prophylactic” or “therapeutic” purpose. When provided prophylactically, the agent(s) are provided in advance of any symptoms indicative of the organisms growth. The prophylactic administration of the agent(s) serves to prevent, attenuate, or decrease the rate of onset of any subsequent infection. When provided therapeutically, the agent(s) are provided at (or shortly after) the onset of an indication of infection. The therapeutic administration of the compound(s) serves to attenuate the pathological symptoms of the infection and to increase the rate of recovery.

The agents of the present invention are administered to a subject, such as a mammal, or a patient, in a pharmaceutically acceptable form and in a therapeutically effective concentration. A composition is said to be “pharmacologically acceptable” if its administration can be tolerated by a recipient patient. Such an agent is said to be administered in a “therapeutically effective amount” if the amount administered is physiologically significant. An agent is physiologically significant if its presence results in a detectable change in the physiology of a recipient patient.

The agents of the present invention can be formulated according to known methods to prepare pharmaceutically useful compositions, whereby these materials, or their functional derivatives, are combined in a mixture with a pharmaceutically acceptable carrier vehicle. Suitable vehicles and their formulation, inclusive of other human proteins, e.g., human serum albumin, are described, for example, in REMINGTON'S PHARMACEUTICAL SCIENCES, 16th Ed., Osol, A., Ed., Mack Publishing, Easton, Pa. (1980). In order to form a pharmaceutically acceptable composition suitable for effective administration, such compositions will contain an effective amount of one or more of the agents of the present invention, together with a suitable amount of carrier vehicle.

Additional pharmaceutical methods may be employed to control the duration of action. Control release preparations may be achieved through the use of polymers to complex or absorb one or more of the agents of the present invention. The controlled delivery may be effectuated by a variety of well known techniques, including formulation with macromolecules such as, for example, polyesters, polyamino acids, polyvinyl, pyrrolidone, ethylenevinylacetate, methylcellulose, carboxymethylcellulose, or protamine, sulfate, adjusting the concentration of the macromolecules and the agent in the formulation, and by appropriate use of methods of incorporation, which can be manipulated to effectuate a desired time course of release. Another possible method to control the duration of action by controlled release preparations is to incorporate agents of the present invention into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization with, for example, hydroxymethylcellulose or gelatine-microcapsules and poly(methylmethacylate) microcapsules, respectively, or in colloidal drug delivery systems, for example, liposomes, albumin microspheres, microemulsions, nanoparticles, and nanocapsules or in macroemulsions. Such techniques are disclosed in REMINGTON'S PHARMACEUTICAL SCIENCES (1980).

The invention further provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In addition, the agents of the present invention may be employed in conjunction with other therapeutic compounds.

The present invention also provides vaccines comprising one or more polypeptides of the present invention. Heterogeneity in the composition of a vaccine may be provided by combining E. faecalis polypeptides of the present invention. Multi-component vaccines of this type are desirable because they are likely to be more effective in eliciting protective immune responses against multiple species and strains of the Enterococcus genus than single polypeptide vaccines.

Multi-component vaccines are known in the art to elicit antibody production to numerous immunogenic components. See, e.g., Decker et al. (1996) J. Infect. Dis. 174:S270-275. In addition, a hepatitis B, diphtheria, tetanus, pertussis tetravalent vaccine has recently been demonstrated to elicit protective levels of antibodies in human infants against all four pathogenic agents. See, e.g., Axistegui, J. et al. (1997) Vaccine 15:7-9.

The present invention in addition to single-component vaccines includes multi-component vaccines. These vaccines comprise more than one polypeptide, immunogen or antigen. Thus, a multi-component vaccine would be a vaccine comprising more than one of the E. faecalis polypeptides of the present invention.

Further within the scope of the invention are whole cell and whole viral vaccines. Such vaccines may be produced recombinantly and involve the expression of one or more of the E. faecalis polypeptides described in SEQ ID NOS:1-982. For example, the E. faecalis polypeptides of the present invention may be either secreted or localized intracellular, on the cell surface, or in the periplasmic space. Further, when a recombinant virus is used, the E. faecalis polypeptides of the present invention may, for example, be localized in the viral envelope, on the surface of the capsid, or internally within the capsid. Whole cells vaccines which employ cells expressing heterologous proteins are known in the art. See, e.g., Robinson, K. et al. (1997) Nature Biotech. 15:653-657; Sirard, J. et al. (1997) Infect. Immun. 65:2029-2033; Chabalgoity, J. et al. (1997) Infect. Immun. 65:2402-2412. These cells may be administered live or may be killed prior to administration. Chabalgoity, J. et al., supra, for example, report the successful use in mice of a live attenuated Salmonella vaccine strain which expresses a portion of a platyhelminth fatty acid-binding protein as a fusion protein on its cells surface.

A multi-component vaccine can also be prepared using techniques known in the art by combining one or more E. faecalis polypeptides of the present invention, or fragments thereof, with additional non-Enterococcal components (e.g., diphtheria toxin or tetanus toxin, and/or other compounds known to elicit an immune response). Such vaccines are useful for eliciting protective immune responses to both members of the Enterococcus genus and non-Enterococcal pathogenic agents.

The vaccines of the present invention also include DNA vaccines. DNA vaccines are currently being developed for a number of infectious diseases. See, et al., Boyer, et al. (1997) Nat. Med. 3:526-532; reviewed in Spier, R. (1996) Vaccine 14:1285-1288. Such DNA vaccines contain a nucleotide sequence encoding one or more E. faecalis polypeptides of the present invention oriented in a manner that allows for expression of the subject polypeptide. For example, the direct administration of plasmid DNA encoding B. burgdorgeri OspA has been shown to elicit protective immunity in mice against borrelial challenge. See, Luke et al. (1997) J. Infect. Dis. 175:91-97.

The present invention also relates to the administration of a vaccine which is co-administered with a molecule capable of modulating immune responses. Kim et al. (1997) Nature Biotech. 15:641-646, for example, report the enhancement of immune responses produced by DNA immunizations when DNA, sequences encoding molecules which stimulate the immune response are co-administered. In a similar fashion, the vaccines of the present invention may be co-administered with either nucleic acids encoding immune modulators or the immune modulators themselves. These immune modulators include granulocyte macrophage colony stimulating factor (GM-CSF) and CD86.

The vaccines of the present invention may be used to confer resistance to Enterococcal infection by either passive or active immunization. When the vaccines of the present invention are used to confer resistance to Enterococcal infection through active immunization, a vaccine of the present invention is administered to an animal to elicit a protective immune response which either prevents or attenuates a Enterococcal infection. When the vaccines of the present invention are used to confer resistance to Enterococcal infection through passive immunization, the vaccine is provided to a host animal (e.g., human, dog, or mouse), and the antisera elicited by this antisera is recovered and directly provided to a recipient suspected of having an infection caused by a member of the Enterococcus genus.

The ability to label antibodies, or fragments of antibodies, with toxin molecules provides an additional method for treating Enterococcal infections when passive immunization is conducted. In this embodiment, antibodies, or fragments of antibodies, capable of recognizing the E. faecalis polypeptides disclosed herein, or fragments thereof, as well as other Enterococcus proteins, are labeled with toxin molecules prior to their administration to the patient. When such toxin derivatized antibodies bind to Enterococcus cells, toxin moieties will be localized to these cells and will cause their death.

The present invention thus concerns and provides a means for preventing or attenuating a Enterococcal infection resulting from organisms which have antigens that are recognized and bound by antisera produced in response to the polypeptides of the present invention. As used herein, a vaccine is said to prevent or attenuate a disease if its administration to an animal results either in the total or partial attenuation (i.e., suppression) of a symptom or condition of the disease, or in the total or partial immunity of the animal to the disease.

The administration of the vaccine (or the antisera which it elicits) may be for either a “prophylactic” or “therapeutic” purpose. When provided prophylactically, the compound(s) are provided in advance of any symptoms of Enterococcal infection. The prophylactic administration of the compound(s) serves to prevent or attenuate any subsequent infection. When provided therapeutically, the compound(s) is provided upon or after the detection of symptoms which indicate that an animal may be infected with a member of the Enterococcus genus. The therapeutic administration of the compound(s) serves to attenuate any actual infection. Thus, the E. faecalis polypeptides, and fragments thereof, of the present invention may be provided either prior to the onset of infection (so as to prevent or attenuate an anticipated infection) or after the initiation of an actual infection.

The polypeptides of the invention, whether encoding a portion of a native protein or a functional derivative thereof, may be administered in pure form or may be coupled to a macromolecular carrier. Example of such carriers are proteins and carbohydrates. Suitable proteins which may act as macromolecular carrier for enhancing the immunogenicity of the polypeptides of the present invention include keyhole limpet hemacyanin (KLH) tetanus toxoid, pertussis toxin, bovine serum albumin, and ovalbumin. Methods for coupling the polypeptides of the present invention to such macromolecular carriers are disclosed in Harlow et al., ANTIBODIES: A LABORATORY MANUAL, (Cold Spring Harbor Laboratory Press, 2nd ed. 1988).

A composition is said to be “pharmacologically or physiologically acceptable” if its administration can be tolerated by a recipient animal and is otherwise suitable for administration to that animal. Such an agent is said to be administered in a “therapeutically effective amount” if the amount administered is physiologically significant. An agent is physiologically significant if its presence results in a detectable change in the physiology of a recipient patient.

While in all instances the vaccine of the present invention is administered as a pharmacologically acceptable compound, one skilled in the art would recognize that the composition of a pharmacologically acceptable compound varies with the animal to which it is administered. For example, a vaccine intended for human use will generally not be co-administered with Freund's adjuvant. Further, the level of purity of the E. faecalis polypeptides of the present invention will normally be higher when administered to a human than when administered to a non-human animal.

As would be understood by one of ordinary skill in the art, when the vaccine of the present invention is provided to an animal, it may be in a composition which may contain salts, buffers, adjuvants, or other substances which are desirable for improving the efficacy of the composition. Adjuvants are substances that can be used to specifically augment a specific immune response. These substances generally perform two functions: (1) they protect the antigen(s) from being rapidly catabolized after administration and (2) they nonspecifically stimulate immune responses.

Normally, the adjuvant and the composition are mixed prior to presentation to the immune system, or presented separately, but into the same site of the animal being immunized. Adjuvants can be loosely divided into several groups based upon their composition. These groups include oil adjuvants (for example, Freund's complete and incomplete), mineral salts (for example, ALK(SO₄)₂, AlNa(SO₄)₂, AlNH₄(SO₄), silica, kaolin, and carbon), polynucleotides (for example, poly IC and poly AU acids), and certain natural substances (for example, wax D from Mycobacterium tuberculosis, as well as substances found in Corynebacterium parvum, or Bordetella pertussis, and members of the genus Brucella. Other substances useful as adjuvants are the saponins such as, for example, Quil A. (Superfos A/S, Denmark). Preferred adjuvants for use in the present invention include aluminum salts, such as AlK(SO₄)₂, AlNa(SO₄)₂, and AlNH₄(SO₄). Examples of materials suitable for use in vaccine compositions are provided in REMINGTON'S PHARMACEUTICAL SCIENCES 1324-1341 (A. Osol, ed, Mack Publishing Co, Easton, Pa., (1980) (incorporated herein by reference).

The therapeutic compositions of the present invention can be administered parenterally by injection, rapid infusion, nasopharyngeal absorption (intranasopharangeally), dermoabsorption, or orally. The compositions may alternatively be administered intramuscularly, or intravenously. Compositions for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

Therapeutic compositions of the present invention can also be administered in encapsulated form. For example, intranasal immunization using vaccines encapsulated in biodegradable microsphere composed of poly(DL-lactide-co-glycolide). See, Shahin, R. et al. (1995) Infect. Immun. 63:1195-1200. Similarly, orally administered encapsulated Salmonella typhimurium antigens can also be used. Allaoui-Attarki, K. et al. (1997) Infect. Immun. 65:853-857. Encapsulated vaccines of the present invention can be administered by a variety of routes including those involving contacting the vaccine with mucous membranes (e.g., intranasally, intracolonicly, intraduodenally).

Many different techniques exist for the timing of the immunizations when a multiple administration regimen is utilized. It is possible to use the compositions of the invention more than once to increase the levels and diversities of expression of the immunoglobulin repertoire expressed by the immunized animal. Typically, if multiple immunizations are given, they will be given one to two months apart.

According to the present invention, an “effective amount” of a therapeutic composition is one which is sufficient to achieve a desired biological effect. Generally, the dosage needed to provide an effective amount of the composition will vary depending upon such factors as the animal's or human's age, condition, sex, and extent of disease, if any, and other variables which can be adjusted by one of ordinary skill in the art.

The antigenic preparations of the invention can be administered by either single or multiple dosages of an effective amount. Effective amounts of the compositions of the invention can vary from 0.01-1,000 μg/ml per dose, more preferably 0.1-500 μg/ml pcr dose, and most preferably 10-300 μg/ml per dose.

6. Shot-Gun Approach to Megabase DNA Sequencing

The present invention further demonstrates that a large genome can be sequenced using a random shotgun approach. This procedure, described in detail in the examples that follow, has eliminated the up front cost of isolating and ordering overlapping or contiguous subclones prior to the start of the sequencing protocols.

Certain aspects of the present invention are described in greater detail in the examples that follow. The examples are provided by way of illustration. Other aspects and embodiments of the present invention are contemplated by the inventors, as will be clear to those of skill in the art from reading the present disclosure.

### ILLUSTRATIVE EXAMPLES

Libraries and Sequencing

1. Shotgun Sequencing Probability Analysis

The overall strategy for a shotgun approach to whole genome sequencing follows from the Lander and Waterman (Landerman and Waterman, Genomics 2:231 (1988)) application of the equation for the Poisson distribution. According to this treatment, the probability, P0, that any given base in a sequence of size L, in nucleotides, is not sequenced after a certain amount, n, in nucleotides, of random sequence has been determined can be calculated by the equation P0=e−m, where m is L/n, the fold coverage. For instance, for a genome of 2.8 Mb, m=1 when 2.8 Mb of sequence has been randomly generated (1×coverage). At that point, P0=e−1=0.37. The probability that any given base has not been sequenced is the same as the probability that any region of the whole sequence L has not been determined and, therefore, is equivalent to the fraction of the whole sequence that has yet to be determined. Thus, at one-fold coverage, approximately 37% of a polynucleotide of size L, in nucleotides has not been sequenced. When 14 Mb of sequence has been generated, coverage is 5× for a 2.8 Mb and the unsequenced fraction drops to 0.0067 or 0.67%. 5× coverage of a 2.8 Mb sequence can be attained by sequencing approximately 17,000 random clones from both insert ends with an average sequence read length of 410 bp.

Similarly, the total gap length, G, is determined by the equation G=Le−m, and the average gap size, g, follows the equation, g=L/n. Thus, 5× coverage leaves about 240 gaps averaging about 82 bp in size in a sequence of a polynucleotide 2.8 Mb long.

The treatment above is essentially that of Lander and Waterman, Genomics 2: 231 (1988).

2. Random Library Construction

In order to approximate the random model described above during actual sequencing, a nearly ideal library of cloned genomic fragments is required. The following library construction procedure was developed to achieve this end.

Enterococcus faecalis DNA is prepared by phenol extraction. A mixture containing 200 μg DNA in 1.0 ml of 300 mM sodium acetate, 10 mM Tris-HCl, 1 mM Na-EDTA, 50% glycerol is processed through a nebulizer (IPI Medical Products) with a stream of nitrogen adjusted to 35 Kpa for 2 minutes. The sonicated DNA is ethanol precipitated and redissolved in 500 μl TE buffer.

To create blunt-ends, a 100 μl aliquot of the resuspended DNA is digested with 5 units of BAL31 nuclease (New England BioLabs) for 10 min at 30° C. in 200 μl BAL31 buffer. The digested DNA is phenol-extracted, ethanol-precipitated, redissolved in 100 μl TE buffer, and then size-fractionated by electrophoresis through a 1.0% low melting temperature agarose gel. The section containing DNA fragments 1.6-2.0 kb in size is excised from the gel, and the LGT agarose is melted and the resulting solution is extracted with phenol to separate the agarose from the DNA. DNA is ethanol precipitated and redissolved in 20 μl of TE buffer for ligation to vector.

A two-step ligation procedure is used to produce a plasmid library with 97% inserts, of which >99% were single inserts. The first ligation mixture (50 ul) contains 2 μg of DNA fragments, 2 μg pUC18 DNA (Pharmacia) cut with SmaI and dephosphorylated with bacterial alkaline phosphatase, and 10 units of T4 ligase (GIBCO/BRL) and is incubated at 14° C. for 4 hr. The ligation mixture then is phenol extracted and ethanol precipitated, and the precipitated DNA is dissolved in 20 μl TE buffer and electrophoresed on a 1.0% low melting agarose gel. Discrete bands in a ladder are visualized by ethidium bromide-staining and UV illumination and identified by size as insert (1), vector (v), v+I, v+2i, v+3i, etc. The portion of the gel containing v+I DNA is excised and the v+I DNA is recovered and resuspended into 20 μl TE. The v+I DNA then is blunt-ended by T4 polymerase treatment for 5 min. at 37° C. in a reaction mixture (50 ul) containing the v+I linears, 500 μM each of the 4 dNTPs, and 9 units of T4 polymerase (New England BioLabs), under recommended buffer conditions. After phenol extraction and ethanol precipitation the repaired v+I linears are dissolved in 20 μl TE. The final ligation to produce circles is carried out in a 50 μl reaction containing 5 μl of v+I linears and 5 units of T4 ligase at 14° C. overnight. After 10 min. at 70° C. the following day, the reaction mixture is stored at −20° C.

This two-stage procedure results in a molecularly random collection of single-insert plasmid recombinants with minimal contamination from double-insert chimeras (<1%) or free vector (<3%).

Since deviation from randomness can arise from propagation the DNA in the host, E. coli host cells deficient in all recombination and restriction functions (A. Greener, Strategies 3 (1):5 (1990)) are used to prevent rearrangements, deletions, and loss of clones by restriction. Furthermore, transformed cells are plated directly on antibiotic diffusion plates to avoid the usual broth recovery phase which allows multiplication and selection of the most rapidly growing cells.

Plating is carried out as follows. A 100 μl aliquot of Epicurian Coli SURE II Supercompetent Cells (Stratagene 200152) is thawed on ice and transferred to a chilled Falcon 2059 tube on ice. A 1.7 μl aliquot of 1.42 M beta-mercaptoethanol is added to the aliquot of cells to a final concentration of 25 mM. Cells are incubated on ice for 10 min. A 1 μl aliquot of the final ligation is added to the cells and incubated on ice for 30 min. The cells are heat pulsed for 30 sec. at 42° C. and placed back on ice for 2 min. The outgrowth period in liquid culture is eliminated from this protocol in order to minimize the preferential growth of any given transformed cell. Instead the transformation mixture is plated directly on a nutrient rich SOB plate containing a 5 ml bottom layer of SOB agar (5% SOB agar: 20 g tryptone, 5 g yeast extract, 0.5 g NaCl, 1.5% Difco Agar per liter of media). The 5 ml bottom layer is supplemented with 0.4 ml of 50 mg/ml ampicillin per 100 ml SOB agar. The 15 ml top layer of SOB agar is supplemented with 1 ml X-Gal (2%), 1 ml MgCl2 (1 M), and 1 ml MgSO4/100 ml SOB agar. The 15 μl top layer is poured just prior to plating. Our titer is approximately 100 colonies/10 μl aliquot of transformation.

All colonies are picked for template preparation regardless of size. Thus, only clones lost due to “poison” DNA or deleterious gene products are deleted from the library, resulting in a slight increase in gap number over that expected.

3. Random DNA Sequencing

High quality double stranded DNA plasmid templates are prepared using a “boiling bead” method developed in collaboration with Advanced Genetic Technology Corp. (Gaithersburg, Md.) (Adams et al., Science 252:1651 (1991); Adams et al., Nature 355:632 (1992)). Plasmid preparation is performed in a 96-well format for all stages of DNA preparation from bacterial growth through final DNA purification. Template concentration is determined using Hoechst Dye and a Millipore Cytofluor. DNA concentrations are not adjusted, but low-yielding templates are identified where possible and not sequenced.

Templates are also prepared from an Enterococcus faecalis lambda genomic library in the vector DASH II (Stratagene). In particular, Enterococcus faecalis DNA (>100 kb) is partially digested in a reaction mixture (200 ul) containing 50 μg DNA, 1× Sau3AI buffer, 20 units Sau3AI for 6 min. at 23° C. The digested DNA was phenol-extracted and fractionated by sucrose density gradient centrifugation. Fractions of the sucrose gradient containing 15 to 25 kb are recovered in a final volume of 6 ul. One μl of fragments is used with 1 μl of lambda DASHII vector (Stratagene) in the recommended ligation reaction. One μl of the ligation mixture is used per packaging reaction following the recommended protocol with the Gigapack II XL Packaging Extract (Stratagene, #227711). Phage are plated directly without amplification from the packaging mixture (after dilution with 500 μl of recommended SM buffer and chloroform treatment). Yield is about 2.5×10³ pfu/ul. An amplified library is prepared by infecting restructure NM539 host E. coli cells eitn approximately 1×10⁴ phage particles and recovering the progeny phages particles. The recovered phage is stored frozen in 7% dimethylsulfoxide. The phage titer is approximately 1×10⁹ pfu/ml.

For high throughput sequencing of individual lambda phage clones, liquid lysates (100 μl) are prepared from randomly selected plaques (from the unamplified library) and template is prepared by long-range PCR using T7 and T3 vector-specific primers.

Sequencing reactions are carried out on plasmid and/or PCR templates using the AB Catalyst LabStation with Applied Biosystems PRISM Ready Reaction Dye Primer Cycle Sequencing Kits for the M13 forward (M13-21) and the M13 reverse (M13RP1) primers (Adams et al., Nature 368:474 (1994)). Dye terminator sequencing reactions are carried out on the lambda templates on a Perkin-Elmer 9600 Thermocycler using the Applied Biosystems Ready Reaction Dye Terminator Cycle Sequencing kits. T7 and T3 primers are used to sequence the ends of the inserts from the Lambda DASH II library. Sequencing reactions are performed by eight individuals using an average of fourteen AB 373 DNA Sequencers per day. All sequencing reactions are analyzed using the Stretch modification of the AB 373, primarily using a 34 cm well-to-read distance. The overall sequencing success rate very approximately is about 85% for M13-21 and M13RP1 sequences and 65% for dye-terminator reactions. The average usable read length is 485 bp for M13-21 sequences, 445 bp for M13RP1 sequences, and 375 bp for dye-terminator reactions.

Richards et al., Chapter 28 in AUTOMATED DNA SEQUENCING AND ANALYSIS, M. D. Adams, C. Fields, J. C. Venter, Eds., Academic Press, London, (1994) described the value of using sequence from both ends of sequencing templates to facilitate ordering of contigs in shotgun assembly projects of lambda and cosmid clones. We balance the desirability of both-end sequencing (including the reduced cost of lower total number of templates) against shorter read-lengths for sequencing reactions performed with the M13RP1 (reverse) primer compared to the M13-21 (forward) primer. Approximately one-half of the templates are sequenced from both ends. Random reverse sequencing reactions are done based on successful forward sequencing reactions. Some M13RP1 sequences are obtained in a semi-directed fashion: M13-21: sequences pointing outward at the ends of contigs are chosen for M13RP1 sequencing in an effort to specifically order contigs.

4. Protocol for Automated Cycle Sequencing

The sequencing was carried out using ABI Catalyst robots and AB 373 Automated DNA Sequencers. The Catalyst robot is a publicly available sophisticated pipetting and temperature control robot which has been developed specifically for DNA sequencing reactions. The Catalyst combines pre-aliquoted templates and reaction mixes consisting of deoxy- and dideoxynucleotides, the thermostable Taq DNA polymerase, fluorescently-labelled sequencing primers, and reaction buffer. Reaction mixes and templates are combined in the wells of an aluminum 96-well thermocycling plate. Thirty consecutive cycles of linear amplification (i.e.., one primer synthesis) steps are performed including denaturation, annealing of primer and template, and extension; i.e., DNA synthesis. A heated lid with rubber gaskets on the thermocycling plate prevents evaporation without the need for an oil overlay.

Two sequencing protocols are used: one for (lye-labelled primers and a second for dye-labelled dideoxy chain terminators. The shotgun sequencing involves use of four dye-labelled sequencing primers, one for each of the four terminator nucleotide. Each dye-primer is labelled with a different fluorescent dye, permitting the four individual reactions to be combined into one lane of the 373 DNA Sequencer for electrophoresis, detection, and base-calling. ABI currently supplies pre-mixed reaction mixes in bulk packages containing all the necessary non-template reagents for sequencing. Sequencing can be done with both plasmid and PCR-generated templates with both dye-primers and dye-terminators with approximately equal fidelity, although plasmid templates generally give longer usable sequences.

Thirty-two reactions are loaded per AB373 Sequencer each day, for a total of 960 samples. Electrophoresis is run overnight following the manufacturer's protocols, and the data is collected for twelve hours. Following electrophoresis and fluorescence detection, the ABI 373 performs automatic lane tracking and base-calling. The lane-tracking is confirmed visually. Each sequence electropherogram (or fluorescence lane trace) is inspected visually and assessed for quality. Trailing sequences of low quality are removed and the sequence itself is loaded via software to a Sybase database (archived daily to 8 mm tape). Leading vector polylinker sequence is removed automatically by a software program. Average edited lengths of sequences from the standard ABI 373 are around 400 bp and depend mostly on the quality of the template used for the sequencing reaction. ABI 373 Sequencers converted to Stretch Liners provide a longer electrophoresis path prior to fluorescence detection and increase the average number of usable bases to 500-600 bp.

Informatics

1. Data Management

A number of information management systems for a large-scale sequencing lab have been developed. (For review see, for instance, Kerlavage et al., Proceedings of the Twenty-Sixth Annual Hawaii International Conference on System Sciences, IEEE Computer Society Press, Washington D.C., 585 (1993)) The system used to collect and assemble the sequence data was developed using the Sybase relational database management system and was designed to automate data flow wherever possible and to reduce user error. The database stores and correlates all information collected during the entire operation from template preparation to final analysis of the genome. Because the raw output of the ABI 373 Sequencers was based on a Macintosh platform and the data management system chosen is based on a Unix platform, it was necessary to design and implement a variety of multi-user, client-server applications which allow the raw data as well as analysis results to flow seamlessly into the database with a minimum of user effort.

2. Assembly

An assembly engine (TIGR Assembler) developed for the rapid and accurate assembly of thousands of sequence fragments is employed to generate contigs. The TIGR assembler simultaneously clusters and assembles fragments of the genome. In order to obtain the speed necessary to assemble more than 104 fragments, the algorithm builds a hash table of 10 bp oligonucleotide subsequences to generate a list of potential sequence fragment overlaps. The number of potential overlaps for each fragment determines which fragments are likely to fall into repetitive elements. Beginning with a single seed sequence fragment, TIGR Assembler extends the current contig by attempting to add the best matching fragment based on oligonucleotide content. The contig and candidate fragment are aligned using a modified version of the Smith-Waterman algorithm which provides for optimal gapped alignments (Waterman, M. S., Methods in Enzymology 164:765 (1988)). The contig is extended by the fragment only if strict criteria for the quality of the match are met. The match criteria include the minimum length of overlap, the maximum length of an unmatched end, and the minimum percentage match. These criteria are automatically lowered by the algorithm in regions of minimal coverage and raised in regions with a possible repetitive element. The number of potential overlaps for each fragment determines which fragments are likely to fall into repetitive elements. Fragments representing the boundaries of repetitive elements and potentially chimeric fragments are often rejected based on partial mismatches at the ends of alignments and excluded from the current contig. TIGR Assembler is; designed to take advantage of clone size information coupled with sequencing from both ends of each template. It enforces the constraint that sequence fragments from two ends of the same template point toward one another in the contig and are located within a certain range of base pairs (definable for each clone based on the known clone size range for a given library).

The process resulted in 982 contigs as represented by SEQ ID NOs:1-982.

3. Identifying Genes

The predicted coding regions of the Enterococcus faecalis genome were initially defined with the program GeneMark, which finds ORFs using a probabilistic classification technique. The predicted coding region sequences were used in searches against a database of all Enterococcus faecali nucleotide sequences front GenBank (March, 1997), using the BLASTN search method to identify overlaps of 50 or more nucleotides with at least a 95% identity. Those ORFs with nucleotide sequence matches are shown in Table 1. The ORFs without such matches were translated to protein sequences and compared to a non-redundant database of known proteins generated by combining the Swiss-prot, PIR and GenPept databases. ORFs that matched a database protein with BLASTP probability less than or equal to 0.01 are shown in Table 2. The table also lists assigned functions based on the closest match in the databases. ORFs that did not match protein or nucleotide sequences in the databases at these levels are shown in Table 3.

Illustrative Applications

1. Production of an Antibody to a Enterococcus faecalis Protein

Substantially pure protein or polypeptide is isolated from the transfected or transformed cells using any one of the methods known in the art. The protein can also be produced in a recombinant prokaryotic expression system, such as E. coli, or can be chemically synthesized. Concentration of protein in the final preparation is adjusted, for example, by concentration on an Amicon filter device, to the level of a few micrograms/ml. Monoclonal or polyclonal antibody to the protein can then be prepared as follows.

2. Monoclonal Antibody Production by Hybridoma Fusion

Monoclonal antibody to epitopes of any of the peptides identified and isolated as described can be prepared from murine hybridomas according to the classical method of Kohler, G. and Milstein, C., Nature 256:495 (1975) or modifications of the methods thereof. Briefly, a mouse is repetitively inoculated with a few micrograms of the selected protein over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, E., Meth. Enzymol. 70:419 (1980), and modified methods thereof. Selected positive clones can be expanded and their monoclonal antibody product harvested for use. Detailed procedures for monoclonal antibody production are described in Davis, L. et al., Basic Methods in Molecular Biology, Elsevier, New York. Section 21-2 (1989).

3. Polyclonal Antibody Production by Immunization

Polyclonal antiserum containing antibodies to heterogenous epitopes of a single protein can be prepared by immunizing suitable animals with the expressed protein described above, which can be unmodified or modified to enhance immunogenicity. Effective polyclonal antibody production is affected by many factors related both to the antigen and the host species. For example, small molecules tend to be less immunogenic than others and may require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis, J. et al., J. Clin. Endocrinol. Metab. 33:988-991 (1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology, Wier, D., ed, Blackwell (1973). Plateau concentration of antibody is usually in the range of 0.1 to 0.2 mg/ml of serum (about 12M). Affinity of the antisera for the antigen is determined by preparing competitive binding curves, as described, for example, by Fisher, D., Chap. 42 in: Manual of Clinical Immunology, second edition, Rose and Friedman, eds., Amer. Soc. For Microbiology, Washington, D.C. (1980)

Antibody preparations prepared according to either protocol are useful in quantitative immunoassays which determine concentrations of antigen-bearing substances in biological samples; they are also used semi-quantitatively or qualitatively to identify the presence of antigen in a biological sample. In addition, antibodies are useful in various animal models of enterococcal disease as a means of evaluating the protein used to make the antibody as a potential vaccine target or as a means of evaluating the antibody as a potential immunotherapeutic or immunoprophylactic reagent.

4. Preparation of PCR Primers and Amplification of DNA

Various fragments of the Enterococcus faecalis genome, such as those of Tables 1-3 and SEQ ID NOS:1-982 can be used, in accordance with the present invention, to prepare PCR primers for a variety of uses. The PCR primers are preferably at least 15 bases, and more preferably at least 18 bases in length. When selecting a primer sequence, it is preferred that the primer pairs have approximately the same G/C ratio, so that melting temperatures are approximately the same. The PCR primers and amplified DNA of this Example find use in the Examples that follow.

5. Isolation of a Selected DNA Clone From the Deposited Sample of E. faecalis

Three approaches can be used to isolate a E. faecalis clone comprising a polynucleotide of the present invention from any E. faecalis genomic DNA library. The E. faecalis strain V586 has been deposited as a convenient source for obtaining a E. faecalis strain although a wide varity of strains E. faecalis strains can be used which are known in the art.

E. faecalis genomic DNA is prepared using the following method. A 20 ml overnight bacterial culture grown in a rich medium (e.g., Trypticase Soy Broth, Brain Heart Infusion broth or Super broth), pelleted, ished two times with TES (30 mM Tris-pH 8.0, 25 mM EDTA, 50 mM NaCl), and resuspended in 5 ml high salt TES (2.5M NaCl). Lysostaphin is added to final concentration of approx 50 ug/ml and the mixture is rotated slowly 1 hour at 37 C. to make protoplast cells. The solution is then placed in incubator (or place in a shaking water bath) and warmed to 55 C. Five hundred micro liter of 20% sarcosyl in TES (final concentration 2%) is then added to lyse the cells. Next, guanidine HCl is added to a final concentration of 7M (3.69 g in 5.5 ml). The mixture is swirled slowly at 55 C. for 60-90 min (solution should clear). A CsCl gradient is then set up in SW41 ultra clear tubes using 2.0 ml 5.7M CsCl and overlaying with 2.85M CsCl. The gradient is carefully overlayed with the DNA-containing GuHCl solution. The gradient is spun at 30,000 rpm, 20 C. for 24 hr and the lower DNA band is collected. The volume is increased to 5 ml with TE buffer. The DNA is then treated with protease K (10 ug/ml) overnight at 37 C., and precipitated with ethanol. The precipitated DNA is resuspended in a desired buffer.

In the first method, a plasmid is directly isolated by screening a plasmid E. faecalis genomic DNA library using a polynucleotide probe corresponding to a polynucleotide of the present invention. Particularly, a specific polynucleotide with 30-40 nucleotides is synthesized using an Applied Biosystems DNA synthesizer according to the sequence reported. The oligonucleotide is labeled, for instance, with ³²P-γ-ATP using T4 polynucleotide kinase and purified according to routine methods. (See, e.g., Maniatis et al., Molecular Cloning: A Laboratory Manual, Cold Spring Harbor Press, Cold Spring, N.Y. (1982).) The library is transformed into a suitable host, as indicated above (such as XL-1 Blue (Stratagene)) using techniques known to those of skill in the art. See, e.g., Sambrook et al. MOLECULAR CLONING: A LABORATORY MANUAL (Cold Spring Harbor, N.Y. 2nd ed. 1989); Ausubel et al., CURRENT PROTOCALS IN MOLECULAR BIOLOGY (John Wiley and Sons, N.Y. 1989). The transformants are plated on 1.5% agar plates (containing the appropriate selection agent, e.g., ampicillin) to a density of about 150 transformants (colonies) per plate. These plates are screened using Nylon membranes according to routine methods for bacterial colony screening. See, e.g., Sambrook et al. MOLECULAR CLONING: A LABORATORY MANUAL (Cold Spring Harbor, N.Y. 2nd ed. 1989); Ausubel et al., CURRENT PROTOCALS IN MOLECULAR BIOLOGY (John Wiley and Sons, N.Y. 1989) or other techniques known to those of skill in the art.

Alternatively, two primers of 15-25 nucleotides derived from the 5′ and 3′ ends of a polynucleotide of SEQ ID NOS:1-982 arc synthesized and used to amplify the desired DNA by PCR using a E. faecalis genomic DNA prep as a template. PCR is carried out under routine conditions, for instance, in 25 μl of reaction mixture with 0.5 ug of the above DNA template. A convenient reaction mixture is 1.5-5 mM MgCl₂, 0.01% (w/v) gelatin, 20 μM each of dATP, dCTP, dGTP, dTTP, 25 pmol of each primer and 0.25 Unit of Taq polymerase. Thirty five cycles of PCR (denaturation at 94° C. for 1 min. annealing at 55° C. for 1 min; elongation at 72° C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Finally, overlapping oligos of the DNA sequences of SEQ ID NOS:1-982 can be chemically synthesized and used to generate a nucleotide sequence of desired length using PCR methods known in the art.

6(a). Expression and Purification Enterococcal polypeptides in E. coli

The bacterial expression vector pQE60 was used for bacterial expression of some of the polypeptide fragements of the present invention which were used in the soft tissue and systemic infection models discussed below. (QIAGEN, Inc., 9259 Eton Avenue, Chatsworth, Calif., 91311). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin (QIAGEN, Inc., supra) and suitable single restriction enzyme cleavage sites. These elements are arranged such that an inserted DNA fragment encoding a polypeptide expresses that polypeptide with the six His residues (i.e., a “6× His tag”) covalently linked to the carboxyl terminus of that polypeptide.

The DNA sequence encoding the desired portion of a E. faecalis protein of the present invention was amplified from E. faecalis genomic DNA using PCR oligonucleotide primers which anneal to the 5′ and 3′ sequences coding for the portions of the E. faecalis polynucleotide shown in SEQ ID NOS:1-982. Additional nucleotides containing restriction sites to facilitate cloning in the pQE60 vector are added to the 5′ and 3′ sequences, respectively.

For cloning the mature protein, the 5′ primer has a sequence containing an appropriate restriction site followed by nucleotides of the amino terminal coding sequence of the desired E. faecalis polynucleotide sequence in SEQ ID NOS:1-982. One of ordinary skill in the art would appreciate that the point in the protein coding sequence where the 5′ and 3′ primers begin may be varied to amplify a DNA segment encoding any desired portion of the complete protein shorter or longer than the mature form. The 3′ primer has a sequence containing an appropriate restriction site followed by nucleotides complementary to the 3′ end of the polypeptide coding sequence of SEQ ID NOS:1-982, excluding a stop codon, with the coding sequence aligned with the restriction site so as to maintain its reading frame with that of the six His codons in the pQE60 vector.

The amplified E. faecalis DNA fragment and the vector pQE60 were digested with restriction enzymes which recognize the sites in the primers and the digested DNAs were then ligated together. The E. faecalis DNA was inserted into the restricted pQE60 vector in a manner which places the E. faecalis protein coding region downstream from the IPTG-inducible promoter and in-frame with an initiating AUG and the six histidine codons.

The ligation mixture was transformed into competent E. coli cells using standard procedures such as those described by Sambrook et al., supra.. E. coli strain M15/rep4, containing multiple copies of the plasmid pREP4, which expresses the lac repressor and confers kanamycin resistance (“Kanr”), was used in carrying out the illustrative example described herein. This strain, which was only one of many that are suitable for expressing a E. faecalis polypeptide, is available commercially (QIAGEN, Inc., supra). Transformants were identified by their ability to grow on LB agar plates in the presence of ampicillin and kanamycin. Plasmid DNA was isolated from resistant colonies and the identity of the cloned DNA confirmed by restriction analysis, PCR and DNA sequencing.

Clones containing the desired constructs were grown overnight (“O/N”) in liquid culture in LB media supplemented with both ampicillin (100 μg/ml) and kanamycin (25 μg/ml). The O/N culture was used to inoculate a large culture, at a dilution of approximately 1:25 to 1:250. The cells were grown to an optical density at 600 nm (“OD600”) of between 0.4 and 0.6. Isopropyl-β-D-thiogalactopyranoside (“IPTG”) was then added to a final concentration of 1 mM to induce transcription from the lac repressor sensitive promoter, by inactivating the lacI repressor. Cells subsequently were incubated further for 3 to 4 hours. Cells then were harvested by centrifugation.

The cells were then stirred for 3-4 hours at 4° C. in 6M guanidine-HCl, pH 8. The cell debris was removed by centrifugation, and the supernatant containing the E. faecalis polypeptide was loaded onto a nickel-nitrilo-tri-acetic acid (“Ni-NTA”) affinity resin column (QIAGEN, Inc., supra). Proteins with a 6× His tag bind to the Ni-NTA resin with high affinity were purified in a simple one-step procedure (for details see: The QIAexpressionist, 1995, QIAGEN, Inc., supra). Briefly the supernatant was loaded onto the column in 6 M guanidine-HCl, pH 8, the column was first washed with 10 volumes of 6 M guanidine-HCl, pH 8, then washed with 10 volumes of 6 M guanidine-HCl pH 6, and finally the E. faecalis polypeptide was eluted with 6 M guanidine-HCl, pH 5.

The purified protein was then renatured by dialyzing it against phosphate-buffered saline (PBS) or 50 mM Na-acetate, pH 6 buffer plus 200 mM NaCl. Alternatively, the protein could be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins can be eluted by the addition of 250 mM immidazole. Immidazole was removed by a final dialyzing step against PBS or 50 mM sodium acetate pH 6 buffer plus 200 mM NaCl. The purified protein was stored at 4° C. or frozen at −80° C.

Some of the polypeptide of the present invention were prepared using a non-denaturing protein purification method. For these polypeptides, the cell pellet from each liter of culture was resuspended in 25 mls of Lysis Buffer A at 4° C. (Lysis Buffer A=50 mM Na-phosphate, 300 mM NaCl, 10 mM 2-mercaptoethanol, 10% Glycerol, pH 7.5 with 1 tablet of Complete EDTA-free protease inhibitor cocktail (Boehringer Mannheim #1873580) per 50 ml of buffer). Absorbance at 550 nm was approximately 10-20 O.D./ml. The suspension was then put through three freeze/thaw cycles from −70° C. (using a ethanol-dry ice bath) up to room temperature. The cells were lysed via sonication in short 10 sec bursts over 3 minutes at approximately 80 W while kept on ice. The sonicated sample was then centrifuged at 15,000 RPM for 30 minutes at 4° C. The supernatant was passed through a column containing 1.0 ml of CL-4B resin to pre-clear the sample of any proteins that may bind to agarose non-specifically, and the flow-through fraction was collected.

The pre-cleared flow-through was applied to a nickel-nitrilo-tri-acetic acid (“Ni-NTA”) affinity resin column (Quiagen, Inc., supra). Proteins with a 6× His tag bind to the Ni-NTA resin with high affinity and can be purified in a simple one-step procedure. Briefly, the supernatant was loaded onto the column in Lysis Buffer A at 4° C., the column was first washed with 10 volumes of Lysis Buffer A until the A280 of the eluate returns to the baseline. Then, the column was washed with 5 volumes of 40 mM Imidazole (92% Lysis Buffer A/8% Buffer B) (Buffer B=50 mM Na-Phosphate, 300 mM NaCl, 10% Glycerol, 10 mM 2-mercaptoethanol, 500 mM Imidazole, pH of the final buffer should be 7.5). The protein was eluted off of the column with a series of increasing Imidazole solutions made by adjusting the ratios of Lysis Buffer A to Buffer B. Three different concentrations were used: 3 volumes of 75 mM Imidazole, 3 volumes of 150 mM Imidazole, 5 volumes of 500 mM Imidazole. The fractions containing the purified protein were analyzed using 8%, 10% or 14% SDS-PAGE depending on the protein size. The purified protein was then dialyzed 2× against phosphate-buffered saline (PBS) in order to place it into an easily workable buffer. The purified protein was stored at 4° C. or frozen at −80°.

The following alternative method may be used to purify E. faecalis expressed in E coli when it is present in the form of inclusion bodies. Unless otherwise specified, all of the following steps are conducted at 4-10° C.

Upon completion of the production phase of the E. coli fermentation, the cell culture is cooled to 4-10° C. and the cells are harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogereous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the E. faecalis polypeptide-containing supernatant is incubated at 4° C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded E. faecalis polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 mm of the effluent is continuously monitored. Fractions are collected and further analyzed by SDS-PAGE.

Fractions containing the E. faecalis polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the E. faecalis polypeptide (determined, for instance, by 16% SDS-PAGE) are then pooled.

The resultant E. faecalis polypeptide exhibits greater than 95% purity after the above refolding and purification steps. No major contaminant bands are observed from Commassie blue stained 16% SDS-PAGE gel when 5 μg of purified protein is loaded. The purified protein is also tested for endotoxin/LPS contamination, and typically the LPS content is less than 0.1 ng/ml according to LAL assays.

6(b). Alternative Expression and Purification Enterococcal Polypeptides in E. coli

The vector pQE10 was alternatively used to clone and express some of the polypeptides of the present invention for use in the soft tissue and systemic infection models discussed below. The difference being such that an inserted DNA fragment encoding a polypeptide expresses that polypeptide with the six His residues (i.e., a “6× His tag”) covalently linked to the amino terminus of that polypeptide. The bacterial expression vector pQE10 (QIAGEN, Inc., 9259 Eton Avenue, Chatsworth, Calif., 91311) was used in this example. The components of the pQE10 plasmid are arranged such that the inserted DNA sequence encoding a polypeptide of the present invention expresses the polypeptide with the six His residues (i.e., a “6× His tag”)) covalently linked to the amino terminus.

The DNA sequences encoding the desired portions of a polypeptide of SEQ ID NOS:1-982 were amplified using PCR oligonucleotide primers from genomic E. faecalis DNA. The PCR primers anneal to the nucleotide sequences encoding the desired amino acid sequence of a polypeptide of the present invention. Additional nucleotides containing restriction sites to facilitate cloning in the pQE10 vector were added to the 5′ and 3′ primer sequences, respectively.

For cloning a polypeptide of the present invention, the 5′ and 3′ primers were selected to amplify their respective nucleotide coding sequences. One of ordinary skill in the art would appreciate that the point in the protein coding sequence where the 5′ and 3′ primers begins may be varied to amplify a DNA segment encoding any desired portion of a polypeptide of the present invention. The 5′ primer was designed so the coding sequence of the 6× His tag is aligned with the restriction site so as to maintain its reading frame with that of E. faecalis polypeptide. The 3′ was designed to include an stop codon. The amplified DNA fragment was then cloned, and the protein expressed, as described above for the pQE60 plasmid.

The DNA sequences encoding the amino acid sequences of SEQ ID NOS:1-982 may also be cloned and expressed as fusion proteins by a protocol similar to that described directly above, wherein the pET-32b(+) vector (Novagen, 601 Science Drive, Madison, Wis. 53711) is preferentially used in place of pQE10.

The above methods are not limited to the polypeptide fragements actually produced. The above method, like the methods below, can be used to produce either full length polypeptides or desired fragements therof.

6(c). Alternative Expression and Purification of Enterococcal Polypeptides in E. coli

The bacterial expression vector pQE60 is used for bacterial expression in this example (QIAGEN, Inc., 9259 Eton Avenue, Chatsworth, Calif., 91311). However, in this example, the polypeptide coding sequence is inserted such that translation of the six His codons is prevented and, therefore, the polypeptide is produced with no 6× His tag.

The DNA sequence encoding the desired portion of the E. faecalis amino acid sequence is amplified from an E. faecalis genomic DNA prep the deposited DNA clones using PCR oligonucleotide primers which anneal to the 5′ and 3′ nucleotide sequences corresponding to the desired portion of the E. faecalis polypeptides. Additional nucleotides containing restriction sites to facilitate cloning in the pQE60 vector are added to the 5′ and 3′ primer sequences.

For cloning a E. faecalis polypeptides of the present invention, 5′ and 3′ primers are selected to amplify their respective nucleotide coding sequences. One of ordinary skill in the art would appreciate that the point in the protein coding sequence where the 5′ and 3′ primers begin may be varied to amplify a DNA segment encoding any desired portion of a polypeptide of the present invention. The 3′ and 5′ primers contain appropriate restriction sites followed by nucleotides complementary to the 5′ and 3′ ends of the coding sequence respectively. The 3′ primer is additionally designed to include an in-frame stop codon.

The amplified E. faecalis DNA fragments and the vector pQE60 are digested with restriction enzymes recognizing the sites in the primers and the digested DNAs are then ligated together. Insertion of the E. faecalis DNA into the restricted pQE60 vector places the E. faecalis protein coding region including its associated stop codon downstream from the IPTG-inducible promoter and in-frame with an initiating AUG. The associated stop codon prevents translation of the six histidine codons downstream of the insertion point.

The ligation mixture is transformed into competent E. coli cells using standard procedures such as those described by Sambrook et al. E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing E. faecalis polypeptide, is available commercially (QIAGEN, Inc., supra). Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

To purify the E. faecalis polypeptide, the cells are then stirred for 3-4 hours at 4° C. in 6M guanidine-HCl, pH 8. The cell debris is removed by centrifugation, and the supernatant containing the E. faecalis polypeptide is dialyzed against 50 mM Na-acetate buffer pH 6, supplemented with 200 mM NaCl. Alternatively, the protein can be successfully refolded by dialyzing it against 500 mM NaCl, 20% glycerol, 25 mM Tris/HCl pH 7.4, containing protease inhibitors. After renaturation the protein can be purified by ion exchange, hydrophobic interaction and size exclusion chromatography. Alternatively, an affinity chromatography step such as an antibody column can be used to obtain pure E. faecalis polypeptide. The purified protein is stored at 4° C. or frozen at −80° C.

The following alternative method may be used to purify E. faecalis polypeptides expressed in E coli when it is present in the form of inclusion bodies. Unless otherwise specified, all of the following steps are conducted at 4-10° C.

Upon completion of the production phase of the E. coli fermentation, the cell culture is cooled to 4-10 ° C. and the cells are harvested by continuous centrifugation at 15,000 rpm (Heracus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells ware then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the E. faecalis polypeptide-containing supernatant is incubated at 4° C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded E. faecalis polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 mm of the effluent is continuously monitored. Fractions are collected and further analyzed by SDS-PAGE.

Fractions containing the E. faecalis polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the E. faecalis polypeptide (determined, for instance, by 16% SDS-PAGE) are then pooled.

The resultant E. faecalis polypeptide exhibits greater than 95% purity after the above refolding and purification steps. No major contaminant bands are observed from Commassie blue stained 16% SDS-PAGE gel when 5 μg of purified protein is loaded. The purified protein is also tested for endotoxin/LPS contamination, and typically the LPS content is less than 0.1 ng/ml according to LAL assays.

6(d). Cloning and Expression of E. faecalis in Other Bacteria

E. faecalis polypeptides can also be produced in: E. faecalis using the methods of S. Skinner et al., (1988) Mol. Microbiol. 2:289-297 or J. I. Moreno (1996) Protein Expr. Purif. 8(3):332-340; Lactobacillus using the methods of C. Rush et al., 1997 Appl. Microbiol. Biotechnol. 47(5):537-542; or in Bacillus subtilis using the methods Chang et al., U.S. Pat. No. 4,952,508.

7. Cloning and Expression in COS Cells

A E. faecalis expression plasmid is made by cloning a portion of the DNA encoding a E. faecalis polypeptide into the expression vector pDNAI/Amp or pDNAIII (which can be obtained from Invitrogen, Inc.). The expression vector pDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) followed by a termination codon and polyadenylation signal arranged so that a DNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson et al. 1984 Cell 37:767. The fusion of the HA tag to the target protein allows easy detection and recovery of the recombinant protein with an antibody that recognizes the HA epitope. pDNAIII contains, in addition, the selectable neomycin marker.

A DNA fragment encoding a E. faecalis polypeptide is cloned into the polylinker region of the vector so that recombinant protein expression is directed by the CMV promoter. The plasmid construction strategy is as follows. The DNA from a E. faecalis genomic DNA prep is amplified using primers that contain convenient restriction sites, much as described above for construction of vectors for expression of E. faecalis in E. coli. The 5′ primer contains a Kozak sequence, an AUG start codon, and nucleotides of the 5′ coding region of the E. faecalis polypeptide. The 3′ primer, contains nucleotides complementary to the 3′ coding sequence of the E. faecalis DNA, a stop codon, and a convenient restriction site.

The PCR amplified DNA fragment and the vector, pDNAI/Amp, are digested with appropriate restriction enzymes and then ligated. The ligation mixture is transformed into an appropriate E. coli strain such as SURE™ (Stratagene Cloning Systems, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the fragment encoding the E. faecalis polypeptide

For expression of a recombinant E. faecalis polypeptide, COS cells are transfected with an expression vector, as described above, using DEAE-dextran, as described, for instance, by Sambrook et al. (supra). Cells are incubated under conditions for expression of E. faecalis by the vector.

Expression of the E. faecalis-HA fusion protein is detected by radiolabeling and immunoprecipitation, using methods described in, for example Harlow et al., supra.. To this end, two days after transfection, the cells are labeled by incubation in media containing ³⁵S-cysteine for 8 hours. The cells and the media are collected, and the cells are washed and the lysed with detergent-containing RIPA buffer: 150 mM NaCl, 1% NP-40, 0.1% SDS, 1% NP-40, 0.5% DOC, 50 mM TRIS, pH 7.5, as described by Wilson et al. (supra ). Proteins are precipitated from the cell lysate and from the culture media using an HA-specific monoclonal antibody. The precipitated proteins then are analyzed by SDS-PAGE and autoradiography. An expression product of the expected size is seen in the cell lysate, which is not seen in negative controls.

8. Cloning and Expression in CHO Cells

The vector pC4 is used for the expression of E. faecalis polypeptide in this example. Plasmid pC4 is a derivative of the plasmid pSV2-dhfr (ATCC Accession No. 37146). The plasmid contains the mouse DHFR gene under control of the SV40 early promoter. Chinese hamster ovary cells or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented. See, e.g., Alt et al., 1978, J. Biol. Chem. 253:1357-1370; Hamlin et al., 1990, Biochem. et Biophys. Acta, 1097:107-143; Page et al., 1991, Biotechnology 9:64-68. Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains the strong promoter of the long terminal repeat (LTR) of the Rouse Sarcoma Virus, for expressing a polypeptide of interest, Cullen, et al. (1985) Mol. Cell. Biol. 5:438-447; plus a fragment isolated from the enhancer of the immediate early gene of human cytomegalovirus (CMV), Boshart, et al., 1985, Cell 41:521-530. Downstream of the promoter are the following single restriction enzyme cleavage sites that allow the integration of the genes: Bam HI, Xba I, and Asp 718. Behind these cloning sites the plasmid contains the 3′ intron and polyadenylation site of the rat preproinsulin gene. Other high efficiency promoters can also be used for the expression, e.g., the human β-actin promoter, the SV40 early or late promoters or the long terminal repeats from other retroviruses, e.g., HIV and HTLVI. Clontech's Tet-Off and Tet-On gene expression systems and similar systems can be used to express the E. faecalis polypeptide in a regulated way in mammalian cells (Gossen et al., 1992, Proc. Natl. Acad. Sci. USA 89:5547-5551. For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with the restriction enzymes and then dephosphorylated using calf intestinal phosphates by procedures known in the art. The vector is then isolated from a 1% agarose gel. The DNA sequence encoding the E. faecalis polypeptide is amplified using PCR oligonucleotide primers corresponding to the 5′ and 3′ sequences of the desired portion of the gene. A 5′ primer containing a restriction site, a Kozak sequence, an AUG start codon, and nucleotides of the 5′ coding region of the E. faecalis polypeptide is synthesized and used. A 3′ primer, containing a restriction site, stop codon, and nucleotides complementary to the 3′ coding sequence of the E. faecalis polypeptides is synthesized and used. The amplified fragment is digested with the restriction endonucleases and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. Five μg of the expression plasmid pC4 is cotransfected with 0.5 μg of the plasmid pSVneo using a lipid-mediated transfection agent such as Lipofectin™ or LipofectAMINE.™ (LifeTechnologies Gaithersburg, Md.). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

9. Quantitative Murine Soft Tissue Infection Model for E. faecalis

Compositions of the present invention, including polypeptides and peptides, are assayed for their ability to function as vaccines or to enhance/stimulate an immune response to a bacterial species (e.g., E. faecalis) using the following quantitative murine soft tissue infection model. Mice (e.g., NIH Swiss female mice, approximately 7 weeks old) are first treated with a biologically protective effective amount, or immune enhancing/stimulating effective amount of a composition of the present invention using methods known in the art, such as those discussed above. See, e.g., Harlow et al., ANTIBODIES: A LABORATORY MANUAL, (Cold Spring Harbor Laboratory Press, 2nd ed. 1988). An example of an appropriate starting dose is 20 ug per animal.

The desired bacterial species used to challenge the mice, such as E. faecalis, is grown as an overnight culture. The culture is diluted to a concentration of 5×10⁸ cfu/ml, in an appropriate media, mixed well, serially diluted, and titered. The desired doses are further diliuted 1:2 with sterilized Cytodex 3 microcarrier beads preswollen in sterile PBS (3 g/100 ml). Mice are anesthetize briefly until docile, but still mobile and injected with 0.2 ml of the Cytodex 3 bead/bacterial mixture into each animal subcutaneously in the inguinal region. After four days, counting the day of injection as day one, mice are sacrificed and the contents of the abscess is excised and placed in a 15 ml conical tube containing 1.0 ml of sterile PBS. The contents of the abscess is then enzymatically treated and plated as follows.

The abscess is first disrupted by vortexing with sterilized glass beads placed in the tubes. 3.0 mls of prepared enzyme mixture (1.0 ml Collagenase D (4.0 mg/ml), 1.0 ml Trypsin (6.0 mg/ml) and 8.0 mls PBS) is then added to each tube followed by a 20 min. incubation at 37 C. The solution is then centrifuged and the supernatant drawn off. 0.5 ml dH20 is then added and the tubes are vortexed and then incubated for 10 min. at room temperature. 0.5 ml media is then added and samples are serially diluted and plated onto agar plates, and grown overnight at 37 C. Plates with distinct and separate colonies are then counted, compared to positive and negative control samples, and quantified. The method can be used to identify composition and determine appropriate and effective doses for humans and other animals by comparing the effective doses of compositions of the present invention with compositions known in the art to be effective in both mice and humans. Doses for the effective treatment of humans and other animals, using compositions of the present invention, are extrapolated using the data from the above experiments of mice. It is appreciated that further studies in humans and other animals may be needed to determine the most effective doses using methods of clinical practice known in the art.

10. Murine Systemic Neutropenic Model for E. faecalis Infection Compositions of the present invention, including polypeptides and peptides, are assayed for their ability to function as vaccines or to enhance/stimulate an immune response to a bacterial species (e.g., E. faecalis) using the following qualitative murine systemic neutropenic model. Mice (e.g., NIH Swiss female mice, approximately 7 weeks old) are first treated with a biologically protective effective amount, or immune enhancing/stimulating effective amount of a composition of the present invention using methods known in the art, such as those discussed above. See, e.g., Harlow et al., ANTIBODIES: A LABORATORY MANUAL, (Cold Spring Harbor Laboratory Press, 2nd ed. 1988). An example of an appropriate starting dose is 20 ug per animal. Mice are then injected with 250-300 mg/kg cyclophosphamide intraperitonially. Counting the day of C.P. injection as day one, the mice are left untreated for 5 days to begin recovery of PMNL'S.

The desired bacterial species used to challenge the mice, such as E. faecalis, is grown as an overnight culture. The culture is diluted to a concentration of 5×10⁸ cfu/ml, in an appropriate media, mixed well, serially diluted, and titered. The desired doses are further diliuted 1:2 in 4% Brewer's yeast in media. Mice are injected with the bacteria/brewer's yeast challenge intraperitonially. The Brewer's yeast solution alone is used as a control. The mice are then monitered twice daily for the first week following challenge, and once a day for the next week to ascertain morbidity and mortality. Mice remaining at the end of the experiment are sacrificed. The method can be used to identify compositions and determine appropriate and effective doses for humans and other animals by comparing the effective doses of compositions of the present invention with compositions known in the art to be effective in both mice and humans. Doses for the effective treatment of humans and other animals, using compositions of the present invention, are extrapolated using the data from the above experiments of mice. It is appreciated that further studies in humans and other animals may be needed to determine the most effective doses using methods of clinical practice known in the art.

The disclosure of all publications (including patents, patent applications, journal articles, laboratory manuals, books, or other documents) cited herein are hereby incorporated by reference in their entireties.

The present invention is not to be limited in scope by the specific embodiments described herein, which are intended as single illustrations of individual aspects of the invention. Functionally equivalent methods and components are within the scope of the invention, in addition to those shown and described herein and will become apparant to those skilled in the art from the foregoing description and accompanying drawings. Such modifications are intended to fall within the scope of the appended claims.

TABLE 1
E. faecalis-Coding regions containing known sequences

| Contig | Orf | Start | Stop | | | Percent | HSP nt |
|---|---|---|---|---|---|---|---|
| ID | ID | (nt) | (nt) | Match Accession | Match Gene Name | Indent | length |
| 3 | 2 | 423 | 1226 | gb\|U24692\| | “Enterococcus faecalis pyrimidine | 99 | 229 |
| | | | | | biosynthesis D (pyrD) gene, complete cds” | | |
| 47 | 14 | 17085 | 16216 | gb\|M81466\| | “Enterococcus faecalis RecA protein (recA) | 98 | 308 |
| | | | | | gene, partial cds” | | |
| 52 | 1 | 50 | 1441 | emb\|X62755\|SFNPRG | S.faecalis npr gene for NADH peroxidase | 98 | 1374 |
| 52 | 2 | 2456 | 1494 | emb\|X62755\|SFNPRG | S.faecalis npr gene for NADH peroxidase | 100 | 209 |
| 61 | 1 | 2 | 358 | gb\|U35369\| | “Enterococcus faecalis vancomycin | 99 | 318 |
| | | | | | resistance genes, response regulator | | |
| | | | | | (vanRB), protein histidine kinase (vanSB), | | |
| | | | | | D,D-carboxypeptidase (vanYB), putative D- | | |
| | | | | | 2-hydroxyacid dehydrogenase (vanHB), D- | | |
| | | | | | Ala:D-Lac ligase (vanB), and putative D,D- | | |
| | | | | | dipeptidase (vanX>” | | |
| 61 | 2 | 467 | 1975 | gb\|U35369\| | “Enterococcus faecalis vancomycin | 98 | 1297 |
| | | | | | resistance genes, response regulator | | |
| | | | | | (vanRB), protein histidine kinase (vanSB), | | |
| | | | | | D,D-carboxypeptidase (vanYB), putative D- | | |
| | | | | | 2-hydroxyacid dehydrogenase (vanHB), D- | | |
| | | | | | Ala:D-Lac ligase (vanB), and putative D,D- | | |
| | | | | | dipeptidase (vanX>” | | |
| 61 | 3 | 1749 | 1967 | gb\|U35369\| | “Enterococcus faecalis vancomycin | 100 | 136 |
| | | | | | resistance genes, response regulator | | |
| | | | | | (vanRB), protein histidine kinase (vanSB), | | |
| | | | | | D,D-carboxypeptidase (vanYB), putative D- | | |
| | | | | | 2-hydroxyacid dehydrogenase (vanHB), | | |
| | | | | | Ala:D-Lac ligase (vanB), and putative D,D- | | |
| | | | | | dipeptidase (vanX>” | | |
| 61 | 4 | 1990 | 2949 | gb\|U35369\| | “Enterococcus faecalis vancomycin | 100 | 960 |
| | | | | | resistance genes, response regulator | | |
| | | | | | (vanRB), protein histidine kinase (vanSB), | | |
| | | | | | D,D-carboxypeptidase (vanYB), putative D- | | |
| | | | | | 2-hydroxyacid dehydrogenase (vanHB), D | | |
| | | | | | Ala:D-Lac ligase (vanB), and putative D,D- | | |
| | | | | | dipeptidase (vanX>” | | |
| 61 | 5 | 2112 | 2399 | gb\|U35369\| | “Enterococcus faecalis vancomycin | 100 | 288 |
| | | | | | resistance genes, response regulator | | |
| | | | | | (vanRB), protein histidine kinase (vanSB), | | |
| | | | | | D,D-carboxypeptidase (vanYB), putative D- | | |
| | | | | | 2-hydroxyacid dehydrogenase (vanHB), D- | | |
| | | | | | Ala:D-Lac ligase (vanB), and putative D,D- | | |
| | | | | | dipeptidase (vanX>” | | |
| 61 | 6 | 2922 | 3794 | gb\|U35369\| | “Enterococcus faecalis vancomycin | 100 | 873 |
| | | | | | resistance genes, response regulator | | |
| | | | | | (vanRB), protein histidine kinase (vanSB), | | |
| | | | | | D,D-carboxypeptidase (vanYB), putative D- | | |
| | | | | | 2-hydroxyacid dehydrogenase (vanHB), D- | | |
| | | | | | Ala:D-Lac ligase (vanB), and putative D,D- | | |
| | | | | | dipeptidase (vanX>” | | |
| 61 | 7 | 3671 | 4762 | gb\|U35369\| | “Enterococcus faecalis vancomycin | 99 | 1092 |
| | | | | | resistance genes, response regulator | | |
| | | | | | (vanRB), protein histidine kinase (vanSB), | | |
| | | | | | D,D-carboxypeptidase (vanYB), putative D- | | |
| | | | | | 2-hydroxyacid dehydrogenase (vanHB), D- | | |
| | | | | | Ala:D-Lac ligase (vanB), and putative D,D- | | |
| | | | | | dipeptidase (vanX>” | | |
| 61 | 8 | 4312 | 3860 | gb\|U35369\| | “Enterococcus faecalis vancomycin | 100 | 453 |
| | | | | | resistance genes, response regulator | | |
| | | | | | (vanRB), protein histidine kinase (vanSB), | | |
| | | | | | D,D-carboxypeptidase (vanYB), putative D- | | |
| | | | | | 2-hydroxyacid dehydrogenase (vanHB), D- | | |
| | | | | | Ala:D-Lac ligase (vanB), and putative D,D- | | |
| | | | | | dipeptidase (vanX>” | | |
| 61 | 9 | 4653 | 5783 | gb\|U35369\| | “Enterococcus faecalis vancomycin | 100 | 1131 |
| | | | | | resistance genes, response regulator | | |
| | | | | | (vanRB), protein histidine kinase (vanSB), | | |
| | | | | | D,D-carboxypeptidase (vanYB), putative D- | | |
| | | | | | 2-hydroxyacid dehydrogenase (vanHB), D- | | |
| | | | | | Ala:D-Lac ligase (vanB), and putative D,D- | | |
| | | | | | dipeptidase (vanX>” | | |
| 61 | 10 | 5750 | 6397 | gb\|U35369\| | “Enterococcus faecalis vancomyc2-fl | 99 | 648 |
| | | | | | resistance genes, response regulator | | |
| | | | | | (vanRB), protein histidine kinase (vanSB), | | |
| | | | | | D,D-carboxypeptidase (vanYB), putative D- | | |
| | | | | | 2-hydroxyacid dehydrogenase (vanHB), D- | | |
| | | | | | Ala:D-Lac ligase (vanB), and putative D,D- | | |
| | | | | | dipeptidase (vanX>” | | |
| 61 | 11 | 7158 | 6784 | gb\|U35369\| | “Enterococcus faecalis vancomycin | 100 | 161 |
| | | | | | resistance genes, response regulator | | |
| | | | | | (vanRB), protein histidine kinase (vanSB), | | |
| | | | | | D,D-carboxypeptidase (vanYB), putative D- | | |
| | | | | | 2-hydroxyacid dehydrogenase (vanHB), D- | | |
| | | | | | Ala:D-Lac ligase (vanB), and putative D,D- | | |
| | | | | | dipeptidase (vanX>” | | |
| 67 | 1 | 3 | 809 | gb\|U24692” | “Enterococcus faecalis pyrimidine | 98 | 807 |
| | | | | | biosynthesis D (pyrD) gene, complete cds” | | |
| 67 | 2 | 781 | 1512 | gb\|U24692\| | “Enterococcus faecalis pyrimidine | 93 | 92 |
| | | | | | biosynthesis D (pyrD) gene, complete cds” | | |
| 69 | 1 | 1 | 228 | gb\|U60038\| | “Enterococcus faecalis major cold-shock | 100 | 136 |
| | | | | | protein (cspA) gene, partial cds” | | |
| 72 | 15 | 15814 | 19737 | emb\|X62656\|EFASP1 | “E.faecalis plasmid pPD1 aspl and URFs | 92 | 2504 |
| | | | | | pd57, pd125 and pd113 genes” | | |
| 72 | 16 | 19739 | 20155 | emb\|X62657\|EFORF3 | E.faecalis plasmid pAD1 DNA for orf3 | 96 | 341 |
| 75 | 1 | 3 | 365 | emb\|Z19137\|EFPTSHGN | E.faecalis of ptsH gene encoding HPr | 100 | 267 |
| 83 | 12 | 8766 | 7432 | emb\|X78425\|EFPBP5 | E.faecalis pbp5 gene | 98 | 416 |
| 83 | 13 | 8869 | 9699 | emb\|X78425\|EFPBP5 | E.faecalis pbp5 gene | 99 | 819 |
| 83 | 14 | 9612 | 10913 | emb\|X78425\|EFPBP5 | E.faecalis pbp5 gene | 99 | 1203 |
| 83 | 15 | 10943 | 11746 | emb\|X78425\|EFPBP5 | E.faecalis pbp5 gene | 97 | 286 |
| 84 | 2 | 1657 | 3558 | emb\|X86176\|EFRPODDNE | E.faecalis dnaE and rpoD gene | 99 | 797 |
| 84 | 3 | 3649 | 4773 | emb\|X86176\|EFRPODDNE | E.faecalis dnaE and rpoD gene | 99 | 1125 |
| 84 | 4 | 4913 | 7000 | emb\|X86176\|EFRPODDNE | E.faecalis dnaE and rpoD gene | 99 | 301 |
| 104 | 2 | 4018 | 2900 | gb\|U36195\| | “Enterococcus faecalis pyrAa gene, partial | 93 | 310 |
| | | | | | cds” | | |
| 108 | 7 | 5875 | 5183 | gb\|M58002\| | “Streptococcus faecalis bacterial cell | 98 | 252 |
| | | | | | wall hydrolase gene, complete cds” | | |
| 145 | 8 | 8193 | 7234 | gb\|U03756\| | “Enterococcus faecalis endocarditis | 99 | 960 |
| | | | | | specific antigen gene, complete cds” | | |
| 145 | 9 | 8836 | 8147 | gb\|U03756\| | “Enterococcus faecalis endocarditis | 100 | 132 |
| | | | | | specific antigen gene, complete cds” | | |
| 147 | 3 | 2096 | 3418 | emb\|X68847\|SFNOXAA | S.faecalis nox gene for NADH oxidase | 99 | 1301 |
| 154 | 4 | 2160 | 2492 | emb\|X17O92\|PPRRA | Plasmid pAM-beta-1 (from S.faecalis) | 93 | 294 |
| | | | | | replication region DNA | | |
| 154 | 10 | 5935 | 6294 | gb\|U17153\| | “Enterococcus faecalis plasmid pjh1 | 99 | 355 |
| | | | | | tetracycline resistant (tetL) gene, | | |
| | | | | | complete cds” | | |
| 154 | 11 | 6279 | 6584 | gb\|U17153\| | “Enterococcus faecalis plasmid pjh1 | 98 | 89 |
| | | | | | tetracycline resistant (tetL) gene, | | |
| | | | | | complete cds” | | |
| 154 | 12 | 7882 | 7097 | gb\|U86375\| | “Enterococcus faecalis ermB regulator and | 99 | 736 |
| | | | | | adenine methylase (ermB) genes, complete | | |
| | | | | | cds” | | |
| 154 | 13 | 8750 | 8043 | gb\|U17153\| | “Enterococcus faecalis plasmid pjh1 | 99 | 498 |
| | | | | | tetracycline resistant (tetL) gene, | | |
| | | | | | complete cds” | | |
| 159 | 1 | 158 | 1483 | gb\|M58002\| | “Streptococcus faecalis bacterial cell | 98 | 1323 |
| | | | | | wall hydrolase gene, complete cds” | | |
| 159 | 2 | 807 | 157 | gb\|M58002\| | “Streptococcus faecalis bacterial cell | 99 | 651 |
| | | | | | wall hydrolase gene, complete cds” | | |
| 159 | 3 | 1395 | 2192 | gb\|M58002\| | “Streptococcus faecalis bacterial cell | 93 | 350 |
| | | | | | wall hydrolase gene, complete cds” | | |
| 216 | 2 | 282 | 1841 | gb\|M90060\| | “Streptococcus faecalis H+ ATPase a | 81 | 1558 |
| | | | | | (atpB),b (atpF),c (atpE),alpha (atpA), | | |
| | | | | | beta (atpD),gamma (atpG),delta (atpH),and | | |
| | | | | | epsilon (atpC) subunits, complete cds” | | |
| 216 | 4 | 2809 | 2967 | gb\|M90060\| | “Streptococcus faecalis H+ATPase a | 86 | 132 |
| | | | | | (atpB),b (atpF),c (atpE),alpha (atpA), | | |
| | | | | | beta (atpD) ,gamma (atpG) ,delta (atpH) ,and | | |
| | | | | | epsilon (atpC) subunits, complete cds” | | |
| 216 | 5 | 2940 | 4244 | gb\|M90060\| | “Streptococcus faecalis H+ ATPase a | 83 | 1293 |
| | | | | | (atpB),b (atpF),c (atpE),alpha (atpA), | | |
| | | | | | beta (atpD) ,gamma (atpG) ,delta (atpH) ,and | | |
| | | | | | epsilon (atpC) subunits, complete cds” | | |
| 238 | 3 | 1814 | 2218 | gb\|M38386\| | “Streptococcus faecalis mtlF enzymeIII, | 96 | 302 |
| | | | | | mannitol-mtlD-phosphate- dehydrogenase” | | |
| 238 | 4 | 2182 | 2670 | gb\|M38386\| | “Streptococcus faecalis mtlF enzymeIII, | 98 | 480 |
| | | | | | mannitol-mtlD-phosphate- dehydrogenase” | | |
| 238 | 5 | 2634 | 3839 | gb\|M38386\| | “Streptococcus faecalis mtlF enzymeIII, | 96 | 459 |
| | | | | | mannitol-mtlD-phosphate- dehydrogenase” | | |
| 261 | 2 | 1397 | 510 | emb\|Z12296\|EFSPREG | E.faecalis sprE gene for serine proteinase | 98 | 888 |
| | | | | | homologue | | |
| 261 | 3 | 2474 | 1413 | dbj\|D85393\|ENEGE1E | “Enterococcus faecalis DNA for gelatinase, | 98 | 1051 |
| | | | | | complete cds” | | |
| 261 | 4 | 2974 | 2417 | dbj\|D85393\|ENEGE1E | “Enterococcus faecalis DNA for gelatinase, | 97 | 516 |
| | | | | | complete cds” | | |
| 275 | 3 | 1472 | 1044 | gb\|L23802\| | “Enterococcus faecalis pore forming, cell | 98 | 422 |
| | | | | | wall enzyme, regulatory, and | | |
| | | | | | dehydroquinase homologue proteins | | |
| | | | | | (ebsA,ebsB,ebsC,and ebsD) genes, complete | | |
| | | | | | cds with repeat region” | | |
| 275 | 4 | 1581 | 2018 | gb\|L23802\| | “Enterococcus faecalis pore forming, cell | 97 | 438 |
| | | | | | wall enzyme, regulatory, and | | |
| | | | | | dehydroguinase homologue proteins | | |
| | | | | | (ebsA, ebsB, ebsC, and ebsD) genes, complete | | |
| | | | | | cds with repeat region” | | |
| 275 | | 5 2789 | 2148 | gb\|L23802\| | “Enterococcus faecalis pore forming, cell | 98 | 642 |
| | | | | | wall enzyme, regulatory, and | | |
| | | | | | dehydroquinase homologue proteins | | |
| | | | | | (ebsA, ebsB, ebsC, and ebsD) genes, complete | | |
| | | | | | cds with repeat region” | | |
| 275 | 6 | 3475 | 2660 | gb\|L23802\| | “Enterococcus faecalis pore forming, cell | 98 | 790 |
| | | | | | wall enzyme, regulatory, and | | |
| | | | | | dehydroquinase homologue proteins | | |
| | | | | | (ebsA, ebsB, ebsC, and ebsD) genes, complete | | |
| | | | | | cds with repeat region” | | |
| 287 | 2 | 1565 | 558 | emb\|X17092\|PPRRA | Plasmid pAM-beta-1 (from S.faecalis) | 97 | 991 |
| | | | | | replication region DNA | | |
| 287 | 3 | 2049 | 1582 | emb\|X17092\|PPRRA | Plasmid pAM-beta-1 (from S.faecalis) | 97 | 461 |
| | | | | | replication region DNA | | |
| 287 | 6 | 2639 | 3346 | gb\|U17153\| | “Enterococcus faecalis plasmid pjh1 | 99 | 498 |
| | | | | | tetracycline resistant (tetL) gene, | | |
| | | | | | complete cds” | | |
| 294 | 11 | 4519 | 4211 | gb\|U17153\| | “Enterococcus faecalis plasmid pjh1 | 100 | 50 |
| | | | | | tetracycline resistant (tetL) gene, | | |
| | | | | | complete cds” | | |
| 302 | 1 | 1 | 1755 | emb\|X62658\|EFSEA1 | E.faecalis plasmid pAD1 seal gene and orfy | 83 | 1755 |
| 302 | 2 | 2310 | 2687 | emb\|X17214\|SFPASA1 | S. faecalis plasmid pAD1 asal gene for | 100 | 378 |
| | | | | | aggregation substance and ORF 1 | | |
| 302 | 3 | 2865 | 3329 | emb\|X17214\|SPPASA1 | S. faecalis plasmid pAD1 asal gene for | 99 | 463 |
| | | | | | aggregation substance and ORF 1 | | |
| 316 | 4 | 2724 | 2110 | gb\|M13771\| | “Streptococcus faecalis 6′-aminoglycoside | 100 | 248 |
| | | | | | acetyltransferase phosphotransferase | | |
| | | | | | (AAC(6′)-APH(2′)) bifunctional resistance | | |
| | | | | | protein, complete cds” | | |
| 346 | 5 | 2224 | 2880 | emb\|X62755\|SFNPRG | S.faecalis npr gene for NADH peroxidase | 98 | 351 |
| 349 | 2 | 686 | 907 | dbj\|D78257\|D78257 | “Enterococcus faecalis plasmid pYI17 genes | 83 | 200 |
| | | | | | for BacA, BacB, ORF3, ORF4, ORF5, ORF6, | | |
| | | | | | ORF7, ORF8, ORF9, ORF10, ORF11,partial | | |
| | | | | | cds” | | |
| 355 | 1 | 3 | 1166 | emb\|X17214\|SFPASA1 | S. faecalis plasmid pAD1 asal gene for | 97 | 1100 |
| | | | | | aggregation substance and ORF 1 | | |
| 355 | 2 | 1102 | 1548 | emb\|X17214\|SFPASA1 | S. faecalis plasmid pAD1 asal gene for | 94 | 432 |
| | | | | | aggregation substance and ORF 1 | | |
| 355 | 3 | 1663 | 2037 | emb\|X62657\|EFORF3 | E.faecalis plasmid pAD1 DNA for orf3 | 99 | 337 |
| 355 | 4 | 2035 | 2445 | emb\|X96977\|EFPAD1ORF | “E.faecalis plasmid pAD1, open reading | 99 | 411 |
| | | | | | frames” | | |
| 355 | 5 | 2558 | 2851 | emb\|X96977\|EFPAD1ORF | “E.faecalis plasmid pAD1, open reading | 96 | 280 |
| | | | | | frames” | | |
| 355 | 6 | 2838 | 3299 | emb\|X96977\|EFPAD1ORF | “E.faecalis plasmid pAD1, open reading | 97 | 430 |
| | | | | | frames” | | |
| 355 | 7 | 3236 | 3739 | emb\|X96977\|EFPAD1ORF | “E.faecalis plasmid pAD1, open reading | 97 | 279 |
| | | | | | frames” | | |
| 355 | 8 | 3696 | 4529 | emb\|X96977\|EFPAD1ORF | “E.faecalis plasmid pAD1, open reading | 97 | 537 |
| | | | | | frames” | | |
| 355 | 9 | 4587 | 5870 | emb\|X96977\|EFPAD1ORF | “E.faecalis plasmid pAD1, open reading | 98 | 718 |
| | | | | | frames” | | |
| 355 | 10 | 5843 | 6490 | emb\|X96977\|EFPAD1OR9 | “E.faecalis plasmid pAD1, open reading | 99 | 224 |
| | | | | | frames” | | |
| 355 | 11 | 6471 | 6890 | emb\|X96977\|EFPAD1ORF | “E.faecalis plasmid pAD1, open reading | 96 | 361 |
| | | | | frames” | | | |
| 355 | 12 | 6881 | 7204 | emb\|X96977\|EFPAD1ORF | “E.faecalis plasmid pAD1, open reading | 98 | 324 |
| | | | | | frames” | | |
| 355 | 13 | 7191 | 8231 | emb\|X96977\|EFPAD1ORF | “E.faecalis plasmid pAD1, open reading | 98 | 984 |
| | | | | | frames” | | |
| 355 | 14 | 8218 | 8496 | emb\|X96977\|EFPAD1ORF | “E.faecalis plasmid pAD1, open reading | 99 | 279 |
| | | | | | frames” | | |
| 355 | 15 | 8412 | 8885 | emb\|X96977\|EFPAD1ORF | “E.faecalis plasmid pAD1, open reading | 100 | 474 |
| | | | | | frames” | | |
| 355 | 17 | 9479 | 9952 | emb\|X96977\|EFPAD1ORF | “E.faecalis plasmid pADl, open reading | 98 | 417 |
| | | | | | frames” | | |
| 365 | 1 | 3 | 380 | gb\|M13771\| | “Streptococcus faecalis 6′-aminoglycoside | 100 | 248 |
| | | | | | acetyltransferase phosphotransferase | | |
| | | | | | (AAC(6′)-APH(2′)) bifunctional resistance | | |
| | | | | | protein, complete cds” | | |
| 370 | 1 | 1 | 1299 | dbj\|D78016\|ENEPPD1A | “Enterococcus faecalis Plasmid pPD1 genes | 73 | 1267 |
| | | | | | for REPB, REPA, TRAC, TRAB, TRAA, iPD1, | | |
| | | | | | TRAE, TRAF, complete cds and partial cds” | | |
| 407 | 3 | 963 | 2162 | gb\|U38590\| | “Enterococcus faecalis plasmid pCF10 PrgN, | 98 | 257 |
| | | | | | PrgO, and PrgP genes, complete cds” | | |
| 407 | 5 | 3811 | 4131 | gb\|U38590\| | “Enterococcus faecalis plasmid pCF10 PrgN, | 86 | 317 |
| | | | | | PrgO, and PrgP genes, complete cds” | | |
| 417 | 1 | 42 | 419 | gb\|UOO681\| | “Enterococcus faecalis plasmid pADi TraB | 98 | 304 |
| | | | | | (traB) gene, complete cds (traC) and | | |
| | | | | | (repA) genes, partial cds” | | |
| 417 | 2 | 313 | 41 | gb\|U00681\| | “Enterococcus faecalis plasmid pADl TraB | 97 | 198 |
| | | | | | (traB) gene, complete cds (traC) and | | |
| | | | | | (repA) genes, partial cds” | | |
| 417 | 3 | 440 | 754 | gb\|U00681\| | “Enterococcus faecalis plasmid pAD1 TraB | 100 | 219 |
| | | | | | (traB) gene, complete cds (traC) and | | |
| | | | | | (repA) genes, partial cds” | | |
| 426 | 1 | 112 | 462 | emb\|Z49243\|EF4110SOD | E.faecalis partial sod gene for superoxide | 98 | 291 |
| | | | | | dismutase (strain = BM4110) | | |
| 426 | 2 | 628 | 419 | emb\|Z49243\|EF4110SOD | E.faecalis partial sod gene for superoxide | 100 | 148 |
| | | | | | dismutase (strain = BM4110) | | |
| 426 | 3 | 456 | 725 | emb\|Z49243\|EF4110SOD | E.faecalis partial sod gene for superoxide | 100 | 148 |
| | | | | | dismutase (strain = BM4110) | | |
| 429 | 1 | 840 | 79 | emb\|X62658\|EFSEA1 | E.faecalis plasmid pADl seal gene and orfy | 98 | 737 |
| 429 | 2 | 1087 | 767 | emb\|X62658\|EFSEA1 | E.faecalis plasmid pAD1 seal gene and orfy | 99 | 321 |
| 429 | 4 | 2765 | 2460 | gb\|U17153\| | “Enterococcus faecalis plasmid pjh1 | 98 | 89 |
| | | | | | tetracycline resistant (tetL) gene, | | |
| | | | | | complete cds” | | |
| 429 | 5 | 3166 | 2750 | gb\|U17153\| | “Enterococcus faecalis plasmid pjhl | 99 | 413 |
| | | | | | tetracycline resistant (tetL) gene, | | |
| | | | | | complete cds” | | |
| 435 | 5 | 2731 | 2324 | gb\|M38052\| | “Enterococcus faecalis cytolysin B | 97 | 97 |
| | | | | | transport protein gene, complete cds” | | |
| 459 | 2 | 1330 | 1067 | gb\|M1377\| | “Streptococcus faecalis 6′-aminoglycoside | 99 | 248 |
| | | | | | acetyltransferase phosphotransferase | | |
| | | | | | (AAC(6′)-APH(2′)) bifunctional resistance | | |
| | | | | | protein, complete cds” | | |
| 506 | 1 | 1242 | 4 | emb\|X17214\|SFPASA1 | S. faecalis plasmid pADi asal gene for | 99 | 1144 |
| | | | | | aggregation substance and ORF 1 | | |
| 514 | 3 | 1496 | 1113 | gb\|M13771\| | “Streptococcus faecalis 6′-aminoglycoside | 100 | 248 |
| | | | | | acetyltransferase phosphotransferase | | |
| | | | | | (AAC(6′)-APH(2′)) bifunctional resistance | | |
| | | | | | protein, complete cds” | | |
| 527 | 2 | 1733 | 1371 | gb\|U17153\| | “Enterococcus faecalis plasmid pjhl | 98 | 153 |
| | | | | | tetracycline resistant (tetL) gene, | | |
| | | | | | complete cds” | | |
| 544 | 1 | 309 | 4 | gb\|U38590\| | “Enterococcus faecalis plasmid pCF10 PrgN, | 95 | 306 |
| | | | | | PrgO, and PrgP genes, complete cds” | | |
| 561 | 1 | 3 | 761 | dbj\|D78016\|ENEPPD1A | “Enterococcus faecalis Plasmid pPD1 genes | 77 | 528 |
| | | | | | for REPB, REPA, TRAC, TRAB, TRAA, iPD1, | | |
| | | | | | TRAE, TRAF, complete cds and partial cds” | | |
| 561 | 2 | 772 | 1566 | gb\|U00681\| | “Enterococcus faecalis plasmid pAD1 TraB | 99 | 795 |
| | | | | | (traB) gene, complete cds (traC) and | | |
| | | | | | (repA) genes, partial cds” | | |
| 566 | 3 | 874 | 2037 | dbj\|D78016\|ENEPPD1A | “Enterococcus faecalis Plasmid pPD1 genes | 90 | 1160 |
| | | | | | for REPB, REPA, TRAC, TRAB, TRAA, iPD1, | | |
| | | | | | TRAE, TPAF, complete cds and partial cds” | | |
| 581 | 1 | 398 | 3 | emb\|X96977\|EFPAD1ORF | “E.faecalis plasmid pAD1, open reading | 100 | 393 |
| | | | | | frames” | | |
| 581 | 2 | 908 | 540 | emb\|X96977\|EFPAD1ORF | “E.faecalis plasmid pAD1, open reading | 100 | 369 |
| | | | | | frames” | | |
| 597 | 1 | 573 | 7 | gb\|M38052\| | “Enterococcus faecalis cytolysin B | 99 | 566 |
| | | | | | transport protein gene, complete cds” | | |
| 597 | 2 | 1247 | 516 | gb\|M38052\| | “Enterococcus faecalis cytolysin B | 97 | 701 |
| | | | | | transport protein gene, complete cds” | | |
| 604 | 7 | 3265 | 2903 | gb\|U17153\| | “Enterococcus faecalis plasmid pjhl | 100 | 143 |
| | | | | | tetracycline resistant (tetL) gene, | | |
| | | | | | complete cds” | | |
| 618 | 1 | 1 | 534 | gb\|M13771\| | “Streptococcus faecalis 6′-aminoglycoside | 99 | 470 |
| | | | | | acetyltransferase phosphotransferase | | |
| | | | | | (AAC(6′)-APH(2′)) bifunctional resistance | | |
| | | | | | protein, complete cds” | | |
| 622 | 1 | 864 | 16 | gb\|M13771\| | “Streptococcus faecalis 6′-aminoglycoside | 99 | 849 |
| | | | | | acetyltransferase phosphotransferase | | |
| | | | | | (AAC(6′)-APH(2′)) bifunctional resistance | | |
| | | | | | protein, complete cds” | | |
| 622 | 2 | 1317 | 862 | gb\|M13771\| | “Streptococcus faecalis 6′-aminoglycoside | 99 | 256 |
| | | | | | acetyltransferase phosphotransferase | | |
| | | | | | (AAC(6′)-APH(2′)) bifunctional resistance | | |
| | | | | | protein, complete cds” | | |
| 622 | 3 | 1586 | 1311 | gb\|M13771\| | “Streptococcus faecalis l 6′-aminoglycoside | 99 | 248 |
| | | | | | acetyltransferase phosphotransferase | | |
| | | | | | (AAC(6′)-APH(2′)) bifunctional resistance | | |
| | | | | | protein, complete cds” | | |
| 624 | 6 | 5641 | 8001 | gb\|U66286\| | “Enterococcus faecalis gyrase A (gyrA) | 98 | 219 |
| | | | | | gene, partial cds” | | |
| 635 | 1 | 516 | 953 | dbj\|D78257\|D78257 | “Enterococcus faecalis plasmid pYI17 genes | 94 | 404 |
| | | | | | for BacA, BacB, ORF3, ORF4, ORF5, ORF6, | | |
| | | | | | ORF7, ORF8, ORF9, ORF10, ORF11,partial | | |
| | | | | | cds38 | | |
| 635 | 2 | 920 | 1222 | dbj\|D78257\|D78257 | “Enterococcus faecalis plasmid pYI17 genes | 83 | 299 |
| | | | | | for BacA, BacB, ORF3, ORF4, ORF5, ORF6, | | |
| | | | | | ORF7, ORF8, ORF9, ORF10, ORF11,partial | | |
| | | | | | cds” | | |
| 637 | 1 | 3 | 545 | emb\|X62656\|EFASP1 | “E.faecalis plasmid pPD1 asp1 and URFs | 92 | 506 |
| | | | | | pd57, pd125 and pd113 genes | | |
| 658 | 2 | 1198 | 365 | gb\|M38052\| | “Enterococcus faecalis cytolysin B | 100 | 819 |
| | | | | | transport protein gene, complete ods” | | |
| 658 | 3 | 1446 | 1189 | gb\|M38052\| | “Enterococcus faecaliscytolysin B | 98 | 258 |
| | | | | | transport protein gene, complete cds” | | |
| 664 | 1 | 490 | 65 | emb\|X62658\|EFSEA1 | E.faecalis plasmid pAD1 seal gene and orfy | 88 | 423 |
| 664 | 2 | 737 | 417 | emb\|X62658\|EFSEA1 | E.faecalis plasmid pAD1 seal gene and orfy | 94 | 321 |
| 743 | 1 | 561 | 4 | dbj\|78016\|ENEPPD1A | “Enterococcus faecalis Plasmid pPD1 genes | 87 | 305 |
| | | | | | for REPB, REPA, TRAC, TRAB, TRAA, iPD1, | | |
| | | | | | TRAE, TRAF, complete cds and partial cds” | | |
| 747 | 2 | 1139 | 324 | gb\|M38052\| | “Enterococcus faecalis cytolysin B | 99 | 691 |
| | | | | | transport protein gene, complete cds” | | |
| 747 | 3 | 577 | 783 | gb\|M38052\| | “Enterococcus faecalis cytolysin B | 100 | 207 |
| | | | | | transport protein gene, complete cds” | | |
| 747 | 4 | 1474 | 1133 | gb\|M13771\| | “Streptococcus faecalis 6′-aminoglycoside | 99 | 248 |
| | | | | | acetyltransferase phosphotransferase | | |
| | | | | | (AAC(6′)-APH(2′)) bifunctional resistance | | |
| | | | | | protein, complete cds” | | |
| 777 | 1 | 401 | 3 | gb\|M38052\| | Enterococcus faecalis cytolysin B | 100 | 335 |
| | | | | | transport protein gene, complete cds” | | |
| 816 | 1 | 793 | 512 | gb\|M13771\| | “Streptococcus faecalis 6-aminoglycoside | 100 | 243 |
| | | | | | acetyltransferase phosphotransferase | | |
| | | | | | (AAC(6′)-APH(2′)) bifunctional resistance | | |
| | | | | | protein, complete cds“ | | |
| 842 | 1 | 418 | 89 | emb\|X17214\|SFPASA1 | S. faecalis plasmid pAD1 asal gene for | 91 | 303 |
| | | | | | aggregation substance and ORF 1 | | |
| 842 | 2 | 856 | 605 | emb\|X62658\|EFSEA1 | E.faecalis plasmid pAD1 seal gene and orfy | 92 | 246 |
| 847 | 1 | 1481 | 3 | emb\|X62658\|EFSEA1 | E.faecalis plasmid pAD1 seal gene and orfy | 92 | 1479 |
| 864 | 1 | 36 | 1106 | emb\|X62658\|EFSEA1 | E.faecalis plasmid pAD1 seal gene and orfy | 93 | 945 |
| 864 | 2 | 1571 | 3550 | emb\|X62656\|EFASP1 | “E.faecalisplasmid pPD1 asp1 and URFs | 96 | 1979 |
| | | | | | pd57, pd125 and pd113 genes” | | |
| 872 | 1 | 263 | 3 | gb\|U17153\| | “Enterococcus faecalis plasmid pjh1 | 98 | 261 |
| | | | | | tetracycline resistant (tetL) gene, | | |
| | | | | | complete cds” | | |
| 874 | 1 | 833 | 693 | dbj\|D31675\|ENE16RNA8 | “Enterococcus faecalis 16S ribosomal RNA, | 100 | 98 |
| | | | | | partial sequence” | _________ | |
| 878 | 1 | 302 | 30 | gb\|U17153\| | “Enterococcus faecalis plasmid pjh1 | 94 | 94 |
| | | | | | tetracycline resistant (tetL) gene, | | |
| | | | | | complete cds” | | |
| 878 | 2 | 263 | 445 | gb\|U17153\| | “Enterococcus faecalis plasmid pjh1 | 99 | 181 |
| | | | | | tetracycline resistant (tetL) gene, | | |
| | | | | | complete cds” | | |
| 921 | 1 | 748 | 26 | emb\|X62658\|EFSEA1 | E.faecalis plasmid pAD1 seal gene and orfy | 95 | 612 |
| 929 | 1 | 484 | 2 | emb\|X62658\|EFSEA1 | E.faecalis plasmid pAD1 seal gene and orfy | 99 | 409 |
| 946 | 1 | 3 | 422 | emb\|X62657\|EFORF3 | E.faecalis plasmid pAD1 DNA for orf3 | 99 | 341 |
| 946 | 2 | 420 | 830 | emb\|X96977\|EFPAD1ORF | “E.faecalis plasmid pAD1, open reading | 98 | 411 |
| | | | | | frames” | | |
| 946 | 3 | 866 | 1123 | emb\|X96977\|EFPAD1ORF | “E.faecalis plasmid pAD1, open reading | 96 | 230 |
| | | | | | frames” | | |
| 947 | 1 | 112 | 498 | emb\|X62656\|EFASP1 | “E.faecalis plasmid pPD1 asp1 and URFs | 96 | 378 |
| | | | | | pd57, pd125 and pd113 genes” | | |
| 951 | 1 | 484 | 26 | emb\|X62658\|EFSEA1 | E.faecalis plasmid pAD1 seal gene and orfy | 95 | 353 |
| 956 | 1 | 3 | 545 | emb\|X62656\|EFASP1 | “E.faecalis plasmid pPD1 asp1 and URFs | 96 | 543 |
| | | | | | pd57, pd125 and pd113 genes” | | |
| 956 | 2 | 524 | 721 | emb\|X62656\|EFASP1 | “E.faecalis plasmid pPD1 asp1 and URFs | 94 | 161 |
| | | | | | pd57, pd125 and pd113 genes” | | |
| 957 | 1 | 616 | 2 | emb\|X96977\|EFPAD1ORF | “E.faecalis plasmid pAD1, open reading | 99 | 615 |
| | | | | | frames” | | |
| 957 | 2 | 42 | 686 | emb\|X96977\|EFPAD1ORF | “E.facalis plasmid pAD1, open reading | 99 | 595 |
| | | | | | frames” | | |
| 968 | 1 | 1 | 456 | emb\|X62656\|EFASP1 | “E.faecalis plasmid pPD1 asp1 and URFs | 96 | 366 |
| | | | | | pd57, pd125 and pd113 genes” | | |
| 968 | 2 | 339 | 641 | emb\|X62656\|EFASP1 | “E.faecalis plasmid pPD1 asp1 and URFs | 95 | 158 |
| | | | | | pd57, pd125 and pd113 genes | | |
| 968 | 3 | 395 | 658 | emb\|X62656\|EFASP1 | “E.faecalis plasmid pPD1 asp1 and URFs | 94 | 126 |
| | | | | | pd57, pd125 and pd113 genes” | | |
| 977 | 1 | 5 | 943 | emb\|X17214\|SFPASA1 | S. faecalis plasmid pAD1 asal gene for | 99 | 847 |
| | | | | | aggregation substance and ORF 1 | | |
| 982 | 1 | 376 | 2 | emb\|X62658\|EFSEA1 | E.faecalis plasmid pAD1 seal gene and orfy | 95 | 365 |
| 985 | 1 | 85 | 471 | emb\|X62656\|EFASP1 | “E.faecalis plasmid pPD1 asp1 and URFs | 91 | 362 |
| | | | | | pd57, pd125 and pd113 genes” | | |

TABLE 2
E. faecalis - Putative coding regions of novel proteins similar to known proteins

| | | | | | | | |
|---|---|---|---|---|---|---|---|
| Contig | ORF | Start | Stop | | | | |
| ID | ID | (nt) | (nt) | Match accession | Match gene name | % Sim | % Ident |

| | | | | | | | | |
|---|---|---|---|---|---|---|---|---|
| 137 | 3 | 3208 | 2003 | gi\|152947 | transposase [Staphylococcus aureus] | 100 | 100 | |
| 154 | 14 | 9166 | 9750 | gi\|141861 | traA gene product [Plasmid pAD1] | 100 | 100 | |
| 276 | 16 | 11268 | 11047 | gnl\|PID\|e284733 | C34B7.1 [Caenorhabditis elegans] | 100 | 71 | |
| 287 | 1 | 485 | 234 | gi\|152947 | transposase [Staphylococcus aureus] | 100 | 100 | |
| 287 | 7 | 3454 | 3765 | gi\|152947 | transposase [Staphylococcus aureus] | 100 | 100 | |
| 292 | 6 | 3001 | 4185 | gi\|488330 | alpha-amylase [unidentified cloning | 100 | 100 | |
| | | | | | vector] | | | |
| 429 | 3 | 2013 | 1654 | gi\|141863 | regulatory protein [Plasmid pAD1] | 100 | 100 | |
| 604 | 3 | 1243 | 1043 | gi\|559860 | clyLs [Plasmid pAD1] | 100 | 98 | |
| 604 | 4 | 1492 | 1268 | gi\|559859 | clyL1 [PLasmid pAD1] | 100 | 100 | |
| 656 | 7 | 7592 | 6834 | gi\|488339 | alpha-amylase [unidentified cloning | 100 | 100 | |
| | | | | | vector] | | | |
| 658 | 1 | 312 | 4 | gi\|152947 | transposase [Staphylococcus aureus] | 100 | 100 | |
| 674 | 3 | 1236 | 1589 | gi\|1196996 | unknown protein [Transposon Tn10] | 100 | 98 | |
| 700 | 1 | 375 | 4 | gi\|152947 | transposase [Staphylococcus aureus] | 100 | 100 | |
| 961 | 1 | 1 | 450 | gi\|152947 | transposase [Staphylococcus aureus] | 100 | 100 | |
| 72 | 17 | 20153 | 21040 | gi\|150556 | surface protein [Plasmid pCF10] | 99 | 99 | |
| 99 | 5 | 3117 | 1933 | gi\|1006839 | malic enzyme [Streptococcus bovis] | 99 | 99 | |
| 154 | 3 | 1995 | 1491 | gi\|149482 | transposase [Lactococcus lactis] | 99 | 99 | |
| 326 | 3 | 3030 | 1714 | pir\|S16989\|S16989 | dihydrolipaomide S-acetyltransferase (EC | 99 | 98 | |
| | | | | | 2.3.1.12)-Enterococcus faecalis | | | |
| 407 | 6 | 4636 | 4235 | gi\|141859 | replication-associated protein [Plasmid | 99 | 99 | |
| | | | | | pAD1] | | | |
| 692 | 1 | 3 | 485 | gi\|559861 | clyM [Plasmid pAD1] | 99 | 99 | |
| 99 | 6 | 3904 | 3134 | gi\|1146122 | L-malate permease [Streptococcus bovis] | 98 | 98 | |
| 326 | 4 | 3358 | 3002 | pir\|S16989\|S16989 | dihydrolipoamide S-acetyltransferase (EC | 98 | 97 | |
| | | | | | 2.3.1.12)-Enterococcus faecalis | | | |
| 346 | 1 | 606 | 4 | gi\|1146122 | L-malate permease [Streptococcus bovis] | 98 | 98 | |
| 367 | 31 | 14415 | 13999 | gi\|1644226 | ribosomal protein S10 [Bacillus subtilis] | 98 | 88 | |
| 367 | 6 | 2797 | 2495 | gi\|142459 | initiation factor 1 [Bacillus subtilis] | 97 | 88 | |
| 407 | 9 | 5454 | 4894 | gi\|141858 | replication-associated protein [Plasmid | 97 | 97 | |
| | | | | | pAD1] | | | |
| 497 | 6 | 3514 | 3762 | gi\|532552 | ORF19 [Enterococcus faecalis] | 97 | 87 | |
| 558 | 1 | 1 | 399 | gi\|46638 | ORF 2 (AA 1-236) [Staphylococcus aureus] | 97 | 97 | |
| 829 | 1 | 169 | 2 | gnl\|PID\|e283110 | femD [Staphylococcus aureus] | 97 | 86 | |
| 407 | 8 | 4970 | 4599 | gi\|141858 | replication-associated protein [Plasmid | 96 | 96 | |
| | | | | | pAD1] | | | |
| 777 | 2 | 1102 | 380 | gi\|559861 | clyM [Plasmid pAD1] | 96 | 96 | |
| 23 | 33 | 20797 | 21126 | gnl\|PID\|e223402 | DNA topoisomerase IV C submit | 95 | 80 | |
| | | | | | [Streptococcus pneumoniae] | | | |
| 32 | 5 | 3454 | 3071 | gi\|147194 | phnA protein [Escherichia coli] | 95 | 87 | |
| 95 | 8 | 5493 | 6875 | gi\|391682 | Na+ −ATPase beta subunit [Enterococcus | 95 | 89 | |
| | | | | | hirae] | | | |
| 138 | 25 | 16587 | 16745 | gi\|143136 | L-lactate dehydrogenase [Bacillus | 95 | 70 | |
| | | | | | megaterium] | | | |
| 367 | 20 | 9198 | 8797 | gi\|40150 | L14 protein (AA 1-122) [Bacillus subtilis] | 95 | 90 | |
| 367 | 21 | 9519 | 9223 | gi\|1044973 | ribosomal protein L17 [Bacillus subtilis] | 95 | 89 | |
| 439 | 2 | 846 | 1241 | gi\|488334 | alpha-amylase [unidentified cloning | 95 | 94 | |
| | | | | | vector] | | | |
| 604 | 1 | 792 | 4 | gi\|559861 | clyM [Plasmid pAD1] | 95 | 93 | |
| 722 | 1 | 1 | 504 | gi\|47453 | ribosomal protein S12 [Streptococcus | 95 | 94 | |
| | | | | | pneumoniae] | | | |
| 17 | 8 | 7317 | 7676 | gi\|532554 | ORF21 [Enterococcus faecalis] | 94 | 86 | |
| 95 | 2 | 1288 | 1791 | gi\|416405 | Na+−ATPase K subunit [Enterococcus hirae] | 94 | 88 | |
| 97 | 3 | 2481 | 1432 | gi\|1750264 | heat shock protein 70 [Streptococcus | 94 | 90 | |
| | | | | | pneumoniae] | | | |
| 117 | 5 | 2700 | 3842 | gi\|467376 | unknown [Bacillus subtilis] | 94 | 89 | |
| 327 | 3 | 3283 | 3762 | gi\|153566 | ORF (19K protein) [Enterococcus faecalis] | 94 | 87 | |
| 327 | 5 | 4782 | 5054 | gi\|153568 | H+ ATPase [Enterococcus faecalis] | 94 | 82 | |
| 387 | 4 | 3608 | 1728 | gi\|153661 | translational initiation factor IF2 | 94 | 88 | |
| | | | | | [Enterococcus faecium] sp\|P18311\|IF2_ENTFC | | | |
| | | | | | INITIATION FACTOR IF-2. | | | |
| 455 | 1 | 2 | 259 | gi\|532549 | ORF16 [Enterococcus faecali] | 94 | 82 | |
| 97 | 2 | 1444 | 677 | gi\|450684 | dnaK gene product [Lactococcus lactis] | 93 | 83 | |
| 188 | 2 | 1690 | 1911 | gi\|43865 | nifJ gene product [Klebsiella pneumoniae] | 93 | 78 | |
| 216 | 6 | 4234 | 4680 | gi\|153574 | H+ ATPase [Enterococcus faecalis] | 93 | 86 | |
| 298 | 2 | 2798 | 1221 | gi\|143012 | GMP synthetase [Bacillus subtilis] | 93 | 86 | |
| 329 | 2 | 1538 | 771 | gi\|153826 | adhesin B [Streptococcus sanguis] | 93 | 83 | |
| 367 | 15 | 7675 | 7247 | gi\|1044978 | ribosomal protein S8 [Bacillus subtilis] | 93 | 82 | |
| 722 | 2 | 527 | 1030 | gi\|1644222 | ribosomal protein S7 [Bacillus subtilis] | 93 | 83 | |
| 803 | 1 | 657 | 151 | gi\|1196998 | unknown protein [Transposon Tn10] | 93 | 93 | |
| 962 | 1 | 130 | 636 | gi\|152947 | transposase [Staphylococcus aureus] | 93 | 92 | |
| 237 | 12 | 6056 | 6385 | gi\|963038 | Arpυ [Enterococcus hirae] | 92 | 76 | |
| 309 | 4 | 8218 | 4541 | gi\|402363 | RNA polymerase beta-subunit [Bacillus | 92 | 82 | |
| | | | | | subtilis] sp \|P37870\| RPOB_BACSU DNA- | | | |
| | | | | | DIRECTED RNA POLYMERASE BETA CHAIN (EC | | | |
| | | | | | .7.7.6) (TRANSCRIPTASE BETA CHAIN) (RNA | | | |
| | | | | | POLYMERASE BETA SUBUNIT). | | | |
| 329 | 4 | 2529 | 1717 | gi\|310632 | hydrophobic membrane protein | 92 | 78 | |
| | | | | | [Streptococcus gordonii] | | | |
| | | | | | sp\|P42361\|P29K_STRGC 29 KD MEMBRANE | | | |
| | | | | | PROTEIN IN PSAA 5′REGION ORF1). | | | |
| 367 | 4 | 1942 | 1544 | gi\|142462 | ribosomal protein S11 [Bacillus subtilis] | 92 | 82 | |
| 367 | 8 | 3648 | 3457 | pir\|C44859\|C44859 | adenylate kinase - Bacillus sp. (fragment) | 92 | 88 | |
| 367 | 12 | 6183 | 5641 | gi\|1044981 | ribosomal protein S5 [Bacillus subtilis] | 92 | 81 | |
| 367 | 17 | 8427 | 7885 | pir51 A29102\|R5BS5F | ribosomal protein L5 - Bacillus | 92 | 83 | |
| | | | | | stearothermophilus | | | |
| 527 | 1 | 1404 | 373 | gi\|153092 | replication protein [Staphylococcus | 92 | 81 | |
| | | | | | aureus] | | | |
| 701 | 1 | 2 | 352 | gi\|143793 | tyrosyl-tRNA synthetase [Bacillus | 92 | 74 | |
| | | | | | caldotenax] | | | |
| 23 | 28 | 17420 | 17566 | sp\|P45692\|EUTX_SAL | ETHANOLAMINE UTILIZATION PROTEIN EUTX | 91 | 73 | |
| | | | | TY | (FRAGMENT). | | | |
| 57 | 5 | 4129 | 4701 | gi\|15958l0 | type-I signal peptidase SpsB | 91 | 67 | |
| | | | | | [Staphylococcus aureus] | | | |
| 57 | 12 | 13281 | 13970 | gnl\|PID\|e254999 | phenylalany-tRNA synthetase beta subunit | 91 | 75 | |
| | | | | | [Bacillus subtilis] | | | |
| 156 | 5 | 4609 | 6474 | gi\|1303804 | YqeQ [Bacillus subtilis] | 91 | 79 | |
| 216 | 3 | 1848 | 2765 | gi\|153572 | H+ ATPase [Enterococcus faecalis] | 91 | 81 | |
| 367 | 24 | 10802 | 10128 | gi\|1165309 | S3 [Bacillus subtilis] | 91 | 78 | |
| 415 | 1 | 452 | 883 | pir\|B56272\|B56272 | probable pheromone-responsive regulatory | 91 | 90 | |
| | | | | | protein R - Enterococcus faecalis plasmid | | | |
| | | | | | pCF10 | | | |
| 466 | 2 | 1313 | 2065 | gi\|142443 | adenylosuccinate synthetase [Bacillus | 91 | 79 | |
| | | | | | subtilis]sp\|P29726\|PURA_BACSU | | | |
| | | | | | ADENYLOSUCCINATE SYNTHETASE (EC 6.3.4.4) | | | |
| | | | | | IMP--ASPARTATE LIGASE). | | | |
| 545 | 1 | 1 | 345 | gi\|532549 | ORF16 [Enterococcus faecalis] | 91 | 80 | |
| 572 | | 1 8 | 652 | gi\|347998 | uracil phosphoribosyltransferase | 91 | 78 | |
| | | | | | [Streptococcus salivarius] | | | |
| | | | | | sp\|P36399\|UPP_STRSL PROBABLE URACIL | | | |
| | | | | | PHOSPHORIBOSYLTRANSFERASE (EC .4.2.9) (UMP | | | |
| | | | | | PYROPHOSPHORYLASE) (UPRTASE). | | | |
| 599 | 1 | 8 | 343 | gi\|42029 | ORF1 gene product [Escherichia coli] | 91 | 75 | |
| 600 | 2 | 585 | 779 | pir\|B48396\|B48396 | ribosomal protein L33 - Bacillus | 91 | 81 | |
| | | | | | stearothermophilus | | | |
| 652 | 1 | 394 | 2 | gi\|535662 | transposase [Insertion sequence IS1251] | 91 | 81 | |
| 1 | 4 | 3465 | 2557 | gi\|1644224 | elongation factor Tu [Bacillus subtilis] | 90 | 83 | |
| 17 | 19 | 14844 | 17297 | gi\|532549 | ORF16 [Enterococcus faecalis] | 90 | 77 | |
| 52 | 3 | 2650 | 2811 | gi\|473902 | alpha-acetolactate synthase [Lactococcus | 90 | 68 | |
| | | | | | lactis] | | | |
| 74 | 9 | 5870 | 5469 | gi\|1653508 | hypothetical protein [Synechocystis sp.] | 90 | 52 | |
| 75 | 3 | 1177 | 2091 | gi\|153615 | phosphoenolpyruvate:sugar | 90 | 83 | |
| | | | | | phosphotransferase system enzyme I | | | |
| | | | | | Streptococcus salivarius] | | | |
| 117 | 10 | 6591 | 8126 | gi\|924848 | inosine monophosphate dehydrogenase | 90 | 80 | |
| | | | | | [Streptococcus pyogenes] pir\|JC4372 \|JC4372 | | | |
| | | | | | IMP dehydrogenase (EC 1.1.1.205) - | | | |
| | | | | | Streptococcus yogenes | | | |
| 276 | 1 | 577 | 95 | gi\|530798 | LysB [Bacteriophage phi-LC3] | 90 | 72 | |
| 287 | 5 | 2611 | 2441 | gi\|1333835 | copS gene product [Streptococcus pyogenes] | 90 | 78 | |
| 290 | 1 | 1 | 708 | gi\|897795 | 30S ribosomal protein [Pediococcus | 90 | 75 | |
| | | | | | acidilactici] sp\|P49668\|RS2_PEDAC 30S | | | |
| | | | | | RIBOSOMAL PROTEIN S2. | | | |
| 309 | 3 | 4401 | 1093 | gnl\|PID\|e187579 | DNA-directed RNA polymerase [Listeria | 90 | 81 | |
| | | | | | innocua] | | | |
| 367 | 22 | 9731 | 9513 | pir\|A02825\|R5BS29 | ribosomal protein L29 - Bacillus | 90 | 76 | |
| | | | | | stearothermophilus | | | |
| 452 | 4 | 2224 | 2508 | gi\|434759 | ORF [Homo sapiens] | 90 | 54 | |
| 455 | 2 | 2776 | 323 | gi\|532549 | ORF16 [Enterococcus faecalis] | 90 | 77 | |
| 623 | 1 | 3 | 221 | gi\|460259 | enolase [Bacillus subtilis] | 90 | 80 | |
| 624 | 5 | 3612 | 5615 | gnl\|PID\|e2O8213 | DNA gyrase [Streptococcus pneumoniae] | 90 | 81 | |
| 853 | 2 | 752 | 282 | gnl\|PID\|e13389 | translation initiation factor IF3 (AA 1- | 90 | 82 | |
| | | | | | 172) [Bacillus stearothermophilus] | | | |
| 966 | 1 | 1 | 462 | gi\|532549 | ORF16 [Enterococcus faccalis] | 90 | 83 | |
| 1 | 3 | 2596 | 2219 | gi\|1661195 | elongation factor-Tu [Streptococcus | 89 | 78 | |
| | | | | | mutans] | | | |
| 1 | 5 | 4314 | 3556 | gi\|1644223 | elongation factor G [Bacillus subtilis] | 89 | 79 | |
| 23 | 21 | 13990 | 14295 | gi\|466518 | pduA [Salmonella typhimurium] | 89 | 75 | |
| 23 | 32 | 19927 | 20799 | gnl\|PID\|e208211 | DNA topoisomerase IV [Streptococcus | 89 | 83 | |
| | | | | | pneumoniae] | | | |
| 42 | 2 | 349 | 1989 | gi\|287871 | groEL gene product [Lactococcus lactis] | 89 | 79 | |
| 45 | 15 | 11835 | 12167 | gi\|150554 | surface exclusion protein [Plasmid pCF10] | 89 | 68 | |
| 53 | 2 | 685 | 1797 | gnl\|PID\|e221213 | ClpX protein [Bacillus subtilis] | 89 | 81 | |
| 86 | 4 | 3374 | 4024 | gi\|537286 | triosephosphate isomerase [Lactococcus | 89 | 78 | |
| | | | | | lactis] | | | |
| 95 | 7 | 3677 | 5506 | gi\|912449 | Na+ −ATPase alpha subunit [Enterococcus | 89 | 80 | |
| | | | | | hirae] | | | |
| 128 | 18 | 11348 | 11013 | gi\|466473 | cellobiose phosphotransferase enzyme II′ | 89 | 60 | |
| | | | | | [Bacillus tearothermophilus] | | | |
| 132 | 1 | 180 | 2180 | gi\|153854 | uvs402 protein [Streptococcus pneumoniae] | 89 | 78 | |
| 342 | 1 | 783 | 4 | gi\|1041115 | TRAC [Plasmid pPD1] | 89 | 79 | |
| 367 | 23 10146 | 9691 | sp\|P14577\|RL16_{—BAC} | 50S RIBOSOMAL PROTEIN L16. | 89 | 80 | | |
| | | | | SU | | | | |
| 367 | 27 | 12377 | 11541 | gi\|1165306 | L2 [Bacillus subtilis] | 89 | 79 | |
| 435 | 4 | 2424 | 2215 | gi\|559863 | clyA [Plasmid pA1] | 89 | 89 | |
| 466 | 3 | 1972 | 2736 | gi\|467328 | adenylosuccinate synthetase [Bacillus | 89 | 75 | |
| | | | | | subtilis] | | | |
| 512 | 3 | 999 | 1607 | gi\|1477776 | ClpP [Bacillus subtilis] | 89 | 73 | |
| 518 | 1 | 1 | 174 | gi\|786163 | Ribosomal Protein L10 [Bacillus subtilis] | 89 | 76 | |
| 604 | 2 | 1000 | 713 | gi\|559861 | clyM [Plasmid pAD1] | 89 | 89 | |
| 615 | 2 | 888 | 691 | gi\|467469 | unknown [Bacillus subtilis] | 89 | 75 | |
| 677 | 2 | 992 | 429 | gi\|1389732 | S-adenosylmethionine synthetase [Bacillus | 89 | 76 | |
| | | | | | subtilis] | | | |
| 677 | 3 | 1315 | 950 | gi\|1020317 | S-adenosylmethionine synthetase | 89 | 73 | |
| | | | | | [Staphylococcus aureus] | | | |
| 722 | 3 | 1102 | 1278 | pir\|PW0010\|PW0010 | translation elongation factor G - Bacillus | 89 | 72 | |
| | | | | | stearothermophilus (fragment) | | | |
| 850 | 1 | 464 | 3 | gi\|142521 | deoxyribodipyrimidine photolyase [Bacillus | 89 | 72 | |
| | | | | | subtilis]gnl\|PID\|e255102 | | | |
| | | | | | deoxyribodipyrimidine photolyase [Bacillus | | | |
| | | | | | ubtilis] | | | |
| 17 | 5 | 3711 | 4751 | gi\|532554 | ORF21 [Enterococcus faecalis] | 88 | 72 | |
| 37 | 5 | 3322 | 3717 | gi\|1216488 | uncharacterized open reading frame; | 88 | 75 | |
| | | | | | hypothetical protein displaying similarity | | | |
| | | | | | to a Bacillus subtilis hypothetical | | | |
| | | | | | protein (Ylm [Streptococcus mutans] | | | |
| 39 | 6 | 2454 | 2630 | sp\|P49865\|NTPR_ENT | NTPR PROTEIN (FRAGMENT). | 88 | 77 | |
| | | | | HR | | | | |
| 48 | 3 | 1740 | 2666 | gi\|557492 | dihydroxynapthoic acid (DHNA) synthetase | 88 | 75 | |
| | | | | | [Bacillus subtilis] gi\|143186 | | | |
| | | | | | dihydroxynapthoic acid (DHNA) synthetase | | | |
| | | | | | [Bacillus ubtilis] | | | |
| 63 | 5 | 2753 | 3607 | gi\|1064814 | homologous to sp:PHOP_BACSUB [Bacillus | 88 | 77 | |
| | | | | | subtilis] | | | |
| 86 | 2 | 1004 | 2047 | gi\|153763 | plasmin receptor [Streptococcus pyogenes] | 88 | 79 | |
| 104 | 6 | 6431 | 6213 | gi\|431231 | uracil permease [Bacillus caldolyticus] | 88 | 60 | |
| 110 | 19 | 18174 | 16891 | gi\|217040 | acid glycoprotein [Streptococcus pyogenes] | 88 | 72 | |
| 145 | 10 | 9040 | 8834 | gi\|393268 | 29-kiloDalton protein [Streptococcus | 88 | 71 | |
| | | | | | pneumoniae]sp\|P42362\|P29K_STRPN 29 KD | | | |
| | | | | | MEMBRANE PROTEIN IN PSAA 5′REGION ORF1). | | | |
| 151 | 1 | 1620 | 316 | gi\|143366 | adenylosuccinate lyase (PUR-B) [Bacillus | 88 | 78 | |
| | | | | | subtilis] pir\|C29326\|WZBSDS | | | |
| | | | | | adenylosuccinate lyase (EC 4.3.2.2) - | | | |
| | | | | | Bacillus ubtilis | | | |
| 171 | 10 | 9676 | 10119 | gi\|1591672 | phosphate transport system ATP-binding | 88 | 63 | |
| | | | | | protein [Methanococcus jannaschii] | | | |
| 190 | 3 | 1997 | 975 | gi\|532554 | ORF21 [Enterococcus faecalis] | 88 | 76 | |
| 229 | 6 | 5712 | 5954 | gi\|143648 | ribosomal protein L28 [Bacillus subtilis] | 88 | 70 | |
| 270 | 2 | 895 | 1869 | gi\|1303828 | YqfJ p8 Bacillus subtilis] | 88 | 75 | |
| 275 | 7 | 3761 | 3552 | gi\|425474 | SMDR1 [Schistosoma mansoni] | 88 | 72 | |
| 293 | 1 | 614 | 3 | gi\|1783246 | highly homologous to many ATP-binding | 88 | 80 | |
| | | | | | transport proteins; hypothetical [Bacillus | | | |
| | | | | | subtilis] | | | |
| 367 | 1 | 485 | 72 | gi\|142464 | ribosomal protein L17 [Bacillus subtilis] | 88 | 76 | |
| 367 | 5 | 2335 | 1961 | gi\|1044989 | ribosomal protein S13 [Bacillus subtilis] | 88 | 80 | |
| 367 | 16 | 7887 | 7681 | pir\|S48688\|S48688 | ribosomal protein S14 - Bacillus | 88 | 83 | |
| | | | | | stearothermophilus | | | |
| 598 | 1 | 1006 | 23 | gi\|565287 | transposase-like protein of PS3IS | 88 | 66 | |
| | | | | | [thermophilic bacterium PS3] | | | |
| | | | | | pir\|JC4292\|JC4292 insertion sequence | | | |
| | | | | | element 1341 - thermophilic acterium PS-3 | | | |
| 600 | 3 | 1640 | 882 | gi\|763052 | integrase [Bacteriophage T270] | 88 | 68 | |
| 669 | 1 | 2 | 514 | gi\|153801 | enzyme scr-II [Streptococcus mutans] | 88 | 75 | |
| 808 | 2 | 624 | 394 | gi\|1574781 | exodeoxyribonuclease V (recB) [Haemophilus | 88 | 77 | |
| | | | | | influenzae] | | | |
| 871 | 1 | 714 | 229 | gi\|1574120 | branched-chain-amino-acid transaminase | 88 | 79 | |
| | | | | | [Haemophilus influenzae] | | | |
| 979 | 1 | 1 | 384 | gnl\|PID\|e187579 | DNA-directed RNA polymerase [Listeria | 88 | 78 | |
| | | | | | innocua] | | | |
| 983 | 1 | 34 | 282 | gi\|40026 | homologous to E.coli gidA [Bacillus | 88 | 78 | |
| | | | | | subtilis] | | | |
| 47 | 5 | 6799 | 5810 | gi\|532204 | prs [Listeria monocytogenes] | 87 | 79 | |
| 69 | 3 | 2033 | 750 | gi\|1377831 | unknown [Bacillus subtilis] | 87 | 74 | |
| 73 | 2 | 1432 | 167 | gi\|143434 | Rho Factor [Bacillus subtilis] | 87 | 76 | |
| 76 | 5 | 2412 | 3740 | gi\|496283 | lysin [Bacteriophage Tuc2009] | 87 | 75 | |
| 88 | 3 | 1600 | 2016 | gnl\|PID\|e137596 | heat shock induced protein HtpO | 87 | 75 | |
| | | | | | [Lactobacillus leichmannii] | | | |
| 89 | 7 | 6003 | 5608 | gi\|1695686 | pyruvate carboxylase [Bacillus | 87 | 77 | |
| | | | | | stearothermophilus] | | | |
| 93 | 1 | 283 | 119 | gi\|1124825 | unknown protein [Chlamydia trachomatis] | 87 56 | | |
| 104 | 1 | 2945 | 3 | gnl\|PID\|e199387 | carbamoyl-phosphate synthase | 87 | 75 | |
| | | | | | [Lactobacillus plantarum] | | | |
| 124 | 4 | 3191 | 2274 | gi\|995767 | UDP-glucose pyrophosphorylase | 87 | 76 | |
| | | | | | [Streptococcus pyogenes] | | | |
| 273 | 2 | 608 | 1108 | gi\|1184680 | polynucleotide phosphorylase [Bacillus | 87 | 76 | |
| | | | | | subtilis] | | | |
| 293 | 2 | 1020 | 532 | gi\|153741 | ATP-binding protein [Streptococcus mutans] | 87 | 74 | |
| 326 | 5 | 4534 | 3533 | gi\|143378 | pyruvate decarboxylase (E-1) beta subunit | 87 | 74 | |
| | | | | | [Bacillus subtilis] gi\|1377836 pyruvate | | | |
| | | | | | decarboxylase E-1 beta subunit [Bacillus | | | |
| | | | | | ubtilis] | | | |
| 334 | 3 | 3182 | 3340 | pir\|A36324\|A36324 | growth arrest-specific protein - mouse | 87 | 50 | |
| 337 | 1 | 1382 | 186 | gi\|308861 | GTG start codon [Lactococcus lactis] | 87 | 75 | |
| 338 | 8 | 6925 | 5723 | gi\|149575 | L(+)-lactate dehydrogenase [Lactobacillus | 87 | 73 | |
| | | | | | casei] sp\|P00343\|LDH_LACCA L-LACTATE | | | |
| | | | | | DEHYDROGENASE (EC 1.1.1.27). (SUB −326) | | | |
| 367 | 18 | 8782 | 8450 | pir\|A02819\|R5BS24 | ribosomal protein L24 - Bacillus | 87 | 70 | |
| | | | | | stearothermophilus | | | |
| 388 | 2 | 410 | 183 | gnl\|PID\|e225674 | unknown [Schizosaccharomyces pombe] | 87 | 75 | |
| 440 | 1 | 466 | 1797 | gi\|520754 | putative [Bacillus subtilis] | 87 | 75 | |
| 508 | 1 | 694 | 137 | gi\|496558 | orfX [Bacillus subtilis] | 87 | 73 | |
| 654 | 3 | 530 | 802 | pir\|A47079\|A47079 | heat shock protein DnaJ - Lactococcus | 87 | 70 | |
| | | | | | lactis | | | |
| 18 | 1 | 3 | 413 | gi\|46912 | ribosomal protein L13 [Staphylococcus | 86 | 70 | |
| | | | | | carnosus] | | | |
| 18 | 2 | 406 | 819 | pir\|S08564\|R3BS9 | ribosomal protein S9 - Bacillus | 86 | 73 | |
| | | | | | stearothermophilus | | | |
| 50 | 1 | 84 | 1148 | gi\|452398 | threonine synthase [Bacillus sp.] | 86 | 74 | |
| 74 | 14 | 10547 | 10080 | gi\|1314299 | ORF6; putative glutamyl-tRNA-transferase; | 86 | 74 | |
| | | | | | similar to glutamyl-tRNA-transferase from | | | |
| | | | | | Bacillus subtilis [Listeria monocytogenes] | | | |
| 95 | 5 | 3176 | 3406 | gi\|487276 | Na+ −ATPase subunit C [Enterococcus hirae] | 86 | 62 | |
| 114 | 8 | 9216 | 10313 | gi\|853776 | peptide chain release factor 1 [Bacillus | 86 | 69 | |
| | | | | | subtilis] pir\|S55437\|S55437 peptide chain | | | |
| | | | | | release factor 1 - Bacillus ubtilis | | | |
| 115 | 2 | 501 | 899 | gi\|551879 | ORF 1 [Lactococcus lactis] | 86 | 70 | |
| 164 | 26 | 25639 | 25842 | pir\|S34762\|S34762 | L-serine dehydratase beta chain - | 86 | 81 | |
| | | | | | Clostridium sp | | | |
| 243 | 2 | 2143 | 1082 | gi\|143607 | sporulation protein [Bacillus subtilis] | 86 | 70 | |
| 255 | 1 | 2 | 196 | gi\|755604 | unknown [Bacillus subtilis] | 86 | 64 | |
| 257 | 3 | 3565 | 983 | gi\|928832 | 0RF259; putative [Lactococcus lactis phage | 86 | 66 | |
| | | | | | BK5-T] | | | |
| 273 | 3 | 943 | 1314 | gi\|1184680 | polynucleotide phosphorylase [Bacillus | 86 | 65 | |
| | | | | | subtilis] | | | |
| 288 | 2 | 554 | 1087 | gi\|153033 | tagatose 6-phosphate isomerase | 86 | 74 | |
| | | | | | [Staphylococcus aureus] pir\|B38158\|B38158 | | | |
| | | | | | galactose-6-phosphate isomerase 19K chain | | | |
| | | | | | - taphylococcus aureus | | | |
| 327 | 7 | 5183 | 5722 | gi\|153569 | H+ ATPase [Enterococcus faecalis] | 86 | 71 | |
| 345 | 7 | 5111 | 5620 | gi\|1314294 | ORF1; putative 17 kDa protein [Listeria | 86 | 63 | |
| | | | | | monocytogenes) | | | |
| 350 | 3 | 1900 | 2781 | gi\|511015 | dihydroorotate dehydrogenase A | 86 | 73 | |
| | | | | | [Lactococcus lactis] sp\|P54321\|PYDA_LACLC | | | |
| | | | | | DIHYDROOROTATE DEHYDROCENASE A (EC | | | |
| | | | | | 1.3.3.1) DIHYDROOROTATE OXIDASE A) | | | |
| | | | | | (DHODEHASE A). | | | |
| 383 | 3 | 3328 | 4233 | gi\|1657517 | hypothetical protein [Escherichia coli] | 86 | 59 | |
| 367 | 25 | 11216 | 10851 | gi\|116538 | L22 [Bacillus subtilis] | 86 | 68 | |
| 367 | 26 | 11534 | 11220 | gi\|1165307 | S19 [Bacillus subtilis] | 86 | 77 | |
| 367 | 30 | 13995 | 13453 | gi\|1165303 | L3 [Bacillus subtilis] | 86 | 75 | |
| 393 | 1 | 1 | 660 | sp\|P33898\|G3P3_ECO | GLYCERALDEHYDE 3-PHOSPHATE DEHYDROGENASE C | 86 | 77 | |
| | | | | LI | (EC 1.2.1.12) (GAPDH-C). | | | |
| 396 | 1 | 1 | 192 | gi\|944942 | RipX [Bacillus subtilis] | 86 | 77 | |
| 438 | 3 | 1279 | 1560 | gi\|1001878 | CspL protein [Listeria monocytogenes] | 86 | 75 | |
| 510 | 1 | 1008 | 199 | gi\|473795 | ‘ORF’ [Escherichia coli] | 86 | 71 | |
| 510 | 2 | 1912 | 962 | gi\|473794 | ‘ORF’ [Escherichia coli] | 86 | 76 | |
| 539 | 1 | 705 | 4 | gi\|467477 | unknown [Bacillus subtilis] | 86 | 79 | |
| 570 | 2 | 2069 | 1023 | gi\|881511 | Ccpa protein [Lactobacillus casei] | 86 | 72 | |
| 654 | 2 | 240 | 575 | pir\|A47079\|A47079 | heat shock protein DnaJ - Lactococcus | 86 | 77 | |
| | | | | | lactis | | | |
| 677 | 1 | 431 | 102 | gi\|1389732 | S-adenosylmethionine synthetase [Bacillus | 86 | 80 | |
| | | | | | subtilis] | | | |
| 984 | 1 | 1 | 147 | pir\|A56922\|A56922 | transcription factor shn - fruit fly | 86 | 73 | |
| | | | | | (Drosophila melanogaster) | | | |
| 5 | 11 | 7720 | 8487 | gi\|41015 | aspartate-tRNA ligase [Escherichia coli] | 85 | 71 | |
| 34 | 2 | 2133 | 1711 | gi\|47828 | pyruvate kinase [Bacillus | 85 | 75 | |
| | | | | | stearothermophilus] | | | |
| 97 | 4 | 2666 | 2517 | pir\|S39341\|S3934l | grpE protein - Lactococcus lactis | 85 | 66 | |
| 103 | 2 | 1263 | 946 | gi\|143364 | phosphoribosyl aminoimidazole carboxylase | 85 | 68 | |
| | | | | | I (PUR-E) [Bacillus ubtilis] | | | |
| 103 | 3 | 1465 | 1169 | gi\|143364 | phosphoribosyl aminoimidazole carboxylase | 85 | 67 | |
| | | | | | I (PUR-E) [Bacillus ubtilis] | | | |
| 129 | 3 | 2395 | 3258 | gi\|143766 | (thrSv) (EC 6.1.1.3) [Bacillus subtilis] | 85 | 67 | |
| 129 | 4 | 3240 | 4445 | gi\|143766 | (thrSv) (EC 6.1.1.3) [Bacillus subtilis] | 85 | 78 | |
| 188 | 1 | 86 | 1447 | gnl\|PID\|e214721 | glutamine synthetase [Staphylococcus | 85 | 71 | |
| | | | | | aureus] | | | |
| 217 | 3 | 673 | 1086 | gi\|520540 | unknown [Bacillus subtilis] | 85 | 72 | |
| 241 | 2 | 1715 | 1086 | gi\|495089 | recombinase [Staphylococcus aureus] | 85 | 68 | |
| 285 | 2 | 712 | 993 | gi\|40014 | pot. ORF 446 (aa 1-446) [Bacillus | 85 | 77 | |
| | | | | | subtilis] | | | |
| 293 | 3 | 1149 | 1595 | gi\|755604 | unknown [Bacillus subtilis] | 85 | 66 | |
| 300 | 2 | 2738 | 2220 | gi\|289261 | comE ORF2 [Bacillus subtilis] | 85 | 72 | |
| 305 | 2 | 1853 | 2695 | pir\|S09411\|S09411 | spoIIIE protein - Bacillus subtilis | 85 | 70 | |
| 322 | 1 | 1 | 171 | gi\|153562 | aspartate beta-semialdehyde dehydrogenase | 85 | 67 | |
| | | | | | (EC 1.2.1.11) Streptococcus mutans] | | | |
| 327 | 4 | 4056 | 4784 | gi\|153567 | H+ ATPase [Enterococcus faecalis] | 85 | 66 | |
| 367 | 10 | 5417 | 4959 | pir\|A02795\|R5BS15 | ribosomal protein L15 - Bacillus | 85 | 76 | |
| | | | | | stearothermophilus | | | |
| 383 | 3 | 3168 | 2953 | gnl\|PID\|e274577 | csp [Lactobacillus plantarum] | 85 | 79 | |
| 404 | 3 | 3069 | 2101 | gi\|143402 | recombination protein (ttg start codon) | 85 | 72 | |
| | | | | | [Bacillus subtilis] gi\|1303923 RecN | | | |
| | | | | | [Bacillus subtilis] | | | |
| 469 | 1 | 2 | 724 | gi\|508979 | GTP-binding protein [Bacillus subtilis] | 85 | 78 | |
| 488 | 1 | 1 | 996 | gi\|532548 | ORF15 [Enterococcus faecalis] | 85 | 67 | |
| 535 | 5 | 6468 | 4849 | gi\|634107 | kdpB [Escherichia coli] | 85 | 68 | |
| 584 | 3 | 732 | 562 | gi\|467374 | single strand DNA binding protein | 85 | 75 | |
| | | | | | [Bacillus subtilis]sp\|P37455\|SSB_BACSU | | | |
| | | | | | SINGLE-STRAND BINDING PROTEIN (SSB) HELIX- | | | |
| | | | | | DESTABILIZING PROTEIN). | | | |
| 695 | 1 | 78 | 500 | gi\|499384 | orf189 [Bacillus subtilis] | 85 | 75 | |
| 836 | 1 | 1 | 357 | gi\|153801 | enzyme scr-II [Streptococcus mutans] | 85 | 69 | |
| 17 | 20 | 17212 | 18813 | gi\|532548 | ORF15 [Enterococcus faecalis] | 84 | 68 | |
| 23 | 31 | 18728 | 19987 | gnl\|PID\|e208211 | DNA topoisomerase IV [Streptococcus | 84 | 68 | |
| | | | | | pneumoniae] | | | |
| 34 | 3 | 3112 | 2144 | gi\|143312 | 6-phospho-1-fructokinase (gtg start codon; | 84 | 69 | |
| | | | | | EC 2.7.1.11) [Bacillus tearothermophilus] | | | |
| 36 | 1 | 1 | 1152 | gi\|1644223 | elongation factor G [Bacillus subtilis] | 84 | 73 | |
| 49 | 12 | 6730 | 8190 | gi\|456319 | 74kDa protein [Bacteriophage FC1] | 84 | 65 | |
| 51 | 2 | 1379 | 1663 | gi\|468207 | Submitter comments: A Mg2+ transporting P- | 84 | 71 | |
| | | | | | type ATPase highly omologous with mgtB | | | |
| | | | | | ATPase at 80 min on Salmonella chromosome. | | | |
| | | | | | ediates the influx of Mg2+ only. | | | |
| | | | | | Transcription regulated by xtracellular | | | |
| | | | | | Mg2+ [Salmonella typhimurium] | | | |
| 95 | 6 | 3330 | 3707 | gi\|487277 | Na+ −ATPase subunit C [Enterococcus hirae] | 84 | 64 | |
| 104 | 5 | 6250 | 5459 | gnl\|PID\|e199440 | aspartate carbamoyltransferase, aspartate | 84 | 65 | |
| | | | | | transcarbamylase, | | | |
| | | | | | carbamylaspartotranskinase [Lactobacillus | | | |
| | | | | | plantarum] | | | |
| 105 | 6 | 4605 | 5273 | gi\|467411 | recombination protein [Bacillus subtilis] | 84 | 65 | |
| 114 | 11 | 12278 | 12997 | gi\|556886 | serine hydroxymethyltransferase [Bacillus | 84 | 74 | |
| | | | | | subtilis]pir\|S49363\|S49363 serine | | | |
| | | | | | hydroxymethyltransferase - Bacillus | | | |
| | | | | | ubtilis | | | |
| 117 | 2 | 705 | 1484 | gi\|580906 | B.subtilis genes rpmH, rnpA, 50kd, gidA | 84 | 70 | |
| | | | | | and gidB [Bacillus subtilis] gi\|467381 | | | |
| | | | | | regulation of SpoOJ and 0rf283 (probable) | | | |
| | | | | | [Bacillus ubtilis] | | | |
| 121 | 2 | 1274 | 2119 | gi\|290643 | ATPase [Enterococcus hirae] | 84 | 67 | |
| 121 | 6 | 5016 | 5219 | gi\|153765 | DNA polymerase I [Streptococcus | 84 | 66 | |
| | | | | | pneumoniae] | | | |
| 128 | 27 | 22456 | 20453 | gi\|437916 | isoleucyl-tRNA synthetase [Staphylococcus | 84 | 71 | |
| | | | | | aureus] | | | |
| 130 | 1 | 2 | 133 | gi\|1237013 | ORF2 [Bacillus subtilis] | 84 | 74 | |
| 138 | 35 | 26712 | 25777 | gi\|143795 | transfer RNA-Tyr synthetase [Bacillus | 84 | 69 | |
| | | | | | subtilis] | | | |
| 164 | 28 | 26378 | 27277 | gnl\|PID\|e247026 | orf6 [Lactobacillus sake] | 84 | 72 | |
| 171 | 1 | 158 | 2719 | gi\|499335 | secA protein [Staphylococcus carnosus] | 84 | 68 | |
| 210 | 5 | 4870 | 3884 | gi\|950062 | hypothetical yeast protein 1 [Mycoplasma | 84 | 75 | |
| | | | | | capricolum] pir\|S48578\|S48578 hypothetical | | | |
| | | | | | protein - Mycoplasma capricolum SGC3) | | | |
| | | | | | (fragment) | | | |
| 217 | 7 | 5222 | 3546 | gi\|143597 | CTP synthetase [Bacillus subtilis] | 84 | 68 | |
| 243 | 1 | 1088 | 126 | gi\|143608 | sporulation protein [Bacillus subtilis] | 84 | 70 | |
| 275 | 1 | 578 | 48 | gi\|1103865 | formyl-tetrahydrofolate synthetase | 84 | 72 | |
| | | | | | [Streptococcus mutans] | | | |
| 281 | 1 | 333 | 698 | gi\|1303962 | YqjK [Bacillus subtilis] | 84 | 68 | |
| 292 | 23 | 18340 | 18038 | gi\|142988 | membrane transport protein [Bacillus | 84 | 61 | |
| | | | | | stearothermophilus] pir\|A42478\|A42478 | | | |
| | | | | | glutamine transport protein glnQ - | | | |
| | | | | | [Bacillus tearothermophilus] | | | |
| 309 | 2 | 1114 | 722 | gi\|1644219 | RNA polymerase beta′ subunit [Bacillus | 84 | 72 | |
| | | | | | subtilis] | | | |
| 315 | 1 | 668 | 3 | gi\|149601 | thymidylate synthase (EC 2.1.1.45) | 84 | 72 | |
| | | | | | [Lactobacillus casei] | | | |
| 334 | 6 | 5375 | 6862 | gi\|1354211 | PET112-like protein [Bacillus subtilis] | 84 | 71 | |
| 338 | 10 | 7585 | 10479 | gi\|467444 | transcription-repair coupling factor | 84 | 68 | |
| | | | | | [Bacillus subtilis] sp\|P37474\|MFD_BACSU | | | |
| | | | | | TRANSCRIPTION-REPAIR COUPLING FACTOR | | | |
| | | | | | (TRCF). | | | |
| 338 | 14 | 12713 | 13018 | gi\|467448 | unknown [Bacillus subtilis] | 84 | 64 | |
| 340 | 3 | 1068 | 2273 | gi\|40046 | phosphoglucose isomerase A (AA 1-449) | 84 | 69 | |
| | | | | | [Bacillus stearothermophilus] | | | |
| | | | | | ir\|S15936\|NUBSSA glucose-6-phosphate | | | |
| | | | | | isornerase (EC 5.3.1.9) A - cillus | | | |
| | | | | | stearothermophilus | | | |
| 375 | 2 | 1430 | 1780 | gi\|1402531 | ORE10 [Enterococcus faecalis] | 84 | 64 | |
| 381 | 1 | 2 | 1279 | gnl\|PID\|e208212 | DNA topoisomerase IV [Streptococcus | 84 | 67 | |
| | | | | | pneumoniae] | | | |
| 421 | 1 | 5 | 151 | gi\|710632 | beta-glucosidase [Bacillus subtilis] | 84 | 73 | |
| 421 | 3 | 1229 | 1465 | gi\|710632 | beta-glucosidase [Bacillus subtilis] | 84 | 65 | |
| 445 | 1 | 1080 | 190 | gi\|46985 | glucose-1-phosphate thymidylyltransferase | 84 | 71 | |
| | | | | | [Salmonella enterica] ir\|S23342\|S23342 | | | |
| | | | | | hypothetical protein 6.1 - Salmonella | | | |
| | | | | | choleraesuis p\|P55254\|RFBA_SALAN GLUCOSE- | | | |
| | | | | | 1-PHOSPHATE THYMIDYLYLTRANSFERASE (EC | | | |
| | | | | | 7.7.24) (DTDP-GLUCOSE SYNTHASE) (DTDP- | | | |
| | | | | | GLUCOSE PYROPHOSPHO | | | |
| 466 | 9 | 10467 | 11006 | gi\|147403 | mannose permease subunit II-P-Man | 84 | 61 | |
| | | | | | [Escherichia coli] | | | |
| 497 | 2 | 469 | 1680 | gi\|1220529 | methyl transferase [Streptococcus | 84 | 72 | |
| | | | | | pneumoniae] | | | |
| 545 | 2 | 309 | 2171 | gi\|532548 | ORF15 [Enterococcus faecalis] | 84 | 68 | |
| 550 | 5 | 2744 | 2265 | gi\|455528 | ORF2 [Streptococcus thermophilus | 84 | 54 | |
| | | | | | bacteriophage] | | | |
| 637 | 5 | 2679 | 3545 | gnl\|PID\|e236571 | cell wall anchoring signal [Enterococcus | 84 | 72 | |
| | | | | | faecalis] | | | |
| 653 | 3 | 1023 | 736 | gi\|1408584 | LtrC [Lactococcus lactis lactis] | 84 | 72 | |
| 674 | 1 | 763 | 254 | gi\|467452 | unknown [Bacillus subtilis] | 84 | 66 | |
| 788 | 1 | 165 | 500 | gi\|1196907 | daunorubicin resistance protein | 84 | 66 | |
| | | | | | [Streptomyces peucetius] | | | |
| 675 | 1 | 1 | 621 | gi\|467470 | lysyl-tRNA thynthetase [Bacillus subtilis] | 83 | 71 | |
| 763 | 2 | 374 | 640 | gi\|145851 | envM [Escherichia coli] | 83 | 61 | |
| 774 | 1 | 658 | 2 | gi\|1256145 | YbbP [Bacillus subtilis] | 83 | 60 | |
| 3 | 1 | 58 | 327 | gi\|312443 | carbamoyl-phosphate synthase (glutamine- | 82 | 70 | |
| | | | | | hydrolysing) [Bacillus aldolyticus] | | | |
| 5 | 10 | 6389 | 7708 | sp\|P30053\|SY_STREQ | HISTIDYL-TRNA SYNTHETASE (EC 6.1.1.21) | 82 | 71 | |
| | | | | | (HISTIDINE--TRNA LIGASE) (HISRS). | | | |
| 27 | 4 | 1906 | 1145 | gi\|1303960 | YgjI [Bacillus subtilis] | 82 | 71 | |
| 32 | 2 | 1333 | 965 | gi\|1303839 | YqfR [Bacillus subtilis] | 82 | 60 | |
| 34 | 1 | 1643 | 324 | gnl\|PID\|e218042 | pyruvate kinase [Lactobacillus | 82 | 68 | |
| | | | | | delbrueckii] | | | |
| 55 | 9 | 4182 | 5054 | gi\|1685110 | tetrahydrofolate | 82 | 70 | |
| | | | | | dehydrogenase/cyclohydrolase | | | |
| | | | | | [Streptococcus thermophilus] | | | |
| 62 | 7 | 4644 | 4210 | gi\|143723 | putative [Bacillus subtilis] | 82 | 66 | |
| 88 | 2 | 995 | 1624 | gi\|535349 | CodW [Bacillus subtilis] | 82 | 66 | |
| 94 | 7 | 4790 | 3432 | gi\|1146247 | asparaginyl-tRNA synthetase [Bacillus | 82 | 67 | |
| | | | | | subtilis] | | | |
| 110 | 23 | 21590 | 20742 | gi\|467403 | seryl-tRNA synthetase [Bacillus subtilis] | 82 | 69 | |
| 114 | 7 | 8623 | 9228 | gi\|703442 | thyrmidine kinase [Streptococcus gordonii] | 82 | 68 | |
| 123 | 6 | 4499 | 4996 | gi\|467356 | unknown [Bacillus subtilis] | 82 | 68 | |
| 130 | 3 | 1413 | 2381 | gi\|308851 | ATP binding protein [Lactococcus lactis] | 82 | 64 | |
| 144 | 3 | 3292 | 2339 | gnl\|PID\|e183449 | putative ATP-binding protein of ABC-type | 82 | 62 | |
| | | | | | [Bacillus subtilis] | | | |
| 144 | 7 | 5331 | 5110 | gi\|335495 | A23R; putative [Vaccinia virus] | 82 | 47 | |
| 159 | 4 | 2533 | 5010 | gi\|143148 | transfer RNA-Leu synthetase [Bacillus | 82 | 71 | |
| | | | | | subtilis] | | | |
| 159 | 6 | 5845 | 5387 | gi\|467354 | unknown [Bacillus subtilis] | 82 | 55 | |
| 171 | 8 | 8510 | 9349 | gi\|1591672 | phosphate transgport system ATP-binding | 82 | 61 | |
| | | | | | protein [Methanococcus jannaschii] | | | |
| 222 | 5 | 2158 | 3402 | gi\|143444 | RNase PH [Bacillus subtilis] | 82 | 66 | |
| 254 | 6 | 1621 | 1112 | gi\|49316 | ORF2 gene product [Bacillus subtilis] | 82 | 61 | |
| 279 | 12 | 9839 | 8442 | gi\|1237019 | Srb [Bacillus subtilis] | 82 | 67 | |
| 288 | 1 | 22 | 546 | gi\|149393 | lacA [Lactococcus lactis] | 82 | 73 | |
| 345 | 8 | 5608 | 8118 | gi\|442360 | ClpC adenosine triphosphatase [Bacillus | 82 | 63 | |
| | | | | | subtilis] | | | |
| 367 | 3 | 1472 | 1110 | gi\|142463 | RNA polymerase alpha-core-subunit | 82 | 75 | |
| | | | | | [Bacillus subtilis] | | | |
| 367 | 9 | 4961 | 3660 | gi\|44073 | SecY protein [Lactococcus lactis] | 82 | 65 | |
| 367 | 28 | 12719 | 12411 | pir\|A02815\|R5BS23 | ribosomal protein L23 - Bacillus | 82 | 66 | |
| | | | | | stearothermophilus | | | |
| 367 | 29 | 13330 | 12701 | gi\|1165304 | L4 [Bacillus subtilis] | 82 | 67 | |
| 379 | 5 | 4396 | 3107 | gi\|887820 | UUG start; possible frameshift at end? | 82 | 71 | |
| | | | | | [Escherichia coli] | | | |
| 393 | 2 | 1145 | 711 | gi\|1303993 | YqkL [Bacillus subtilis] | 82 | 67 | |
| 416 | 1 | 3 | 650 | gi\|475113 | sucrase [Pediococcus pentosaceus] | 82 | 69 | |
| 477 | 1 | 1 | 1209 | gi\|309663 | signaling protein [Plasmid pCF10] | 82 | 62 | |
| 497 | 7 | 3760 | 4275 | gi\|532551 | ORF18 [Enterococcus faecalis] | 82 | 67 | |
| 535 | 3 | 4275 | 1666 | gi\|1747434 | KdpD [Clostridium acetobutylicum] | 82 | 62 | |
| 587 | 1 | 488 | 108 | gi\|1303840 | YgfS [Bacillus subtilis] | 82 | 71 | |
| 623 | 2 | 122 | 1348 | gi\|460259 | enolase [Bacillus subtilis] | 82 | 67 | |
| 656 | 1 | 1 | 1908 | gi\|1184680 | polynucleotide phosphorylase [Bacillus | 82 | 69 | |
| | | | | | subtilis] | | | |
| 687 | 1 | 227 | 1252 | gi\|40218 | PRPP synthetase (AA 1-317) [Bacillus | 82 | 64 | |
| | | | | | subtilis] | | | |
| 728 | 1 | 3 | 527 | gi\|1146183 | putative [Bacillus subtilis] | 82 | 65 | |
| 741 | 1 | 3 | 704 | gi\|153804 | sucrose-6-phosphate hydrolase | 82 | 66 | |
| | | | | | [Streptococcus mutans] | | | |
| 846 | 1 | 458 | 3 | gnl\|PID\|e221400 | tex gene product [Bordetella pertussis] | 82 | 76 | |
| 865 | 1 | 18 | 308 | gi\|416006 | orf CJ01.2 [Campylobacter jejuni] | 82 | 57 | |
| 876 | 1 | 207 | 689 | gi\|1064795 | function unknown [Bacillus subtilis] | 82 | 62 | |
| 925 | 1 | 436 | 128 | gi\|1773195 | hypothetical [Escherichia coli] | 82 | 74 | |
| 983 | 2 | 280 | 474 | gi\|40026 | homologous to E.coli gidA [Bacillus | 82 | 78 | |
| | | | | | subtilis] | | | |
| 12 | 3 | 4778 | 5788 | gi\|1100074 | tryptophanyl-tRNA synthetase [Clostridium | 81 | 68 | |
| | | | | | longisporum] | | | |
| 31 | 4 | 2984 | 4456 | gi\|849026 | hypothetical 54.6-kDa protein [Bacillus | 81 | 68 | |
| | | | | | subtilis] | | | |
| 34 | 6 | 6707 | 6910 | gi\|606067 | ORF_f444 [Escherichia coli] | 81 | 54 | |
| 37 | 1 | 1 | 144 | gi\|1303854 | YggG [Bacillus subtilis] | 81 | 59 | |
| 37 | 3 | 2671 | 1958 | gi\|40056 | phoP gene product [Bacillus subtilis]81 | 61 | | |
| 57 | 3 | 1733 | 3220 | gi\|1657506 | hypothetical protein [Escherichia coli] | 81 | 66 | |
| 60 | 5 | 5564 | 4440 | gi\|143370 | phosphoribosylpyrophosphate | 81 | 63 | |
| | | | | | amidotransferase (PUR-F; EC 2.4.2.14) | | | |
| | | | | | Bacillus subtilis] | | | |
| 73 | 3 | 2706 | 1450 | gi\|853767 | UDP-N-acetylglucosamine 1- | 81 | 61 | |
| | | | | | carboxyvinyltransferase [Bacillus ubtilis] | | | |
| 88 | 4 | 1977 | 2732 | gnl\|PID\|e137596 | heat shock induced protein HtpO | 81 | 67 | |
| | | | | | [Lactobacillus leichniannii] | | | |
| 88 | 5 | 2723 | 3040 | gi\|535350 | CodX [Bacillus subtilis] | 81 | 65 | |
| 101 | 4 | 3091 | 2435 | gi\|1109687 | ProZ [Bacillus subtilis] | 81 | 60 | |
| 101 | 7 | 5884 | 4661 | gi\|1109684 | ProV [Bacillus subtilis] | 81 | 64 | |
| 101 | 9 | 7501 | 7965 | gi\|1001768 | queuosine biosynthesis protein QueA | 81 | 47 | |
| | | | | | [Synechocystis sp.] | | | |
| 116 | 5 | 2766 | 3395 | gi\|1146234 | dihydrodipicolinate reductase [Bacillus | 81 | 66 | |
| | | | | | subtilis] | | | |
| 121 | 5 | 4811 | 5074 | gi\|153765 | DNA polymerase I [Streptococcus | 81 | 64 | |
| | | | | | pneumoniae] | | | |
| 121 | 7 | 5203 | 7488 | gi\|153765 | DNA polymerase I [Streptococcus | 81 | 70 | |
| | | | | | pneumoniae] | | | |
| 127 | 5 | 5103 | 3826 | gi\|290561 | o188 [Escherichia coli] | 81 | 48 | |
| 147 | 1 | 299 | 1279 | gi\|467462 | cysteine synthetase A [Bacillus subtilis] | 81 | 65 | |
| 147 | 2 | 1370 | 1861 | gnl\|PID\|e281583 | hypothetical 16.4 kd protein [Bacillus | 81 | 63 | |
| | | | | | subtilis] | | | |
| 154 | 1 | 168 | 638 | gi\|149533 | coniugated bile acid hydrolase | 81 | 66 | |
| | | | | | [Lactobacillus plantarum] | | | |
| 154 | 2 | 1074 | 1277 | gnl\|PID\|e242898 | aBIR [Lactococcus lactis] | 81 | 59 | |
| 158 | 14 | 13790 | 12324 | gi\|558559 | pyrimidine nucleoside phosphorylase | 81 | 71 | |
| | | | | | [Bacillus subtilis] | | | |
| 164 | 5 | 2469 | 3035 | gi\|727436 | putative 20-kDa protein [Lactococcus | 81 | 61 | |
| | | | | | lactis] | | | |
| 223 | 8 | 5293 | 6153 | gn1\|PID\|e254976 | hypothetical protein [Bacillus subtilis] | 81 | 66 | |
| 238 | 1 | 185 | 937 | gi\|622991 | mannitol transport protein [Bacillus | 81 | 68 | |
| | | | | | stearotherinophilus]sp\|P50852 PTMB_BACST | | | |
| | | | | PTS SYSTEM, MANNITOL-SPECIFIC IIBC | | | | |
| | | | | | COMPONENT EIIBC-MTL) (MANNITOL-PERMEASE | | | |
| | | | | | IIBC COMPONENT) (PHOSPHOTRANSFERASE NZYME | | | |
| | | | | | II, BC COMPONENT) (EC 2.7.1.69) (EII-MTL). | | | |
| 276 | 7 | 3109 | 2819 | pir\|A41207\|A41207 | collagen 13, nonfibrillar - freshwater | 81 | 77 | |
| | | | | | sponge (Ephvdatia muelleri) (fragrnent) | | | |
| 307 | 2 | 1983 | 3617 | gi\|153742 | dextran glucosidase [Streptococcus mutans] | 81 | 69 | |
| 322 | 2 | 122 | 286 | gi\|296147 | Asd protein [Bacillus subtilis] | 81 | 63 | |
| 326 | 6 | 5352 | 4513 | gi\|40041 | pyruvate dehydrogenase (lipoamide) | 81 | 69 | |
| | | | | | [Bacillus stearothermophilus] | | | |
| | | | | | ir\|S10798 DEBSPF pyruvate dehydrogenase | | | |
| | | | | | (lipoamide) (EC 1.2.4.1) pha chain - | | | |
| | | | | | Bacillus stearothermophilus | | | |
| 329 | 3 | 1774 | 1448 | gi\|1117994 | surface antigen A variant precursor | 81 | 72 | |
| | | | | | [Streptococcus pneumoniae] | | | |
| 346 | 3 | 1056 | 1199 | gi\|536970 | ORF_fS43 [Escherichia coli] | 81 | 43 | |
| 362 | 4 | 1131 | 2213 | gi\|1001826 | cadmium-transporting ATPase [Synechocystis | 81 | 64 | |
| | | | | | sp.] | | | |
| 391 | 3 | 1345 | 575 | gi\|1184967 | ScrR [Streptococcus mutans] | 81 | 66 | |
| 441 | 3 | 1873 | 3447 | gi\|1742675 | Phosphotransferase system enzyme II (EC | 81 | 64 | |
| | | | | | 2.7.1.69) MalX [Escherichia coli] | | | |
| 556 | 2 | 1062 | 493 | gi\|1553037 | RecN [Bacillus subtilis] | 81 | 66 | |
| 710 | 2 | 361 | 816 | gi\|1303840 | YgfS [Bacillus subtilis] | 81 | 68 | |
| 804 | 1 | 403 | 2 | gi\|149533 | conjugated bile acid hydrolase | 81 | 68 | |
| | | | | | [Lactobacillus_plantarum] | | | |
| 5 | 7 | 3311 | 4255 | gi\|407881 | stringent response-like protein | 80 | 62 | |
| | | | | | [Streptococcus equisimilis] | | | |
| | | | | | pir\|S39975\|S39975 stringent response-like | | | |
| | | | | | protein - Streptococcus quisimilis | | | |
| 17 | 10 | 8283 | 8438 | gi\|1326394 | B0218.7 gene product [Caenorhabditis | 80 | 53 | |
| | | | | | elegans] | | | |
| 17 | 15 | 12258 | 12776 | gi\|532551 | ORF18 [Enterococcus faecalis] | 80 | 63 | |
| 22 | 1 | 3 | 2180 | gi\|44027 | Tma protein [Lactococcus lactis] | 80 | 70 | |
| 37 | 6 | 3707 | 5140 | pir\|B47154\|B47154 | signal recognition particle 54K chain | 80 | 64 | |
| | | | | | homolog Ffh - Bacillus subtilis | | | |
| 42 | 1 | 2 | 259 | gi\|1066157 | chaperonin-10 [Thermus aquaticus | 80 | 66 | |
| | | | | | thermophilus] | | | |
| 49 | 16 | 11106 | 11309 | gi\|1136430 | similar to hypothetical protein YM49959.11C | 80 | 53 | |
| | | | | | of S.cerevisiae. [Homo sapiens] | | | |
| 60 | 4 | 4465 | 3407 | gi\|143371 | phosphoribosyl aminoimidazole synthetase | 80 | 62 | |
| | | | | | (PUR-M) [Bacillus subtilis] | | | |
| | | | | | pir\|H29326\|AJBSCL | | | |
| | | | | | phosphoribosylformyiglycinamidine cyclo- | | | |
| | | | | | ligase EC 6.3.3.1) Bacillus subtilis | | | |
| 60 | 9 | 9023 | 8745 | pir\|E29326\|E29326 | hypothetical protein (pur operon) - | 80 | 50 | |
| | | | | | Bacillus subtilis | | | |
| 66 | 1 | 1 | 783 | gi\|520753 | DNA topoisornerase I [Bacillus subtilis] | 80 | 66 | |
| 80 | 3 | 2519 | 1821 | gnl\|PID\|e236074 | beta-phosphoglucomutase [Lactococcus | 80 | 62 | |
| | | | | | lactis] | | | |
| 83 | 9 | 6268 | 5378 | gi\|1070079 | R08B4.1 [Caenorhabditis elegans] | 80 | 72 | |
| 89 | 18 | 19093 | 18845 | gi\|39451 | type III restriction endonuclease | 80 | 72 | |
| | | | | | [Bacillus cereus] ir\|S15518\|JC1116 type | | | |
| | | | | | III site-specific deoxyribonuclease (EC | | | |
| | | | | | 1.21.5) - Bacillus cereus (fragment) | | | |
| 97 | 1 | 366 | 4 | gi\|148506 | dnaJ [Erysipelothrix rhusiopathiae] | 80 | 70 | |
| 107 | 2 | 1094 | 591 | sp\|P37214\|ERA_STRM | GTP-BINDING PROTEIN ERA HOMOLOG. | 80 | 64 | |
| | | | | U | | | | |
| 114 | 3 | 1474 | 5076 | gi\|43863 | pyruvate-flavodoxin oxidoreductase | 80 | 62 | |
| | | | | | [Kiebsiella pneumoniae] ir\|S01997\|QQKBFP | | | |
| | | | | | pyruvate (flavodoxin) dehydrogenase (EC | | | |
| | | | | | 1.2.99.-) Klebsiella pneumoniae | | | |
| 117 | 3 | 1456 | 2367 | gi\|40031 | spoOJ93 gene product [Bacillus subtilis] | 80 | 56 | |
| 126 | 3 | 1857 | 709 | gi\|551854 | ORF2 [Erwinia herbicola] | 80 | 68 | |
| 128 | 28 | 23265 | 22447 | gi\|437916 | isoleucyl-tRNA synthetase [Staphylococcus | 80 | 63 | |
| | | | | | aureus] | | | |
| 133 | 10 | 9128 | 9856 | gi\|520844 | orf4 [Bacillus subtilis] | 80 | 63 | |
| 158 | 4 | 3926 | 2703 | gi\|944943 | phosphopentomutase [Bacillus subtilis] | 80 | 64 | |
| 172 | 5 | 3732 | 3920 | sp\|P20182\|YT14_STR | HYPOTHETICAL 29.1 KD PROTEIN IN TRANSPOSON 80 63 | | | |
| | | | | FR | TN4556. | | | |
| 180 | 16 | 15548 | 16393 | gi\|1773200 | hypothotical protein [Escherichia coli] | 80 | 66 | |
| 181 | 10 | 8597 | 7407 | gi\|143806 | AroF [Bacillus subtilis] | 80 | 64 | |
| 194 | 4 | 1580 | 1957 | gi\|47394 | 5-oxoprolyl-peptidase [Streptococcus | 80 | 66 | |
| | | | | | pyogenes] | | | |
| 213 | 5 | 3515 | 4078 | gnl\|PID\|e199384 | pyrR gene product [Lactobacillus | 80 | 65 | |
| | | | | | plantarum] | | | |
| 217 | 11 | 7724 | 8395 | gi\|1561567 | Unknown [Bacillus subtilis] | 80 | 65 | |
| 218 | 6 | 4843 | 5331 | gi\|1574120 | branched-chain-amino-acid transaminase | 80 | 64 | |
| | | | | | [Haemophilus influenzae] | | | |
| 225 | 8 | 6092 | 5829 | gi\|530459 | similar to phosphotransferase EII | 80 | 52 | |
| | | | | | [Mycoplasma capricolum] | | | |
| 229 | 2 | 1170 | 178 | gi\|1502419 | P1sX [Bacillus subtilis] | 80 | 59 | |
| 243 | 3 | 2545 | 2150 | gi\|1732315 | transport system permease homolog | 80 | 64 | |
| | | | | | [Listeria monocytogenes] | | | |
| 275 | 2 | 694 | 939 | gi\|1256629 | cold-shock protein [Bacillus subtilis] | 80 | 65 | |
| 307 | 3 | 3607 | 3888 | gi\|1321625 | exo-alpha-1, 4-glucosidase [Bacillus | 80 | 73 | |
| | | | | | stearothermophilus | | | |
| 322 | 3 | 284 | 1090 | gi\|142828 | aspartate semialdehyde dehydrogenase | 80 | 62 | |
| | | | | | [Bacillus subtilis] sp\|Q04797\|DHAS_BACSU | | | |
| | | | | | ASPARTATE- SEMIALDEHYDE DEHYDROGENASE (EC | | | |
| | | | | | .2.1.11) | (ASA DEHYDROGENASE). | | |
| 349 | 1 | 2 | 616 | gi\|495089 | recombinase [Staphylococcus aureus] | 80 | 65 | |
| 367 | 7 | 3511 | 2924 | gi\|44074 | adenylate kinase [Lactococcus lactis] | 80 | 64 | |
| 386 | 7 | 4305 | 5306 | gi\|149396 | lacD [Lactococcus lactis] | 80 | 64 | |
| 394 | 3 | 2642 | 3757 | pir\|B39096\|B39096 | alkaline phosphatase (EC 3.1.3.1) III | 80 | 64 | |
| | | | | | precursor - Bacillus subtilis | | | |
| 399 | 17 | 12070 | 13488 | gi\|1591862 | oxaloacetate decarboxylase, alpha subunit | 80 | 61 | |
| | | | | | [Methanococcus jannaschii] | | | |
| 399 | 24 | 22979 | 24907 | gi\|40026 | homologous to E.coli gidA [Bacillus | 80 | 67 | |
| | | | | | subtilis] | | | |
| 435 | 3 | 2217 | 2032 | gi\|559863 | clyA [Plasmid pAD1] | 80 | 78 | |
| 466 | 1 | 3 | 1208 | gi\|467330 | replicativo DNA helicaso [Bacillus | 80 | 61 | |
| | | | | | subtilis] | | | |
| 475 | 4 | 3402 | 2947 | gi\|532547 | ORF14 [Enterococcus faecalis] | 80 | 68 | |
| 491 | 4 | 3844 | 4392 | gi\|473892 | large-conductance mechanosensitive channel | 80 | 56 | |
| | | | | | [Escherichia coli] gi\|473420 yhdC | | | |
| | | | | | [Escherichia coli] | | | |
| 605 | 2 | 1252 | 338 | gi\|580875 | ipa-57d gene product [Bacillus subtilis] | 80 | 69 | |
| 615 | 1 | 760 | 14 | gi\|467469 | unknown [Bacillus subtilis] | 80 | 66 | |
| 668 | 1 | 117 | 587 | pir\|S16974\|R5BS7F | ribosomal protein L9 - Bacillus | 80 | 71 | |
| | | | | | stearothermophilus | | | |
| 684 | 2 | 694 | 464 | gi\|786314 | Highly similar to Glycogen debranching | 80 | 33 | |
| | | | | | enzyme 4-alpha-glucanotransferase, Swiss | | | |
| | | | | | Prot. accession number P35573) | | | |
| | | | | | Saccharomyces cerevisiae] | | | |
| 767 | 1 | 1 | 480 | gi\|41828 | istB gene product [Escherichia coli] | 80 | 52 | |
| 818 | 1 | 1 | 357 | gi\|743856 | intrageneric coaggregation-relevant | 80 | 66 | |
| | | | | | adhesin [Streptococcus gordonii] | | | |
| 833 | 1 | 325 | 95 | gi\|1561567 | Unknown [Bacillus subtilis] | 80 | 68 | |
| 934 | 1 | 394 | 56 | gi\|1001706 | ABC transporter subunit [Synechocystis | 80 | 63 | |
| | | | | | sp.] | | | |
| 948 | 1 | 465 | 4 | gi\|1773196 | similar to B. stearothermophilus N- | 80 | 59 | |
| | | | | | carbamyl-L-amino acid amidohydrolase | | | |
| | | | | | [Escherichia coli] | | | |
| 949 | 1 | 61 | 411 | gi\|1330380 | Similar to cystathionine gamma-lyase | 80 | 61 | |
| | | | | | [Caenorhabditis elegans] | | | |
| 20 | 2 | 468 | 1262 | gi\|1256698 | chitinase [Serratia marcescens] | 79 | 67 | |
| 22 | 3 | 2420 | 3238 | gi\|467460 | unknown [Bacillus subtilis] | 79 | 59 | |
| 24 | 1 | 39 | 1109 | gi\|1303821 | YgfE [Bacillus subtilis] | 79 | 61 | |
| 26 | 1 | 214 | 873 | gi\|403984 | deoxyguanosine kinase/deoxyadenosine | 79 | 68 | |
| | | | | | kinase(I) subunit Lactobacillus | | | |
| | | | | | acidophilus] | | | |
| 47 | 8 | 10268 | 8106 | gi\|153657 | mismatch repair protein [Streptococcus | 79 | 63 | |
| | | | | | pneumoniae] pir\|A33589\|A33589 mismatch | | | |
| | | | | | repair protein hexB - Streptococcus | | | |
| | | | | | neumoniae | | | |
| 48 | 9 | 9905 | 9198 | gi\|290566 | f213 [Escherichia coli] | 79 | 53 | |
| 58 | 4 | 4677 | 3694 | gi\|1653179 | hydrogenase subunit [Synechocystis sp.] | 79 | 52 | |
| 63 | 6 | 3605 | 5443 | gi\|1064813 | homologous to sp:PHOR_BACSU [Bacillus | 79 | 55 | |
| | | | | | subtilis] | | | |
| 88 | 8 | 5493 | 4771 | gnl\|PID\|e208252 | unidentified [Streptococcus pneumoniae] | 79 | 57 | |
| 146 | 8 | 6649 | 5609 | gi\|153676 | tagatose 1,6-aldolase [Streptococcus | 79 | 63 | |
| | | | | | mutans] | | | |
| 149 | 4 | 2554 | 1976 | gi\|1216490 | DNA/pantothenate metabolism flavoprotein | 79 | 64 | |
| | | | | | [Streptococcus mutans] | | | |
| 158 | 2 | 1859 | 1143 | gi\|1276873 | DeoD [Streptococcus thermophilus] | 79 | 67 | |
| 179 | 19 | 19022 | 18417 | gi\|467372 | 3′-exo-deoxyribonuclease [Bacillus | 79 | 61 | |
| | | | | | subtilis] | | | |
| 222 | 2 | 982 | 230 | gi\|142988 | membrane transport protein [Bacillus | 79 | 59 | |
| | | | | | stearothemophilus] pir\|A42478\|A42478 | | | |
| | | | | | glutamine transport protein glnQ - | | | |
| | | | | | Bacillus tearothermophilus | | | |
| 228 | 6 | 4060 | 3401 | gi\|413950 | ipa-26d gene product [Bacillus subtilis] | 79 | 55 | |
| 229 | 3 | 3270 | 1219 | gnl\|PID\|e186699 | MmsA [Streptococcus pneumoniae] | 79 | 62 | |
| 238 | 7 | 5750 | 5100 | gi\|596046 | L8003.16 gene product [Saccharomyces | 79 | 55 | |
| | | | | | cerevisiae] | | | |
| 269 | 10 | 6664 | 5489 | gi\|1303788 | YgeH [Bacillus subtilis] | 79 | 63 | |
| 274 | 1 | 1 | 1143 | gi\|153062 | helicase [Staphylococcus aureus] | 79 | 65 | |
| 290 | 9 | 7364 | 8779 | gi\|466882 | pps1; B1496_c2_189 [Mycobacterium leprae] | 79 | 64 | |
| 292 | 22 | 18122 | 17595 | gi\|1303951 | YgiZ [Bacillus subtilis] | 79 | 61 | |
| 316 | 3 | 864 | 2003 | gi\|1146207 | putative [Bacillus subtilis] | 79 | 58 | |
| 326 | 2 | 1772 | 360 | gi\|40044 | dihydrolipoamide dehydrogenase [Bacillus | 79 | 65 | |
| | | | | | stearothermophilus] ir\|S13839\|813839 | | | |
| | | | | | dihydrolipoamide dehydrogenase (EC | | | |
| | | | | | 1.8.1.4) - cillus stearothermophilus | | | |
| 363 | 5 | 5738 | 7180 | gi\|1657519 | hypothetical protein [Eseherichia coli] | 79 | 63 | |
| 367 | 11 | 5668 | 5447 | gi\|216337 | ORE for L30 ribosmnal protein [Bacillus | 79 | 63 | |
| | | | | | subtilis] | | | |
| 375 | 5 | 4346 | 3393 | gi\|1644203 | unknown [Bacillus subtilis] | 79 | 62 | |
| 406 | 2 | 666 | 1481 | gi\|49316 | ORF2 gene product [Bacillus subtilis] | 79 | 58 | |
| 460 | 7 | 4973 | 5860 | gi\|1276664 | acetyl-CoA carboxylase carboxytransferase | 79 | 62 | |
| | | | | | beta subunit [Porphyra purpurea] | | | |
| 486 | 1 | 380 | 3 | gi\|1256618 | transport protein [Bacillus subtilis] | 79 | 63 | |
| 488 | 3 | 987 | 1997 | gi\|532547 | ORE14 [Enterococcus faecalis] | 79 | 69 | |
| 500 | 2 | 1358 | 681 | gi\|535662 | transposase [Insertion sequence IS1251] | 79 | 75 | |
| 523 | 3 | 1803 | 820 | gi\|142981 | ORF5; This ORF includes a region (aa23- | 79 | 62 | |
| | | | | | 103) containing a potential ron-sulphur | | | |
| | | | | | centre homologous to a region of | | | |
| | | | | | Rhodospirillum rubrum nd Chromatium | | | |
| | | | | | vinosum; putative [Bacillus | | | |
| | | | | | stearothermophilus] pir\|PQ0299\|PQ0299 | | | |
| | | | | | hypothetical protein 5 (gidA 3′ region) - | | | |
| 552 | 2 | 2401 | 902 | gi\|887851 | ORF_o479 [Escherichia coli] | 79 | 63 | |
| 587 | 2 | 622 | 434 | gi\|1303840 | YgfS [Bacillus subtilis] | 79 | 66 | |
| 612 | 1 | 1 | 378 | gi\|1064791 | function unknown [Bacillus subtilis] | 79 | 56 | |
| 654 | 1 | 2 | 286 | pir\|A47079\|A47079 | heat shock protein DnaJ - Lactococcus | 79 | 75 | |
| | | | | | lactis | | | |
| 701 | 2 | 325 | 534 | gi\|143793 | tyrosyl-tRNA synthetase [Bacillus | 79 | 63 | |
| | | | | | caldotenax] | | | |
| 708 | 2 | 369 | 566 | gi\|488430 | alcohol dehydrogenase 2 [Entamoeba | 79 66 | | |
| | | | | | histolytica] | | | |
| 840 | 1 | 140 | 1078 | gi\|1573250 | aspartate aminotransferase (aspC) | 79 | 65 | |
| | | | | | [Haemophilus influenzae] | | | |
| 5 | 9 | 5555 | 6049 | gi\|407880 | ORF1 [Streptococcus equisimilis] | 78 | 58 | |
| 33 | 4 | 3755 | 4597 | gi\|1742846 | NH(3)-dependent NAD(+) synthetase (EC | 78 | 64 | |
| | | | | | 6.3.5.1) (Nitrogen-regulatory protein) | | | |
| | | | | | [Escherichia coli] | | | |
| 60 | 7 | 8100 | 5854 | gi\|143369 | phosphoribosylformyl glycinamidine | 78 | 62 | |
| | | | | | synthetase II (PUR-Q) [Bacillus ubtilis] | | | |
| 65 | 4 | 3407 | 2625 | gi\|1661179 | high affinity branched chain amino acid | 78 | 67 | |
| | | | | | transport protein [Streptococcus mutans] | | | |
| 76 | 7 | 5760 | 4747 | gi\|1161061 | dioxygenase [Methylobacterium extorguens] | 78 | 62 | |
| 81 | 11 | 7141 | 6824 | gi\|1072380 | ORF3 [Lactococcus lactis] | 78 | 67 | |
| 83 | 5 | 2559 | 2843 | gi\|1256896 | L9606.1 gene product [Saccharomyces | 78 | 52 | |
| | | | | | cerevisiae] | | | |
| 85 | 4 | 4298 | 3288 | gi\|142612 | branched chain alpha-keto acid | 78 | 61 | |
| | | | | | dehydrogenase El-beta [Bacillus ubtilis] | | | |
| 85 | 8 | 6723 | 6307 | gi\|1303941 | YqiV [Bacillus subtilis] | 78 | 62 | |
| 88 | 10 | 6477 | 6689 | gi\|222585 | nucleocapsid protein [Sialodacryoadenitis | 78 | 57 | |
| | | | | | virus] | | | |
| 93 | 5 | 1838 | 2641 | gi\|405133 | putative [Bacillus subtilis] | 78 | 51 | |
| 117 | 1 | 3 | 707 | gi\|40027 | homologous to E.coli gidB [Bacillus | 78 | 64 | |
| | | | | | subtilis] | | | |
| 117 | 11 | 9624 | 8338 | gi\|467403 | seryl-tRNA synthetase [Bacillus subtilis] | 78 | 63 | |
| 132 | 2 | 2323 | 2024 | gi\|683484 | fusion protein [Mumps virus] | 78 | 63 | |
| 133 | 3 | 2241 | 3413 | gi\|405622 | unknown [Bacillus subtilis] | 78 | 63 | |
| 150 | 2 | 568 | 1425 | gnl\|PID\|e185373 | ceuD gene product [Campylobacter coil] | 78 | 52 | |
| 155 | 2 | 604 | 1182 | gi\|285628 | transcription antitermination factor NusG | 78 | 61 | |
| | | | | | [Bacillus subtilis] pir\|S39859\|539859 | | | |
| | | | | | transcription antitermination factor NusG | | | |
| | | | | | - acillus subtilis | | | |
| 156 | 2 | 308 | 2629 | gi\|1573874 | ATP-dependent protease binding subunit | 78 | 59 | |
| | | | | | (clpB) [Haemophilus influenzae] | | | |
| 158 | 3 | 2719 | 1868 | gi\|1638804 | purine nucleoside phosphorylase [Bacillus | 78 | 64 | |
| | | | | | stearothermophilus] | | | |
| 160 | 5 | 2058 | 3050 | gi\|1161061 | dioxygenase [Methylobacterium extorguens] | 78 | 60 | |
| 161 | 3 | 1466 | 3295 | gnl\|PID\|e280490 | unknown [Streptococcus pneumoniae] | 78 | 62 | |
| 169 | 1 | 2 | 2206 | gi\|1072361 | pyruvate-formate-lyase [Clostridium | 78 | 61 | |
| | | | | | pasteurianum] | | | |
| 171 | 2 | 2833 | 3897 | sp\|P28367\| | PROBABLE PEPTIDE CHAIN RELEASE FACTOR 2 | 78 | 64 | |
| | | | | RF2_BACS | (RF-2) (FRAGMENT). | | | |
| | | | | U | | | | |
| 180 | 15 | 14851 | 15567 | gi\|1773199 | hypothetical proteinh [Escherichia coli] | 78 | 67 | |
| 185 | 1 | 1142 | 3 | pir\|C33496\|C33496 | hisC homolog - Bacillus subtilis | 78 | 59 | |
| 188 | 3 | 1863 | 4178 | gnl\|PID\|e256969 | nifJ gene product [Enterobacter | 78 | 62 | |
| | | | | | agglomerans] | | | |
| 216 | 7 | 5136 | 5600 | gnl\|PID\|e276830 | UDP-N-acetylglucosamine 1- | 78 | 60 | |
| | | | | | carboxyvinyltransferase [Bacillus | | | |
| | | | | | subtilis] | | | |
| 216 | 8 | 5531 | 6508 | gnl\|PID\|e276830 | UDP-N-acetylglucosamine 1- | 78 | 63 | |
| | | | | | carboxyvinyltransferase [Bacillus | | | |
| | | | | | subtilis] | | | |
| 238 | 26 | 24515 | 25387 | gi\|396681 | rhamnulose-1-phosphate aldolase | 78 | 56 | |
| | | | | | [Escherichia coli] | | | |
| 256 | 6 | 4189 | 6237 | gi\|467427 | methionyl-tRNA synthetase [Bacillus | 78 | 67 | |
| | | | | | subtills] | | | |
| 292 | 4 | 2063 | 2353 | gi\|1742823 | Proton/sodium-glutamate symport protein | 78 | 62 | |
| | | | | | (Glutamate-aspartate carrier protein) | | | |
| | | | | | [Escherichia coli] | | | |
| 305 | 1 | 268 | 1872 | gi\|143582 | spoIIIEA protein [Bacillus subtilis] | 78 | 58 | |
| 337 | 2 | 2332 | 1448 | gi\|308861 | GTG start codon [Lactococcus lactis] | 78 | 63 | |
| 338 | 2 | 606 | 1466 | gi\|1773142 | similar to the 20.2kd protein in TETB-EXOA | 78 | 66 | |
| | | | | | region of B. subtilis [Escherichia coli] | | | |
| 362 | 1 | 109 | 429 | gi\|150719 | cadmium resistance protein [Plasmid pI258] | 78 | 51 | |
| 379 | 3 | 2878 | 1922 | gi\|887824 | ORF_o310 [Escherichia coli] | 78 | 60 | |
| 446 | 2 | 962 | 1636 | gi\|537235 | Kenn Rudd identifies as gpmB [Escherichia | 78 | 43 | |
| | | | | | coli] | | | |
| 495 | 5 | 3038 | 3502 | gi\|634107 | kdpB [Escherichia coli] | 78 | 58 | |
| 502 | 3 | 3077 | 1470 | gi\|1652592 | peptide-chain-release factor 3 | 78 | 58 | |
| | | | | | [Synechocystis sp.] | | | |
| 523 | 1 | 2 | 616 | gi\|289288 | lexA [Bacillus subtilis] | 78 | 59 | |
| 571 | 1 | 99 | 365 | gnl\|PID\|e249644 | YneP [Bacillus subtilis] | 78 | 65 | |
| 573 | 3 | 1258 | 1971 | gi\|1731683 | component II of heptaprenyl diphosphate | 78 | 50 | |
| | | | | | synthase [Bacillus stearothermophilus] | | | |
| 575 | 2 | 434 | 168 | gi\|58831 | The experimental evidence that this | 78 | 47 | |
| | | | | | sequence codes for a complete gag otein is | | | |
| | | | | | that transfection of the viral genome | | | |
| | | | | | results in oduction of infectious virus | | | |
| | | | | | [Cas-Br-E murine leukemia virus] | | | |
| | | | | | p\|P27460\|GAG_MLVCB GAG POLYPROTEIN | | | |
| | | | | | (CONTAINS: CORE PROTEIN P15; N | | | |
| 607 | 1 | 148 | 708 | gi\|530410 | Ala-tRNA synthetase [Mycoplasma | 78 | 63 | |
| | | | | | capricolum] | | | |
| 655 | 2 | 300 | 899 | gi\|147404 | mannose permease subunit II-M-Man | 78 | 60 | |
| | | | | | [Escherichia coli] | | | |
| 704 | 1 | 181 | 2 | gi\|467430 | unknown [Bacillus subtilis] | 78 | 63 | |
| 708 | 1 | 1 | 378 | gi\|443985 | alcohol dehydrogenase [Entamoeba | 78 | 61 | |
| | | | | | histolytica] | | | |
| 732 | 1 | 661 | 2 | gi\|1064791 | function umknown [Bacillus subtilis] | 78 | 55 | |
| 785 | 1 | 2 | 679 | gi\|556014 | DP-N-acetyl muramate-alanine ligase | 78 | 59 | |
| | | | | | [Bacillus subtilis] | | | |
| 786 | 1 | 2 | 172 | gi\|536992 | SugES [Escherichia coli] | 78 | 60 | |
| 820 | 2 | 1602 | 1144 | gi\|153749 | UDPglucose 4-epimerase [Streptococcus | 78 | 60 | |
| | | | | | thermophilus] pir\|A44509\|A44509 UDPglucose | | | |
| | | | | | 4-epimerase (EC 5.1.3.2) - treptococcus | | | |
| | | | | | thermophilus | | | |
| 887 | 1 | 337 | 2 | gi\|495046 | tripeptidase [Lactococcus lactis] | 78 | 70 | |
| 970 | 2 | 395 | 234 | gi\|1652190 | Fat protein [Synechocystis sp.] | 78 | 51 | |
| 4 | 7 | 6069 | 5656 | gi\|1573482 | high affinity ribose transport protein | 77 | 51 | |
| | | | | | (rbsD) [Haemophilus influenzae] | | | |
| 45 | 16 | 12065 | 14047 | gi\|666069 | orf2 gene product [Lactobacillus | 77 | 51 | |
| | | | | | leichmannii] | | | |
| 49 | 13 | 8199 | 9992 | gnl\|PID\|e228615 | homologous to yqcC of the skin element | 77 | 59 | |
| | | | | | [Bacillus subtilis] | | | |
| 60 | 2 | 2895 | 1300 | gi\|143373 | phosphoribosyl aminoimidazole carboxy | 77 | 63 | |
| | | | | | formyl ormyltransferase/inosine | | | |
| | | | | | monophosphate cyclohydrolase (PUR-H(J)) | | | |
| | | | | | Bacillus subtilis] | | | |
| 70 | 6 | 5118 | 3874 | gi\|912464 | No definition line found [Escherichia | 77 | 53 | |
| | | | | | coli] | | | |
| 70 | 7 | 5172 | 5756 | gi\|288413 | glutamate dehydrogenase (NADP+) | 77 | 65 | |
| | | | | | [Corynebacterium glutamicum] | | | |
| | | | | | pir\|S32227\|S32227 glutamate dehydrogenase | | | |
| | | | | | (NADP+) (EC 1.4.1.4) - orynebacterium | | | |
| | | | | | glutamicum | | | |
| 74 | 10 | 7303 | 5864 | gi\|289284 | cysteinyl-tRNA synthetase [Bacillus | 77 | 62 | |
| | | | | | subtilis] | | | |
| 74 | 12 | 9559 | 8078 | gi\|289282 | glutamyl-tRNA synthetase [Bacillus | 77 | 57 | |
| | | | | | subtilis] | | | |
| 88 | 6 | 3013 | 3843 | gi\|535351 | CodY [Bacillus subtilis] | 77 | 57 | |
| 89 | 6 | 5749 | 2510 | gi\|1695686 | pyruvate carboxylase [Bacillus | 77 | 62 | |
| | | | | | stearothemophilus] | | | |
| 91 | 1 | 396 | 728 | gi\|1184044 | L-glutamine:D-fructose-6-P | 77 | 66 | |
| | | | | | amidotransferase precursor [Thermus | | | |
| | | | | | aguaticus thermophilus] | | | |
| 98 | 4 | 3992 | 5710 | gi\|984804 | transmembrane protein [Bacillus subtilis] | 77 | 56 | |
| 124 | 1 | 2 | 940 | gnl\|PID\|e199002 | prolidase PepQ [Lactobacillus deibrueckii] | 77 | 60 | |
| 158 | 5 | 4845 | 4171 | gi\|435297 | unknown [Lactococcus lactis] | 77 | 48 | |
| 162 | 6 | 7426 | 5882 | gi\|142992 | glycerol kinase (glpK) (BC 2.7.1.30) | 77 | 60 | |
| | | | | | [Bacillus subtilis] pir\|B45868\|B45868 | | | |
| | | | | | glycerol kinase (EC 2.7.1.30) - Bacillus | | | |
| | | | | | subtilis sp\|P18157\|GLPK_BACSU GLYCEROL | | | |
| | | | | | KINASE (EC 2.7.1.30) (ATP:GLYCEROL - | | | |
| | | | | | PHOSPHOTRANSFERASE) (GLYCEROKINASE) (GK). | | | |
| 164 | 1 | 179 | 1102 | gi\|882532 | ORF_o294 [Escherichia coli] | 77 | 57 | |
| 164 | 22 | 24158 | 23646 | gi\|1573564 | hypothetical [Haemophilus influenzae] | 77 | 36 | |
| 171 | 6 | 6656 | 7639 | gi\|1303855 | YggH [Bacillus subtilis] | 77 | 59 | |
| 171 | 9 | 9198 | 9683 | gi\|1591672 | phosphate transport system ATP-binding | 77 | 57 | |
| | | | | | protein [Methanococcus jannaschii] | | | |
| 202 | 4 | 2967 | 3422 | gi\|147782 | ruvA protein (gtg start) [Escherichia | 77 | 50 | |
| | | | | | coli] | | | |
| 202 | 6 | 3662 | 4693 | gi\|147783 | ruvB protein [Escherichia coli] | 77 | 58 | |
| 213 | 1 | 3 | 1046 | gi\|1103865 | formyl-tetrahydrofolate synthetase | 77 | 63 | |
| | | | | | [Streptococcus mutans] | | | |
| 217 | 10 | 6870 | 7742 | gi\|414014 | ipa-90d gene product [Bacillus subtilis] | 77 | 50 | |
| 223 | 5 | 4171 | 4902 | gnl\|PID\|e254974 | autolysin response regulator [Bacillus | 77 | 55 | |
| | | | | | subtilis] | | | |
| 223 | 7 | 5024 | 5473 | gnl\|PID\|e254975 | hypothetical protein [Bacillus subtilis] | 77 | 58 | |
| 228 | 10 | 7747 | 6035 | gi\|467409 | DNA polymerase III subunit [Bacillus | 77 | 61 | |
| | | | | | subtilis] | | | |
| 229 | 15 | 16711 | 14261 | gnl\|PID\|e290286 | priA [Bacillus subtilis] | 77 | 62 | |
| 232 | 3 | 1742 | 1437 | gi\|142708 | comG3 gene product [Bacillus subtilis] | 77 | 50 | |
| 238 | 25 | 23174 | 24511 | pir\|B48649\|B48649 | L-rhamnose isomerase (EC 5.3.1.14) | 77 | 59 | |
| | | | | | Escherichia coli | | | |
| 238 | 32 | 29472 | 28708 | gi\|451072 | di-tripeptide transporter [Lactococcus | 77 | 56 | |
| | | | | | lactis] | | | |
| 244 | 4 | 3591 | 2809 | gi\|1773173 | similar to M. jannaschii MJ0938 | 77 | 60 | |
| | | | | | [Escherichia coli] | | | |
| 269 | 5 | 3890 | 3522 | gi\|1303793 | YgeL [Bacillus subtilis] | 77 | 55 | |
| 276 | 6 | 2840 | 2328 | pir\|PC1127\|PC1127 | hypothetical 110 protein (lytA 5′ region) | 77 | 50 | |
| | | | | | - Lactococcus lactis phage US3 (fragment) | | | |
| 291 | 1 | 119 | 916 | gi\|556014 | UDP-N-acetyl muramate-alanine ligase | 77 | 63 | |
| | | | | | [Bacillus subtilis] | | | |
| 304 | 2 | 941 | 2020 | gnl\|PID\|e285001 | CTORF239 [Staphylococcus aureus] | 77 | 62 | |
| 305 | 4 | 3618 | 4394 | gi\|709993 | hypothetical protein [Bacillus subtilis] | 77 | 54 | |
| 327 | 8 | 5697 | 6005 | gi\|153570 | H+ ATPase [Enterococcus faecalis] | 77 | 61 | |
| 341 | 4 | 1206 | 1937 | gi\|1303951 | YqiZ [Bacillus subtilis] | 77 | 62 | |
| 360 | 1 | 429 | 4 | gi\|897754 | nonstructural protein NSP3 [Human | 77 | 38 | |
| | | | | | rotavirus] | | | |
| 362 | 3 | 541 | 1239 | gi\|1001826 | cadmium-transporting ATPase [Synechocystis | 77 | 60 | |
| | | | | | sp.] | | | |
| 363 | 9 | 13917 | 12652 | gi\|1574390 | C4-dicarboxylate transport protein | 77 | 55 | |
| | | | | | [Haemophilus influenzae] | | | |
| 367 | 14 | 7218 | 6679 | pir\|A02766\|RSBS0F | ribosomal protein L6 - Bacillus | 77 | 63 | |
| | | | | | stearothermophilus | | | |
| 386 | 8 | 5456 | 5776 | gnl\|PID\|e281578 | hypothetical 12.2 kd protein [Bacillus | 77 | 61 | |
| | | | | | subtilis] | | | |
| 394 | 4 | 3706 | 4167 | pir\|B39096\|B39096 | alkaline phosphatase (EC 3.1.3.1) III | 77 | 55 | |
| | | | | | precursor - Bacillus subtilis | | | |
| 402 | 1 | 710 | 3 | gi\|533105 | unknown [Bacillus subtilis] | 77 | 59 | |
| 408 | 2 | 1357 | 584 | gi\|666983 | putative ATP binding subunit [Bacillus | 77 | 58 | |
| | | | | | subtilis] | | | |
| 460 | 6 | 3562 | 4938 | gi\|1055246 | biotin carboxylase [Bacillus subtilis] | 77 | 60 | |
| 466 | 7 | 8657 | 9253 | gi\|147402 | mannose permease subunit III-Man | 77 | 61 | |
| | | | | | [Escherichia coli] | | | |
| 475 | 5 | 3794 | 3234 | gi\|532547 | ORF14 [Enterococcus faecalis] | 77 | 68 | |
| 498 | 1 | 1 | 603 | gi\|410137 | ORFX13 [Bacillus subtilis] | 77 | 58 | |
| 515 | 1 | 107 | 574 | gi\|1303815 | YgeY [Bacillus subtilis] | 77 | 60 | |
| 518 | 6 | 2980 | 4518 | gi\|1402515 | membrane-spanning transporter protein | 77 | 56 | |
| | | | | | [Clostridium perfringens] | | | |
| 523 | 5 | 2527 | 2333 | gi\|149601 | thymidylate synthase (EC 2.1.1.45) | 77 | 66 | |
| | | | | | [Lactobacillus casei] | | | |
| 526 | 2 | 1782 | 436 | gi\|1750124 | xylose isomerase [Bacillus subtilis | 77 | 62 | |
| 552 | 7 | 6809 | 6135 | gi\|534045 | antiterminator [Bacillus subtilis] | 77 | 51 | |
| 607 | 3 | 778 | 936 | gi\|1015321 | alanyl-tRNA synthetase [Homo sapiens] | 77 | 51 | |
| 624 | 3 | 2289 | 2555 | gnl\|PID\|e187971 | orf121 gene product [Lactococcus lactis] | 77 | 57 | |
| 781 | 1 | 15 | 485 | gi\|580883 | ipa-88d gene product [Bacillus subtilis] | 77 | 65 | |
| 850 | 2 | 895 | 572 | gi\|142520 | thioredoxin [Bacillus subtilis] | 77 | 59 | |
| 853 | 1 | 186 | 4 | gi\|39962 | ribosomal protein L35 (AA 1-66) [Bacillus | 77 | 66 | |
| | | | | | stearothermophilus] ir\|S05347\|R5BS35 | | | |
| | | | | | ribosomal protein L35 - Bacillus | | | |
| | | | | | earothermophilus | | | |
| 944 | 1 | 2 | 172 | gi\|425467 | transposase [Lactobacillus helveticus] | 77 | 50 | |
| 10 | 1 | 1 | 258 | gnl\|PID\|e234078 | hom [Lactococcus lactis] | 76 | 63 | |
| 12 | 4 | 7650 | 5842 | gnl\|PID\|e254877 | unknown [Mycobacterium tuberculosis] | 76 | 57 | |
| 17 | 29 | 29022 | 28153 | gi\|1500003 | mutator mutT protein [Methanococcus | 76 | 47 | |
| | | | | | jannaschii] | | | |
| 23 | 15 | 8897 | 10285 | gi\|153960 | ethanolamine ammonia-lyase (eutB) | 76 | 64 | |
| | | | | | [Salmonella typhimurium] pir\|A36570\|A36570 | | | |
| | | | | | ethanolamine ammonia-lyase (EC 4.3.1.7) | | | |
| | | | | | 55K chain Salmonella typhimurium | | | |
| 29 | 2 | 1024 | 500 | gi\|40011 | ORF17 (AA 1-161) [Bacillus subtilis] | 76 | 61 | |
| 33 | 1 | 14 | 1552 | gi\|148304 | beta-1,4-N-acetylmuramoylhydrolase | 76 | 60 | |
| | | | | | [Enterococcus hirae] pir\|A42296\|A42296 | | | |
| | | | | | lysozyme 2 (EC 3.2.1.-) precursor - | | | |
| | | | | | Enterococcus irae (ATCC 9790) | | | |
| 34 | 7 | 7432 | 6965 | gi\|44067 | ORF1 C-terminal [Lactococcus lactis] | 76 | 59 | |
| 45 | 8 | 3708 | 4166 | gi\|1303698 | BltD [Bacillus subtilis] | 76 | 56 | |
| 47 | 9 | 12849 | 10270 | gi\|1002520 | MutS [Bacillus subtilis] | 76 | 59 | |
| 55 | 8 | 3614 | 4105 | gi\|1303915 | YghZ [Bacillus subtilis] | 76 | 53 | |
| 55 | 11 | 6385 | 6642 | gi\|216583 | ORF1 [Escherichia coli] | 76 | 45 | |
| 57 | 14 | 17283 | 16597 | gi\|1183887 | integral membrane protein [Bacillus | 76 | 56 | |
| | | | | | subtilis] | | | |
| 59 | 6 | 3112 | 2426 | gi\|392872 | repressor protein [Pasteurella multocida] | 76 | 47 | |
| 64 | 1 | 1242 | 46 | gi\|483941 | blt gene product [Bacillus subtilis] | 76 | 55 | |
| 67 | 3 | 1370 | 2146 | gnl\|PID\|e199390 | orotate phosphoribosyltransferase | 76 | 57 | |
| | | | | | [Lactobacillus plantarum] | | | |
| 69 | 2 | 837 | 334 | gi\|1377831 | unknown [Bacillus subtilis] | 76 | 57 | |
| 70 | 1 | 164 | 1588 | gi\|895751 | putative 6-phospho-beta-glucosidase | 76 | 60 | |
| | | | | | [Bacillus subtilis] pir\|S57762\|S57762 | | | |
| | | | | | probable 6-phospho-beta-glucosidase - | | | |
| | | | | | Bacillus ubtilis | | | |
| 74 | 11 | 7826 | 7269 | pir\|E53402\|E53402 | serine O-acetyltransferase (EC 2.3.1.30) - | 76 | 54 | |
| | | | | | Bacillus stearothermophilus | | | |
| 74 | 13 | 10073 | 9588 | gi\|289281 | unknown [Bacillus subtilis] | 76 | 60 | |
| 85 | 11 | 7809 | 7102 | gi\|457634 | butyrate kinase [Clostridium | 76 | 61 | |
| | | | | | acetobutylicum] | | | |
| 94 | 8 | 6036 | 4801 | gi\|142538 | aspartate aminotransferase [Bacillus sp.] | 76 | 57 | |
| 94 | 14 | 17174 | 12801 | gi\|40060 | DNA polymerase III (AA 1-1437) [Bacillus | 76 | 62 | |
| | | | | | subtilis] p\|P13267\|DP3A_BACSU DNA | | | |
| | | | | | POLYMERASE III, ALPHA CHAIN (EC 2.7.7.7). | | | |
| 94 | 15 | 19140 | 17407 | gi\|1573733 | prolyl-tRNA synthetase (proS) [Haemophilus | 76 | 54 | |
| | | | | | influenzae] | | | |
| 95 | 1 | 1 | 1290 | gi\|472918 | v-type Na-ATPase [Enterococcus hirae] | 76 | 59 | |
| 95 | 4 | 2367 | 3194 | gi\|487276 | Na+ ″ATPase subunit C [Enterococcus hirae] | 76 | 48 | |
| 99 | 1 | 1 | 171 | gi\|1353874 | unknown [Rhodobacter capsulatus] | 76 | 52 | |
| 100 | 5 | 5414 | 5064 | gi\|1591962 | M. jannaschii predicted coding region | 76 | 46 | |
| | | | | | MJ1322 [Methanococcus jannaschii] | | | |
| 100 | 27 | 23165 | 21198 | gi\|216151 | DNA polymerase (gene L; ttg start codon) | 76 | 62 | |
| | | | | | [Bacteriophage SPO2] gi\|579197 SPO2 DNA | | | |
| | | | | | polymerase (aa 1-648) [Bacteriophage SPO2] | | | |
| | | | | | pir\|A21498\|DJBPS2 DNA-directed DNA | | | |
| | | | | | polymerase (EC 2.7.7.7) - phage PO2 | | | |
| 106 | 1 | 1511 | 264 | gi\|1750108 | YnbA [Bacillus subtilis] | 76 | 61 | |
| 116 | 4 | 2480 | 2854 | gi\|755602 | unknown [Bacillus subtilis] | 76 | 60 | |
| 116 | 6 | 3299 | 3625 | gi\|1146234 | dihydrodipicolinate reductase [Bacillus | 76 | 56 | |
| | | | | | subtilis] | | | |
| 122 | 5 | 3029 | 3619 | gi\|467436 | unknown [Bacillus subtilis] | 76 | 52 | |
| 123 | 10 | 9109 | 10389 | gi\|1773196 | similar to B. stearothermophilus N- | 76 | 61 | |
| | | | | | carbamyl-L-amino acid amidohydrolase | | | |
| | | | | | [Escherichia coli] | | | |
| 124 | 5 | 4087 | 3182 | gi\|974332 | NAD(P)H-dependent dihydroxyacetone- | 76 | 58 | |
| | | | | | phosphate reductase [Bacillus ubtilis] | | | |
| 130 | 5 | 3341 | 4294 | gi\|308853 | transmembrane protein [Lactococcus lactis] | 76 | 55 | |
| 132 | 3 | 2265 | 5117 | gi[1673889 | (AE000022) Mycoplasma pneumoniae, | 76 | 59 | |
| | | | | | excinuclease ABC subunit A; similar to | | | |
| | | | | | Swiss-Prot Accession Number P07671, from | | | |
| | | | | | E. coli [Mycoplasma pneumoniae] | | | |
| 138 | 34 | 25849 25409 | gi\|143795 | transfer RNA-Tyr synthetase [Bacillus | 76 | 56 | | |
| | | | | | subtilis] | | | |
| 139 | 1 | 3 | 350 | gnl\|PID\|e191395 | mobilisation protein [Lactococcus lactis] | 76 | 65 | |
| 141 | 1 | 2 | 544 | gi\|662792 | single-stranded DNA binding protein | 76 | 64 | |
| | | | | | [unidentified eubacterium] | | | |
| 155 | 9 | 7612 | 7058 | gnl\|PID\|e247026 | orf6 [Lactobacillus sake] | 76 | 57 | |
| 164 | 4 | 1889 | 2416 | gi\|727436 | putative 20-kDa protein [Lactococcus | 76 | 55 | |
| | | | | | lactis] | | | |
| 181 | 5 | 3475 | 2288 | gi\|1147744 | PSR [Enterococcus hirae] | 76 | 53 | |
| 181 | 8 | 6281 | 4986 | gi\|683583 | 5-enolpyruvylshikimate-3-phosphate | 76 | 62 | |
| | | | | | synthase [Lactococcus lactis] | | | |
| | | | | | pir\|S52580\|S52580 3-phosphoshikirnate 1- | | | |
| | | | | | carboxyvinyltransferase (EC .5.1.19) - | | | |
| | | | | | Lactococcus lactis | | | |
| 197 | 7 | 7662 | 8102 | gi\|1783253 | homologous to many ATP-binding transport | 76 | 58 | |
| | | | | | proteins; hypothetical [Bacillus subtilis] | | | |
| 222 | 16 | 10780 | 11298 | gi\|1591856 | hypothetical protein (SP:P15889) | 76 | 64 | |
| | | | | | [Methanococcus jannaschii] | | | |
| 229 | 1 | 1 | 138 | gi\|148316 | NaH-antiporter protein [Enterococcus | 76 | 47 | |
| | | | | | hirae] | | | |
| 233 | 6 | 3946 | 3341 | gi\|1591652 | hypothetical protein (SP:P31065) | 76 | 60 | |
| | | | | | [Methanococcus jannaschii] | | | |
| 238 | 2 | 844 | 1848 | gi\|622991 | mannitol transport protein [Bacillus | 76 | 64 | |
| | | | | | stearothermophilus] sp\|P508521\|PTMB_BACST | | | |
| | | | | | PTS SYSTEM, MANNITOL-SPECIFIC IIBC | | | |
| | | | | | COMPONENT EIIBC-MTL) (MANNITOL- PERMEASE | | | |
| | | | | | IIBC COMPONENT) (PHOSPHOTRANSFERASE NZYME | | | |
| | | | | | II, BC COMPONENT) (EC 2.7.1.69) (EII-MTL). | | | |
| 238 | 9 | 7235 | 7957 | gi\|1592142 | ABC transporter, probable ATP-binding | 76 | 49 | |
| | | | | | subunit [Methanococcus jannaschii] | | | |
| 249 | 2 | 543 | 1235 | gi\|143156 | membrane bound protein [Bacillus subtilis] | 76 | 45 | |
| 262 | 3 | 4131 | 2692 | gnl\|PID\|e281591 | catalase [Bacillus subtilis] | 76 | 65 | |
| 265 | 1 | 2 | 400 | gi\|141858 | replication-associated protein [Plasmid | 76 | 52 | |
| | | | | | pAD1] | | | |
| 271 | 13 | 8175 | 10844 | gi\|397973 | Mg2+ transport ATPase [Salmonella | 76 | 57 | |
| | | | | | typhimurium] | | | |
| 323 | 4 | 4128 | 4568 | gnl\|PID\|e249023 | T19B10.3 [Caenorhabditis elegans] | 76 | 60 | |
| 329 | 5 | 3270 | 2560 | gi\|310631 | ATP binding protein [Streptococcus | 76 | 54 | |
| | | | | | gordonii] | | | |
| 356 | 1 | 971 | 3 | gi\|971479 | orf3 gene product [Lactobacillus | 76 | 52 | |
| 371 | 1 | 1564 | 944 | gi\|1750125 | xylulose kinase [Bacillus subtilis] | 76 | 57 | |
| 375 | 6 | 5137 | 4238 | gi\|1644202 | unknown [Bacillus subtilis] | 76 | 58 | |
| 382 | 2 | 508 | 2769 | gi\|442360 | ClpC adenosine triphosphatase [Bacillus | 76 | 60 | |
| | | | | | subtilis] | | | |
| 399 | 11 | 7811 | 8845 | gi\|1572970 | acetate:SH-citrate lyase ligase (AMP) | 76 | 54 | |
| | | | | | [Haemophilus influenzae] | | | |
| 399 | 13 | 9126 | 10034 | gi\|1572968 | citrate lyase beta chain (acyl lyase | 76 | 57 | |
| | | | | | subunit) (citE) [Haemophilus influenzae] | | | |
| 485 | 1 | 3 | 1262 | gi\|564018 | dihydrofolate synthetase [Streptococcus | 76 | 54 | |
| | | | | | pneumoniae] | | | |
| 486 | 2 | 970 | 344 | gi\|1256617 | adenine phosphoribosyltransferase | 76 | 61 | |
| | | | | | [Bacillus subtilis] | | | |
| 536 | 1 | 220 | 2 | gi\|437389 | transposase [Lactococcus lactis] | 76 | 59 | |
| 552 | 3 | 3969 | 2491 | gi\|882609 | 6-phospho-beta-glucosidase [Escherichia | 76 | 63 | |
| | | | | | coli | | | |
| 634 | 2 | 697 | 918 | gi\|1022725 | unknown [Staphylococcus haemolyticus] | 76 | 52 | |
| 684 | 3 | 1191 | 688 | gi\|1256653 | DNA-binding protein [Bacillus subtilis] | 76 | 65 | |
| 752 | 1 | 1111 | 929 | gi\|407907 | ORF2 [Staphylococcus xylosus] | 76 | 46 | |
| 822 | 1 | 548 | 237 | gi\|144313 | 6.0 kd ORF [Plasmid ColE1] | 76 | 73 | |
| 923 | 1 | 2 | 421 | gi\|153843 | trypsin-resistant surface T6 protein | 76 | 57 | |
| | | | | | (tee6) precursor [Streptococcus yogenes] | | | |
| 953 | 2 | 534 | 187 | gi\|1592339 | hypothetical protein (PIR:S52522) | 76 | 44 | |
| | | | | | [Methanococcus jannaschii] | | | |
| 965 | 2 | 564 | 343 | gi\|1098898 | CTRP [Plasmodium falciparum] | 76 | 69 | |
| 7 | 4 | 3754 | 4161 | gi\|495046 | tripeptidase [Lactococcus lactis] | 75 | 61 | |
| 25 | 1 | 2 | 580 | gi\|1575577 | DNA-binding response regulator [Thermotoga | 75 | 57 | |
| | | | | | maritima] | | | |
| 45 | 7 | 3090 | 3350 | gi\|1673663 | (AE000003) Mycoplasma pneumoniae, | 75 | 35 | |
| | | | | | E07_orf166 Protein [Mycoplasma pneumoniae] | | | |
| 47 | 6 | 7526 | 6957 | gi\|1673843 | (AE000019) Mycoplasma pneumoniae, pilB | 75 | 58 | |
| | | | | | homolog; similar to GenBank Accession | | | |
| | | | | | Number E64124, from H. influenzae | | | |
| | | | | | [Mycoplasma pneumoniae] | | | |
| 51 | 1 | 15 | 1520 | sp\|P39168\|ATM_ECO | MG(2+) TRANSPORT ATPASE, P-TYPE 1 (EC | 75 | 58 | |
| | | | | LI | 3.6.1.-). | | | |
| 54 | 11 | 3761 | 3579 | gi\|1504026 | similar to C.elegans protein (Z37093) | 75 | 56 | |
| | | | | | [Homo sapiens] | | | |
| 55 | 5 | 1648 | 2562 | gi\|1303901 | YghT [Bacillus subtilis] | 75 | 58 | |
| 56 | 8 | 5873 | 5358 | gi\|895749 | putative cellobiose phosphotransferase | 75 | 49 | |
| | | | | | enzyme II″ [Bacillus ubtilis] | | | |
| 58 | 2 | 2707 | 1916 | gi\|1658403 | formate dehydrogenase alpha subunit | 75 | 58 | |
| | | | | | [Moorella thermoacetica] | | | |
| 71 | 1 | 110 | 1429 | gi\|1304007 | LysA [Bacillus subtilis] | 75 | 58 | |
| 74 | 5 | 3436 | 3074 | gi\|467433 | unknown [Bacillus subtilis] | 75 | 61 | |
| 74 | 8 | 5491 | 4631 | gi\|467483 | unknown [Bacillus subtilis] | 75 | 60 | |
| 77 | 1 | 3 | 992 | gi\|1653966 | 47 kD protein [Synechocystis sp.] | 75 | 34 | |
| 81 | 1 | 26 | 862 | gi\|1064809 | homologous to sp:HTRA_ECOLI [Bacillus | 75 | 55 | |
| | | | | | subtilis] | | | |
| 89 | 11 | 11651 | 9801 | gi\|1573881 | hypothetical [Haemophilus influenzae] | 75 | 51 | |
| 96 | 3 | 2521 | 1643 | gi\|1531619 | NodB [Rhizobium sp.] | 75 | 54 | |
| 98 | 9 | 11494 | 10199 | gi\|1573043 | hypothetical [Haemophilus influenzae] | 75 | 53 | |
| 110 | 12 | 11326 | 10283 | gi\|1184121 | auxin-induced protein [Vigna radiata] | 75 | 51 | |
| 117 | 13 | 11200 | 9944 | gi\|457635 | vancomycin histidine protein kinase | 75 | 51 | |
| | | | | | [Enterococcus faecium] gi\|801884 vanS | | | |
| | | | | | [Transposon Tn1546] | | | |
| 122 | 6 | 3812 | 5206 | gi\|467439 | temperature sensitive cell division | 75 | 59 | |
| | | | | | [Bacillus subtilis] | | | |
| 128 | 12 | 8262 | 7921 | gi\|466473 | cellobiose phosphotransferase enzyme II′ | 75 | 48 | |
| | | | | | [Bacillus tearothermophilus] | | | |
| 128 | 38 | 31848 | 30733 | gi\|216300 | peptidoglycan synthesis enzyme [Bacillus | 75 | 56 | |
| | | | | | subtilis] sp\|P37585 MURG_BACSU MURO | | | |
| | | | | | PROTEIN UPD-N-ACETYLGLUCOSAMINE--N- | | | |
| | | | | | ACETYLMURAMYL- | | | |
| | | | | | PENTAPEPTIDE) PYROPHOSPHORYL-UNDECAPRENOL | | | |
| | | | | | N-ACETYLGLUCOSAMINE RANSFERASE). | | | |
| 129 | 2 | 1916 | 2134 | gnl\|PID\|e267624 | Unknown, highly similar to Pseudomonas | 75 | 47 | |
| | | | | | putida 4-oxalocrotonate tautomerase | | | |
| | | | | | [Bacillus subtilis] | | | |
| 130 | 4 | 2375 | 3343 | gi\|495179 | transmembrane protein [Lactococcus lactis] | 75 | 55 | |
| 133 | 1 | 3 | 1514 | gnl\|PID\|e254877 | unknown [Mycobacterium tuberculosis] | 75 | 54 | |
| 158 | 13 | 12326 | 11634 | gi\|809660 | deoxyribose-phosphate aldolase [Bacillus | 75 | 66 | |
| | | | | | subtilis] pir\|S49455\|S49455 deoxyribose- | | | |
| | | | | | phosphate aldolase (EC 4.1.2.4) - acillus | | | |
| | | | | | subtilis | | | |
| 162 | 13 | 14285 | 12543 | gi\|1653222 | cation-transporting ATPase PacL | 75 | 60 | |
| | | | | | [Synechocystis sp.] | | | |
| 170 | 2 | 1280 | 921 | sp\|P07999\|DHGB_BAC | GLUCOSE 1-DEHYDROGENASE B (EC 1.1.1.47). 75 | 62 | | |
| | | | | | ME | | | |
| 171 | 7 | 7618 | 8523 | gi\|1303856 | YagI [Bacillus subtilisi | 75 | 52 | |
| 179 | 14 | 14668 | 15255 | gi\|457177 | alkyl hydroperoxide reductase [Salmonella | 75 | 55 | |
| | | | | | typhimurium] sp\|P19479\|AHPC_SALTY ALKYL | | | |
| | | | | | HYDROPEROXIDE REDUCTASE C22 PROTEIN (EC | | | |
| | | | | | .6.4.-). {SUB 2-187)} | | | |
| 181 | 6 | 4470 | 3604 | gi\|683585 | prephenate dehydratase [Lactococcus | 75 | 49 | |
| | | | | | lactis] | | | |
| 191 | 1 | 183 | 560 | gnl\|PID\|e261991 | putative orf [Bacillus subtilis] | 75 | 57 | |
| 197 | 3 | 2117 | 3592 | gi\|1783250 | h omologous to cytochrome d ubiquinol | 75 | 60 | |
| | | | | | oxidase subunit I; hypothetical [Bacillus | | | |
| | | | | | subtilis] | | | |
| 215 | 3 | 2545 | 2201 | gn\|PID\|e284996 | ORF136 [Staphylococcus aureus] | 75 | 54 | |
| 216 | 1 | 2 | 256 | gi\|153570 | H+ ATPase [Enterococcus faecalis] | 75 | 53 | |
| 223 | 4 | 2406 | 4193 | gi\|862312 | lytS gene product [Staphylococcus aureus] | 75 | 56 | |
| 227 | 5 | 3004 | 3567 | gi\|144729 | butanol dehydrogenase [Clostridium | 75 | 53 | |
| | | | | | acetobutylicum] sp\|Q04944\|ADHA_CLOAB NADH- | | | |
| | | | | | DEPENDENT BUTANOL DEHYDROGENASE A (EC | | | |
| | | | | | .1.1.-) | (BDH I). | | |
| 228 | 9 | 6032 | 5700 | gi\|467410 | unknown [Bacillus subtilis] | 75 | 59 | |
| 229 | 16 | 17081 | 16848 | gi\|207398 | tropomyosin T class IVd alpha-3 [Rattus | 75 | 42 | |
| | | | | | norvegicus] | | | |
| 238 | 8 | 6038 | 7237 | gi\|141927 | czcB gene product [Alcaligenes eutrophus] | 75 | 39 | |
| 244 | 10 | 7795 | 7460 | gi\|467419 | unknown [Bacillus subtilis] | 75 | 56 | |
| 247 | 1 | 7 | 1431 | gi\|577569 | PepV [Lactobacillus delbrueckii] | 75 | 54 | |
| 250 | 5 | 3416 | 3201 | gi\|1580783 | sperm receptor [Strongylocentrotus | 75 | 50 | |
| | | | | | purpuratus | | | |
| 256 | 1 | 2 | 562 | gi\|709991 | hypothetical protein [Bacillus subtilis] | 75 | 56 | |
| 262 | 2 | 1031 | 2479 | gi\|142783 | DNA photolyase [Bacillus firmus] | 75 | 59 | |
| 263 | 1 | 222 | 890 | gi\|148304 | beta-1,4-N-acetylmuramoylhydrolase | 75 | 60 | |
| | | | | | [Enterococcus hirae] pir\|A42296\|A42296 | | | |
| | | | | | lysozyme 2 (EC 3.2.1.-) precursor - | | | |
| | | | | | Enterococcus irae (ATCC 9790) | | | |
| 266 | 5 | 2224 | 1982 | gnl\|PID\|e253211 | ORF YDLO65c [Saccharomyces cerevisiae] | 75 | 50 | |
| 269 | 2 | 1477 | 707 | gi\|1736647 | ORF_ID:o347#4; similar to [SwissProt | 75 | 61 | |
| | | | | | Accession Number P44634] [Escherichia | | | |
| | | | | | coli] | | | |
| 276 | 11 | 7415 | 4593 | gnl\|PID\|e221269 | tail protein [Bacteriophage CP-1] | 75 | 54 | |
| 279 | 17 | 14992 | 14651 | gi\|1389549 | ORF3 [Bacillus subtilis] | 75 | 61 | |
| 292 | 11 | 7829 | 8470 | gi\|160693 | sporozoite surface protein [Plasmodium | 75 | 50 | |
| | | | | | yoelii] | | | |
| 295 | 2 | 489 | 1157 | gi\|533099 | endonuclease III [Bacillus subtilis] | 75 | 59 | |
| 307 | 4 | 3804 | 4889 | gi\|1321625 | exo-alpha-1, 4-glucosidase [Bacillus | 75 | 60 | |
| | | | | | stearothermophilus] | | | |
| 322 | 4 | 1088 | 1996 | gi\|310303 | mosA [Rhizobium meliloti] | 75 | 63 | |
| 331 | 1 | 1 | 294 | gi[1016092 | ribosomal protein S14 [Cyanophora | 75 | 57 | |
| | | | | | paradoxa] | | | |
| 334 | 7 | 6860 | 7969 | gi\|409286 | bmrU [Bacillus subtilis] | 75 | 45 | |
| 340 | 1 | 3 | 743 | gi\|288413 | glutamate dehydrogenase (NADP+) | 75 | 60 | |
| | | | | | [Corynebacterium glutamicum] | | | |
| | | | | | pir\|S32227\|S32227 glutamate dehydrogenase | | | |
| | | | | | (NADP+) (EC 1.4.1.4) - orynebacterium | | | |
| | | | | | glutamicum | | | |
| 343 | 2 | 1497 | 778 | gi\|46602 | putative transposase (AA 1 - 224) | 75 | 54 | |
| | | | | | [Staphylococcus aureus] ir\|S12093\|S12093 | | | |
| | | | | | probable IS431mec protein - Staphylococcus | | | |
| | | | | | aureus p\|P19380\|TRA2_STAAU TRANSPOSASE FOR | | | |
| | | | | | INSERTION SEQUENCE-LIKE ELEMENT 431MEC. | | | |
| 372 | 3 | 865 | 1629 | gi\|146282 | gut operon repressor (gutR) [Escherichia | 75 | 58 | |
| | | | | | coli] | | | |
| 372 | 7 | 6614 | 5307 | gnl\|PID\|e255128 | trigger factor [Bacillus subtilis] | 75 | 62 | |
| 387 | 3 | 1721 | 1353 | gi\|580902 | ORF6 gene product [Bacillus subtilis] | 75 | 53 | |
| 399 | 30 | 28774 | 29805 | gi\|146278 | glucitol-specific enzyme II (gutA) | 75 | 61 | |
| | | | | | [Escherichia coli] pir\|A26725\|WQEC2S | | | |
| | | | | | phosphotransferase system enzyme II (EC | | | |
| | | | | | .7.1.69), sorbitol-specific, factor II - | | | |
| | | | | | Escherichia coli sp\|P05705\|PTHB_ECOLI PTS | | | |
| | | | | | SYSTEM, GLUCITOL/SORBITOL-SPECIFIC IIBC | | | |
| | | | | | OMPONENT (EIIBC-GUT) | | | |
| 399 | 33 | 31077 | 32768 | gi\|517205 | 67 kDa Myosin-crossreactive streptococcal | 75 | 59 | |
| | | | | | antigen [Streptococcus yogenes] | | | |
| 404 | 6 | 4994 | 4332 | gi\|1303921 | YqiF [Bacillus subtilis] | 75 | 64 | |
| 404 | 7 | 4984 | 4829 | gi\|1303921 | YgiF [Bacillus subtilis] | 75 | 60 | |
| 419 | 1 | 320 | 3 | gi\|496283 | lysin [Bacteriophage Tuc2009] | 75 | 67 | |
| 431 | 3 | 1139 | 759 | sp\|P46351\|YZGD_BAC | HYPOTHETICAL 45.4 KD PROTEIN IN THIAMINASE | 75 | 60 | |
| | | | | SU | I 5′REGION. | | | |
| 473 | 1 | 166 | 2 | gnl\|PID\|e229299 | R04D3.8[Caenorhabditis elegans] | 75 | 35 | |
| 481 | 1 | 1 | 351 | gi\|1573766 | phosphoglyceromutase (gpmA) [Haemophilus | 75 | 64 | |
| | | | | | influenzae] | | | |
| 492 | 1 | 440 | 3 | gi\|806487 | ORF211; putative [Lactococcus lactis] | 75 | 57 | |
| 595 | 1 | 705 | 181 | gi\|147485 | queA [Escherichia coli] | 75 | 51 | |
| 619 | 2 | 879 | 319 | gi\|1063246 | low homology to P14 protein of Heamophilus | 75 | 59 | |
| | | | | | influenzar and 14.2 kDa protein of | | | |
| | | | | | Escherichia coli [Bacillus subtilis] | | | |
| 663 | 1 | 15 | 1544 | gi\|475112 | enzyme IIabc [Pediococcus pentosaceus] | 75 | 54 | |
| 701 | 4 | 662 | 946 | gi\|143793 | tyrosyl-tRNA synthetase [Bacillus | 75 | 60 | |
| | | | | | caldotenax] | | | |
| 719 | 1 | 970 | 419 | gi\|727436 | putative 20-kDa protein [Lactococcus | 75 | 56 | |
| | | | | | lactis] | | | |
| 886 | 1 | 101 | 409 | gi\|143150 | levR [Bacillus subtilis] | 75 | 59 | |
| 939 | 1 | 403 | 191 | gi\|425467 | transposase [Lactobacillus helveticus] | 75 | 53 | |
| 984 | 2 | 66 | 227 | gi\|1652190 | Fat protein [Synechocystis sp.] | 75 | 48 | |
| 17 | 2 | 2592 | 2924 | gi\|532556 | ORF23 [Enterococcus faecalis] | 74 | 53 | |
| 17 | 25 | 24449 | 25639 | gi\|1458228 | mutY homolog [Homo sapiens] | 74 | 50 | |
| 21 | 7 | 4729 | 5229 | gi\|726320 | putative protein of unknown function | 74 | 57 | |
| | | | | | encoded by the IS200-like lement [Yersinia | | | |
| | | | | | pestis] | | | |
| 32 | 9 | 5819 | 4488 | gi\|1498962 | M. jannaschii predicted coding region | 74 | 41 | |
| | | | | | MJ0188 [Methanococcus jannaschii] | | | |
| 38 | 1 | 707 | 3 | gi\|142152 | sulfate permease (gtg start codon) | 74 | 53 | |
| | | | | | [Synechococcus PCC6301] pir\|A30301\|GRYCS7 | | | |
| | | | | | sulfate transport protein - Synechococcus | | | |
| | | | | | sp. PCC 7942) | | | |
| 44 | 1 | 1 | 927 | gi\|1377823 | aminopeptidase [Bacillus subtilis] | 74 | 63 | |
| 60 | 8 | 8747 | 8070 | gi\|143368 | phosphoribosylformyl glycinamidine | 74 | 63 | |
| | | | | | synthetase I (PUR-L; gtg start odon) | | | |
| | | | | | [Bacillus subtilis] | | | |
| 72 | 8 | 7388 | 7119 | gnl\|PID\|e209004 | glutaredoxin-like protein [Lactococcus | 74 | 53 | |
| | | | | | lactis] | | | |
| 91 | 4 | 1031 | 2257 | gi\|726480 | L-glutamine-D-fructose-6-phosphate | 74 | 58 | |
| | | | | | amidotransferase [Bacillus ubtilis] | | | |
| 105 | 7 | 5553 | 5855 | gi\|467418 | unknown [Bacillus subtilis] | 74 | 63 | |
| 110 | 18 | 16903 | 15842 | gi\|45288 | arcB (AA 11336) [Pseudomonas aeruginosa] | 74 | 57 | |
| 112 | 3 | 1112 | 636 | gi\|887824 | ORF_o310 [Esoherichia coli] | 74 | 53 | |
| 123 | 8 | 6105 | 7619 | gi\|1773191 | similar to Pseudomonas sp. ORF5 | 74 | 60 | |
| | | | | | [Escherichia coli] | | | |
| 128 | 1 | 2 | 1315 | gi\|143961 | pyruvate phosphate dikinase [Clostridium | 74 | 58 | |
| | | | | | symbiosum] pir\|A36231\|KIQAPO | | | |
| | | | | | pyruvate, orthophosphate dikinase (EC | | | |
| | | | | | 2.7.9.1) - lostridium symbiosum | | | |
| 128 | 26 | 18866 | 20401 | gi\|1303961 | YgjJ [Bacillus subtilis] | 74 | 57 | |
| 150 | 5 | 4653 | 5303 | gi\|495046 | tripeptidase [Lactococcus lactis] | 74 | 53 | |
| 159 | 8 | 7500 | 6850 | gi\|581098 | GlnQ (AA 1-240); gtg start [Escherichia | 74 | 53 | |
| | | | | | coli ] | | | |
| 179 | 1 | 1259 | 57 | gi\|537080 | ribonucleoside triphosphate reductase | 74 | 62 | |
| | | | | | [Escherichia coli] pir\|A47331\|A47331 | | | |
| | | | | | oxygen-sensitive ribonucleoside- | | | |
| | | | | | triphosphate eductase (BC 1.17.4.-)- | | | |
| | | | | | Escherichia coli | | | |
| 183 | 2 | 1669 | 224 | gi\|1146200 | DNA or RNA helicase, DNA-dependent ATPase | 74 | 53 | |
| | | | | | [Bacillus subtilis] | | | |
| 213 | 4 | 2265 | 3200 | gi\|1373157 | orf-X; hypothetical protein; Method: | 74 | 63 | |
| | | | | | conceptual translation supplied by author | | | |
| | | | | | [Bacillus subtilis] | | | |
| 229 | 13 | 13774 | 12806 | gnl\|PID\|e290288 | Met-tRNAi formyl transferase [Bacillus | 74 | 55 | |
| | | | | | subtilis] | | | |
| 238 | 31 | 28648 | 28052 | gi\|451072 | di-tripeptide transporter [Lactococcus | 74 | 56 | |
| 244 | 8 | 6409 | 5552 | gi\|467422 | unknown [Bacillus subtilis] | 74 | 60 | |
| 249 | 1 | 7 | 411 | gi\|1591758 | diaminopimelate epimerase [Methanococcus | 74 | 51 | |
| | | | | | jannaschii] | | | |
| 270 | 3 | 1832 | 3955 | gi\|1303829 | YgfK [Bacillus subtilis] | 74 | 55 | |
| 276 | 3 | 1668 | 1357 | gi\|496282 | holin [Bacteriophage Tuc2009] | 74 | 54 | |
| 288 | 9 | 5807 | 5076 | gi\|530063 | glycerol uptake facilitator [Streptococcus | 74 | 60 | |
| | | | | | pneumoniae] sp\|P52281\|GLPF_STRPN GLYCEROL | | | |
| | | | | | UPTAKE FACILITATOR PROTEIN. | | | |
| 292 | 21 | 16780 | 17547 | gi\|1573646 | Mg(2+) transport ATPase protein C (mgtC) | 74 | 42 | |
| | | | | | (SP:P22037) [Haemophilus influenzae] | | | |
| 297 | 1 | 682 | 11 | gnl\|PID\|e255093 | hypothetical protein [Bacillus subtilis] | 74 | 54 | |
| 298 | 3 | 3562 | 3095 | gi\|1303970 | YqjS [Bacillus subtilis] | 74 | 46 | |
| 321 | 10 | 5081 | 6028 | pir\|A32950\|A32950 | probable reductase protein - Leishmania | 74 | 56 | |
| | | | | | major | | | |
| 327 | 2 | 904 | 3285 | gi\|1573876 | virulence associated protein homolog | 74 | 53 | |
| | | | | | (vacB) [Haemophilus influenzae] | | | |
| 334 | 5 | 3942 | 5432 | gi\|1652678 | amidase [Synechocystis sp.] | 74 | 57 | |
| 341 | 13 | 13007 | 12069 | gi\|39881 | ORF 311 (AA 1-311) [Bacillus subtilis] | 74 | 53 | |
| 362 | 7 | 3529 | 5274 | gnl\|PID\|e255093 | hypothetical protein [Bacillus subtilis] | 74 | 58 | |
| 376 | 3 | 1282 | 2346 | gi\|1773090 | transfer RNA-guanine transglycosylase | 74 | 59 | |
| | | | | | [Escherichia coli] | | | |
| 421 | 2 | 48 | 1400 | gi\|710632 | beta-glucosidase [Bacillus subtilis] | 74 | 58 | |
| 471 | 1 | 815 | 3 | gi\|854234 | cymG geno product [Klebsiella oxytoca] | 74 | 53 | |
| 480 | 2 | 263 | 607 | gi\|1303994 | YgkM [Bacillus subtilis] | 74 | 48 | |
| 518 | 7 | 4409 | 5002 | gi\|145821 | EBG enzyme alpha subunit [Escherichia | 74 | 47 | |
| | | | | | coli] | | | |
| 539 | 8 | 6607 | 7179 | gi[1165295 | D3703.8p [Saccharomyces cerevisiae] | 74 | 57 | |
| 542 | 1 | 750 | 4 | gi[1064810 | function unknown [Bacillus subtilis] | 74 | 56 | |
| 559 | 1 | 1204 | 5 | gi\|43821 | nifJ protein (AA 1-1171) [Klebsiella | 74 | 58 | |
| | | | | | pneumoniae] p\|P03833\|NIFJ_KLEPN PYRUVATE- | | | |
| | | | | | FLAVODOXIN OXIDOREDUCTASE (BC -.-.-) | | | |
| 579 | 3 | 1373 | 1624 | gi[1237013 | ORF2 [Bacillus subtilis] | 74 | 46 | |
| 624 | 4 | 2518 | 3669 | gi[467394 | recombination protein [Bacillus subtilis] | 74 | 56 | |
| 688 | 1 | 623 | 3 | gi[662880 | novel hemolytic factor [Bacillus cereus] | 74 | 48 | |
| 763 | 1 | 106 | 441 | gi\|153955 | envM protein [Salmonella typhimurium] | 74 | 46 | |
| 811 | 1 | 3 | 158 | gi\|309662 | pheromone binding protein [Plasmid pCF10] | 74 | 57 | |
| 852 | 1 | 2 | 601 | gi\|309662 | pheromone binding protein [Plasmid pCF10] | 74 | 53 | |
| 935 | 1 | 976 | 2 | gi\|467403 | seryl-tRNA synthetase [Bacillus subtilis] | 74 | 59 | |
| 22 | 2 | 2178 | 2471 | gi\|467460 | unknown [Bacillus subtilis] | 73 | 61 | |
| 24 | 2 | 1126 | 3150 | gi\|1303822 | YqfF [Bacillus subtilis] | 73 | 54 | |
| 33 | 6 | 6638 | 6970 | gi\|536971 | ORF_o76 [Esoherichia coli] | 73 | 56 | |
| 48 | 1 | 621 | 1241 | gnl\|PID\|e274111 | aggregation promoting protein | 73 | 67 | |
| | | | | | [Lactobacillus gasseri] | | | |
| 48 | 6 | 5327 | 7225 | gi\|1185289 | 2-succinyl-6-hydroxy-2,4-cyclohexadiene-1- | 73 | 56 | |
| | | | | | carboxylate synthase [Bacillus subtilis] | | | |
| 50 | 2 | 1097 | 2008 | gi\|1498295 | homoserine kinase homolog [Streptococcus | 73 | 55 | |
| | | | | | pneumoniae] | | | |
| 52 | 4 | 2793 | 4334 | gi\|473902 | alpha-acetolactate synthase [Lactococcus | 73 | 59 | |
| | | | | | lactis] | | | |
| 55 | 1 | 1 | 261 | gi\|396365 | alternate name yjbA [Escherichia coli] | 73 | 36 | |
| 60 | 6 | 5935 | 5549 | gi\|551881 | amidophosphoribosyltransferase | 73 | 57 | |
| | | | | | [Lactobacillus casei] pir\|PC1136\|PC1136 | | | |
| | | | | | purF protein - Lactobacillus casei | | | |
| | | | | | (fragment) sp\|P35853\|PUR1_LACCA | | | |
| | | | | | AMIDOPHOSPHORIBOSYLTRANSFEFASE (SC | | | |
| | | | | | 2.4.2.14) GLUTAMINE | | | |
| | | | | | PHOSPHORIBOSYLPYROPHOSPHATE | | | |
| | | | | | AMIDOTRANSFERASE) (ATASE) FRAGMENT | | | |
| 74 | 2 | 477 | 1355 | gnl\|PID\|e233567 | unknown [Mycobacterium tuberculosis] | 73 | 54 | |
| 81 | 19 | 14213 | 13845 | gi\|606073 | ORF_o169 [Escherichia coli] | 73 | 52 | |
| 93 | 7 | 2861 | 4075 | gi\|405134 | acetate kinase [Bacillus subtilis] | 73 | 56 | |
| 100 | 1 | 1057 | 2 | gi\|1353561 | ORF44 [Bacteriophage rlt] | 73 | 52 | |
| 100 | 41 | 28872 | 28627 | gi\|188492 | heat shock-induced protein [Homo sapiens] | 73 | 42 | |
| 104 | 4 | 5558 | 5274 | gi\|312440 | aspartate carbamoyltransferase [Bacillus | 73 | 55 | |
| | | | | | caldolyticus] pir\|S34318\|S34318 aspartate | | | |
| | | | | | carbamoyltransferase (EC 2.1.3.2) - | | | |
| | | | | | acillus caldolyticus | | | |
| 119 | 5 | 3264 | 3638 | gi\|473707 | positive regulator for virulence factors | 73 | 39 | |
| | | | | | [Clostridium perfringens] | | | |
| 123 | 17 | 16156 | 15665 | gi\|1303703 | YrkD [Bacillus subtilis] | 73 | 37 | |
| 123 | 18 | 16133 | 16465 | gi\|1303893 | YghL [Bacillus subtilis] | 73 | 43 | |
| 124 | 3 | 2165 | 1722 | gi\|486661 | TMnm related protein [Saccharomyces | 73 | 45 | |
| | | | | | cerevisiae] | | | |
| 127 | 6 | 5778 | 5101 | gi\|290561 | o188 [Escherichia coli] | 73 | 48 | |
| 128 | 10 | 6896 | 7201 | pir\|S37387\|s37387 | internalin A precursor - Listeria | 73 | 53 | |
| | | | | | monocytogenes | | | |
| 137 | 2 | 980 | 1954 | gi\|1276882 | EpsI [Streptococcus thermophilus] | 73 | 56 | |
| 141 | 3 | 942 | 2777 | gi\|467336 | unknown [Bacillus subtilis] | 73 | 49 | |
| 146 | 7 | 5611 | 4739 | gi\|149395 | lacC [Lactococcus lactis] | 73 | 56 | |
| 154 | 6 | 3566 | 4621 | gi\|1354775 | pfoS/R [Treponema pallidum] | 73 | 46 | |
| 155 | 8 | 7136 | 6726 | gnl\|PID\|e247026 | orf6 [Lactobacillus sake] | 73 | 61 | |
| 158 | 8 | 8693 | 7119 | gi\|1674275 | (AE000056) Mycoplasma pneumonlae, | 73 | 45 | |
| | | | | | hypothetical ABC transporter (yjcW) | | | |
| | | | | | homolog; similar to Swiss-Prot Accession | | | |
| | | | | | Number P32721, from E. coli [Mycoplasma | | | |
| | | | | | pneumoniae] | | | |
| 162 | 4 | 4039 | 3305 | gi\|142997 | glycerol uptake facilitator [Bacillus | 73 | 55 | |
| | | | | | subtilis] | | | |
| 165 | 4 | 3962 | 3105 | gi\|882736 | ORFf278 [Escherichia coli] | 73 | 58 | |
| 171 | 3 | 3952 | 4689 | gnl\|PID\|e63527 | FtsE [Mycobacterium tuberculosis] | 73 | 56 | |
| 171 | 5 | 5673 | 6596 | gi\|1303854 | YqgG [Bacillus subtilis] | 73 | 59 | |
| 179 | 9 | 9302 | 10414 | gnl\|PID\|e254984 | hypothetical protein [Bacillus subtilis] | 73 | 55 | |
| 180 | 1 | 24 | 1151 | gi\|43985 | nifS-like gene [Lactobacillus delbrueckii] | 73 | 56 | |
| 181 | 12 | 10036 | 9674 | gnl\|PID\|e220317 | chorismate mutase [Staphylococcus xylosus] | 73 | 50 | |
| 181 | 13 | 10713 | 10003 | gi\|39813 | phospho-2-dehydro-3-deoxyheptonate | 73 | 56 | |
| | | | | | aldolase [Bacillus subtilis] | | | |
| | | | | | ir\|S21418\|S21418 phospho-2-dehydro-3- | | | |
| | | | | | deoxyheptonate aldolase (EC 1.2.15) - | | | |
| | | | | | Bacillus subtilis | | | |
| 183 | 3 | 2716 | 1667 | gi\|1146199 | putative [Bacillus subtilis] | 73 | 36 | |
| 198 | 1 | 869 | 108 | gi\|142854 | homologous to E. coli radC gene product | 73 | 47 | |
| | | | | | and to unidentified protein rom | | | |
| | | | | | Staphylococcus aureus [Bacillus subtilis] | | | |
| 210 | 1 | 956 | 3 | gnl\|PID\|e281310 | acetyl coenzyme A acetyltransferase | 73 | 54 | |
| | | | | | (thiolase) [Thermoanaerobacterium | | | |
| | | | | | thermosaccharolyticum] | | | |
| 230 | 1 | 1 | 171 | gi\|304143 | S-layer protein [Bacillus circulans] | 73 | 46 | |
| 235 | 1 | 715 | 2 | gi\|1732315 | transport system permease homolog | 73 | 49 | |
| | | | | | [Listeria monocytogenes] | | | |
| 235 | 2 | 888 | 676 | gi\|551726 | sporulation protein [Bacillus subtilis] | 73 | 54 | |
| 242 | 4 | 3290 | 3517 | gnl\|PID\|e236570 | orf6 gene product [Enterococcus faecalis] | 73 | 30 | |
| 242 | 8 | 5914 | 6492 | gi\|1742340 | HipB protein. [Escherichia coli] | 73 | 49 | |
| 250 | 3 | 3037 | 2411 | gi\|1174238 | TipB [Pseudomonas fluorescens] | 73 | 57 | |
| 254 | 5 | 1124 | 792 | gi\|580900 | ORF3 gene product [Bacillus subtilis] | 73 | 52 | |
| 269 | 9 | 5507 | 5154 | gi\|1303790 | YqeI [Bacillus subtilis] | 73 | 60 | |
| 269 | 12 | 7989 | 7345 | gi\|285621 | undefined open reading frame [Bacillus | 73 | 54 | |
| | | | | | stearothermophilus] | | | |
| 284 | 1 | 1 | 915 | gi\|455528 | ORF2 [Streptococcus thermophilus | 73 | 54 | |
| | | | | | bacteriophage] | | | |
| 290 | 3 | 1932 | 2678 | gnl\|PID\|e248883 | unknown [Mycobacterium tuberculosis] | 73 | 57 | |
| 295 | 8 | 4521 | 4739 | gi\|145478 | putative [Escherichia coli] | 73 | 56 | |
| 296 | 1 | 2 | 1846 | gnl\|PID\|e249642 | transketolase [Bacillus subtilis] | 73 | 59 | |
| 310 | 4 | 3488 | 3036 | gi\|1591900 | nucleoside diphosphate kinase | 73 | 48 | |
| | | | | | [Methanococcus jannaschii] | | | |
| 313 | 1 | 17 | 778 | gi\|1658371 | cyclic beta-1,2-glucan modification | 73 | 60 | |
| | | | | | protein [Rhizobium meliloti] | | | |
| 314 | 3 | 2642 | 2067 | gi\|1330343 | C34D4.12 gene product [Caenorhabditis | 73 | 56 | |
| | | | | | elegans | | | |
| 325 | 1 | 492 | 4 | gi\|407908 | EIIscr [Staphylococcus xylosus] | 73 | 56 | |
| 345 | 19 | 20549 | 21901 | gi\|443691 | glutathione reductase [Streptococcus | 73 | 59 | |
| | | | | | thermophilus] | | | |
| 359 | 4 | 3280 | 2252 | gi\|1001478 | hypothetical protein [Synechocystis sp.] | 73 | 50 | |
| 374 | 1 | 884 | 3 | gi\|435123 | PacL [Synechococcus sp.] | 73 | 58 | |
| 379 | 6 | 5676 | 4339 | gi\|887822 | possible frameshift at end to join to next | 73 | 57 | |
| | | | | | ORF? [Escherichia coli] | | | |
| 383 | 4 | 3815 | 3387 | gi\|1651732 | mutator MutT protein [Synechocystis sp.] | 73 | 52 | |
| 392 | 4 | 3454 | 5202 | gi\|294587 | minimal change nephritis transmembrane | 73 | 56 | |
| | | | | | glycoprotein [Rattus orvegicus] | | | |
| 394 | 5 | 4267 | 5250 | gi\|49011 | amidinotransferase II [Streptomyces | 73 | 42 | |
| | | | | | griseus] | | | |
| 395 | 10 | 4252 | 4608 | gi\|1591139 | M. jannaschii predicted coding region | 73 | 48 | |
| | | | | | MJ0435 [Methanococcus jannaschii] | | | |
| 397 | 1 | 885 | 4 | gnl\|PID\|e249658 | GriA [Bacillus subtilis] | 73 | 56 | |
| 399 | 15 | 10007 | 11569 | gi\|565619 | citrate lyase alpha-subunit [Klebsiella | 73 | 54 | |
| | | | | | pneumoniae] pir\|S60776\|560776 citrate | | | |
| | | | | | (pro-3S)-lyase (EC 4.1.3.6) alpha chain - | | | |
| | | | | | lebsiella pneumoniae | | | |
| 416 | 2 | 660 | 1649 | gi\|475114 | regulatory protein [Pediococcus | 73 | 50 | |
| | | | | | pentosaceus] | | | |
| 436 | 6 | 4124 | 3540 | gi\|727436 | putative 20-kDa protein [Lactococcus | 73 | 53 | |
| | | | | | lactis] | | | |
| 446 | 3 | 1618 | 4260 | gi\|882711 | exonuclease V alpha-subunit [Escherichia | 73 | 48 | |
| | | | | | coli] | | | |
| 462 | 1 | 819 | 43 | gi\|1399011 | immunogenic secreted protein precursor | 73 | 63 | |
| | | | | | (Streptococcus pyogenes ] | | | |
| 482 | 5 | 3181 | 2501 | gi\|1072419 | glcB gene product [Staphylococcus | 73 | 55 | |
| | | | | | carnosus] | | | |
| 495 | 4 | 1340 | 3031 | gi\|146547 | kdpA [Escherichia coli] | 73 | 55 | |
| 523 | 4 | 2354 | 1821 | pir\|A00392\|RDSODF | dihydrofolate reductase (EC 1.5.1.3) - | 73 | 54 | |
| | | | | | Enterococcus faecium | | | |
| 543 | 5 | 3099 | 2893 | gi\|19743 | nsGRP-2 [Nicotiana sylvestris] | 73 | 53 | |
| 567 | 1 | 9 | 740 | gi\|1147601 | cyclophilin isoform 4 [Caenorhabditis | 73 | 54 | |
| | | | | | elegans] | | | |
| 629 | 1 | 945 | 4 | gi\|1006620 | ABC transporter [Synechocystis sp.] | 73 | 46 | |
| 714 | 2 | 344 | 556 | gi\|1045872 | ATP-binding protein [Mycoplasma | 73 | 61 | |
| | | | | | genitalium] | | | |
| 747 | 1 | 320 | 3 | gi\|437389 | transposase [Lactococcus lactis] | 73 | 56 | |
| 764 | 1 | 3 | 515 | gi\|532554 | ORF21 [Enterococcus faecalis] | 73 | 50 | |
| 766 | 1 | 683 | 3 | gi\|1673788 | (AE000015) Mycoplasma pneumoniae, | 73 | 52 | |
| | | | | | fructose-bisphosphate aldolase; similar to | | | |
| | | | | | Swiss-Prot Accession Number P13243, from | | | |
| | | | | | B. subtilis [Mycoplasma pneumoniae] | | | |
| 880 | 1 | 198 | 4 | gi\|309661 | regulatory protein [Plasmid pCF10] | 73 | 50 | |
| 897 | 1 | 3 | 170 | gi\|807976 | unknown [Saccharomyces cerevisiae] | 73 | 57 | |
| 5 | 1 | 223 | 2 | gnl\|PID\|e255315 | unknown [Mycobacterium tuberculosis] | 72 | 56 | |
| 8 | 5 | 4158 | 4799 | gi\|587088 | shikimate kinase [Bacillus subtilis] | 72 | 54 | |
| 19 | 6 | 2600 | 2833 | gi\|34844 | embryonic myosin heavy chain (AA 1 - 1940) | 72 | 38 | |
| | | | | | [Homo sapiens] ir\|S04090\|S04090 myosin | | | |
| | | | | | heavy chain, skeletal muscle, embryonic - | | | |
| | | | | | man | | | |
| 19 | 25 | 12872 | 14605 | gnl\|PID\|e242896 | orf5 [Bacteriophage A2] | 72 | 52 | |
| 21 | 4 | 2777 | 2598 | gi\|54115 | skeletal muscle chloride channel [Mus | 72 | 45 | |
| | | | | | musculus domesticus] | | | |
| 23 | 7 | 3702 | 4847 | gi\|144714 | NADPH-dependent butanol dehydrogenase | 72 | 48 | |
| | | | | | [Clostridium acetobutylicum] | | | |
| | | | | | pir\|JU0053\|JJU0053 NADPH-dependent butanol | | | |
| | | | | | dehydrogenase - lostridium acetobutylicum | | | |
| 32 | 1 | 1073 | 3 | gi\|1303839 | YqfR [Bacillus subtilis] | 72 | 50 | |
| 39 | 8 | 4137 | 3244 | pir\|A32950\|A32950 | probable reductase protein - Leishmania | 72 | 55 | |
| | | | | | major | | | |
| 43 | 3 | 969 | 1919 | gi\|290494 | o287 [Escherichia coli] | 72 | 46 | |
| 45 | 2 | 911 | 1567 | gi\|1039479 | ORFU [Lactococcus lactis] | 72 | 50 | |
| 55 | 6 | 2549 | 2896 | gi\|755602 | unknown [Bacillus subtilis] | 72 | 51 | |
| 55 | 7 | 3178 | 3660 | gi\|1303914 | YghY [Bacillus subtilis] | 72 | 49 | |
| 60 | 1 | 1302 | 34 | gi\|143374 | phosphoribosyl glycinamide synthetase | 72 | 59 | |
| | | | | | (PUR-D; gtg start codon) Bacillus | | | |
| | | | | | subtilis] | | | |
| 60 | 3 | 3422 | 2838 | gi\|143372 | phosphoribosyl glycinamide | 72 | 48 | |
| | | | | | formyltransferase (PUR-N) [Bacillus | | | |
| | | | | | ubtilis] | | | |
| 60 | 10 | 9771 | 9010 | gi\|143367 | phosphoribosyl aminoidazole | 72 | 57 | |
| | | | | | succinocarboxamide synthetase (PUR-C; tg | | | |
| | | | | | start codon) [Bacillus subtilis] | | | |
| 70 | 5 | 3615 | 3833 | sp\|P43672\|YCBH_ECO | HYPOTHETICAL 14.4 KD PROTEIN IN PYRD-PQIA | 72 | 48 | |
| | | | | LI | INTERGENIC REGION. | | | |
| 79 | 2 | 632 | 841 | gi\|1652343 | ABC transporter [Synechocystis sp.] | 72 | 47 | |
| 85 | 2 | 1843 | 770 | gi\|1354775 | pfoS/R [Treponema pallidum] | 72 | 45 | |
| 87 | 1 | 2 | 745 | gi\|42029 | ORF1 gene product [Escherichia coli] | 72 | 47 | |
| 88 | 1 | 124 | 1047 | gi\|535348 | CodV [Bacillus subtilis] | 72 | 50 | |
| 88 | 7 | 3862 | 4752 | gi\|149413 | ORF [Lactococcus lactis] | 72 | 51 | |
| 91 | 2 | 611 | 877 | gi\|726480 | L-glutamine-D-fructose-6-phosphate | 72 | 57 | |
| | | | | | amidotransferase [Bacillus ubtilis] | | | |
| 98 | 16 | 16302 | 15163 | gi\|147326 | transport protein [Escherichia coli] | 72 | 57 | |
| 101 | 6 | 4676 | 4023 | gi\|1109685 | ProW [Bacillus subtilis] | 72 | 53 | |
| 104 | 3 | 5331 | 3982 | gi\|312441 | dihydroorotase [Bacillus caldolyticus] | 72 | 58 | |
| 114 | 10 | 11165 | 12205 | gi\|556881 | Similar to Saccharomyces cerevisiae SUA5 | 72 | 60 | |
| | | | | | protein [Bacillus subtilis] | | | |
| | | | | | pir\|S49358\|S49358 ipc-29d protein - | | | |
| | | | | | Bacillus subtilis sp\|P39153\|YWLC_BACSU | | | |
| | | | | | HYPOTHETICAL 37.0 KD PROTEIN IN SPOIIR- | | | |
| | | | | | GLYC NTERGENIC REGION. | | | |
| 128 | 19 | 14325 | 11560 | gi\|143150 | levR [Bacillus subtilis] | 72 | 58 | |
| 130 | 2 | 382 | 1437 | gi\|308850 | ATP binding protein [Lactoccus lactis] | 72 | 55 | |
| 135 | 4 | 5012 | 3693 | gi\|413940 | ipa-16d gene product [Bacillus subtilis] | 72 | 56 | |
| 150 | 6 | 5114 | 5878 | gi\|495046 | tripeptidase [Lactococcus lactis] | | | |
| 154 | 9 | 5850 | 5677 | gi\|425467 | transposase [Lactobacillus helveticus] | 72 | 52 | |
| 168 | 4 | 1375 | 1563 | gi\|1652869 | NADH dehydrogenase [Synechocystis sp.] | 72 | 55 | |
| 173 | 5 | 2879 | 4024 | gnl\|PID\|e254877 | unknown [Mycobacterium tuberculosis] | 72 | 57 | |
| 179 | 2 | 1608 | 2399 | gi\|709993 | hypothetical protein [Bacillus subtilis] | 72 | 45 | |
| 179 | 6 | 7584 | 7844 | gi\|1161934 | DltC [Lactobacillus casei] | 72 | 54 | |
| 180 | 21 | 19948 | 21105 | gi\|1773197 | similar to M. fervidus malate | 72 | 55 | |
| | | | | | dehydrogenase [Escherichia coli] | | | |
| 182 | 1 | 3 | 413 | gi\|1146182 | putative [Bacillus subtilis] | 72 | 48 | |
| 200 | 23 | 13106 | 12789 | gi\|1707358 | polyprotein precurser [Soybean mosaic | 72 | 34 | |
| | | | | | virus] | | | |
| 204 | 6 | 2462 | 2289 | gi\|1200525 | dihydrolipoamide acetyltransferase | 72 | 61 | |
| | | | | | [Pseudomonas aeruginosa] | | | |
| 204 | 9 | 6374 | 5187 | gi\|1732040 | alcohol dehydrogenase [Actinobacillus | 72 | 56 | |
| | | | | | pleuropneumoniae] | | | |
| 205 | 1 | 463 | 71 | gi\|42029 | ORF1 gene product [Escherichia coli] | 72 | 57 | |
| 210 | 7 | 6433 | 5279 | gi\|142978 | glycerol dehydrogenase [Bacillus | 72 | 46 | |
| | | | | | stearothermophilus] | pir I JQ1474 I JQ1474 | | |
| | | | | | glycerol dehydrogenase (EC 1.1.1.6) - | | | |
| | | | | | Bacillus tearothermophilus | | | |
| 213 | 6 | 4086 | 5141 | gi\|431231 | uracil permease [Bacillus caldolyticus] | 72 | 51 | |
| 223 | 1 | 99 | 833 | gi\|1573615 | ATP-binding protein (abc) [Haemophilus | 72 | 47 | |
| | | | | | influenzae] | | | |
| 227 | 1 | 26 | 886 | gi\|1070015 | protein-dependent [Bacillus subtilis] | 72 | 52 | |
| 228 | 4 | 2047 | 2481 | gi\|467339 | unknown [Bacillus subtilis] | 72 | 50 | |
| 238 | 17 14728 | 15582 | gi\|882736 | ORF_f278 [Escherichia coli] | 72 | 59 | | |
| 250 | 6 | 4169 | 4765 | gi\|437389 | transposase [Lactococcus lactis] | 72 | 56 | |
| 258 | 7 | 5296 | 7089 | gi\|192185 | acid beta-galactosidase [Mus musculus] | 72 | 53 | |
| 266 | 3 | 2024 | 1773 | gi\|145149 | ORFd [Escherichia coli] | 72 | 50 | |
| 269 | 8 | 5142 | 4477 | gi\|1303791 | YgeJ [Bacillus subtilis] | 72 | 45 | |
| 276 | 13 | 9843 | 8152 | gnl\|PID\|e59644 | predicted 86.4kd protein; 52Kd observed | 72 | 48 | |
| | | | | | [Mycobacteriophage 15] | | | |
| 278 | 2 | 965 | 1573 | gi\|425467 | transposase [Lactobacillus helveticus] | 72 | 52 | |
| 279 | 2 | 1305 | 340 | gnl\|PID\|e198981 | ttg start [Campylobacter coli] | 72 | 47 | |
| 283 | 4 | 1668 | 2045 | gi\|1353563 | ORF46 [Bacteriophage rlt] | 72 | 48 | |
| 286 | 2 | 789 | 2606 | gi\|1651216 | Pz-peptidase [Bacillus licheniformis] | 72 | 52 | |
| 290 | 4 | 2676 | 3239 | gi\|1653645 | ribosome releasing factor [Synechocystis | 72 | 56 | |
| | | | | | sp.] | | | |
| 301 | 2 | 1762 | 899 | gi\|606013 | CG Site No. 829 [Escherichia coli] | 72 | 57 | |
| 362 | 2 | 377 | 688 | gi\|1001826 | cadmium-transporting ATPase [Synechocystis | 72 | 53 | |
| | | | | | sp.] | | | |
| 369 | 1 | 582 | 142 | gi\|153745 | mannitol-specific enzyme III | 72 | 47 | |
| | | | | | [Streptococcus mutans]pir\|B44798\|844798 | | | |
| | | | | | mannitol-specific factor III, MtlF - | | | |
| | | | | | treptococcus mutans | | | |
| 379 | 2 | 1934 | 1527 | gi\|1055071 | C23G10.2 gene product [Caenorhabditis | 72 | 51 | |
| | | | | | elegans] | | | |
| 384 | 2 | 694 | 1098 | gi\|1208474 | hypothetical protein [Synechocystis sp.] | 72 | 49 | |
| 388 | 1 | 291 | 4 | gi\|1673836 | (AE000018) Mycoplasma pneumoniae, | 72 | 43 | |
| | | | | | osmotically inducible protein; similar to | | | |
| | | | | | Swiss-Prot Accession Number P23929, from | | | |
| | | | | | E. coli [Mycoplasma pneumoniae] | | | |
| 401 | 6 | 3995 | 5137 | gi\|508242 | ORF 6, putative Galf synthesis pathway | 72 | 62 | |
| | | | | | protein [Escherichia coli] gi\|510253 orf6 | | | |
| | | | | | [Escherichia coli] | | | |
| 404 | 2 | 2119 | 776 | gi\|466474 | cellobiose phosphotransferase enzyme II′ | 72 | 48 | |
| | | | | | [Bacillus tearothermophilus] | | | |
| 416 | 4 | 3461 | 1980 | gi\|710632 | beta-glucosidase [Bacillus subtilis] | 72 | 55 | |
| 416 | 7 | 6285 | 5551 | gnl\|PID\|e269549 | Unknown [Bacillus subtilis] | 72 | 52 | |
| 419 | 3 | 759 | 505 | gi\|928830 | ORF75; putative [Lactococcus lactis phage | 72 | 47 | |
| | | | | | BK5-T] | | | |
| 441 | 4 | 3420 | 4676 | gi\|1732195 | beta-cystathionase [Vibrio furnissii] | 72 | 54 | |
| 460 | 3 | 1385 | 2641 | gi\|1652389 | beta ketoacyl-acyl carrier protein | 72 | 55 | |
| | | | | | synthase [Synechocystis sp.] | | | |
| 460 | 5 | 3129 | 3560 | gnl\|PID\|e289141 | similar to hydroxymyristoyl-(acyl carrier | 72 | 54 | |
| | | | | | protein) dehydratase [Bacillus subtilis] | | | |
| 460 | 8 | 5817 | 6023 | gi\|285621 | undefined open reading frame [Bacillus | 72 | 57 | |
| | | | | stearothermophilus] | | | | |
| 462 | 2 | 1591 | 785 | gi\|148304 | beta-1,4-N-acetylmuramoylhydrolase | 72 | 51 | |
| | | | | | [Enterococcus hirae] pir\|A42296\|A42296 | | | |
| | | | | | lysozyme 2 (EC 3.2.1.-) precursor - | | | |
| | | | | | Enterococcus irae (ATCC 9790) | | | |
| 467 | 1 | 2 | 706 | gi\|148711 | 6-aminohexanoate-cyclic-dimer hydrolase | 72 | 50 | |
| | | | | | [Flavobacterium sp.] gi\|488343 6- | | | |
| | | | | | aminohexanoate-cyclic-dimer hydrolase | | | |
| | | | | | [Flavobacterium p.] | | | |
| 469 | 3 | 1144 | 1419 | gi\|466474 | cellobiose phosphotransferase enzyme II″ | 72 | 48 | |
| | | | | | [Bacillus tearothermophilusi] | | | |
| 493 | 1 | 1124 | 240 | sp\|IP5O848IYPW&BAC | HYPOTHETICAL 58.2 KD PROTEIN IN KDGT-XPT | 72 | 58 | |
| SU | INTERGENIC REGION. | | | | | | | |
| 536 | 2 | 379 | 218 | gi\|437389 | transposase [Lactococcus lactis] | 72 | 58 | |
| 543 | 1 | 574 | 86 | gi\|290513 | f470 [Escherichia coli] | 72 | 47 | |
| 592 | 1 | 57 | 680 | gi\|987092 | ABC-transporter [Streptomyces | 72 | 55 | |
| | | | | | hygroscopicus] | | | |
| 666 | 2 | 551 | 967 | gi\|1064786 | function unknown [Bacillus subtilis] | 72 | 48 | |
| 762 | 1 | 974 | 273 | gi\|304928 | pantothenate synthetase [Escherichia coli] | 72 | 55 | |
| 792 | 1 | 401 | 3 | pir\|A36933\|A36933 | diacyiglycerol kinase homolog - | 72 | 50 | |
| | | | | | Streptococcus mutans | | | |
| 873 | 1 | 183 | 4 | gnl\|PID\|e258329 | oxaloacetate decarboxylase alpha-chain | 72 | 55 | |
| | | | | | [Legionella pneumophila] | | | |
| 4 | 4 | 3799 | 3155 | gi\|496943 | ORF [Saccharomyces cerevisiae] | | | |
| 10 | 2 | 180 | 977 | gnl\|PID\|e234078 | hom [Lactococcus lactis] | 71 | 49 | |
| 16 | 7 | 4922 | 6097 | gi\|534982 | phosphoglucomutase [Spinacia oleracea] | 71 | 54 | |
| 21 | 6 | 4148 | 3972 | gi\|1736645 | Proline/betaine transporter (Proline | 71 | 50 | |
| | | | | | porter II) (PPII) . [Escherichia coli] | | | |
| 23 | 27 | 16452 | 17459 | gi\|1408503 | yxeR gene product [Bacillus subtilis] | 71 | 52 | |
| 25 | 7 | 5812 | 6669 | gi\|413943 | ipa-19d gene product [Bacillus subtilis] | 71 | 58 | |
| 31 | 1 | 80 | 946 | gi\|534045 | antiterminator [Bacillus subtilis] | 71 | 47 | |
| 39 | 3 | 755 | 1297 | sp\|P09997\|YIDA_ECO | HYPOTHETICAL 29.7 KD PROTEIN IN IBPA-GYRB | 71 | 50 | |
| | | | | LI | INTERGENIC REGION. | | | |
| 39 | 7 | 2537 | 3193 | pir\|C43748\|C43748 | hypothetical protein (pepX 3′ region) - | 71 | 54 | |
| | | | | | Lactococcus lactis subsp. lactis | | | |
| 45 | 10 | 5119 | 5484 | gi\|606044 | ORF_o130; Geneplot suggests frameshift, | 71 | 51 | |
| | | | | | none found [Escherichia oil] | | | |
| 48 | 10 | 11722 | 10148 | gi\|20432 | 4-cournarate:CoA ligase Pc4Cl-1 (AA 1-544) | 71 | 39 | |
| | | | | | [Petroselinum crispum] ir\|S0l667\|S01667 4- | | | |
| | | | | | coumarate--CoA ligase (EC 6.2.1.12) (clone | | | |
| | | | | | 4CL-1) - parsley | | | |
| 55 | 4 | 1470 | 1709 | gi\|1303901 | YqhT [Bacillus subtilis] | 71 | 54 | |
| 57 | 10 | 12899 | 13060 | gi\|40053 | phenylalanyl-tRNA synthetase alpha subunit | 71 | 45 | |
| | | | | | [Bacillus subtilis] ir\|S11730\|YFBSA | | | |
| | | | | | phenylalanine--tRNA ligase (EC 6.1.1.20) | | | |
| | | | | | alpha ain - Bacillus subtilis | | | |
| 58 | 3 | 3743 | 2571 | gi\|1658403 | formate dehydrogenase alpha subunit | 71 | 51 | |
| | | | | | [Moorella thermoacetica] | | | |
| 68 | 11 | 8225 | 8602 | gi\|793910 | surface antigen [Homo sapiens] | 71 | 49 | |
| 74 | 4 | 2908 | 2042 | gi\|467435 | unknown [Bacillus subtilis] | 71 | 55 | |
| 85 | 3 | 3267 | 1966 | gi\|142613 | branched chain alpha-keto acid | 71 | 56 | |
| | | | | | dehydrogenase E2 [Bacillus subtilis] | | | |
| | | | | | gi\|1303944 BfmBB [Bacillus subtilis] | | | |
| 111 | 8 | 5737 | 4253 | gi\|1256135 | YbbF [Bacillus subtilis] | 71 | 50 | |
| 111 | 9 | 6590 | 5730 | gi\|1573762 | glucokinase regulator [Haemophilus | 71 | 53 | |
| | | | | | influenzae] | | | |
| 120 | 1 | 111 | 353 | gnl\|PID\|e235823 | unknown [Schizosaccharmyces pombe] | 71 | 52 | |
| 123 | 11 | 10387 | 11196 | gi\|1773195 | hypothetical [Escherichia coli] | 71 | 55 | |
| 151 | 3 | 4045 | 3098 | gi\|1256618 | transport protein [Bacillus subtilis] | 71 | 51 | |
| 172 | 6 | 3949 | 4806 | gi\|1262288 | CdsA [Brucella abortus] | 71 | 56 | |
| 172 | 7 | 5264 | 6448 | gi\|40100 | rodC (tag3) polypeptide (AA 1-746) | 71 | 52 | |
| | | | | | [Bacillus subtilis] ir\|S06049\|S06049 rode | | | |
| | | | | | protein - Bacillus subtilis | | | |
| | | | | | p\|P13485\|TAGF_BACSU TEICHOIC ACID | | | |
| | | | | | BIOSYNTHESIS PROTEIN F. | | | |
| 190 | 7 | 3454 | 3122 | gi\|532556 | ORF23 [Enterococcus faecalis] | 71 | 52 | |
| 195 | 24 | 9850 | 11871 | gi\|405564 | traE [Plasmid pSK41] | 71 | 45 | |
| 215 | 4 | 3361 | 2711 | gi\|1573086 | uridine kinase (uridine monophosphokinase) | 71 | 51 | |
| | | | | | (udk) [Haemophilus influenzae] | | | |
| 218 | 2 | 1456 | 2613 | gnl\|PID\|e254644 | membrane protein [Streptococcus | 71 | 41 | |
| | | | | | pneumoniae] | | | |
| 222 | 3 | 1205 | 2053 | gnl\|PID\|e255114 | glutamate racemase [Bacillus subtilis] | 71 | 56 | |
| 222 | 4 | 1611 | 1387 | gi\|1001195 | phosphate transport system permease | 71 | 57 | |
| | | | | | protein PstA [Synechocystis sp.] | | | |
| 222 | 14 | 8852 | 9853 | gi\|466720 | No definition line found [Escherichia | 71 | 53 | |
| | | | | | coli] | | | |
| 238 | 22 | 19256 | 20578 | gi\|595299 | YgiK [Salmonella typhimurium] | 71 | 50 | |
| 255 | 3 | 2692 | 1061 | gnl\|PID\|e254877 | unknown [Mycobacterium tuberculosis] | 71 | 55 | |
| 265 | 5 | 2960 | 1581 | gi\|1039479 | ORFU [Lactococcus lactis] | 71 | 58 | |
| 276 | 2 | 1359 | 538 | gi\|496283 | lysin [Bacteriophage Tuc2009] | 71 | 63 | |
| 290 | 5 | 3552 | 4379 | gi\|1016162 | ABC transporter subunit [Cyanophora | 71 | 49 | |
| | | | | | paradoxa] | | | |
| 290 | 7 | 5659 | 6912 | gi\|1001708 | NifS [Synechocystis sp.] | 71 | 56 | |
| 292 | 3 | 948 | 2156 | gn1\|PID\|e233874 | hypothetical protein [Bacillus subtilis] | 71 | 55 | |
| 318 | 4 | 3229 | 2285 | gi\|1256138 | YbbI [Bacillus subtilis] | 71 | 54 | |
| 333 | 1 | 145 | 741 | gi\|293011 | unknown protein [Lactococcus lactis] | 71 | 50 | |
| 344 | 1 | 76 | 396 | gi\|853775 | unknown [Bacillus subtilis] | 71 | 53 | |
| 350 | 1 | 138 | 1394 | gi\|1652389 | beta ketoacyl-acyl carrier protein | 71 | 57 | |
| | | | | | synthase [Synechocystis sp.] | | | |
| 363 | 4 | 4184 | 5674 | gi\|1657518 | similar to fdrA gene of E. coli | 71 | 54 | |
| | | | | | [Escherichia coli] | | | |
| 364 | 5 | 5319 | 6563 | gi\|1657522 | hypothetical protein [Escherichia coli] | 71 | 46 | |
| 367 | 13 | 6539 | 6162 | gi\|44225 | ribosomal protein L18 (AA 1-116) | 71 | 51 | |
| | | | | | [Mycoplasma capricolum] ir\|S02847\|R5YM18 | | | |
| | | | | | ribosomal protein L18 - Mycoplasma | | | |
| | | | | | capricolum GC3) | | | |
| 379 | 7 | 6884 | 5655 | gi\|887821 | ORF_o398 [Escherichia coli] | 71 | 50 | |
| 399 | 9 | 6528 | 7664 | gi\|154198 | oxaloacetate decarboxylase [Salmonella | 71 | 50 | |
| | | | | | typhimurium] pir\|C44465\|C44465 sodium ion | | | |
| | | | | | pump oxaloacetate decarboxylase ubunit | | | |
| | | | | | beta - Salmonella typhimurium | | | |
| 399 | 18 | 13540 | 14778 | gi\|143165 | malic enzyme (EC 1.1.1.38) [Bacillus | 71 | 46 | |
| | | | | | stearothermophilus] pir\|A33307\|DEBSXS | | | |
| | | | | | malate dehydrogenase oxaloacetate- | | | |
| | | | | | decarboxylating) (EC 1.1.1.38) - Bacillus | | | |
| | | | | | tearothermophilus | | | |
| 404 | 4 | 3769 | 3029 | gi\|143402 | recombination protein (ttg start codon) | 71 | 48 | |
| | | | | | [Bacillus subtilis] gi\|1303923 RecN | | | |
| | | | | | [Bacillus subtilis] | | | |
| 464 | 1 | 1532 | 216 | gi\|895749 | putative cellobiose phosphotransferase | 71 | 40 | |
| | | | | | enzyme II″ [Bacillus ubtilis] | | | |
| 464 | 3 | 2088 | 2846 | gi\|1486242 | unknown [Bacillus subtilis] | 71 | 39 | |
| 481 | 2 | 954 | 409 | gi\|144729 | butanol dehydrogenase [Clostridium | 71 | 58 | |
| | | | | | acetobutylicum] sp\|Q04944\|ADHA_CLOAB NADH- | | | |
| | | | | | DEPENDENT BUTANOL DEHYDROGENASE A (EC | | | |
| | | | | | .1.1.-) (BDH I). | | | |
| 482 | 4 | 2503 | 1841 | gi\|1072418 | gicA gene product [Staphylococcus | 71 | 58 | |
| | | carnosus] | | | | | | |
| 496 | 2 | 1636 | 848 | gi\|1001226 | methionine aminopeptidase [Synechocystis | 71 | 51 | |
| | | | | | sp.] | | | |
| 503 | 2 | 1624 | 650 | gi\|39478 | ATP binding protein of transport ATPases | 71 | 49 | |
| | | | | | [Bacillus firmus] ir\|S15486\|S15486 ATP- | | | |
| | | | | | binding protein - Bacillus firmus | | | |
| | | | | | p\|26946\|YATR_BACFI HYPOTHETICAL ABC | | | |
| | | | | | TRANSPORTER ATP-BINDNG OTEIN. | | | |
| 513 | 2 | 1590 | 982 | gnl\|PID\|e202290 | unknown [Lactobacillus sake] | 71 | 46 | |
| 530 | 1 | 2 | 1534 | gi\|1542974 | AbcA [Thermoanaerobacterium | 71 | 52 | |
| | | | | | thermosulfurigenes] | | | |
| 537 | 1 | 706 | 365 | gi\|929972 | ORFB; similar to B. anthracis SterneL | 71 | 57 | |
| | | | | | element ORFB; putative S150-like | | | |
| | | | | | transposase [Bacillus anthracis] | | | |
| 553 | 1 | 304 | 1287 | gi\|1653479 | regulatory components of sensory | 71 | 48 | |
| | | | | | transduction system [Synechocystis sp.] | | | |
| 573 | 9 | 5560 | 5090 | gi\|143799 | MtrA [Bacillus subtilis] | 71 | 59 | |
| 583 | 1 | 21 | 341 | gi\|1064791 | function umknown [Bacillus subtilis] | 71 | 50 | |
| 584 | 2 | 638 | 276 | gi\|662792 | single-stranded DNA binding protein | 71 | 58 | |
| | | | | | [unidentified eubacterium] | | | |
| 585 | 1 | 282 | 809 | gi\|666972 | ORF 168 [Synechococcus sp.] | 71 | 46 | |
| 611 | 1 | 985 | 2 | gi\|1039479 | ORFU [Lactococcus lactis] | 71 | 55 | |
| 616 | 1 | 350 | 3 | gi\|1088272 | nitrogen fixation protein [Bacillus | 71 | 52 | |
| | | | | | cereus] | | | |
| 624 | 1 | 61 | 399 | gi\|40014 | pot. ORF 446 (aa 1-446) [Bacillus | 71 | 53 | |
| | | | | | subtilis] | | | |
| 624 | 2 | 608 | 1732 | gi\|40015 | pot. ORF 378 (aa 1-378) [Bacillus | 71 | 51 | |
| | | | | | subtilis] | | | |
| 659 | 1 | 76 | 582 | gi\|1591045 | hypothetical protein (SP:P31466) | 71 | 51 | |
| | | | | | [Methanococcus jannaschii] | | | |
| 668 | 2 | 836 | 1030 | gi\|467330 | replicative DNA helicase [Bacillus | 71 | 60 | |
| | | | | | subtilis] | | | |
| 683 | 1 | 582 | 118 | gnl\|PID\|e264663 | CinA [Streptococcus pneumoniae] | 71 | 55 | |
| 701 | 3 | 411 | 797 | gi\|143795 | transfer RNA-Tyr synthetase [Bacillus | 71 | 51 | |
| | | | | | subtilis] | | | |
| 720 | 1 | 1 | 351 | gi\|1595810 | type-I signal peptidase SpsB | 71 | 55 | |
| | | | | | [Staphylococcus aureus] | | | |
| 724 | 2 | 1020 | 415 | gnl\|PID\|e239621 | ORF YNL218w [Saccharomyces cerevisiae] | 71 | 51 | |
| 790 | 2 | 658 | 383 | gi\|1783253 | homologous to many ATP-binding transport | 71 | 48 | |
| | | | | | proteins; hypothetical [Bacillus subtilis] | | | |
| 799 | 1 | 505 | 906 | gi\|580866 | ipa-12d gene product [Bacillus subtilis] | 71 | 45 | |
| 974 | 2 | 139 | 333 | gi\|1778531 | H10021 homolog [Escherichia coli] | | | |
| 980 | 1 | 156 | 497 | gi\|437389 | transposase [Lactococcus lactis] | | | |
| 4 | 3 | 3170 | 2418 | gi\|1001805 | hypothetical protein [Synechocystis sp.] | 70 | 55 | |
| 17 | 21 | 18642 | 21527 | gi\|145821 | EBG enzyme alpha subunit [Escherichia | 70 | 53 | |
| | | | | | coli] | | | |
| 19 | 8 | 2894 | 3952 | gi\|1353527 | ORF10 [Bacteriophage rlt] | 70 | 58 | |
| 23 | 6 | 2640 | 3230 | gi\|699336 | C. freundli orfW homologue [Mycobacterium | 70 | 43 | |
| | | | | | leprae] sp\|P53523\|Y02Y_MYCLE HYPOTHETICAL | | | |
| | | | | | 20.9 KD PROTEIN U471A. | | | |
| 27 | 3 | 1011 | 493 | gi\|1001644 | regulatory components of sensory | 70 | 44 | |
| | | | | | transduction system [Synechocystis sp.] | | | |
| 31 | 2 | 1095 | 1337 | gi\|1100076 | PTS-dependent enzyme II [Clostridium | 70 | 55 | |
| | | | | | longisporum] | | | |
| 32 | 10 | 6527 | 5817 | gi\|1591789 | M. jannaschii predicted coding region | 70 | 51 | |
| | | | | | MJ1163 [Methanococcus jannaschii] | | | |
| 33 | 7 | 6930 | 7235 | gi\|536972 | ORF_o90a [Escherichia coli] | 70 | 45 | |
| 35 | 2 | 500 | 2533 | gi\|43819 | nagE gene product [Klebsiella pneumoniae] | 70 | 50 | |
| 47 | 13 | 15837 | 14512 | gi\|150209 | ORF 1 [Mycoplasma mycoides] | 70 | 44 | |
| 49 | 15 | 10409 | 11179 | gi\|853751 | N-acetylmuramoyl-L-alanine amidase | 70 | 54 | |
| | | | | | [Bacteriophage A511] | | | |
| 57 | 7 | 8365 | 12189 | gi\|142440 | ATP-dependent nuclease [Bacillus subtilis] | 70 | 48 | |
| 57 | 16 | 18656 | 18033 | gi\|388565 | major cell-binding factor [Campylobacter | 70 | 52 | |
| | | | | | jejuni] | | | |
| 59 | 9 | 4985 | 7060 | gnl\|PID\|e254877 | unknown [Mycobacterium tuberculos] | 70 | 49 | |
| 72 | 6 | 6771 | 4600 | gi\|557567 | ribonucleotide reductase R1 subunit | 70 | 53 | |
| | | | | | [Mycobacterium tuberculosis] | | | |
| | | | | | sp\|P50640\|RIR1_MYCTU RIBONUCLEOSIDE- | | | |
| | | | | | DIPHOSPHATE REDUCTASE ALPHA HAIN (EC | | | |
| | | | | | 1.17.4.1) (RIBONUCLEOTIDE REDUCTASE) (R1 | | | |
| | | | | | SUBUNIT) FRAGMENT). | | | |
| 76 | 8 | 5960 | 6343 | gi\|1063251 | no homologous protein [Bacillus subtilis] | 70 | 52 | |
| 81 | 16 | 12529 | 11723 | gi\|1732200 | PTS permease for rnannose subunit IIPMan | 70 | 52 | |
| | | | | | [Vibrio furnissii] | | | |
| 98 | 7 | 8974 | 7874 | gi\|1573045 | hypothetical [Haemophilus influenzae] | 70 | 46 | |
| 110 | 2 | 1353 | 502 | gi\|1399848 | unknown [Synechococcus PCC7942] | 70 | 52 | |
| 123 | 7 | 5009 | 5527 | gi\|143284 | negative regulator pal 1 [Bacillus | 70 | 51 | |
| | | | | | subtilis] | | | |
| 123 | 22 | 19729 20412 gi\|1591493 | glutamine transport ATP-binding protein Q | 70 48 | | | | |
| | | | | | [Methanococcus jannaschii] | | | |
| 133 | 6 | 5905 | 6498 | gi\|746399 | transcription elongation factor | 70 | 50 | |
| | | | | | [Escherichia coli] | | | |
| 134 | 1 | 1 | 384 | gi\|1146242 | aspartate 1-decarboxylase [Bacillus | 70 | 49 | |
| | | | | | subtilis] | | | |
| 138 | 10 | 8543 | 7953 | gi\|467371 | LACI family of transcriptional repreesor | 70 | 50 | |
| | | | | | (probable) [Bacillus ubtilis] | | | |
| 160 | 3 | 1263 | 1520 | gi\|1468939 | meso-2,3-butanediol dehydrogenase (D- | 70 | 45 | |
| | | | | | acetoin forming) [Klebsiella pneumoniae] | | | |
| 174 | 3 | 2279 | 1572 | gi\|413931 | ipa-7d gene product [Bacillus subtilis] | 70 | 44 | |
| 177 | 2 | 2104 | 1022 | gnl\|PID\|e186242 | D-mannonate hydrolase [Thermotoga | 70 | 52 | |
| | | | | | neapolitana] | | | |
| 178 | 2 | 1320 | 532 | gi\|499659 | K+ channel protein [Panulirus interruptus] | 70 | 51 | |
| 180 | 18 | 17770 | 18729 | gi\|887824 | ORF_o310 [Escherichia coli] | 70 | 50 | |
| 180 | 22 | 21072 | 22526 | gi\|1573294 | hypothetical [Haemophilus influenzae] | 70 | 40 | |
| 181 | 9 | 7409 | 6279 | sp\|P20692\|TYRA_BAC | PREPHENATE DEHYDROGENASE (EC 1.3.1.12) | 70 | 49 | |
| | | | | | SU | (PDH). | | |
| 197 | 5 | 4529 | 6340 | gi\|1783252 | homologous to many ATP-binding transport | 70 | 47 | |
| | | | | | proteins including Swissprot:CYDD_ECOLI; | | | |
| | | | | | hypothetical [Bacillus subtilis] | | | |
| 200 | 21 | 12419 | 11820 | gi\|290943 | HindIII modification methyltransferase | 70 | 47 | |
| | | | | | [Haemophilus influenzae] | | | |
| | | | | | sp\|P43871\|MTH3_HAEIN MODIFICATION | | | |
| | | | | | METHYLASE HINDIII (SC 2.1.1.72) ADENINE- | | | |
| | | | | | SPECIFIC METHYLTRANSFERASE HINDIII) | | | |
| | | | | | (M.HINDIII) | | | |
| 210 | 4 | 3877 | 3269 | gi\|602683 | orfC [Mycoplasma capricolum] | 70 | 47 | |
| 217 | 2 | 405 | 707 | gi\|153767 | ORF [Streptococcus pneumoniae] | 70 | 56 | |
| 222 | 8 | 4940 | 6046 | gi\|537033 | ORF_f356 [Escherichia coli] | 70 | 54 | |
| 222 | 15 | 9825 | 10553 | gi\|537039 | ORF_o228a [Escherichia coli] | 70 | 56 | |
| 227 | 4 | 1871 | 2893 | gi\|1070014 | protein-dependent [Bacillus subtilis] | 70 | 44 | |
| 228 | 2 | 1343 | 792 | gi\|1742730 | Protein AraJ precursor. [Escherichia coli] | 70 | 50 | |
| 228 | 5 | 3470 | 2574 | gi\|1573390 | hypothetical [Haemophilus influenzae] | 70 | 54 | |
| 231 | 2 | 2470 | 1238 | gi\|1574085 | H. influenzae predicted coding region | 70 | 48 | |
| | | | | | HI1048 [Haemophilus influenzae] | | | |
| 235 | 4 | 2779 | 2138 | gi\|309662 | pheromone binding protein [Plasmid pCF10] | 70 | 46 | |
| 239 | 4 | 5807 | 6409 | gi\|682765 | mccB gene product [Escherichia coli] | 70 | 41 | |
| 248 | 1 | 3 | 350 | gi\|143725 | putative [Bacillus subtilis] | 70 | 52 | |
| 254 | 4 | 838 | 497 | gi\|49318 | ORF4 gene product [Bacillus subtilis] | 70 | 48 | |
| 256 | 3 | 1737 | 2612 | gi\|596092 | putative multiple membrane domain protein; | 70 | 51 | |
| | | | | | possible TTG initiation odon at position | | | |
| | | | | | 1064, near putative RBS at position 1052 | | | |
| | | | | | Streptococcus pyogenes] | | | |
| 279 | 15 | 14547 | 14224 | gi\|1389549 | ORF3 [Bacillus subtilis] | 70 | 50 | |
| 283 | 6 | 2279 | 3190 | gi\|853751 | N-acetylmuralmoyl-L-alanine amidase | 70 | 52 | |
| | | | | | [Bacteriophage A511] | | | |
| 292 | 8 | 5557 | 6534 | gi\|474195 | This ORF is homologous to a 40.0 kd 70 | 50 | | |
| | | | | | hypothetical protein in the htrB ′ region | | | |
| | | | | | from E. coli, Accession Number X61000 | | | |
| | | | | | [Mycoplasma-like rganism] | | | |
| 294 | 8 | 2776 | 3375 | gi\|1750126 | YncB [Bacillus subtilis] | 70 | 47 | |
| 294 | 10 | 3742 | 4020 | gi\|984581 | YafQ [Escherichia coli] | 70 | 50 | |
| 299 | 1 | 905 | 132 | gi\|606309 | ORF_o265; gtg start [Escherichia coli] | 70 | 40 | |
| 300 | 3 | 3200 | 2784 | gi\|289260 | comE ORF1 [Bacillus subtilis] | 70 | 50 | |
| 301 | 9 | 8564 | 7590 | gi\|1303865 | YqgR [Bacillus subtilis] | 70 | 52 | |
| 336 | 2 | 661 | 921 | gi\|202864 | [Rat alternatively spliced mRNA.], gene | 70 | 47 | |
| | | | | | product [Rattus norvegicus] | | | |
| 339 | 1 | 269 | 3 | gi\|786163 | Ribosomal Protein L10 [Bacillus subtilis] | 70 | 50 | |
| 351 | 9 | 4760 | 4359 | gi\|799235 | dTDP-6-deoxy-L-lyxo-4-hexulose reductase | 70 | 45 | |
| | | | | | [Escherichia coli] | | | |
| 399 | 28 | 28203 | 28793 | gi\|146278 | glucitol-specfic enzyme II (gutA) | 70 | 52 | |
| | | | | | [Escherichia coli] pir\|A26725\|WQEC2S | | | |
| | | | | | phosphotransferase system enzyme II (EC | | | |
| | | | | | .7.1.69), sorbitol-specific, factor II - | | | |
| | | | | | Escherichia coli sp\|P05705\|PTHB_ECOLI PTS | | | |
| | | | | | SYSTEM, GLUCITOL/SORBITOL-SPECIFIC IIBC | | | |
| | | | | | OMPONENT (EIIBC-GUT) | | | |
| 406 | 1 | 1 | 552 | gi\|49315 | ORF1 gene product [Bacillus subtilis] | 70 | 50 | |
| 436 | 5 | 2417 | 2193 | gi\|773665 | transposase [Lactococcus lactis] | 70 | 36 | |
| 482 | 3 | 1887 | 1660 | gi\|48680 | ptsG-like product [Bacillus subtilis] | 70 | 47 | |
| 529 | 3 | 6587 | 7030 | gi\|1022726 | unknown [Staphylococcus haemolyticus] | 70 | 44 | |
| 535 | 2 | 1702 | 965 | gi\|1747435 | KdpE [Clostridium acetobutylicum] | 70 | 52 | |
| 543 | 2 | 1248 | 547 | gi\|1591045 | hypothetical protein (SP:P31466) | 70 | 47 | |
| | | | | | [Methanococcus jannaschii] | | | |
| 543 | 8 | 4084 | 3878 | gi\|511976 | SERP gene gene product [Plasmodium | 70 | 60 | |
| | | | | | falciparum] | | | |
| 560 | 3 | 1037 | 876 | gi\|558458 | acidic 82 kDa protein [Homo sapiens] | 70 | 40 | |
| 573 | 4 | 1920 | 2258 | gi\|336639 | prephytoene pyrophosphate dehydrogenase | 70 | 32 | |
| | | | | | [Cyanophora paradoxa] gi\|1016130 prenyl | | | |
| | | | | | transferase [Cyanophora paradoxa] | | | |
| | | | | | pir\|A40433\|A40433 prephytoene | | | |
| | | | | | pyrophosphatase dehydrogenase (crtE) | | | |
| | | | | | omolog - Cyanophora paradoxa | | | |
| 599 | 2 | 244 | 573 | gi\|42029 | ORF1 gene product [Escherichia coli] | 70 | 49 | |
| 608 | 3 | 867 | 556 | gi\|475032 | formamidopyrimidine-DNA glycosylase | 70 | 53 | |
| | | | | | [Streptococcus mutans] sp\|P55045\|FPG_STRMU | | | |
| | | | | | FORMAMIDOPYRIMIDINE-DNA GLYCOSYLASE (EC | | | |
| | | | | | .2.2.23) (FAPY-DNA GLYCOSYLASE). | | | |
| 636 | 1 | 2 | 628 | gi\|606309 | ORF_o265; gtg start [Escherichia coli] | 70 | 50 | |
| 670 | 2 | 2157 | 1828 | gi\|1657698 | hyaluronan receptor [Homo sapiens] | 70 | 41 | |
| 702 | 1 | 103 | 870 | gi\|149490 | sucrose-6-phosphate hydrolase [Lactococcus | 70 | 51 | |
| | | | | | lactis] pir\|JH0754\|JH0754 sucrose-6- | | | |
| | | | | | phosphate hydrolase (EC 3.2.1.-) - | | | |
| | | | | | actococcus lactis | | | |
| 726 | 2 | 725 | 480 | gnl\|PID\|e240103 | unknown ORF [Saccharomnyces cerevisiae] | 70 | 41 | |
| 854 | 1 | 1 | 207 | gi\|532653 | thermonuclease [Staphylococcus hyicus] | 70 | 51 | |
| 901 | 1 | 238 | 447 | gi\|172022 | myosin 1 isoform (MYO2) [Saccharomyces | 70 | 20 | |
| | | | | | cerevisiae] | | | |
| 940 | 1 | 1 | 318 | gi\|1039479 | ORFU [Lactococcus lactis] | 70 | 56 | |
| 1 | 2 | 2112 | 1213 | gi\|413976 | ipa-52r gene product [Bacillus subtilis] | 69 | 51 | |
| 8 | 2 | 2196 | 778 | gi\|1510108 | ORF-1 [Agrobacterium tumefaciens] | 69 | 50 | |
| 8 | 9 | 7949 | 6654 | gi\|1196907 | daunorubicin resistance protein | 69 | 44 | |
| | | | | | [Streptomyces peucetius] | | | |
| 16 | 3 | 1618 | 2574 | gi\|1109684 | ProV [Bacillus subtilis] | 69 | 53 | |
| 17 | 26 | 25781 | 26944 | gi\|485275 | 53.6 kDa protein [Streptococus | 69 | 44 | |
| | | | | | pneumoniae] | | | |
| 17 | 35 | 32300 | 32770 | gi\|1574146 | pfs protein (pfs) [Haemophilus influenzae] | 69 | 53 | |
| 23 | 30 | 18107 18538 | gnl\|PID\|e249656 | YneT [Bacillus subtilis] | 69 | 59 | | |
| 25 | 8 | 6653 | 6994 | gi\|413943 | ipa-19d gene product [Bacillus subtilis] | 69 | 46 | |
| 37 | 2 | 2042 | 186 | gi\|143331 | alkaline phosphatase regulatory protein | 69 | 52 | |
| | | | | | [Bacillus subtilis] pir\|A27650\|A27650 | | | |
| | | | | | regulatory protein phoR - Bacillus | | | |
| | | | | | subtilis sp\|P23545\|PHOR_BACSU ALKALINE | | | |
| | | | | | PHOSPHATASE SYNTHESIS SENSOR PROTEIN HOR | | | |
| | | | | | (EC 2.7.3.-). | | | |
| 39 | 2 | 528 | 767 | gi\|1408493 | homologous to SwissProt:YIDA_ECOLI | 69 | 52 | |
| | | | | | hypothetical protein [Bacillus subtilis] | | | |
| 56 | 6 | 4809 | 3457 | gi\|1591610 | probable ATP-dependent helicase | 69 | 45 | |
| | | | | | [Methanococcus jannaschii] | | | |
| 67 | 5 | 3042 | 3938 | gi\|1658188 | oxidative stress transcriptional regulator | 69 | 39 | |
| | | | | | [Erwinia carotovora] | | | |
| 68 | 3 | 684 | 1529 | gnl\|PID\|e214719 | P1cR protein [Bacillus thuringiensis] | 69 | 45 | |
| 72 | 4 | 2099 | 3394 | gi\|882672 | ORF_o313 [Escherichia coli] | 69 | 37 | |
| 81 | 15 | 11820 | 10915 | gi\|1732201 | PTS permease for mannose subunit IIBMan | 69 | 44 | |
| | | | | | [pi Vibria furnissii] | | | |
| 83 | 20 | 14001 | 15800 | gi\|1230668 | Similar to Arginyl-tRNA synthetase (Swiss | 69 | 44 | |
| | | | | | Prot. accession number P11875) | | | |
| | | | | | [Saccharomyces cerevisiae] | | | |
| 85 | 6 | 6309 | 5299 | sp\|P54533\|DLD2_BAC | LIPOAMIDE DEHYDROGENASE COMPONENT (E3) OF | 69 | 46 | |
| | | | | SU | BRANCHED-CHAIN ALPHA-KETO ACID | | | |
| | | | | | DEHYDROGENASE COMPLEX (EC 1.8.1.4) | | | |
| | | | | | (DIHYDROLIPOAMIDE DEHYDROGENASE) (LPD- | | | |
| | | | | | VAL). | | | |
| 86 | 3 | 2084 | 3367 | gi\|143318 | phosphoglycerate kinase [Bacillus | 69 | 53 | |
| | | | | | megaterium] | | | |
| 94 | 2 | 1401 | 751 | gi\|755216 | N-acetylmuramidase [Lactococcus lactis] | 69 | 41 | |
| 94 | 16 | 20498 | 19197 | gi\|1208948 | unknown [Escherichia coli] | 69 | 47 | |
| 98 | 8 | 10201 | 9029 | gi\|563934 | similar to E. coli hypothetical protein: | 69 | 51 | |
| | | | | | PIR Accession Number Q0614] [Bacillus | | | |
| | | | | | subtilis] | | | |
| 109 | 4 | 2350 | 1316 | gi\|396501 | aspartyl-tRNA synthetase [Thermus | 69 | 56 | |
| | | | | | aquaticus thermophilus] pir\|S33743\|533743 | | | |
| | | | | | aspartate--tRNA ligase (EC 6.1.1.12) - | | | |
| | | | | | Thermus quaticus | | | |
| 114 | 1 | 83 | 1522 | gi\|1658402 | formate dehydrogenase beta subunit | 69 | 45 | |
| | | | | | [Moorella thermoacetica] | | | |
| 123 | 9 | 7617 | 8984 | gi\|1773192 | similar to S. cerevisiae dal1 [Escherichia | 69 | 50 | |
| | | | | | coli] | | | |
| 128 | 11 | 7940 | 7578 | gi\|895750 | putative cellobiose phosphotransferase | 69 | 53 | |
| | | | | | enzyme III [Bacillus ubtilis] | | | |
| 130 | 10 | 8764 | 9036 | gi\|1641 | put. Na(+)/glucose co-transporter (AA 1- | 69 | 47 | |
| | | | | | 662) [Oryctolagus cuniculus] \|1717 | | | |
| | | | | | cortical sodium-D-glucose cotransporter | | | |
| | | | | | [Oryctolagus iculus] | | | |
| 138 | 26 | 16721 | 17545 | pir\|A25805\|A25805 | L-lactate dehydrogenase (EC 1.1.1.27) - | 69 | 55 | |
| | | | | | Bacillus subtilis | | | |
| 139 | 2 | 310 | 1083 | gi\|1408587 | relaxase [Lactococcus lactis lactis] | 69 | 46 | |
| 139 | 9 | 5196 | 4984 | gi\|473955 | DNA-binding protein [Lactobacillus sp.] | 69 | 34 | |
| 142 | 9 | 5559 | 4564 | gi\|623073 | ORF360; putative [Bacteriophage LL-H] | 69 | 47 | |
| 155 | 6 | 4658 | 5818 | gi\|1591260 | endoglucanase [Methanococcus jannaschii] | 69 | 48 | |
| 158 | 12 | 11671 | 11201 | gi\|606744 | cytidine deaminase [Bacillus subtilis] | 69 | 52 | |
| 162 | 5 | 5888 | 4032 | gi\|142993 | glycerol-3-phosphate dehydrogenase (glpD) | 69 | 54 | |
| | | | | | (EC 1.1.99.5) [Bacillus ubtilis] | | | |
| 180 | 2 | 1901 | 1203 | gi\|1575577 | DNA-binding response regulator [Thermotoga | 69 | 49 | |
| | | | | | maritima] | | | |
| 197 | 4 | 3571 | 4602 | gi\|1783251 | homologous to cytochrome d ubiquino | 169 | 46 | |
| | | | | | oxidase subunit II; hypothetical [Bacillus | C | | |
| | | | | | subtilis] | | | |
| 197 | 6 | 6283 | 7701 | gi\|1783253 | homologous to many ATP-binding transport. | 69 | 49 | |
| | | | | | proteins; hypothetical [Bacillus subtilis] | | | |
| 222 | 1 | 201 | 10 | gi\|149901 | gene codes for a 19 kDa protein | 69 | 50 | |
| | | | | | [Mycobacterium avium] sp\|P46733\|19KD_MYCAV | | | |
| | | | | | 19 KD LIPOPROTEIN ANTIGEN PRECURSOR. | | | |
| 223 | 28 | 23857 | 24567 | gnl\|PID\|e269548 | Unknown [Bacillus subtilis] | 69 | 53 | |
| 228 | 3 | 2031 | 1285 | gi\|1742730 | Protein AraJ precursor. [Escherichia coli] | 69 | 45 | |
| 229 | 8 | 7390 | 6698 | gi\|1162980 | ribulose-5-phosphate 3-epimer [Spinacia | 69 | 52 | |
| | | | | | oleracea] | | | |
| 238 | 27 | 25243 | 25695 | gi\|305005 | ORF_f104 [Escherichia coli] | 69 | 53 | |
| 253 | 3 | 1067 | 921 | gi\|1591278 | aspartokinase I [Methanococcus jannaschii] | 69 | 39 | |
| 260 | 4 | 2110 | 3105 | gi\|580841 | F1 [Bacillus subtilis] | 69 | 45 | |
| 268 | 3 | 2287 | 1910 | gi\|460026 | repressor protein [Streptococcus | 69 | 48 | |
| | | | | | pneumoniae] | | | |
| 269 | 7 | 4532 | 4083 | gi\|1303792 | YqeK [Bacillus subtilis] | 69 | 50 | |
| 271 | 15 | 11040 | 12236 | gi\|1303805 | YqeR [Bacillus subtilis] | 69 | 48 | |
| 271 | 16 | 12444 | 12809 | gi\|435490 | orf1 gene product [Lactococcus lactis] | 69 | 46 | |
| 281 | 3 | 1277 | 2068 | gi\|1303968 | YgjQ [Bacillus subtilis] | 69 | 50 | |
| 281 | 6 | 5004 | 5534 | gi\|1773151 | adenine phosphoribosyltransferase | 69 | 54 | |
| | | | | | [Escherichia coli] | | | |
| 292 | 24 | 19939 | 18398 | gi\|1652664 | glutamine-binding periplasmic protein | 69 | 45 | |
| | | | | | [Synechocystis sp.] | | | |
| 323 | 3 | 2708 | 4243 | gi\|179401 | beta-D-galactosidase precursor (EC | 69 | 56 | |
| | | | | | 3.2.1.23) [Homo sapiens] gi\|179423 beta- | | | |
| | | | | | galactosidase precursor (EC 3.2.1.23) | | | |
| | | | | | [Homo sapiens] pir\|A32688\|A32611 beta- | | | |
| | | | | | galactosidase (EC 3.2.1.23) precursor - | | | |
| | | | | | uman | | | |
| 330 | 2 | 1388 | 2353 | gi\|1303783 | YgeC [Bacillus subtilis] | 69 | 48 | |
| 332 | 1 | 2 | 223 | gi\|1653594 | hemolysin [Synechocystis sp.] | 69 | 50 | |
| 338 | 9 | 7035 | 7607 | gi\|467442 | stage V sporulation [Bacillus subtilis] | 69 | 55 | |
| 341 | 1 | 1 | 408 | gi\|1477741 | histidine periplasmic binding protein P29 | 69 | 50 | |
| | | | | | [Campylobacter jejuni] | | | |
| 368 | 2 | 972 | 598 | gi\|516826 | rat GCP360 [Rattus rattus] | 69 | 33 | |
| 375 | 4 | 3405 | 2599 | gi\|1215693 | putative orf; GT9_orf434 [Mycoplasma | 69 | 38 | |
| | | | | | pneumoniae] | | | |
| 386 | 1 | 2 | 166 | gi\|1549376 | putative protein [Synechococcus PCC7942] | 69 | 42 | |
| 396 | 4 | 1248 | 1715 | gi\|410132 | ORFX8 [Bacillus subtilis] | 69 | 50 | |
| 398 | 4 | 2763 | 2927 | gi\|466475 | putative phospho-beta-glucosidase | 69 | 55 | |
| | | | | | [Bacillus stearothermophilus] | | | |
| | | | | | pir\|D49898\|D49898 cellobiose | | | |
| | | | | | phosphotransferase system celC - acillus | | | |
| | | | | | stearothermophilus | | | |
| 421 | 5 | 2950 | 3471 | gi\|1574625 | H. influenzae predicted coding region | 69 | 45 | |
| | | | | | H11074 [Haemophilus influenzae] | | | |
| 423 | 4 | 2408 | 2893 | gnl\|PID\|e163522 | rnhB [Haemophilus influenzae] | 69 | 55 | |
| 436 | 3 | 1763 | 1521 | gi\|155032 | ORF B [Plasmid pEa34] | 69 | 37 | |
| 452 | 1 | 3 | 341 | gi\|1591139 | M. jannaschii predicted coding region | 69 | 52 | |
| | | | | | MJ0435 [Methanococcus jannaschii] | 69 | 52 | |
| 470 | 3 | 1816 | 2181 | gi\|437389 | transposase [Lactococcus lactis] | 69 | 56 | |
| 471 | 2 | 2003 | 813 | gi\|854233 | cymF gene product [Klebsiella oxytoca] | 69 | 49 | |
| 478 | 1 | 822 | 4 | gi\|142521 | deoxyribodipyrimidine photolyase [Bacillus | 69 | 63 | |
| | | | | | subtilis] gnl\|PID\|e255102 | | | |
| | | | | | deoxyribodipyrimidine photolyase [Bacillus | | | |
| | | | | | ubtilis] | | | |
| 490 | 4 | 1447 | 1289 | gi\|699379 | glvr-1 protein [Mycobacterium leprae] | 69 | 41 | |
| 518 | 2 | 213 | 605 | pir\|S00076\|RSBS12 | ribosomal protein L12 - Bacillus | 69 | 59 | |
| | | | | | stearotherrnophilus | | | |
| 536 | 4 | 1471 | 1653 | gi\|1146240 | ketopantoate hydroxymethyltransferase | 69 | 53 | |
| | | | | | [Bacillus subtilis] | | | |
| 539 | 5 | 3796 | 5091 | gi\|973231 | gamma-glutamyl phosphate reductase | 69 | 54 | |
| | | | | | [Lycopersicon esculentum] | | | |
| 566 | 1 | 1 | 231 | gi\|45741 | ORFE [Enterococcus faecalis] | 69 | 50 | |
| 579 | 5 | 2729 | 3595 | gi\|145887 | malonyl coenzyme A-acyl carrior protein | 69 | 49 | |
| | | | | | transacylase [Escherichia oli] | | | |
| 583 | 2 | 373 | 912 | gi\|1064791 | function umknown [Bacillus subtilis] | 69 | 55 | |
| 605 | 1 | 254 | 3 | pir\|S39743\|S39743 | hypothetical protein - Bacillus subtilis | 69 | 37 | |
| 630 | 2 | 1659 | 1231 | gi\|153672 | lactose repressor [Streptococcus mutans] | 69 | 47 | |
| 634 | 1 | 36 | 731 | gi\|1022725 | unknown [Staphylococcus haemolyticus] | 69 | 53 | |
| 662 | 1 | 486 | 73 | gi\|467431 | high level kasgamycin resistance [Bacillus | 69 | 55 | |
| | | | | | subtilis] sp\|P37468\|KSGA_BACSU | | | |
| | | | | | DIMETHYLADENOSINE TRANSFERASE (EC 2.1.1.-) | | | |
| | | | | | S-ADENOSYLMETHIONINE-6-N′, N′- | | | |
| | | | | | ADENOSYL(RRNA) DIMETHYLTRANSFERASE) 16S | | | |
| | | | | | RRNA DIMETHYLASE) (HIGH LEVEL KASUGAMYCIN | | | |
| | | | | | RESISTANCE PROTEIN SGA) (K | | | |
| 689 | 1 | 340 | 26 | gi\|1017817 | membrane spanning protein [Streptomyces | 69 | 41 | |
| | | | | | coelicolor] | | | |
| 756 | 2 | 300 | 500 | gi\|520596 | Mre2 protein [Saccharomyces cerevisiae] | 69 | 46 | |
| 792 | 2 | 855 | 460 | gi\|1303823 | YqfG [Bacillus subtilis] | 69 | 55 | |
| 916 | 1 | 4 | 789 | gnl\|PID\|e253114 | ornithine carbamoyltransferase [Pyrococcus | 69 | 57 | |
| | | | | | furiosus] | | | |
| 7 | 3 | 2609 | 3748 | gi\|1303836 | YgfO [Bacillus subtilis] | 68 | 50 | |
| 16 | 5 | 4165 | 4689 | gi\|142450 | ahrC protein [Bacillus subtilis] | 68 | 46 | |
| 17 | 16 | 12826 | 13071 | gi\|222681 | RNA polymerase [Tomato spotted wilt virus] | 68 | 50 | |
| 17 | 32 | 31402 | 31572 | gi\|1303984 | YgkG [Bacillus subtilis] | 68 | 44 | |
| 17 | 33 | 31509 | 32009 | gi\|1303984 | YgkG [Bacillus subtilis] | 68 | 50 | |
| 29 | 1 | 19 | 282 | gi\|1234787 | up-regulated by thyroid hormone in | 68 | 37 | |
| | | | | | tadpoles; expressed specifically in the | | | |
| | | | | | tail and only at metamorphosis; membrane | | | |
| | | | | | bound or extracellular protein; C-terminal | | | |
| | | | | | basic region [Xenopus laevis] | | | |
| 29 | 3 | 1087 | 1950 | gi\|407878 | leucine rich protein [Streptococcus | 68 | 45 | |
| | | | | | equisimilis] | | | |
| 45 | 1 | 204 | 959 | gi\|1039479 | ORFU [Lactococcus lactis] | 68 | 50 | |
| 47 | 7 | 8108 | 7527 | gi\|142853 | homologous to unidentified E. coli protein | 68 | 46 | |
| | | | | | [Bacillus subtilis] gi\|143161 maf | | | |
| | | | | | [Bacillus subtilis] | | | |
| 52 | 6 | 4304 | 5050 | gnl\|PID\|e124050 | alpha-acetolactate decarboxylase | 68 | 53 | |
| | | | | | [Lactococcus lactis] | | | |
| 58 | 5 | 5961 | 4807 | gi\|466365 | potential NAD-reducing hydrogenase subunit | 68 | 49 | |
| | | | | | [Desulfovibrio ructosovorans] | | | |
| 68 | 8 | 4036 | 4743 | gi\|1673727 | (AE000009) Mycoplasma pneumoniae, | 68 | 44 | |
| | | | | | glutamine transport ATP-binding protein; | | | |
| | | | | | similar to Swiss-Prot Accession Number | | | |
| | | | | | P10346, from E. coli [Mycoplasma | | | |
| | | | | | pneumoniae] | | | |
| 72 | 5 | 4441 | 3434 | gi\|1395209 | ribonucleotide reductase R2-2 small | 68 | 52 | |
| subunit [Mycobacterium tuberculosis] | | | | | | | | |
| 80 | 1 | 836 | 3 | gi\|474176 | regulator protein [Staphylococcus xylosus] | 68 | 48 | |
| 81 | 2 | 793 | 1359 | gi\|1064809 | homologous to sp:HTRA_ECOLI [Bacillus | 68 | 48 | |
| | | | | | subtilis] | | | |
| 85 | 9 | 6911 | 6711 | gi\|144893 | butyrate kinase [Clostridium | 68 | 55 | |
| | | | | | acetobutylicum] | | | |
| 89 | 8 | 7184 | 5970 | gi\|1469784 | putative cell division protein ftsW | 68 | 44 | |
| | | | | | [Enterococcus hirae] | | | |
| 91 | 3 | 828 | 1076 | gi\|726480 | L-glutarnine-D-fructose-6-phosphate | 68 | 53 | |
| | | | | | amidotransferase [Bacillus ubtilis] | | | |
| 103 | 1 | 1019 | 3 | gi\|143365 | phosphoribosyl aminoimidazole carboxylase | 68 | 50 | |
| | | | | | II (PUR-K; ttg start odon) [Bacillus | | | |
| | | | | | subtilis] | | | |
| 106 | 2 | 2441 | 1509 | gi\|146860 | delta-2-isopentenyl pyrophosphate | 68 | 47 | |
| | | | | | transferase [Escherichia coli] gi\|537012 | | | |
| | | | | | tRNA delta-2-isopentenylpyrophosphate | | | |
| | | | | | (IPP) transferase Escherichia coli] | | | |
| 112 | 1 | 558 | 100 | gnl\|PID\|e242290 | carbamate kinase [Clostridium perfringens] | 68 | 50 | |
| 116 | 3 | 2383 | 1496 | gi\|755601 | unknown [Bacillus subtilis] | 68 | 42 | |
| 119 | 3 | 2136 | 1201 | gi\|1171125 | thioredoxin reductase [Clostridium | 68 | 49 | |
| | | | | | litorale] | | | |
| 121 | 4 | 3697 | 4650 | gi\|790945 | aryl-alcohol dehydrogenase [Bacillus | 68 | 48 | |
| | | | | | subtilis] | | | |
| 123 | 26 | 24262 | 24801 | gi\|537235 | Kenn Rudd identifies as gpmB [Escherichia | 68 | 51 | |
| | | | | | coli] | | | |
| 123 | 27 | 24887 | 25888 | gi\|143150 | levR [Bacillus subtilis] | 68 | 51 | |
| 126 | 4 | 2773 | 1844 | gi\|551854 | ORF2 [Erwinia herbicola] | 68 | 54 | |
| 131 | 1 | 150 | 1058 | gi\|1387979 | 44% identity over 302 residues with | 68 | 44 | |
| | | | | | hypothetical protein from Synechocystis | | | |
| | | | | | sp, accession D64006_CD; expression | | | |
| | | | | | induced by environmental stress; some | | | |
| | | | | | similarity to glycosyl transferases; two | | | |
| | | | | | potential membrane-spanning helices | | | |
| | | | | | [Bacillus subtil | | | |
| 134 | 3 | 2154 | 1804 | sp\|P39213\|YI91_SHI | INSERTION ELEMENT IS911 HYPOTHETICAL 12.7 | 68 | 43 | |
| | | | | DY | KD PROTEIN. | | | |
| 138 | 19 | 12285 | 12656 | gi\|1438847 | homologue of hypothetical 17.6 kDa protein | 68 | 43 | |
| | | | | | in rplI-cpdB intergenic region of E. coli | | | |
| | | | | | [Bacillus subtilis] | | | |
| 151 | 2 | 2784 | 1654 | gi\|143365 | phosphoribosyl aminoimidazole carboxylase | 68 | 45 | |
| | | | | | II(PUR-K; ttg start odon) [Bacillus | | | |
| | | | | | subtilis] | | | |
| 164 | 23 | 24352 | 24119 | gi\|1573564 | hypothetical [Haemophilus influenzae] | 68 | 40 | |
| 166 | 2 | 970 | 1260 | gi\|151968 | nifS [Rhodobacter sphaeroides] | 68 | 41 | |
| 172 | 2 | 1320 | 2015 | gi\|1208965 | hypothetical 23.3 kd protein [Escherichia | 68 | 46 | |
| | | | | | coli] | | | |
| 175 | 1 | 900 | 451 | gi\|468207 | Submitter comments: A Mg2+ transporting P- | 68 | 47 | |
| | | | | | type ATPase highly omologous with mgtB | | | |
| | | | | | ATPase at 80 min on Salmonella chromosome. | | | |
| | | | | | ediates the influx of Mg2+ only. | | | |
| | | | | | Transcription regulated by xtracellular | | | |
| | | | | | Mg2+ [Salmonella typhimurium] | | | |
| 180 | 14 | 12551 | 14956 | gi\|565641 | FdrA protein [Escherichia coli] | 68 | 49 | |
| 186 | 1 | 3 | 686 | gi\|405804 | transposase [Streptococcus thermophilus] | 68 | 51 | |
| 200 | 1 | 239 | 3 | gi\|468016 | immunoglobulin heavy chain binding protein | 68 | 42 | |
| | | | | | [Giardia intestinalis] | | | |
| 201 | 4 | 4468 | 3686 | gi\|304013 | abcA [Aeromonas salmonicida] | 68 | 50 | |
| 204 | 10 | 6833 | 6468 | gi\|488430 | alcohol dehydrogenase 2 [Entamoeba | 68 | 51 | |
| | | | | | histolytica] | | | |
| 214 | 3 | 3360 | 2491 | gi\|928834 | integrase [Lactococcus lactis phage BK5-T] | 68 | 50 | |
| 229 | 9 | 8277 | 7375 | gi\|1574569 | hypothetical [Haemophilus influenzae] | 68 | 41 | |
| 229 | 14 | 14288 | 13740 | gnl\|P1D\|e290287 | polypeptide deformylase [Bacillus | 68 | 50 | |
| | | | | | subtilis] | | | |
| 230 | 5 | 4593 | 3532 | gi\|143002 | proton glutamate symport protein [Bacillus | 68 | 29 | |
| | | | | | caldotenax] pir\|S26246\|S526246 | | | |
| | | | | | glutamate/aspartate transport protein - | | | |
| | | | | | Bacillus aldotenax | | | |
| 244 | 1 | 1 | 891 | gi\|537080 | ribonucleoside triphosphate reductase | 68 | 54 | |
| | | | | | [Escherichia coli] pir\|A47331\|A47331 | | | |
| | | | | | oxygen-sensitive ribonucleoside- | | | |
| | | | | | triphosphate eductase (EC 1.17.4.-) - | | | |
| | | | | | Escherichia coli | | | |
| 244 | 5 | 4249 | 3551 | gi\|1773172 | hypothetical protein [Escherichia coli] | 68 | 46 | |
| 244 | 7 | 5670 | 5212 | gi\|467423 | unknown [Bacillus subtilis] | 68 | 43 | |
| 264 | 9 | 3925 | 3734 | gi\|914991 | Similar to hemoglobinase [Saccharomyces | 68 | 44 | |
| | | | | | cerevisiae] pir\|S59796\|S59796 hypothetical | | | |
| | | | | | protein D9798.2 - yeast Saccharomyces | | | |
| | | | | | cerevisiae) | | | |
| 271 | 7 | 3484 | 4686 | gi\|1469784 | putative cell division protein ftsW | 68 | 50 | |
| | | | | | [Enterococcus hirae] | | | |
| 271 | 11 | 6817 | 6548 | gi\|413948 | ipa-24d gene product [Bacillus subtilis] | 68 | 50 | |
| 288 | 3 | 1638 | 1333 | gi\|562039 | NADH dehydrogenase, subunit 2 | 68 | 50 | |
| | | | | | [Acanthamoeba castellanii] | | | |
| | | | | | pir\|S53835\|S53835 NADH dehydrogenase chain | | | |
| | | | | | 2 - Acanthamoeba astellanii mitochondrion | | | |
| | | | | | (SGC6) | | | |
| 295 | 6 | 3537 | 4472 | gi\|555668 | glycosylasparaginase precursor | 68 | 41 | |
| | | | | | [Flavobacterium meningosepticum] | | | |
| 296 | 2 | 3143 | 1950 | gi\|1742630 | Bicyclomycin resistance protein | 68 | 34 | |
| | | | | | (Sulfonamide resistance protein) | | | |
| | | | | | [Escherichia coli] | | | |
| 301 | 3 | 3271 | 1760 | gi\|413960 | ipa-36d galT gene product [Bacillus | 68 | 53 | |
| | | | | | subtilis] | | | |
| 315 | 3 | 2230 | 905 | gi\|1653498 | ABC transporter [Synechocystis sp.] | 68 | 47 | |
| 318 | 2 | 1285 | 854 | gi\|43940 | EIII-F Sor PTS [Klebsiella pneumoniae] | 68 | 39 | |
| 320 | 2 | 1178 | 621 | gi\|664842 | sister of P-glycoprotein [Sus scrofa | 68 | 46 | |
| | | | | | domestica] | | | |
| 331 | 2 | 342 | 566 | pir\|B48396\|B48396 | ribosomal protein L33 - Bacillus | 68 | 59 | |
| | | | | | stearothermophilus | | | |
| 336 | 1 | 1 | 663 | gi\|1006591 | cation-transporting ATPase PacL | 68 | 44 | |
| | | | | | [Synechocystis sp.] | | | |
| 338 | 6 | 4004 | 5035 | gi\|155276 | aldehyde dehydrogenase [Vibrio cholerae] | 68 | 51 | |
| 338 | 12 | 10404 | 11165 | gi\|467444 | transcription-repair coupling factor | 68 | 46 | |
| | | | | | [Bacillus subtilis] sp\|P37474\|MF_BACSU | | | |
| | | | | | TRANSCRIPTION-REPAIR COUPLING FACTOR | | | |
| | | | | | (TRCF). | | | |
| 341 | 3 | 743 | 1222 | gi\|1183886 | integral membrane protein [Bacillus | 68 | 45 | |
| | | | | | subtilis] | | | |
| 351 | 6 | 2992 | 2561 | gi\|580881 | ipa-73d gene product [Bacillus subtilis] | 68 | 53 | |
| 363 | 8 | 12517 | 9950 | gi\|1652980 | H(+)-transporting ATPase [Synechocystis | 68 | 46 | |
| | | | | | sp.] | | | |
| 368 | 3 | 1269 | 1736 | gnl\|PID\|e209005 | homologous to ORF2 in nrdEF operons of | 68 | 37 | |
| | | | | | E.coli and S.typhimurium [Lactococcus | | | |
| | | | | | lactis] | | | |
| 386 | 11 | 6564 | 6115 | gi\|765072 | ORF3 [Staphylococcus aureus] | 68 | 46 | |
| 395 | 3 | 935 | 729 | gi\|5521 | ORF 3 (AA 1-90) [Bacteriophaqe phi-105] | 68 | 34 | |
| 399 | 8 | 6073 | 6519 | gi\|153584 | biotin carboxyl carrier protein | 68 | 53 | |
| | | | | | [Streptococcus mutans] | | | |
| | | | | | sp\|P29337\|BCCP_STRMU BIOTIN CARBOXYL | | | |
| | | | | | CARRIER PROTEIN (BCCP). | | | |
| 408 | 3 | 2289 | 1336 | gi\|41572 | GlnP (AA 1-219) [Escherichia coli] | 68 | 40 | |
| 420 | 1 | 559 | 2 | gi\|1592142 | ABC transporter, probable ATP-binding | 68 | 51 | |
| | | | | | subunit [Methanococcus jannaschii] | | | |
| 423 | 2 | 254 | 1294 | gi\|1773109 | similar to S. typhimurium apbA | 68 | 47 | |
| | | | | | [Escherichia coli] | | | |
| 423 | 3 | 1465 | 2421 | gi\|1653032 | hypothetical protein [Synechocystis sp.] | 68 | 40 | |
| 428 | 1 | 859 | 2 | gi\|1652454 | hypothetical protein [Synechocystis sp.] | 68 | 48 | |
| 432 | 7 | 4626 | 3901 | gi\|1573285 | hypothetical [Haemophilus influenzae] | 68 | 55 | |
| 434 | 1 | 90 | 1889 | gi\|1542975 | AbcB [Thermoanaerobacterium | 68 | 50 | |
| | | | | | thermosulfurigenes] | | | |
| 441 | 5 | 4674 | 5156 | gi\|467437 | unknown [Bacillus subtilis] | 68 | 48 | |
| 455 | 4 | 3835 | 4080 | gi\|19815 | luminal binding protein (BiP) [Nicotiana | 68 | 40 | |
| | | | | | tabacum] | | | |
| 530 | 2 | 394 | 546 | gi\|763326 | unknown [Saccharomnyces cerevisiae] | 68 | 42 | |
| 531 | 2 | 810 | 622 | gi\|1146183 | putative [Bacillus subtilis] | 68 | 51 | |
| 537 | 3 | 1353 | 1192 | gi\|929968 | ORFA; similar to B. anthracis WeyAR | 68 | 56 | |
| | | | | | element ORFA; putative ransposase | | | |
| | | | | | [Bacillus anthracis] | | | |
| 539 | 3 | 2725 | 2231 | gi\|1353537 | dUTPase [Bacteriophage rlt] | 68 | 53 | |
| 569 | 1 | 3 | 446 | gi\|146544 | 18 kD protein [Eschenichia coli] | 68 | 47 | |
| 591 | 2 | 656 | 174 | gi\|1039479 | ORFU [Lactococcus lactis] | 68 | 42 | |
| 652 | 2 | 739 | 1032 | gi\|1303715 | YrkP [Bacillus subtilis] | 68 | 50 | |
| 671 | 2 | 436 | 1617 | gi\|413959 | ipa-35d galK gene product [Bacillus | 68 | 50 | |
| | | | | | subtilis] | | | |
| 684 | 1 | 466 | 2 | gnl\|PID\|e248400 | orfRM1 gene product [Bacillus subtilis] | 68 | 40 | |
| 693 | 1 | 2 | 787 | gi\|405804 | transposase [Streptococcus thermophilus] | 68 | 46 | |
| 700 | 2 | 772 | 596 | gi\|153801 | enzyme scr-II [Streptococcus mutans] | 68 | 50 | |
| 735 | 1 | 118 | 609 | gi\|969027 | gamma-aminobutyrate permease [Bacillus | 68 | 40 | |
| | | | | | subtilis] sp\|P46349\|GABP_BACSU GABA | | | |
| | | | | | PERMEASE (4-AMINO BUTYRATE TRANSPORT | | | |
| | | | | | ARRIER) (GAMA-AMINOBUTYRATE PERMEASE). | | | |
| 750 | 1 | 2 | 529 | gi\|893358 | PgsA [Bacillus subtilis] | 68 | 54 | |
| 762 | 2 | 1588 | 950 | gi\|1146240 | ketopantoate hydroxymethyltransferase | 68 | 49 | |
| | | | | | [Bacillus subtilis] | | | |
| 790 | 1 | 407 | 3 | gi\|142224 | attachment protein ChvA (ttg strart codon) | 68 | 55 | |
| | | | | | [Agrobacterium umefaciens] | | | |
| 882 | 1 | 3 | 278 | gi\|57572 | glyceraldehyde-3-phosphate dehydrogenase | 68 | 48 | |
| | | | | | (NADP+) (phosphorylating) attus rattus] | | | |
| 950 | 1 | 140 | 568 | gi\|882736 | ORF_f278 [Escherichia coli] | 68 | 53 | |
| 969 | 2 | 554 | 339 | gi\|1118031 | similar to neural cell adhesion molecules | 68 | 47 | |
| | | | | | and neuroglians in their IG-like C2-type | | | |
| | | | | | domains [Caenorhabditis elegans] | | | |
| 970 | 1 | 297 | 73 | gi\|474404 | cyclophilin [Tolypocladium inflatum] | 68 | 40 | |
| 1 | 1 | 1103 | 3 | gi\|48790 | ORF 3 [Pseudomonas putAda] | 67 | 50 | |
| 29 | 10 | 7156 | 6614 | sp\|P36672\|PTTB_ECO | PTS SYSTEM, TREHALOSE-SPECIFIC IIBC | 67 | 52 | |
| | | | | LI | COMPONENT (EIIBC-TRE) (TREHALOSE- PERMEASE | | | |
| | | | | | IIBC COMPONENT) (PHOSPHOTRANSFERASE ENZYME | | | |
| | | | | | II, BC COMPONENT) (EC 2.7.1.69) (EII-TRE). | | | |
| 48 | 8 | 8035 | 9141 | gi\|975627 | N-acylamino acid racemase [Amycolatopsis | 67 | 48 | |
| | | | | | sp.] | | | |
| 55 | 12 | 6621 | 7439 | gi\|391610 | farnesyl diphosphate synthase [Bacillus | 67 | 47 | |
| | | | | | stearothermophilus] pir\|JX0257\|JX0257 | | | |
| | | | | | geranyltranstransferase (BC 2.5.1.10) - | | | |
| | | | | | Bacillus tearothermophilus | | | |
| 57 | 13 | 13972 | 16401 | gnl\|PID\|e255138 | phenylalanyl-tRNA synthetase beta subunit | 67 | 47 | |
| | | | | | [Bacillus subtilis] | | | |
| 63 | 4 | 1917 | 2729 | gi\|1321629 | MIP related protein of E. coli | 67 | 47 | |
| | | | | | [Escherichia coli] | | | |
| 68 | 12 | 8600 | 8923 | gi\|793910 | surface antigen [Homo sapiens] | 67 | 43 | |
| 72 | 7 | 7138 | 6740 | gnl\|PID\|e209005 | homologous to ORF2 in nrdEF operons of | 67 | 39 | |
| | | | | | E.coli and S.typhimurium [Lactococcus | | | |
| | | | | | lactis] | | | |
| 72 | 10 | 8309 | 9433 | gi\|1199515 | ferrous iron transport protein B | 67 | 41 | |
| | | | | | [Escherichia coli] | | | |
| 85 | 5 | 5315 | 4296 | gi\|142611 | branched chain alpha-keto acid | 67 | 52 | |
| | | | | | dehydrogenase E1-alpha [Bacillus ubtilis] | | | |
| 101 | 5 | 4149 | 3100 | gi\|1109686 | ProX [Bacillus subtilis] | 67 | 48 | |
| 110 | 4 | 2335 | 1292 | gi\|1066343 | mu-crystallin [Homo sapiens] | 67 | 48 | |
| 114 | 12 | 12936 | 13520 | gi\|146218 | serine hydroxymethyltransferase | 67 | 50 | |
| | | | | | [Escherichia coli] | | | |
| 115 | 5 | 3137 | 2010 | gi\|1256150 | YbaR [Bacillus subtilis] | 67 | 47 | |
| 115 | 6 | 3199 | 2792 | gi\|1652593 | hypothetical protein [Synechocystis sp.] | 67 | 45 | |
| 123 | 25 | 22739 | 24208 | gi\|148711 | 6-aminohexanoate-cyclic-dimer hydrolase | 67 | 50 | |
| | | | | | [Flavobacterium sp.] gi\|488343 6- | | | |
| | | | | | aminohexanoate-cyclic-dimer hydrolase | | | |
| | | | | | [Flavobacterium p.] | | | |
| 124 | 6 | 5139 | 4267 | gi\|1016770 | prolipoprotein diacyiglyceryl transferase | 67 | 50 | |
| | | | | | [Staphylococcus aureus] | | | |
| 125 | 2 | 1306 | 221 | gi\|853743 | L-alanoyl-D-glutamate peptidase | 67 | 50 | |
| | | | | | [Bacteriophage A118] | | | |
| 128 | 36 | 29462 | 28737 | gi\|142940 | ftsA [Bacillus subtilis] | 67 | 46 | |
| 138 | 27 | 17602 | 18183 | gi\|1256639 | putative [Bacillus subtilis] | 67 | 50 | |
| 138 | 31 | 21578 | 20097 | gi\|143245 | Na+/H+ antiporter [Bacillus firmus] | 67 | 42 | |
| 138 | 33 | 25165 | 23249 | gi\|1498811 | M. jannaschii predicted coding region | 67 | 45 | |
| | | | | | MJ0050 [Methanococcus jannaschii] | | | |
| 138 | 36 | 28690 | 27362 | gnl\|PID\|e269549 | Unknown [Bacillus subtilis] | 67 | 47 | |
| 144 | 4 | 3271 | 3717 | gi\|1753229 | PKCI [Borrelia burgdorferi] | 67 | 52 | |
| 145 | 3 | 1435 | 2511 | gi\|1573615 | ATP-binding protein (abc) [Haemophilus | 67 | 47 | |
| | | | | | influenzae] | | | |
| 146 | 5 | 4657 | 2804 | gi\|1045034 | beta-galactosidase [Xanthomonas campestris | 67 | 51 | |
| | | | | | pv. manihotis] | | | |
| 149 | 3 | 1978 | 1367 | gi\|806536 | membrane protein [Bacillus | 67 | 51 | |
| | | | | | acidopullulyticus] | | | |
| 156 | 1 | 3 | 365 | gnl\|PID\|e265539 | ClpB-homologue [Thermus aquaticus | 67 | 42 | |
| | | | | | thermophilus] | | | |
| 158 | 15 | 14863 | 13766 gi\|1573487 | rbs repressor (rbsR) [Haemophilus | 67 | 40 | | |
| | | | | | influenzae] | | | |
| 158 | 17 16483 | 15959 | gi\|677850 | hypothetical protein [Staphylococcus | 67 | 51 | | |
| | | | | | aureus] | | | |
| 159 | 7 | 6872 | 6006 | gi\|1303949 | YqiX [Bacillus subtilis] | 67 | 41 | |
| 159 | 9 | 8103 | 7498 | gi\|1303950 | YqiY [Bacillus subtilis] | 67 | 41 | |
| 165 | 11 | 9846 | 9004 | gi\|606079 | ORF_o267 [Escherichia coli] | 67 | 36 | |
| 169 | 2 | 2151 | 3047 | gi\|42371 | pyruvate formate-lyase activating enzyme | 67 | 44 | |
| | | | | | (AA 1-246) [Escherichia li] | | | |
| 179 | 13 | 13648 | 14451 | gnl\|PID\|e257631 | methyltransferase [Lactococcus lactis] | 67 | 45 | |
| 180 | 28 | 28656 | 29801 | gi\|666005 | hypothetical protein [Bacillus subtilis] | 67 | 48 | |
| 194 | 6 | 2774 | 4231 | gi\|143245 | Na+/H+ antiporter [Bacillus firmus] | 67 | 41 | |
| 194 | 10 | 6472 | 8259 | gi\|622991 | mannitol transport protein [Bacillus | 67 | 50 | |
| | | | | | stearothermophilus] sp\|P50852\|PTMB_BACST | | | |
| | | | | | PTS SYSTEM, MANNITOL-SPECIFIC IIBC | | | |
| | | | | | COMPONENT EIIBC-MTL) (MANNITOL- PERMEASE | | | |
| | | | | | IIBC COMPONENT) (PHOSPHOTRANSFERASE NZYME | | | |
| | | | | | II, BC COMPONENT) (EC 2.7.1.69) (EII-MTL). | | | |
| 204 | 5 | 1924 | 3006 | gi\|1235684 | mevalonate pyrophosphate decarboxylase | 67 | 50 | |
| | | | | | [Saccharomyces cerevisiae] | | | |
| 214 | 1 | 42 | 1196 | gi\|606013 | CG Site No. 829 [Escherichia coli] | 67 | 36 | |
| 219 | 2 | 524 | 850 | gnl\|PID\|e257628 | ORF [Lactococcus lactis] | 67 | 42 | |
| 223 | 15 | 13640 | 14407 | gi\|496520 | orf iota [Streptococcus pyogenes] | 67 | 54 | |
| 227 | 3 | 1011 | 1892 | gi\|1070013 | protein-dependent [Bacillus subtilis] | 67 | 37 | |
| 233 | 12 | 9340 | 8339 | gi\|507880 | xanthine dehydrogenase [Gallus gallus] | 67 | 50 | |
| 238 | 10 | 7951 | 9183 | gi\|1653948 | hypothetical protein [Synechocystis sp.] | 67 | 45 | |
| 246 | 3 | 783 | 1430 | gnl\|PID\|e233869 | hypothetical protein [Bacillus subtilis] | 67 | 47 | |
| 256 | 2 | 570 | 1601 | gi\|709992 | hypothetical protein [Bacillus subtilisl | 67 | 36 | |
| 266 | 2 | 1266 | 835 | gi\|963038 | ArpU [Enterococcus hirae] | 67 | 42 | |
| 285 | 1 | 3 | 809 | gi\|40014 | pot. ORF 446 (aa 1-446) [Bacillus | 67 | 53 | |
| | | | | | subtilis] | | | |
| 288 | 10 | 6838 | 5801 | gi\|1651806 | hypothetical protein [Synechocystis sp.] | 67 | 45 | |
| 301 | 10 | 8822 | 8562 | gi\|1303864 | YqgQ [Bacillus subtilis] | 67 | 43 | |
| 312 | 5 | 2377 | 2595 | gi\|709991 | hypothetical protein [Bacillus subtilis] | 67 | 52 | |
| 353 | 1 | 3 | 1472 | gi\|151259 | HMG-CoA reductase (EC 1.1.1.88) | 67 | 48 | |
| | | | | | [Pseudomonas mevalonii] pir\|A44756\|A44756 | | | |
| | | | | | hydroxymethylglutaryl-CoA reductase (EC | | | |
| | | | | | 1.1.1.88) Pseudomonas sp. | | | |
| 359 | 2 | 984 | 439 | gi\|1773190 | similar to E. coli yhaE [Escherichia coli] | 67 | 45 | |
| 359 | 3 | 2244 | 982 | gi\|1001478 | hypothetical protein [Synechocystis sp.] | 67 | 30 | |
| 364 | 8 | 8469 | 7816 | gi\|496943 | ORF [Saccharomyces cerevisiae] | 67 | 50 | |
| 386 | 12 | 6625 | 7833 | gnl\|PID\|e254644 | membrane protein [Streptococcus | 67 | 36 | |
| | | | | | pneumoniae] | | | |
| 394 | 2 | 497 | 2635 | gnl\|PID\|e25593 | hypothetical protein [Bacillus subtilis] | 67 | 45 | |
| 399 | 6 | 5410 | 3971 | gi\|665994 | hypothetical protein [Bacillus subtilis] | 67 | 45 | |
| 414 | 1 | 1 | 1227 | gi\|1621027 | high affinity potassium transporter | 67 | 40 | |
| | | | | | [Debaryomyces occidentalis] | | | |
| 453 | 2 | 618 | 391 | gi\|537189 | ORF_f132 [Escherichia coli] | 67 | 45 | |
| 458 | 1 | 825 | 226 | gnl\|PID\|e189917 | ORF 28.5 [Escherichia coli] | 67 | 45 | |
| 460 | 2 | 644 | 1387 | gi\|1502421 | 3-ketoacyl-acyl carrier protein reductase | 67 | 48 | |
| | | | | | [Bacillus subtilis] | | | |
| 460 | 4 | 2622 | 3131 | gi\|1399830 | biotin carboxyl carrier protein | 67 | 53 | |
| | | | | | [Synechococcus PCC7942] | | | |
| 474 | 1 | 1456 | 77 | gi\|495277 | histidine kinase [Streptococcus | 67 | 54 | |
| | | | | | pneumoniae] | | | |
| 488 | 6 | 3892 | 3032 | gi\|437389 | transposase [Lactococcus lactis] | 67 | 47 | |
| 490 | 1 | 460 | 2 | gi\|1742830 | ORF_ID:o326#2; similar to [SwissProt | 67 | 43 | |
| | | | | | Accession Number P37794] [Eseherichia | | | |
| | | | | | coli] | | | |
| 582 | 1 | 2 | 787 | gi\|1408485 | yxdM gene product [Bacillus subtilis] | 67 | 38 | |
| 629 | 2 | 1280 | 915 | gi\|1006620 | ABC transporter [Synechocystis sp.] | 67 | 50 | |
| 633 | 2 | 941 | 390 | gnl\|PID\|e221400 | tex gene product [Bordetella pertussis] | 67 | 54 | |
| 655 | 1 | 47 | 313 | gi\|147403 | mannose permease subunit Il-P-Man | 67 | 48 | |
| | | | | | [Escherichia coli] | | | |
| 671 | 3 | 1630 | 2415 | sp\|P13226\|GALE_STR | UDP-GLUCOSE 4-EPIMERASE (EC 5.1.3.2) | 67 | 52 | |
| | | | | LI | (GALACTOWALDENASE). | | | |
| 682 | 2 | 1428 | 595 | gi\|147404 | mannose permease subunit II-M-Man | 67 | 42 | |
| | | | | | [Escherichia coli] | | | |
| 704 | 3 | 977 | 411 | gi\|467428 | unknown [Bacillus subtilis] | 67 | 45 | |
| 711 | 1 | 590 | 168 | gi\|471236 | orf3 [Haemophilus influenzae] | 67 | 37 | |
| 784 | 1 | 253 | 2 | gnl\|PID\|e236287 | site-specific DNA-methyltransferase | 67 | 44 | |
| | | | | | [Bacillus_stearothermophilus] | | | |
| 907 | 1 | 209 | 3 | gi\|5119 | topoisomerase I [Schizosaccharomyces | 67 | 42 | |
| | | | | | pombe] | | | |
| 908 | 1 | 275 | 96 | gi\|1591045 | hypothetical protein (SP:P31466) | 67 | 46 | |
| | | | | | [Methanococcus jannaschii] | | | |
| 960 | 1 | 499 | 98 | gi\|405804 | transposase [Streptococcus thermophilus] | 67 | 50 | |
| 963 | 1 | 259 | 2 | pir\|S34632\|S34632 | dnaJ protein homolog - human | 67 | 54 | |
| 964 | 1 | 164 | 628 | bbs\|173803 | CD4+ T cell-stimulating antigen [Listeria | 67 | 49 | |
| | | | | | monocytogenes, 85E0-1167, Peptide Partial, | | | |
| | | | | | 268 aa] [Listeria monocytogenes] | | | |
| 5 | 4 | 1438 | 2403 | gi\|1303810 | YgeT [Bacillus subtilis] | 66 | 50 | |
| 7 | 1 | 24 | 1727 | gi\|145220 | alanyl-tRNA synthetase [Escherichia coli] | 66 | 50 | |
| 7 | 2 | 1858 | 2646 | gi\|687599 | orfA1; transposon insertion into orfA1 | 66 | 58 | |
| | | | | | impairs growth and virulence f L. | | | |
| | | | | | monocytogenes [Listeria monocytogenes] | | | |
| 8 | 1 | 3 | 707 | gi\|1303830 | YgfL [Bacillus subtilis] | 66 | 45 | |
| 9 | 1 | 182 | 1051 | gi\|467399 | IMP dehydrogenase [Bacillus subtilis] | 66 | 51 | |
| 17 | 11 | 8383 | 8598 | gi\|457336 | Pv200 [Plasmodium vivax] | 66 | 42 | |
| 18 | 14 | 5903 | 6136 | gi\|294706 | trfA [Plasmid RK2] | 66 | 50 | |
| 23 | 12 | 5951 | 6895 | gi\|1652472 | ethylene response sensor protein | 66 | 51 | |
| | | | | | [Synechocystis sp.] | | | |
| 23 | 17 | 11198 | 11881 | gi\|466517 | pduB [Salmonella typhimurium] | 66 | 44 | |
| 23 | 19 | 12395 | 13501 | gi\|145206 | pduB [Salmonella typhimurium] | 66 | 47 | |
| 34 | 5 | 5987 | 6232 | gi\|397360 | yNucR endo-exonuclease [Saccharomyces | 66 | 46 | |
| | | | | | cerevisiae] | | | |
| 43 | 2 | 782 | 1018 | gi\|513417 | non-structural polyprotein of pSP6-SFV4 | 66 | 46 | |
| | | | | | [unidentified] | | | |
| 43 | 5 | 3757 | 2324 | gnl\|PID\|e154145 | penicillin binding protein 4 | 66 | 44 | |
| | | | | | [Staphylococcus_aureus] | | | |
| 56 | 4 | 2351 | 1662 | gi\|49272 | Asparaginase [Bacillus licheniformis] | 66 | 44 | |
| 57 | 2 | 950 | 1735 | gi\|1657505 | hypothetical protein [Escherichia coli] | 66 | 46 | |
| 57 | 4 | 3117 | 3932 | gi\|1657507 | hypothetical protein [Escherichia coli] | 66 | 41 | |
| 57 | 8 | 12269 | 12646 | gi\|1622733 | orf108; unknown function [Butyrivibrio | 66 | 44 | |
| | | | | | fibrisolvens] | | | |
| 62 | 2 | 547 | 1302 | gi\|413967 | ipa-43d gene product [Bacillus subtilis] | 66 | 50 | |
| 62 | 5 | 2633 | 1905 | gi\|475110 | fructokinase [Pediococcus pentosace] | 66 | 51 | |
| 74 | 7 | 4661 | 4086 | gi\|467484 | unknown [Bacillus subtilis] | 66 | 47 | |
| 81 | 18 | 13878 | 13717 | gi\|146724 | enzyme III-Man function protein (manX | 66 | 35 | |
| | | | | | (ptsL)) [Escherichia coli] gi\|41976 manX | | | |
| | | | | | gene product (AA 1-315) [Escherichia coli] | | | |
| 94 | 17 | 20780 | 21253 | gi\|142955 | glucose dehydrogenase (EC 1.1.1.47) | 66 | 47 | |
| | | | | | [Bacillus subtilis] pir\|S36090\|S36090 | | | |
| | | | | | glucose 1-dehydrogenase (EC 1.1.1.47) - | | | |
| | | | | | Bacillus ubtilis | | | |
| 98 | 15 | 15165 | 14338 | gi\|147327 | transport protein [Escherichia coli] | 66 | 34 | |
| 105 | 3 | 1726 | 3183 | gnl\|PID\|e205173 | orf1 gene product [Lactobacillus | 66 | 45 | |
| | | | | | helveticus] | | | |
| 110 | 17 | 15811 | 14804 | gi\|887824 | ORF_o310 [Escherichia coli] | 66 | 52 | |
| 112 | 2 | 712 | 443 | gnl\|PID\|e242290 | carbainate kinase [Clostridium perfringens] | 66 | 51 | |
| 123 | 1 | 1 | 540 | gi\|1573538 | H. influenzae predicted coding region | 66 | 39 | |
| | | | | | H10552 [Haemophilus influenzae] | | | |
| 123 | 33 | 30312 | 31460 | gi\|1498930 | M. jannaschii predicted coding region | 66 | 48 | |
| | | | | | MJ0158 [Methanococcus jannaschii] | | | |
| 125 | 8 | 4914 | 4474 | gi\|1736749 | Exopolysaccharide production protein PSS. | 66 | 54 | |
| | | | | | [Escherichia_coli] | | | |
| 128 | 25 | 18201 | 18878 | gnl\|PID\|e255543 | putative iron dependant repressor | 66 | 48 | |
| | | | | | [Staphylococcus epidermidis] | | | |
| 131 | 3 | 2311 | 3213 | gi\|38969 | lacF gene product [Agrobacterium | 66 | 37 | |
| | | | | | radiobacter] | | | |
| 131 | 5 | 3588 | 3394 | gi\|1303823 | YqfG [Bacillus subtilis] | 66 | 29 | |
| 135 | 1 | 1214 | 45 | gi\|1498930 | M. jannaschii predicted coding region | 66 | 48 | |
| | | | | | MJ0158 [Methanococcus jannaschii] | | | |
| 135 | 10 | 7764 | 7405 | gi\|530825 | OVT1 [Onchocerca volvulus] | 66 | 47 | |
| 144 | 13 | 12859 | 10739 | pir\|A40614\|A40614 | penicillin-binding protein pbpF - Bacillus | 66 | 47 | |
| | | | | | subtilis | | | |
| 145 | 5 | 3224 | 4063 | gi\|349531 | lipoprotein [Pasteurella haemolytica] | 66 | 45 | |
| 146 | 2 | 1497 | 619 | gi\|147404 | mannose permease subunit II-M-Man | 66 | 38 | |
| | | | | | [Escherichia coli] | | | |
| 149 | 2 | 1097 | 1282 | gi\|1762962 | FemA [Staphylococcus simulans] | 66 | 38 | |
| 150 | 3 | 1443 | 2417 | gnl\|PID\|e185374 | ceuE gene product [Campylobacter coli] | 66 | 46 | |
| 150 | 8 | 6487 | 6903 | gi\|1377842 | unknown [Bacillus subtilis] | 66 | 43 | |
| 164 | 20 | 21846 | 22646 | gi\|1279769 | FdhC [Methanobacterium thermoformicicum] | 66 | 57 | |
| 164 | 25 | 24555 | 25688 | pir\|A43577\|A43577 | regulatory protein pfoR - Clostridium | 66 | 47 | |
| | | | | | perfringens | | | |
| 178 | 1 | 383 | 3 | gi\|763052 | integrase [Bacteriophage T270] | 66 | 47 | |
| 195 | 19 | 8698 | 8516 | bbs\|169008 | homeobox gene [Drosophila sp.] | 66 | 55 | |
| 207 | 1 | 166 | 1554 | gi\|619724 | MgtE [Bacillus firmus] | 66 | 39 | |
| 207 | 3 | 2312 | 2010 | gi\|1204258 | soluble protein [Escherichia coli] | 66 | 44 | |
| 211 | 3 | 1523 | 1729 | gi\|289932 | MHC class II beta chain [Cyphotilapia | 66 | 66 | |
| | | | | | frontosa] | | | |
| 213 | 3 | 1811 | 2308 | gi\|153045 | prolipoprotein signal peptidase | 66 | 40 | |
| | | | | | [Staphylococcus aureus] pir\|S20433\|S20433 | | | |
| | | | | | lsp protein - Staphylococcus aureus | | | |
| | | | | | sp\|P31024\|LSPA_STAAU LIPOPROTEIN SIGNAL | | | |
| | | | | | PEPTIDASE (EC 3.4.23.36) PROLIPOPROTEIN | | | |
| | | | | | SIGNAL PEPTIDASE) (SIGNAL PEPTIDASE II) | | | |
| | | | | | (SPASE II). | | | |
| 221 | 7 | 2524 | 3468 | gi\|1353527 | ORF10 [Bacteriophage rlt] | 66 | 44 | |
| 222 | 13 | 8272 | 8988 | gi\|466719 | No definition line found [Eschenichia | 66 | 48 | |
| | | | | | coli] | | | |
| 223 | 18 | 15210 | 15971 | gi\|496520 | orf iota [Streptococcus pyogenes] | 66 | 57 | |
| 232 | 5 | 3494 | 2715 | gi\|142706 | comG1 gene product [Bacillus subtilis] | 66 | 41 | |
| 235 | 3 | 1774 | 734 | gi\|580897 | OppB gene product [Bacillus subtilis] | 66 | 47 | |
| 244 | 2 | 906 | 1520 | gi\|15354 | ORF 55.9 [Bacteriophage T4] | 66 | 46 | |
| 259 | 3 | 2355 | 1867 | gi\|56312 | Gephyrin [Rattus norvegicus] | 66 | 55 | |
| 271 | 1 | 1 | 675 | gi\|1574748 | tRNA pseudouridine 55 synthase (truB) | 66 | 53 | |
| | | | | | [Haemophilus influenzae] | | | |
| 277 | 1 | 1 | 927 | gi\|1303799 | YqeN [Bacillus subtilis] | 66 | 45 | |
| 291 | 5 | 4587 | 3547 | gnl\|PID\|e257609 | sugar-binding transport protein | 66 | 46 | |
| | | | | | [Anaerocellum thermophilum] | | | |
| 292 | 25 | 20451 | 19912 | gi\|1649035 | high-affinity periplasmic glutamine | 66 | 50 | |
| | | | | | binding protein [Salmonella typhimurium] | | | |
| 300 | 1 | 2302 | 77 | gi\|289262 | comE ORF3 [Bacillus subtilis] | 66 | 46 | |
| 301 | 4 | 4290 | 3265 | sp\|P13226\|GALE_STR | UDP-GLUCOSE 4-EPIMERASE (EC 5.1.3.2) | 66 | 51 | |
| | | | | LI | (GALACTOWALDENASE). | | | |
| 301 | 5 | 4516 | 4689 | gnl\|PID\|e212164 | PSII, protein N [Odontella sinensis] | 66 | 58 | |
| 314 | 1 | 360 | 4 | gi\|467452 | unknown [Bacillus subtilis] | 66 | 43 | |
| 15 | 4 | 2559 | 2209 | gi\|1653498 | ABC transporter [Synechocystis sp.] | 66 | 44 | |
| 320 | 3 | 2406 | 1081 | gnl\|PID\|e250352 | unknown [Mycobacterium tuberculosis] | 66 | 35 | |
| 332 | 2 | 157 | 921 | gi\|1303875 | YghB [Bacillus subtilis] | 66 | 44 | |
| 334 | 2 | 1001 | 3076 | gi\|1651660 | DNA ligase [Synechocystis sp.] | 66 | 48 | |
| 338 | 1 | 2 | 616 | gi\|845686 | ORF-27 [Staphylococcus aureus] | 66 | 54 | |
| 338 | 7 | 5011 | 5496 | gi\|912476 | No definition line found [Escherichia | 66 | 48 | |
| | | | | | coli] | | | |
| 341 | 5 | 1935 | 3107 | gi\|142538 | aspartate aminotransferase [Bacillus sp.] | 66 | 44 | |
| 343 | 3 | 2548 | 2045 | gnl\|PID\|e289147 | similar to single strand binding protein | 66 | 44 | |
| | | | | | [Bacillus subtilis] | | | |
| 345 | 20 | 22093 | 22461 | gi\|1657795 | dihydroneopterin aldolase | 66 | 45 | |
| | | | | | [Methylobacterium extorquens] | | | |
| 353 | 3 | 2621 | 2379 | gnl\|PID\|e257628 | ORF [Lactococcus lactis] | 66 | 52 | |
| 365 | 4 | 5117 | 4779 | gi\|1742868 | Mutator MutT protein (7,8-dihydro-8- | 66 | 54 | |
| | | | | | oxoguanine-triphosphatase) (8-oxo-dgtpase) | | | |
| | | | | | (EC 3.6.1.-) (DGTP pyrophosphohydrolase). | | | |
| | | | | | [Escherichia coli] | | | |
| 376 | 1 | 3 | 1076 | gi\|1778517 | glycerol dehydrogenase homolog | 66 | 45 | |
| | | | | | [Escherichia coli] | | | |
| 394 | 7 | 5980 | 5648 | gi\|486358 | ORF YKL202w [Saccharomyces cerevisiae] | 66 | 38 | |
| 421 | 4 | 1469 | 2539 | gi\|606375 | ORF_f345 [Escherichia coli] | 66 | 48 | |
| 475 | 6 | 3978 | 3763 | gi\|532547 | ORF14 [Enterococcus faecalis] | 66 | 48 | |
| 491 | 8 | 7710 | 7081 | gi\|1000453 | TreR [Bacillus subtilis] | 66 | 49 | |
| 526 | 1 | 392 | 3 | gi\|1750125 | xylulose kinase [Bacillus subtilis] | 66 | 49 | |
| 552 | 6 | 6147 | 5917 | gi\|1432152 | PTS antiterminator [Klebsiella oxytoca] | 66 | 37 | |
| 571 | 2 | 560 | 1153 | gi\|1773132 | multidrug resistance-like ATP-binding | 66 | 38 | |
| | | | | | protein Mdl [Esoherichia coli] | | | |
| 575 | 3 | 1075 | 539 | gi\|1651722 | guanylate kinase [Synechocystis sp.] | 66 | 48 | |
| 608 | 2 | 631 | 113 | gi\|1213334 | OrfX; hypothetical 22.5 KD protein | 66 | 41 | |
| | | | | | downstream of type IV prepilin leader | | | |
| | | | | | peptidase gene; Method: conceptual | | | |
| | | | | | translation supplied by author [Vibrio | | | |
| | | | | | vulnificus] | | | |
| 640 | 1 | 877 | 2 | sp\|P50487\|YCPX_CLO | HYPOTHETICAL PROTEIN IN CPE 5′REGION | 66 | 36 | |
| | | | | PE | (FRAGMENT) | | | |
| 734 | 1 | 2 | 343 | gi\|1653602 | hypothetical protein [Synechocystis sp.] | 66 | 43 | |
| 802 | 1 | 2 | 292 | gnl\|PID\|e280516 | voltage-gated sodium channel [Mus | 66 | 58 | |
| | | | | | musculus] | | | |
| 812 | 2 | 343 | 531 | gi\|511075 | ORF2 [Streptococcus agalactiae] | 66 | 51 | |
| 823 | 1 | 1 | 393 | gi\|1303843 | YqfV [Bacillus subtilis] | 66 | 42 | |
| 891 | 1 | 82 | 402 | gi\|567769 | ORF5; predicted protein shows similarity | 66 | 52 | |
| | | | | | to ATP-binding transport roteins AmiE and | | | |
| | | | | | AmiF of Streptococcus pneumoniae; | | | |
| | | | | | disruptulon of RF5 leads to aminopterin | | | |
| | | | | | resistance [Streptococcus parasanguis] | 66 | 52 | |
| 5 | 6 | 2630 | 3154 | gi\|1303811 | YqeU [Bacillus subtilis] | 65 | 50 | |
| 16 | 1 | 2 | 628 | gi\|1742303 | Acyl carrier protein phosphodiesterase | 65 | 43 | |
| | | | | | (ACP phosphodiesterase) (fragment), | | | |
| | | | | | [Escherichia coli] | | | |
| 18 | 6 | 3360 | 2518 | gi\|601880 | rep protein [Bacillus borstelensis] | 65 | 40 | |
| 21 | 11 | 7933 | 7706 | gi\|1500521 | M. jannaschii predicted coding region | 65 | 32 | |
| | | | | | MJ1623 [Methanococcus jannaschii] | | | |
| 23 | 20 | 13459 | 13881 | gi\|488430 | alcohol dehydrogenase 2 [Entamoeba | 65 | 43 | |
| | | | | | histolytica] | | | |
| 23 | 25 | 15987 | 16178 | gnl\|PID\|e248966 | F32D8.5 [Caenorhabditis elegans] | 65 | 50 | |
| 27 | 2 | 526 | 302 | gi\|1001644 | regulatory components of sensory | 65 | 44 | |
| | | | | | transduction system [Synechocystis sp.] | | | |
| 29 | 9 | 6770 | 5727 | sp\|P36672\|PTTB_ECO | PTS SYSTEM, TREHALOSE-SPECIFIC IIBC | 65 | 45 | |
| | | | | LI | COMPONENT (EIIBC-TRE) (TREHALOSE- PERMEASE | | | |
| | | | | | IIBC COMPONENT) (PHOSPHOTRANSFERASE ENZYME | | | |
| | | | | | II, BC COMPONENT) (BC 2.7.1.69) (EII-TRE). | | | |
| 31 | 5 | 4611 | 5207 | gi\|171625 | guanylate kinase [Saccharomyces | 65 | 39 | |
| | | | | | cerevisiae] | | | |
| 32 | 7 | 4085 | 3915 | gi\|150158 | 29 kD protein [Mycoplasma genitalium] | 65 | 51 | |
| 33 | 8 | 7396 | 7638 | gi\|1573421 | protein translocation protein, low | 65 | 26 | |
| | | | | | temperature (secG) [Haemophilus | | | |
| | | | | | influenzae] | | | |
| 35 | 1 | 2 | 499 | gi\|1737500 | transcription antiterminator [Bacillus | 65 | 40 | |
| | | | | | stearothermophilus] | | | |
| 45 | 6 | 2537 | 3037 | gi\|511455 | unknown [Coxiella burnetii] | 65 | 37 | |
| 46 | 3 | 1028 | 2254 | gi\|1001642 | dGTP triphosphohydrolase [Synechocystis | 65 | 43 | |
| | | | | | sp.] | | | |
| 47 | 12 | 14524 | 14264 | gi\|150209 | ORF 1 [Mycoplasma mycoides] | 65 | 34 | |
| 50 | 3 | 2866 | 2051 | gi\|1303830 | YgfL [Bacillus subtilis] | 65 | 40 | |
| 57 | 11 | 12955 | 13332 | gnl\|PID\|e254999 | phenylalany-tRNA synthetase beta subunit | 65 | 51 | |
| | | | | | [Bacillus subtilis] | | | |
| 62 | 1 | 2 | 484 | gi\|1573470 | H. influenzae predicted coding region | 65 | 57 | |
| | | | | | H10491 [Haemophilus influenzae] | | | |
| 68 | 1 | 49 | 282 | gi\|1573250 | aspartate aminotransferase (aspC) | 65 | 52 | |
| | | | | | [Haemophilus influenzae] | | | |
| 72 | 2 | 567 | 1325 | gi\|466645 | alternate name yhiD [Escherichia coli] | 65 | 40 | |
| 81 | 5 | 3711 | 2938 | gi\|1732200 | PTS permease for mannose subunit IIPMan | 65 | 43 | |
| | | | | | [Vibria furnissii] | | | |
| 83 | 18 | 12506 | 12745 | pir\|D64042\|D64042 | ribosomal-protein-alanine | 65 | 50 | |
| | | | | | acetyltransferase (rimI) homolog - | | | |
| | | | | | Haemophilus influenzae (strain Rd KW20) | | | |
| 100 | 38 | 28229 | 28032 | gi\|183075 | glial fibrillary acidic protein [Homo | 65 | 43 | |
| | | | | | sapiens] | | | |
| 105 | 1 | 912 | 106 | pir\|S15248\|YQBZCD | fimC protein - Dichelobacter nodosus | 65 | 46 | |
| | | | | | (serotype D) | | | |
| 106 | 5 | 6097 | 5102 | gi\|1143204 | ORF2; Method: conceptual translation | 65 | 44 | |
| | | | | | supplied by author [Shigella sonnei] | | | |
| 109 | 3 | 1165 | 899 | gi\|1573390 | hypothetical [Haemophilus influenzae] | | | |
| 110 | 7 | 5579 | 4257 | pir\|B44514\|B44514 | hypothetical protein 1 (vnfA 5′ region) - | 65 | 43 | |
| | | | | | Azotobacter vinelandii] | | | |
| 120 | 3 | 1249 | 1632 | sp\|P54746\|YBGB_ECO | HYPOTHETICAL PROTEIN IN HRSA 3′REGION | 65 | 48 | |
| | | | | LI | (FRAGMENT). | | | |
| 122 | 2 | 896 | 1654 | gi\|1335913 | unknown [Erysipelothrix rhusiopathiae] | 65 | 48 | |
| 145 | 4 | 2509 | 3210 | gi\|1208965 | hypothetical 23.3 kd protein [Eseherichia | 65 | 40 | |
| | | | | | coli] | | | |
| 149 | 7 | 4407 | 3502 | gi\|145173 | 35 kDa protein [Escherichia coli] | 65 | 46 | |
| 154 | 8 | 5738 | 4926 | gi\|405804 | transposase [Streptococcus thermophilis] | 65 | 47 | |
| 155 | 1 | 306 | 512 | gi\|285627 | E.coli SecE homologous protein [Bacillus | 65 | 48 | |
| | | | | | subtilis] pir\|S39858\|S39858 secE protein | | | |
| | | | | | homolog - Bacillus subtilis | | | |
| | | | | | sp\|Q06799\|SECE_BACSU PREPROTEIN | | | |
| | | | | | TRANSLOCASE SECE SUBUNIT. | | | |
| 158 | 1 | 150 | 1103 | gi\|289272 | ferrichrome-binding protein [Bacillus | 65 | 40 | |
| | | | | | subtilis] | | | |
| 158 | 16 | 14885 | 15946 | gi\|467172 | add; L308_C2_206 [Mycobacterium leprae] | 65 | 36 | |
| 173 | 4 | 2103 | 2912 | gnl\|PID\|e254877 | unknown [Mycobacterium tuberculosis] | 65 | 41 | |
| 173 | 12 | 9749 | 9054 | gi\|1652864 | hypothetical protein [Synechocystis sp.] | 65 | 50 | |
| 179 | 16 | 15674 | 17035 | gi\|1171125 | thioredoxin reductase [Clostridium | 65 | 41 | |
| | | | | | litorale] | | | |
| 180 | 26 | 26911 | 28266 | sp\|P13692\|P54_ENTF | P54 PROTEIN PRECURSOR. | 65 | 39 | |
| | | | | C | | | | |
| 193 | 6 | 2893 | 3795 | gi\|39787 | adaA [Bacillus subtilis] | 65 | 45 | |
| 194 | 5 | 1843 | 2238 | gi\|47394 | 5-oxoprolyl-peptidase [Streptococcus | 65 | 48 | |
| | | | | | pyogenes] | | | |
| 199 | 1 | 894 | 82 | gi\|1591118 | nitrate transport ATP-binding protein | 65 | 46 | |
| | | | | | [Methanococcus jannaschii] | | | |
| 200 | 24 | 13441 | 13136 | gi\|144926 | toxin A [Clostridium difficile] | 65 | 39 | |
| 202 | 3 | 2925 | 1846 | gi\|413968 | ipa-44d gene product [Bacillus subtilis] | 65 | 46 | |
| 203 | 1 | 797 | 3 | gi\|1377832 | unknown [Bacillus subtilis] | 65 | 45 | |
| 204 | 3 | 1065 | 1472 | gi\|1008996 | unknown [Schizosaccharomyces pombe] | 65 | 51 | |
| 205 | 4 | 1029 | 1685 | gi\|148989 | truncated tetracycline resistance | 65 | 42 | |
| | | | | | repressor (non-functional) Haemophilus | | | |
| | | | | | parainfluenzae] | | | |
| 206 | 8 | 5037 | 4807 | pir\|D60110\|D60110 | repetitive protein antigen 3 - Trypanosoma | 65 | 41 | |
| | | | | | cruzi (fragment) | | | |
| 217 | 1 | 411 | 4 | gi\|1146181 | putative [Bacillus subtilis] | 65 | 43 | |
| 217 | 4 | 1092 | 3065 | gi\|984229 | penicillin-binding protein 1a | 65 | 48 | |
| | | | | | [Streptococcus pneumoniae] | | | |
| 223 | 27 | 23445 | 23879 | gnl\|PID\|e269486 | Unknown [Bacillus subtilis] | 65 | 47 | |
| 225 | 6 | 5138 | 3984 | gi\|39956 | IIGlc [Bacillus subtilis] | 65 | 47 | |
| 229 | 5 | 5528 | 5130 | gi\|1303914 | YghY [Bacillus subtilis] | 65 | 33 | |
| 229 | 10 | 10697 | 8517 | gnl\|PID\|e266933 | unknown [Mycobacterium tuberculosis] | 65 | 46 | |
| 233 | 3 | 2413 | 1526 | gi\|1887825 | ORF_f541 [Escherichia coli] | 65 | 46 | |
| 236 | 4 | 6975 | 4789 | gi\|405863 | yohA [Escherichia coli] | 65 | 43 | |
| 237 | 4 | 1460 | 1816 | gi\|305080 | myosin heavy chain [Entamoeba histolytica] | 65 | 42 | |
| 238 | 24 | 21690 | 23228 | gi\|305008 | rhamnulokinase [Escherichia coli] | 65 | 49 | |
| 242 | 3 | 2192 | 3280 | gnl\|PID\|e221269 | tail protein [Bacteriophage CP-1] | 65 | 37 | |
| 244 | 6 | 5172 | 4228 | gi\|1653197 | hypothetical protein [Synechocystis sp.] | 65 | 51 | |
| 259 | 5 | 3684 | 2779 | gi\|559900 | F49E2.1 [Caenorhabditis elegans] | 65 | 39 | |
| 259 | 6 | 4243 | 3749 | gi\|1743887 | molybdopterin cofactor biosynthesis enzyme | 65 | 50 | |
| | | | | | [Bradyrhizobium laponicum] | | | |
| 260 | 1 | 140 | 478 | gi\|895748 | putative cellobiose phosphotransferase | 65 | 55 | |
| | | | | | enzyme II [Bacillus ubtilis] | | | |
| 269 | 6 | 4113 | 3907 | gi\|1303792 | YgeK [Bacillus subtilis] | 65 | 39 | |
| 271 | 12 | 7731 | 6772 | gi\|1657534 | cyn operon transcriptional activator | 65 | 45 | |
| | | | | | [Escherichia coli] | | | |
| 275 | 9 | 6413 | 5361 | gi\|1773132 | multidrug resistance-like ATP-binding | 65 | 48 | |
| | | | | | protein Mdl [Escherichia coli] | | | |
| 276 | 4 | 1813 | 1583 | gi\|1504014 | similar to myosin heavy chain: Containing | 65 | 34 | |
| | | | | | ATP/GTP-binding site motif A(P-loop) [Homo | | | |
| | | | | | sapiens] | | | |
| 279 | 14 | 14254 | 10625 | gi\|1237015 | ORF4 [Bacillus subtilis] | 65 | 45 | |
| 281 | 2 | 692 | 1279 | gi\|1303962 | YgjK [Bacillus subtilis] | 65 | 50 | |
| 295 | 5 | 2279 | 3388 | gi\|436965 | [malA] gene products [Bacillus | 65 | 41 | |
| | | | | | stearothermophilus] pir\|543914\|S43914 | | | |
| | | | | | hypothetical protein 1 - Bacillus | | | |
| | | | | | tearothermophilus | | | |
| 298 | 1 | 63 | 1142 | gi\|928834 | integrase [Lactococcus lactis phage BK5-T] | 65 | 44 | |
| 301 | 8 | 7592 | 7176 | gi\|1303893 | YqhL [Bacillus subtilis] | 65 | 50 | |
| 311 | 3 | 4658 | 5701 | gnl\|PID\|e221269 | tail protein [Bacteriophage CP-1] | 65 | 40 | |
| 326 | 1 | 2 | 247 | gi\|466520 | pocR [Salmonella typhimurium] | 65 | 38 | |
| 329 | 1 | 789 | 523 | gi\|1303895 | YqhN [Bacillus subtilis] | 65 | 36 | |
| 345 | 5 | 3363 | 3641 | gi\|895749 | putative cellobiose phosphotransferase | 65 | 51 | |
| | | | | | enzyme II″ [Bacillus ubtilis] | | | |
| 369 | 3 | 1635 | 1207 | gi\|1480429 | putative transcriptional regulator | 65 | 45 | |
| | | | | | [Bacillus stearothermophilus] | | | |
| 373 | 2 | 815 | 1630 | gi\|1277032 | unknown [Bacillus subtilis] | 65 | 41 | |
| 379 | 9 | 11301 | 8275 | gi\|887828 | was o492p and o826p before splice | 65 | 49 | |
| | | | | | [Escherichia coli] | | | |
| 386 | 13 | 7903 | 8145 | gnl\|PID\|e217382 | M7.9 [Caenorhabditis elegans] | 65 | 39 | |
| 395 | 4 | 1028 | 1231 | gi\|1592033 | M. jannaschii predicted coding region | 65 | 30 | |
| | | | | | MJ1387 [Methanococcus jannaschii] | | | |
| 396 | 3 | 1000 | 1272 | gi\|1045900 | hypothetical protein (GB:L09228_17) | 65 | 44 | |
| | | | | | [Mycoplasma genitalium] | | | |
| 422 | 3 | 2050 | 1262 | gi\|405907 | yejD [Escherichia coli] | 65 | 50 | |
| 438 | 1 | 44 | 358 | gi\|530798 | LysB [Bacteriophage phi-LC3] | 65 | 39 | |
| 460 | 1 | 119 | 646 | gi\|1502420 | malonyl-CoA:Acyl carrier protein | 65 | 46 | |
| | | | | | transacylase [Bacillus subtilis] | | | |
| 463 | 1 | 870 | 121 | gi\|1651917 | tRNA(m1G37)methyltramsferase | 65 | 47 | |
| | | | | | [Synechocystis sp.] | | | |
| 468 | 1 | 2 | 823 | gi\|216457 | ORF [Escherichia coli] | 65 | 46 | |
| 470 | 1 | 34 | 816 | gi\|530798 | LysB [Bacteriophage phi-LC3] | 65 | 47 | |
| 476 | 1 | 21 | 830 | gi\|1006591 | cation-transporting ATPase PacL | 65 | 46 | |
| | | | | | [Synechocystis sp.] | | | |
| 510 | 7 | 4875 | 6092 | gi\|143150 | levR [Bacillus subtilis] | 65 | 46 | |
| 565 | 2 | 686 | 339 | gi\|143833 | PBSX repressor [Bacillus subtilis] | 65 | 51 | |
| 566 | 2 | 198 | 743 | gi\|496501 | RepS [Streptococcus pyogenes] | 65 | 34 | |
| 604 | 5 | 1875 | 2078 | gi\|1590997 | M. jannaschii predicted coding region | 65 | 49 | |
| | | | | | MJ0272 [Methanococcus jannaschii] | | | |
| 608 | 1 | 194 | 3 | gnl\|PID\|e290940 | unknown [Mycobacterium tuberculosis] | 65 | 35 | |
| 648 | 1 | 60 | 953 | gi\|1591145 | hypothetical protein (HI0902) | 65 | 31 | |
| | | | | | [Methanococcus jannaschii] | | | |
| 657 | 4 | 2531 | 1620 | gi\|1500015 | amidase [Methanococcus jannaschii] | 65 | 46 | |
| 691 | 1 | 2 | 718 | gnl\|PID\|e248400 | orfRM1 gene product [Bacillus subtilis] | 65 | 48 | |
| 704 | 2 | 474 | 175 | gi\|467428 | unknown [Bacillus subtilis] | 65 | 50 | |
| 758 | 2 | 408 | 683 | gi\|451201 | ORF1 [Bacillus subtilis] | 65 | 44 | |
| 778 | 1 | 833 | 3 | gi\|410137 | ORFX13 [Bacillus subtilis] | 65 | 40 | |
| 793 | 1 | 1 | 564 | gi\|912436 | oligo-1,6-glucosidase [Bacillus | 65 | 40 | |
| | | | | | thermoglucosidasius] pir\|A41707\|A41707 | | | |
| | | | | | oligo-1,6-glucosidase (BC 3.2.1.10) - | | | |
| | | | | | Bacillus hemoglucosidasius | | | |
| 827 | 1 | 364 | 2 | gi\|852076 | MrgA [Bacillus subtilis] | 65 | 33 | |
| 856 | 1 | 209 | 3 | gi\|1575605 | 4-methyl-5-nitrocatechol oxygenase | 65 | 45 | |
| | | | | | [Burkholderia sp.] | | | |
| 890 | 1 | 966 | 745 | pir\|A44803\|A44803 | pG1 protein - human (fragment) | 65 | 63 | |
| 4 | 1 | 2 | 958 | gnl\|PID\|e265530 | yorfE [Streptococcus pneumoniae] | 64 | 43 | |
| 5 | 8 | 4212 | 5579 | gi\|407881 | stringent response-like protein | 64 | 47 | |
| | | | | | [Streptococcus equisimilis] | | | |
| | | | | | pir\|539975\|539975 stringent response-like | | | |
| | | | | | protein - Streptococcus quisimilis | | | |
| 8 | 4 | 4047 | 3304 | gi\|1573150 | dihydrolipoamide acetyltransferase (acoC) | 64 | 37 | |
| | | | | | [Haemophilus influenzae] | | | |
| 17 | 14 | 11709 | 10393 | gi\|155109 | ORF 1B [Thermus aguaticus thermophilus] | 64 | 37 | |
| 19 | 12 | 6499 | 6801 | gi\|1303755 | YqbO [Bacillus subtilis] | 64 | 32 | |
| 23 | 1 | 1 | 303 | gi\|1022963 | dextransucrase [Leuconostoc mesenteroides] | 64 | 50 | |
| 28 | 4 | 7059 | 6505 | gi\|1568609 | 18kDA protein [Streptococcus pneumoniae] | 64 | 45 | |
| 31 | 3 | 1316 | 2986 | gi\|1100076 | PTS-dependent enzyme II [Clostridium | 64 | 47 | |
| | | | | | longisporum] | | | |
| 47 | 2 | 2665 | 3408 | gi\|1742154 | Phosphoglycolate phosphatase (EC | 64 | 52 | |
| | | | | | 3.1.3.18). [Escherichia coli] | | | |
| 48 | 2 | 1699 | 1310 | gi\|142702 | A competence protein 2 [Bacillus subtilis] | 64 | 41 | |
| 54 | 8 | 2750 | 2352 | gi\|951052 | ORF9, putative [Streptococcus pneumoniae] | 64 | 31 | |
| 57 | 15 | 18035 | 17274 | gi\|1183886 | integral membrane protein [Bacillus | 64 | 40 | |
| | | | | | subtilis] | | | |
| 62 | 4 | 1968 | 1699 | gi\|475110 | fructokinase [Pediococcus pentosaceus] | 64 | 52 | |
| 100 | 42 | 29329 | 29039 | gi\|951048 | excisionase [Streptococcus pneumoniae] | 64 | 37 | |
| 102 | 4 | 3726 | 4805 | gi\|215331 | morphogenesis protein [Bacteriophage phi- | 64 | 43 | |
| | | | | | 29] | | | |
| 106 | 3 | 3296 | 2439 | gi\|1303930 | YgiK [Bacillus subtilis] | 64 | 44 | |
| 123 | 12 | 12960 | 11314 | sp\|P37047\|YAEG_ECO | HYPOTHETICAL 44.3 KD PROTEIN IN HTRA-DAPD | 64 | 40 | |
| | | | | LI | INTERGENIC REGION. | | | |
| 128 | 2 | 1285 | 1614 | gi\|143961 | pyruvate phosphate dikinase [Clostridium | 64 | 52 | |
| | | | | | symbiosum] pir\|A36231\|KIQAPO | | | |
| | | | | | pyruvate, orthophosphate dikinase (EC | | | |
| | | | | | 2.7.9.1) - lostridium symbiosum | | | |
| 128 | 8 | 6178 | 4757 | gi\|40665 | beta-glucosidase [Clostridium | 64 | 41 | |
| | | | | | thermocellum] | | | |
| 133 | 2 | 1748 | 2248 | gi\|1591027 | ferripyochelin binding protein | 64 | 46 | |
| | | | | | [Methanococcus jannaschii] | | | |
| 150 | 1 | 35 | 673 | gnl\|PID\|e185372 | ceuC gene product [Campylobacter coli] | 64 | 38 | |
| 158 | 6 | 6038 | 5040 | gi\|1045801 | hypothetical protein (SP:P32720) | 64 | 35 | |
| | | | | | [Mycoplasma genitalium] | | | |
| 164 | 7 | 3620 | 4903 | gnl\|PID\|e283116 | unknown similar to quinolon resistance | 64 | 41 | |
| | | | | | protein NorA [Bacillus subtilis] | | | |
| 171 | 11 | 10107 | 10784 | gi\|1591668 | phosphate transport system regulatory | 64 | 40 | |
| | | | | | protein [Methanococcus jannaschii] | | | |
| 179 | 4 | 4826 | 6373 | gi\|149535 | D-alanine activating enzyme [Lactobacillus | 64 | 51 | |
| | | | | | casei] | | | |
| 181 | 4 | 2251 | 1364 | gi\|671632 | unknown [Staphylococcus aureus] | 64 | 38 | |
| 190 | 11 | 11302 | 10355 | gi\|599850 | orf1 gene product [Lactobacillus sake] | 64 | 33 | |
| 195 | 37 | 15344 | 16033 | gi\|1736499 | Lysostaphin precursor (BC 3.5.1.-). | 64 | 49 | |
| | | | | | [Escherichia coli] | | | |
| 199 | 4 | 4000 | 5631 | gi\|746574 | similar to M. musculus transport system | 64 | 37 | |
| | | | | | membrane protein, Nramp PIR:A40739) and S. | | | |
| | | | | | cerevisiae SMF1 protein (PIR:A45154) | | | |
| | | | | | Caenorhabditis elegans] | | | |
| 202 | 1 | 1 | 1560 | gi\|309662 | pheromone binding protein [Plasmid pCF10] | 64 | 45 | |
| 204 | 7 | 3000 | 4115 | gi\|1591731 | melvalonate kinase [Methanococcus | 64 | 41 | |
| | | | | | jannaschii] | | | |
| 208 | 1 | 308 | 1090 | gi\|473821 | ‘tetrahydrodipicolinate N- | 64 | 42 | |
| | | | | | succinyltransferase’ [Escherichia coli] | | | |
| | | | | | gi\|1552743 tetrahydrodipicolinate N- | | | |
| | | | | | succinyltransferase Escherichia coli] | | | |
| 216 | 9 | 6501 | 6698 | gi\|47373 | 7 kDa protein [Streptococcus pneumoniae] | 64 | 35 | |
| 221 | 18 | 8268 | 8513 | gi\|1389837 | complement regulatory protein [Trypanosoma | 64 | 28 | |
| | | | | | cruzi] | | | |
| 231 | 4 | 2964 | 2632 | gnl\|PID\|e279941 | muconate cycloisomerase [Rhodococcus | 64 | 37 | |
| | | | | | erythropolis] | | | |
| 234 | 2 | 751 | 302 | gnl\|PID\|e194709 | N-terminal part of a protein of unknown | 64 | 42 | |
| | | | | | function [Chlamydia psittaci] | | | |
| 238 | 18 | 15580 | 16392 | gi\|537108 | ORE_f254 [Escherichia coli] | 64 | 44 | |
| 245 | 1 | 14 | 868 | gi\|153247 | endo-beta-N-acetylglucosaminidase H | 64 | 51 | |
| | | | | | [Streptomyces plicatus]pir\|A00903\|RBSMHP | | | |
| | | | | | mannosyl-glycoprotein ndo-beta-N- | | | |
| | | | | | acetyiglucosaminidase (EC 3.2.1.96) H | | | |
| | | | | | precursor - treptomyces plicatus | | | |
| 272 | 2 | 584 | 1144 | gi\|580781 | signal peptidase [Bacillus licheniformis] | 64 | 47 | |
| 281 | 5 | 2659 | 5019 | gi\|147550 | recJ [Escherichia coli] | 64 | 46 | |
| 290 | 12 | 9496 | 10371 | gi\|45713 | P.putida genes rpmH, rnpA, 9k, 60k, 50k, | 64 | 42 | |
| | | | | | gidA, gidB, uncI and uncB seudomonas | | | |
| | | | | | putida] | | | |
| 298 | 4 | 4029 | 3466 | gi\|147780 | rts gene product [Escherichia coli] | 64 | 43 | |
| 301 | 20 | 16216 | 15977 | gi\|170482 | prosystemin [Solanum lycopersicum] | 64 | 57 | |
| 301 | 21 | 17732 | 17391 | gi\|405804 | transposase [Streptococcus thermophilus] | 64 | 52 | |
| 307 | 1 | 198 | 1964 | gi\|1255196 | BSMA [Bacillus stearothermophilus] | 64 | 48 | |
| 320 | 5 | 3441 | 3070 | gi\|972900 | ArtP [Haemophilus influenzae] | 64 | 38 | |
| 341 | 9 | 7690 | 6413 | gi\|1161380 | IcaA [Staphylococcus epidermidis] | 64 | 30 | |
| 345 | 6 | 3589 | 4848 | gi\|902932 | L-methionine gamma-lyase [Pseudomonas | 64 | 45 | |
| | | | | | putida] | | | |
| 348 | 1 | 453 | 22 | gi\|1591957 | M. jannaschii predicted coding region | 64 | 32 | |
| | | | | | MJ1318 [Methanococcus jannaschii] | | | |
| 350 | 2 | 1372 | 1830 | gnl\|PID\|e289141 | similar to hydroxyrnyristoyl-(acyl carrier | 64 | 44 | |
| | | | | | protein) dehydratase [Bacillus subtilis] | | | |
| 351 | 7 | 3291 | 2917 | gi\|49013 | dTDP-dihydrostreptose synthase | 64 | 46 | |
| | | | | | [Streptomyces griseus] ir\|S18618\|SYSMPG | | | |
| | | | | | dTDP-dihydrostreptose synthase - | | | |
| | | | | | Streptomyces iseus | | | |
| 352 | 2 | 780 | 1028 | gi\|173431 | H+−ATPase [Schizosaccharomyces pombe] | 64 | 38 | |
| 386 | 10 | 5952 | 6161 | gnl\|PID\|e243284 | ORF YGLO56c [Saccharomyces cerevisiae] | 64 | 50 | |
| 398 | 2 | 1233 | 1808 | gi\|147920 | 3-methyladenine-DNA glycosylase I (tag) | 64 | 47 | |
| | | | | | [Escherichia coli] | | | |
| 399 | 12 | 8761 | 9159 | gi\|1778534 | H10024 homolog [Escherichia coli] | 64 | 40 | |
| 409 | 1 | 657 | 1607 | gi\|1773157 | ferrochelatase [Escherichia coli] | 64 | 41 | |
| 446 | 1 | 266 | 775 | gi\|563845 | orf gene product [Bacillus circulans] | 64 | 53 | |
| 462 | 4 | 1714 | 1959 | gi\|169461 | serine proteinase inhibitor [Populus | 64 | 50 | |
| | | | | | trichocarpa × Populus eltoides] | | | |
| 466 | 6 | 5621 | 8539 | gi\|143150 | levR [Bacillus subtilis] | 64 | 43 | |
| 501 | 2 | 891 | 1469 | gi\|467109 | rim; 30S Ribosomal protein S18 alanine | 64 | 44 | |
| | | | | | acetyltransferase; 229_C1_170 | | | |
| | | | | | [Mycobacterium leprae] | | | |
| 512 | 1 | 1 | 279 | gi\|1651948 | hypothetical protein [Synechocystis sp.] | 64 | 35 | |
| 516 | 1 | 466 | 2 | gi\|155027 | 6′-N-acetyltransferase [Transposon Tn2426] | 64 | 35 | |
| 516 | 2 | 556 | 759 | gi\|1653387 | nitrogen assimilation regulatory protein | 64 | 58 | |
| | | | | | [Synechocystis sp.] | | | |
| 523 | 2 | 904 | 662 | gi\|159464 | armadillo protein [Musca domestica] | 64 | 45 | |
| 537 | 2 | 1083 | 844 | gi\|929966 | truncated ORFB due to a basepair deletion; | 64 | 42 | |
| | | | | | similar to B. anthracis terneR element | | | |
| | | | | | ORFB [Bacillus anthracis] | | | |
| 549 | 1 | 309 | 4 | gi\|1279769 | FdhC [Methanobacterium thermoformicicum] | 64 | 48 | |
| 552 | 4 | 5960 | 3945 | gi\|1100076 | PTS-dependent enzyme II [Clostridium | 64 | 47 | |
| | | | | | longisporum] | | | |
| 556 | 1 | 3 | 224 | gi\|727437 | putative 37-kDa protein [Lactococcus | 64 | 49 | |
| | | | | | lactis] | | | |
| 557 | 2 | 767 | 1120 | gnl\|PID\|e257629 | transcription factor [Lactococcus lactis] | 64 | 44 | |
| 602 | 1 | 428 | 156 | gi\|520407 | orf2; GTG start codon [Bacillus | 64 | 50 | |
| | | | | | thuringiensis] | | | |
| 603 | 1 | 1 | 165 | gi\|1621445 | sporulation protein Cse15 [Bacillus | 64 | 32 | |
| | | | | | subtilis] | | | |
| 626 | 1 | 3 | 992 | gi\|1574715 | thioredoxin reductase (trxB) [Haemophilus | 64 | 40 | |
| | | | | | influenzae] | | | |
| 628 | 2 | 240 | 446 | gi\|1165281 | Smg [Borrelia burgdorferi] | 64 | 41 | |
| 723 | 1 | 23 | 829 | gi\|1620648 | surface protein Rib [Streptococcus | 64 | 50 | |
| | | | | | agalactiae] | | | |
| 739 | 1 | 4 | 378 | gi\|143835 | PBSX repressor [Bacillus subtilis] | 64 | 37 | |
| 748 | 1 | 139 | 765 | gi\|498816 | ORF7; homology to regions 4.1 and 4.2 of | 64 | 35 | |
| | | | | | sigma factors [Bacillus ubtilis] | | | |
| 758 | 1 | 3 | 410 | gi\|451201 | ORF1 [Bacillus subtilis] | 64 | 34 | |
| 808 | 1 | 368 | 3 | gi\|142833 | ORF2 [Bacillus subtilis] | 64 | 47 | |
| 818 | 2 | 415 | 663 | gi\|854020 | U41, major DNA binding protein [Human | 64 | 40 | |
| | | | | | herpesvirus 6] | | | |
| 906 | 1 | 2 | 433 | gi\|1303865 | YggR [Bacillus subtilis] | 64 | 44 | |
| 17 | 28 | 28175 | 27612 | gi\|151824 | ORF5 [Plasmid R46] | 63 | 34 | |
| 19 | 18 | 9546 | 9722 | gi\|288661 | ORF5 product [Bacteriophage P2] | 63 | 45 | |
| 39 | 5 | 1841 | 2329 | gi\|1573292 | hypothetical [Haemophilus influenzae] | 63 47 | | |
| 41 | 1 | 1531 | 2 | gi\|580896 | nodB protein (aa 1-219) [Bradyrhizobium | 63 | 43 | |
| | | | | | sp.] | | | |
| 55 | 10 | 5052 | 6410 | gi\|1303917 | YgiB [Bacillus subtilis] | 63 | 42 | |
| 80 | 2 | 1852 | 824 | gi\|38722 | precursor (aa −20 to 381) [Acinetobacter | 63 | 42 | |
| | | | | | calcoaceticus] ir\|A29277\|A29277 aldose 1- | | | |
| | | | | | epimerase (EC 5.1.3.3) - Acinetobacter | | | |
| | | | | | lcoaceticus | | | |
| 81 | 10 | 6724 | 6221 | gi\|1591234 | hypothetical protein (SP:P42297) | 63 | 40 | |
| | | | | | [Methanococcus jannaschii] | | | |
| 81 | 14 | 9175 | 10848 | gi\|309662 | pheromone binding protein [Plasmid pcF10] | 63 | 44 | |
| 86 | 1 | 2 | 1006 | gi\|143316 | [gap]gene products [Bacillus megaterium] | 63 | 43 | |
| 89 | 13 | 12929 | 12639 | gi\|1377841 | unknown [Bacillus subtilis] | 63 | 44 | |
| 98 | 14 | 14365 | 13502 | sp\|P45169\|POTC_HAE | SPERMIDINE/PUTRESCINE TRANSPORT SYSTEM | 63 | 37 | |
| | | | | IN | PERMEASE PROTEIN POTC. | | | |
| 100 | 24 | 20444 | 17985 | gi\|563258 | virulence-associated protein E | 63 | 44 | |
| | | | | | [Dichelobacter nodosus] | | | |
| 102 | 2 | 2441 | 2599 | gi\|1619835 | MOB [Bacillus thuringiensis israelens] | 63 | 28 | |
| 110 | 22 | 19725 | 20705 | gi\|1763011 | lysophospholipase homolog [Homo sapiens] | 63 | 48 | |
| 115 | 1 | 481 | 92 | gi\|467360 | unknown [Bacillus subtilis] | 63 | 38 | |
| 128 | 30 | 25257 | 24397 | gi\|1518679 | orf [Bacillus subtilis] | 63 | 39 | |
| 138 | 18 | 12236 | 11580 | gi\|405516 | This ORF is homologous to nitroreductase | 63 | 39 | |
| | | | | | from Enterobacter cloacae, ccession Number | | | |
| | | | | | A38686, and Salmonella, Accession Number | | | |
| | | | | | P15888 Mycoplasma-like organism] | | | |
| 143 | 2 | 167 | 1096 | pir\|S39416\|S39416 | metallothionein 10-I - blue mussel | 63 | 63 | |
| 158 | 9 | 10023 | 8893 | bbs\|173803 | CD4+ T cell-stimulating antigen [Listeria | 63 | 48 | |
| | | | | | monocytogenes, 85EO-1167, Peptide Partial, | | | |
| | | | | | 268 aa] [Listeria monocytogenes] | | | |
| 164 | 6 | 3041 | 3301 | gi\|1573583 | H. influenzae predicted coding region | 63 | 31 | |
| | | | | | H10594 [Haemophilus influenzae] | | | |
| 164 | 18 | 18502 | 21708 | gi\|1015903 | ORF YJR151c [Saccharomyces cerevisiae] | 63 | 45 | |
| 165 | 3 | 3084 | 2278 | gi\|537108 | ORF_f254 [Escherichia coli] | 63 | 45 | |
| 166 | 1 | 83 | 1045 | gi\|762778 | NifS gene product [Anabaena azollae] | 63 | 49 | |
| 168 | 3 | 638 | 1489 | gi\|805022 | Ndilp [Saccharomyces cerevisiae] | 63 | 32 | |
| 171 | 12 | 10655 | 10810 | gi\|152403 | phosphate regulatory protein [Rhizobium | 63 | 50 | |
| | | | | | meliloti] | | | |
| 172 | 1 | 242 | 1336 | gi\|1552775 | ATP-binding protein [Escherichia coli] | 63 | 45 | |
| 179 | 11 | 11236 | 12111 | gnl\|PID\|e245033 | unknown [Mycobacterium tuberculosis] | 63 | 42 | |
| 179 | 15 | 15289 | 15765 | gi\|1353197 | thioredoxin reductase [Eubacterium | 63 | 44 | |
| | | | | | acidaminophilum] | | | |
| 180 | 3 | 3412 | 1892 | gi\|1064813 | homologous to sp:PHOR_BACSU [Bacillus | 63 | 40 | |
| | | | | | subtilis] | | | |
| 180 | 7 | 7063 | 7926 | gi\|1657516 | hypothetical protein [Escherichia coli] | 63 | 41 | |
| 187 | 1 | 1 | 729 | gi\|1651957 | hypothetical protein [Synechocystis sp.] | 63 | 34 | |
| 195 | 17 | 7717 | 8280 | gi\|431928 | MunI methyltransferase [Mycoplasma sp.] | 63 | 44 | |
| 202 | 8 | 5311 | 6165 | gi\|606162 | ORF_f229 [Escherichia coli] | 63 | 48 | |
| 202 | 10 | 7848 | 8681 | gi\|606018 | ORF_o783 [Escherichia coli] | 63 | 47 | |
| 208 | 3 | 2979 | 2341 | gi\|1006613 | hypothetical protein [Synechocystis sp.] | 63 | 40 | |
| 221 | 3 | 874 | 1146 | gnl\|PID\|e265530 | yorfE [Streptococcus pneumoniae] | 63 | 42 | |
| 227 | 2 | 856 | 1254 | gi\|438459 | homologous to E. coli hydrophobic Fe- | 63 | 41 | |
| | | | | | uptake components FepD, FecD; utative | | | |
| | | | | | [Bacillus subtilis] | | | |
| 231 | 3 | 2618 | 2448 | gi\|606248 | 30S ribosomal subunit protein S3 | 63 | 42 | |
| | | | | | [Escherichia coli] | | | |
| 233 | 9 | 6773 | 6144 | gi\|887827 | ORF_o192 [Escherichia coli] | 63 | 41 | |
| 234 | 1 | 348 | 70 | gi\|494958 | ExpZ [Bacillus subtilis] | 63 | 32 | |
| 240 | 2 | 1230 | 721 | gnl\|PID\|e252616 | DcuC protein [Escherichia coli] | 63 | 38 | |
| 244 | 9 | 7512 | 6508 | gi\|467421 | similar to B. subtilis DnaH [Bacillus | 63 | 43 | |
| | | | | | subtilis] sp\|P37540\|YAAS_BACSU | | | |
| | | | | | HYPOTHETICAL 37.6 KD PROTEIN IN XPAC-ABRB | | | |
| | | | | | NTERGENIC REGION. | | | |
| 255 | 5 | 3600 | 2818 | gi\|1486244 | unknown [Bacillus subtilis] | 63 | 47 | |
| 258 | 1 | 3 | 449 | gi\|1041115 | TRAC [Plasmid pPD1] | 63 | 38 | |
| 259 | 4 | 2842 | 2342 | gnl\|PID\|e290788 | unknown [Mycobacterium tuberculos] | 63 | 42 | |
| 265 | 8 | 3313 | 3480 | gi\|694074 | emml gene product [Streptococcus pyogenes] | 63 | 42 | |
| 276 | 18 | 12505 | 11654 | gi\|601878 | beta-1,3-glucanase bg1H [Bacillus | 63 | 36 | |
| | | | | | circulans] | | | |
| 294 | 5 | 2012 | 2275 | gi\|288661 | ORF5 product [Bacteriophage P2] | 63 | 40 | |
| 301 | 7 | 7063 | 6704 | gnl\|PID\|e290998 | unknown [Mycobacteriurn tuberculos] | 63 | 41 | |
| 345 | 2 | 2279 | 2725 | gi\|413940 | ipa-16d gene product [Bacillus subtilis] | 63 | 39 | |
| 351 | 8 | 4361 | 3306 | gi\|398120 | TDP-glucose oxireductase [Xanthomonas | 63 | 47 | |
| | | | | | campestris] | | | |
| 359 | 1 | 526 | 14 | gi\|1001605 | 3-hydroxyisobutyrate dehydrogenase | 63 | 36 | |
| | | | | | [Synechocystis sp.] | | | |
| 364 | 6 | 6741 | 7277 | gi\|1736473 | ORF_ID:o335#13; similar to [SwissProt | 63 | 42 | |
| | | | | | Accession Number P36088] [Escherichia | | | |
| | | | | | coli] | | | |
| 378 | 2 | 683 | 1414 | gi\|529016 | aminoglycoside 6-adenylyltransferase | 63 | 41 | |
| | | | | | [Bacillus subtilis] pir\|JU0059\|XXBSG | | | |
| | | | | | aminoglycoside 6-adenylyltransferase (EC | | | |
| | | | | | 2.7.7.-) Bacillus subtilis | | | |
| 392 | 2 | 783 | 1646 | gi\|1772644 | orfR gene product [Bacillus subtilis] | 63 | 34 | |
| 399 | 2 | 574 | 1407 | gi\|40023 | B.subtilis genes rpmH, rnpA, 50kd, gidA | 63 | 42 | |
| | | | | | and gidB [Bacillus subtilis] i\|467388 | | | |
| | | | | | stage III sporulation [Bacillus subtilis] | | | |
| | | | | | ir\|S18073\|S18073 spoIIIJ protein - | | | |
| | | | | | Bacillus subtilis | | | |
| 403 | 1 | 754 | 2 | gi\|1303938 | YqiS [Bacillus subtilis] | 63 | 52 | |
| 404 | 5 | 4149 | 3745 | gi\|142450 | ahrC protein [Bacillus subtilis] | 63 | 42 | |
| 430 | 1 | 2 | 1222 | gi\|1046082 | M. genitalium predicted coding region | 63 | 40 | |
| | | | | | MG372 [Mycoplasma genitalium] | | | |
| 432 | 1 | 3 | 1241 | gi\|1001328 | UDP-MurNac-tripeptide synthetase | 63 | 33 | |
| | | | | | [Synechocystis sp.] | | | |
| 432 | 4 | 1970 | 3016 | gi\|1161061 | dioxygenase [Methylobacterium extorquens] | 63 | 41 | |
| 463 | 2 | 1324 | 851 | gi\|1573163 | hypothetical [Haemophilus influenzae] | 63 | 40 | |
| 466 | 4 | 2843 | 3730 | gnl\|PID\|e261988 | putative ORF [Bacillus subtilis] | 63 | 41 | |
| 472 | 1 | 527 | 3 | gi\|556885 | Unknown [Bacillus subtilis] | 63 | 50 | |
| 517 | 3 | 2803 | 1646 | gi\|531265 | lipophilic protein which affects bacterial | 63 | 38 | |
| | | | | | lysis rate and ethicillin resistance level | | | |
| | | | | | [Staphylococcus aureus] pir\|A55856\|A55856 | | | |
| | | | | | llm protein - Staphylococcus aureus | | | |
| 538 | 1 | 206 | 3 | gi\|172657 | serine-protein kinase [Saccharomyces | 63 | 47 | |
| | | | | | cerevisiae] | | | |
| 539 | 4 | 2997 | 3851 | gi\|973230 | gamma-glutatnyl kinase [Lycopersicon | 63 | 43 | |
| | | | | | esculentum] | | | |
| 565 | 3 | 756 | 1010 | gi\|1303724 | YgaF [Bacillus subtilis] | 63 | 51 | |
| 573 | 7 | 4518 | 3709 | gi\|1652352 | dihydropteroate pyrophosphorylase | 63 | 45 | |
| | | | | | [Synechocystis sp.] | | | |
| 579 | 2 | 361 | 1344 | gi\|1573114 | beta-ketoacyl-acyl carrier protein | 63 | 41 | |
| | | | | | synthase III (fabH) [Haemophilus | | | |
| | | | | | influenzae] | | | |
| 593 | 2 | 390 | 1037 | gi\|409286 | bmrU [Bacillus subtilis] | 63 | 33 | |
| 707 | 1 | 647 | 171 | gi\|511596 | interleukin-2 [Canis familiaris] | 63 | 33 | |
| 714 | 1 | 2 | 268 | gnl\|PID\|e213832 | putative inner membrane protein [Bacillus | 63 | 38 | |
| | | | | | licheniformis] | | | |
| 724 | 1 | 562 | 239 | gnl\|PID\|e255315 | unknown [Mycobacterium tuberculosis] | 63 | 49 | |
| 759 | 1 | 681 | 4 | gi\|437639 | [Plasmodium falciparum 3′end.], gene | 63 | 28 | |
| | | | | | product [Plasmodium alciparum] | | | |
| 794 | 1 | 981 | 313 | gi\|451201 | ORF1 [Bacillus subtilis] | 63 | 37 | |
| 811 | 2 | 609 | 184 | gi\|150553 | regulatory protein [Plasmid pCF10] | 63 | 30 | |
| 835 | 1 | 2 | 262 | gi\|1736496 | RpiR protein. [Escherichia coli] | 63 | 41 | |
| 11 | 1 | 2 | 1144 | gi\|143150 | levR [Bacillus subtilis] | 62 | 48 | |
| 12 | 5 | 8710 | 7673 | gi\|1486244 | unknown ]Bacillus subtilis] | 62 | 43 | |
| 15 | 3 | 1167 | 2957 | gi\|1592101 | adenine deaminase [Methanococcus | 62 | 40 | |
| | | | | | jannaschii] | | | |
| 16 | 4 | 2572 | 4092 | gi\|1109685 | ProW [Bacillus subtilis] | 62 | 37 | |
| 23 | 4 | 1279 | 2067 | gi\|41432 | fepC gene product [Escherichia coli] | 62 | 35 | |
| 23 | 26 | 16176 | 16454 | gi\|154499 | carbon dioxide concentrating mechanism | 62 | 41 | |
| | | | | | protein [Synechococcus sp.] | | | |
| | | | | | pir\|C36904\|C36904 carbon dioxide | | | |
| | | | | | concentrating mechanism protein cmL - | | | |
| | | | | | Synechococcus sp. (PCC 7942) | | | |
| 31 | 6 | 5322 | 5774 | gi\|532309 | 25 kDa protein [Escherichia coli] | 62 | 38 | |
| 68 | 4 | 1606 | 2778 | gi\|1732203 | GlcNAc 6-P deacetylase [Vibrio furnissii] | 62 | 44 | |
| 72 | 1 | 1 | 540 | gi\|1573097 | glucosamine-6-phosphate deaminase protein | 62 | 26 | |
| | | | | | (nagB) [Haemophilus influenzae] | | | |
| 76 | 3 | 1937 | 2227 | gi\|928830 | ORF75; putative [Lactococcus lactis phage | 62 | 34 | |
| | | | | | BK5 -T] | | | |
| 83 | 16 | 11700 | 12272 | gi\|1592161 | N-terminal acetyltransferase complex, | 62 | 33 | |
| | | | | | subunit ARD1 [Methanococcus jannaschii] | | | |
| 83 | 19 | 12685 | 13737 | gi\|1653193 | sialoglycoprotease [Synechocystis sp.] | 62 | 42 | |
| 91 | 6 | 3232 | 3789 | gi\|1762962 | FemA [Staphylococcus simulans] | 62 | 37 | |
| 100 | 43 | 29676 | 29317 | gi\|963033 | orf1 gene product [Enterococcus hirae] | 62 | 45 | |
| 101 | 8 | 7410 | 6481 | gi\|1161061 | dioxygenase [Methylobacterium extorguens] | 62 | 45 | |
| 110 | 3 | 653 | 871 | gi\|992683 | mdm2-D [Homo sapiens] | 62 | 37 | |
| 110 | 8 | 8440 | 5810 | gi\|784897 | beta-N-acetylhexosaminidase [Streptococcus | 62 | 46 | |
| | | | | | pneumoniae] pir\|A56390\|A56390 mannosyl- | | | |
| | | | | | glycoprotein ndo-beta-N- | | | |
| | | | | | acetylglucosaminidase (EC 3.2.1.96) | | | |
| | | | | | precursor - treptococcus pneumoniae | | | |
| 111 | 2 | 1057 | 287 | gnl\|PID\|e253280 | ORF YDL238c [Saccharomyces cerevisiae] | 62 | 45 | |
| 114 | 5 | 6886 | 7662 | gi\|152719 | flavocytochrome c [Shewanella | 62 | 37 | |
| | | | | | putrefaciens] | | | |
| 115 | 4 | 1401 | 1994 | gi\|1303978 | YgkA [Bacillus subtilis] | 62 | 46 | |
| 118 | 1 | 545 | 225 | gi\|39431 | oligo-1,6-glucosidase [Bacillus cereus] | 62 | 40 | |
| 119 | 8 | 4625 | 4356 | gi\|1522673 | type I restriction enzyme [Methanococcus | 62 | 33 | |
| | | | | | jannaschii] | | | |
| 120 | 2 | 257 | 1270 | gnl\|PID\|e235823 | unknown [Schizosaccharomyces pombe] | 62 | 41 | |
| 121 | 8 | 7543 | 8034 | gi\|39475 | formamidopyrimidine-DNA glycosylase | 62 | 48 | |
| | | | | | [Bacillus firmus] ir\|A11489\|S11489 | | | |
| | | | | | formamidopyrimidine-DNA glycosidase (EC | | | |
| | | | | | 3.2.2.23) Bacillus firmus | | | |
| 123 | 2 | 1677 | 592 | gi\|882252 | conjugated bile acid hydrolase | 62 | 40 | |
| | | | | | [Clostridium perfringens] | | | |
| | | | | | sp\|P54965\|CBH_CLOPE CHOLOYLOLYCINE | | | |
| | | | | | HYDROLASE (EC 3.5.1.24) CONJUGATED BILE | | | |
| | | | | | ACID HYDROLASE) (CBAH) (BILE SALT | | | |
| | | | | | HYDROLASE). | | | |
| 128 | 16 | 10895 | 9408 | gi\|1742834 | PTS system, cellobiose-specific IIC | 62 | 43 | |
| | | | | | component (EIIC-CEL) (Cellobiose- permease | | | |
| | | | | | IIC component) (Phosphotransferase enzyme | | | |
| | | | | | II, C component) . [Escherichia coli] | | | |
| 128 | 29 | 24254 | 23544 | gi\|1518680 | minicell-associated protein DivIVA | 62 | 37 | |
| | | | | | [Bacillus subtilis] | | | |
| 128 | 35 | 28843 | 28103 | gi\|142940 | ftsA [Bacillus subtilis] | 62 | 42 | |
| 133 | 4 | 3434 | 4165 | gnl\|PID\|e235174 | unknown [Mycobacterium tuberculosis] | 62 | 38 | |
| 134 | 2 | 1679 | 933 | gi\|155032 | ORF B [Plasmid pEa34] | 62 | 36 | |
| 146 | 6 | 4923 | 4651 | gi\|153675 | tagatose 6-P kinase [Streptococcus mutans] | 62 | 48 | |
| 149 | 5 | 3318 | 2527 | gi\|1591587 | pantothenate metabolism flavoprotein | 62 | 35 | |
| | | | | | [Methanococcus jannaschii] | | | |
| 152 | 9 | 4830 | 5747 | gi\|1652461 | lactose transport system permease protein | 62 | 39 | |
| | | | | | LacF [Synechocystis sp.] | | | |
| 163 | 2 | 1341 | 544 | gi\|533098 | DnaD protein [Bacillus subtilis] | 62 | 41 | |
| 164 | 14 | 9567 | 9322 | gi\|1118060 | coded for by C. elegans cDNA yk3d11.5; | 62 | 27 | |
| | | | | | coded for by C. elegans cDNA yk5f4.5 | | | |
| | | | | | [Caenorhabditis elegans] | | | |
| 172 | 8 | 6613 | 7146 | gi\|915199 | ggaB [Bacillus subtilis] | 62 | 33 | |
| 173 | 13 11127 | 9736 | gi\|1653484 | hypothetical protein [Synechocystis sp.] | 62 | 44 | | |
| 177 | 1 | 1077 | 364 | gi\|1572994 | 2-keto-3-deoxy-6-phosphogluconate aldolase | 62 | 38 | |
| | | | | | (eda) [Haemophilus influenzae] | | | |
| 178 | 4 | 1683 | 1318 | gnl\|PID\|e155310 | Orf2 [Bacteriophage TP901-1] | 62 | 51 | |
| 179 | 5 | 6425 | 7576 | gi\|1161933 | DltB [Lactobacillus casei] | 62 | 44 | |
| 180 | 13 | 12470 | 10842 | sp\|P37047\|YAEG_ECO | HYPOTHETICAL 44.3 KD PROTEIN IN HTRA-DAPD | 62 | 38 | |
| | | | | LI | INTERGENIC REGION. | | | |
| 181 | 14 | 11649 | 10735 | gi\|1742758 | Shikimate 5-dehydrogenase (EC 1.1.1.25). | 62 | 41 | |
| | | | | | [Escherichia coli] | | | |
| 197 | 2 | 516 | 1442 | gi\|623476 | transcriptional activator [Providencia | 62 | 34 | |
| | | | | | stuartii] sp\|P43463\|AARP_PROST | | | |
| | | | | | TRANSCRIPTIONAL ACTIVATOR AARP. | | | |
| 206 | 5 | 2728 | 1790 | gnl\|PID\|e265638 | unknown [Mycobacterium tuberculosis] | 62 | 37 | |
| 210 | 2 | 938 | 2290 | gi\|528991 | unknown [Bacillus subtilis] | 62 | 41 | |
| 221 | 15 | 7083 | 7280 | gnl\|PID\|e219154 | K08F4.5 [Caenorhabditis elegans] | 62 | 44 | |
| 222 | 11 | 7141 | 8022 | gi\|537034 | ORF_o488 [Escherichia coli] | 62 | 39 | |
| 223 | 9 | 6924 | 6358 | gnl\|PID\|e283128 | unknown, highly similar to E. coli YecD | 62 | 42 | |
| | | | | | hypothtical 21.8 KD protein in aspS | | | |
| | | | | | 5′region and to isochorismatase [Bacillus | | | |
| | | | | | subtilis] | | | |
| 225 | 4 | 2055 | 2885 | gi\|18724 | pyrroline-5-carboxylate reductase (AA 1- | 62 | 39 | |
| | | | | | 274) [Glycine max] ir\|S10186\|S10186 | | | |
| | | | | | pyrroline-5-carboxylate reductase (EC | | | |
| | | | | | 1.5.1.2) - ybean | | | |
| 229 | 11 | 11428 | 10670 | gnl\|PID\|e235745 | hypothetical protein [Mycobacterium | 62 | 36 | |
| | | | | | leprae] | | | |
| 231 | 1 | 1244 | 3 | gi\|48808 | dciAE gene product [Bacillus subtilis] | 62 | 45 | |
| 233 | 1 | 801 | 4 | gi\|143391 | ORF2 [Bacillus subtilis] | 62 | 42 | |
| 233 | 13 | 10471 | 9431 | gi\|887825 | ORF_f541 [Escherichia coli] | 62 | 35 | |
| 242 | 1 | 3 | 149 | gi\|532549 | ORF16 [Enterococcus faecalis] | 62 | 44 | |
| 255 | 2 | 443 | 1009 | gi\|639789 | ORF9 [Mycoplasma pneumoniae] | 62 | 44 | |
| 266 | 6 | 2349 | 2158 | gnl\|PID\|e194945 | yeast sds22 homolog [Homo sapiens] | 62 | 37 | |
| 270 | 1 | 3 | 314 | gi\|1303827 | YqfI [Bacillus subtilis] | 62 | 35 | |
| 270 | 7 | 5136 | 4447 | gi\|1303958 | YgIG [Bacillus subtilis] | 62 | 41 | |
| 279 | 1 | 271 | 2 | gnl\|PID\|e185372 | ceuC gene product [Campylobacter coli] | 62 | 44 | |
| 301 | 11 | 9598 | 8798 | gi\|1303863 | YggP [Bacillus subtilis] | 62 | 45 | |
| 306 | 2 | 750 | 1202 | gi\|148771 | ribosomal protein HmaS4 [Haloarcula | 62 | 41 | |
| | | | | | marismortui] | | | |
| 308 | 3 | 2328 | 1684 | gnl\|PID\|e238666 | hypothetical protein [Bacillus subtilis] | 62 | 40 | |
| 309 | 5 | 8806 | 8573 | gi\|1591861 | M. jannaschii predicted coding region | 62 | 37 | |
| | | | | | MJ1230 [Methanococcus jannaschii] | | | |
| 318 | 3 | 2278 | 1283 | gi\|1256134 | YbbE [Bacillus subtilis] | 62 | 37 | |
| 321 | 3 | 1433 | 1792 | gi\|606080 | ORF_o290; Geneplot suggests frameshift | 62 | 37 | |
| | | | | | linking to o267, not found Escherichia | | | |
| | | | | | coli] | | | |
| 338 | 13 | 11175 | 12770 | gi\|467446 | similar to SpoVB [Bacillus subtilis] | 62 | 38 | |
| 345 | 11 | 10519 | 11793 | gi\|1736789 | Collagenase precursor (EC 3.4.-.-). | 62 | 40 | |
| | | | | | [Escherichia coli] | | | |
| 345 | 21 | 22459 | 22947 | gi\|1657794 | 6-hydroxymethyl-7,8-dihydropterin | 62 | 47 | |
| | | | | | pyrophosphokinase [Methylobacterium | | | |
| | | | | | extorguens] | | | |
| 358 | 1 | 902 | 36 | gi\|409241 | penicillin-binding protein 2 | 62 | 44 | |
| | | | | | [Staphylococcus aureus] | | | |
| 362 | 6 | 2930 | 3493 | gnl\|PID\|e255091 | hypothetical protein [Bacillus subtilis] | 62 | 37 | |
| 363 | 2 | 3242 | 1581 | gnl\|PID\|e254997 | hypothetical protein [Bacillus subtilis] | 62 | 40 | |
| 365 | 2 | 400 | 1770 | gi\|143150 | levR [Bacillus subtilis] | 62 | 42 | |
| 372 | 5 | 2525 | 4489 | gi\|1045736 | fructose-permease IIBC component | 62 | 43 | |
| | | | | | [Mycoplasma genitalium] | | | |
| 373 | 1 | 3 | 851 | gi\|438462 | transmembrane protein [Bacillus subtilis] | 62 | 36 | |
| 375 | 1 | 2 | 1336 | gi\|732813 | branched-chain amino acid carrier | 62 | 43 | |
| | | | | | [Lactobacillus delbrueckii] | | | |
| | | | | | pir\|S60180\|S60180 branched-chain amino | | | |
| | | | | | acid carrier brnQ - actobacillus | | | |
| | | | | | delbrueckii | | | |
| 375 | 3 | 2592 | 1831 | gi\|1644206 | unknown [Bacillus subtilis] | 62 | 43 | |
| 391 | 2 | 142 | 510 | gi\|151776 | ORF3 [Escherichia coli] | 62 | 31 | |
| 396 | 2 | 254 | 1051 | gi\|410131 | ORFX7 [Bacillus subtilis] | 62 | 41 | |
| 423 | 1 | 197 | 6 | pir\|A33592\|A33592 | repressor protein catM - Acinetobacter | 62 | 38 | |
| | | | | | calcoaceticus | | | |
| 436 | 1 | 704 | 3 | gi\|455376 | unidentified reading frame L (ORFL) | 62 | 32 | |
| | | | | | (putative); putative [Transposon n10] | | | |
| 466 | 8 | 9320 | 10480 | gi\|147402 | mannose permease subunit III-Man | 62 | 44 | |
| | | | | | [Escherichia coli] | | | |
| 488 | 5 | 2175 | 2927 | gi\|532546 | ORF13 [Enterococcus faecalis] | 62 | 40 | |
| 510 | 4 | 2572 | 3078 | gi\|43941 | EIII-B Sor PTS [Klebsiella pneumoniae] | 62 | 35 | |
| 517 | 2 | 1533 | 736 | gi\|559388 | epsX gene product [Acinetobacter | 62 | 53 | |
| | | | | | calcoaceticus] | | | |
| 519 | 1 | 2 | 1084 | gi\|1652876 | hypothetical protein [Synechocystis sp.] | 62 | 41 | |
| 535 | 1 | 353 | 69 | gi\|1196922 | unknown protein [Insertion sequence IS861] | 62 | 33 | |
| 579 | 1 | 1 | 363 | gi\|535052 | involved in protein secretion [Bacillus | 62 | 22 | |
| | | | | | subtilis] | | | |
| 656 | 5 | 5351 | 5956 | gnl\|PID\|e290931 | unknown [Mycobacterium tuberculosis] | 62 | 40 | |
| 666 | 1 | 445 | 128 | gi\|483940 | transcription regulator [Bacillus | 62 | 42 | |
| | | | | | subtilis] | | | |
| 682 | 1 | 597 | 172 | gi\|146724 | enzyme III-Man function protein (manX | 62 | 37 | |
| | | | | | (ptsL)) [Escherichia coli] gi\|41976 manX | | | |
| | | | | | gene product (AA 1-315) [Escherichia coli] | | | |
| 771 | 1 | 3 | 365 | gi\|1773086 | similar to S. typhimurium ProY | 62 | 44 | |
| | | | | | [Escherichia coli] | | | |
| 831 | 1 | 390 | 94 | gnl\|PID\|e255000 | hypothetical protein [Bacillus subtilis] | 62 | 55 | |
| 15 | 5 | 4421 | 5260 | gnl\|PID\|e214719 | PlcR protein [Bacillus thuringiensis] | 61 | 38 | |
| 16 | 6 | 4705 | 4938 | gi\|758425 | complement component C3 [Xenopus | 61 | 44 | |
| | | | | | laevis/gilli] | | | |
| 23 | 16 | 10279 | 11214 | sp\|P19265\|EUTC_SAL | ETHANOLAMINE ANMONIA-LYASE LIGHT CHAIN (EC | 61 | 46 | |
| | | | | TY | 4.3.1.7). | | | |
| 33 | 2 | 1789 | 2205 | gi\|413958 | ipa-34d gene product [Bacillus subtilis] | 61 | 36 | |
| 33 | 5 | 4756 | 6594 | gi\|1001823 | cadmium-transporting ATPase [Synechocystis | 61 | 38 | |
| | | | | | sp.] | | | |
| 37 | 4 | 2813 | 3295 | gi\|1256140 | YbbK [Bacillus subtilis] | 61 | 51 | |
| 37 | 7 | 5973 | 5215 | gnl\|PID\|e269488 | Unknown [Bacillus subtilis] | 61 | 33 | |
| 49 | 4 | 1567 | 1839 | gnl\|PID\|e139445 | major tail protein [Bacteriophage B1] | 61 | 43 | |
| 56 | 1 | 108 | 641 | gi\|1574067 | H. influenzae predicted coding region | 61 | 35 | |
| | | | | | H11034 [Haemophilus influenzae] | | | |
| 59 | 1 | 1 | 1002 | gi\|763513 | ORF4; putative [Streptomyces | 61 | 37 | |
| | | | | | violaceoruber] | | | |
| 69 | 7 | 4837 | 5523 | gnl\|PID\|e254877 | unknown [Mycobacterium tuberculosis] | 61 | 34 | |
| 72 | 11 | 9262 | 10476 | gi\|1591272 | ferrous iron transport protein B | 61 | 45 | |
| | | | | | [Methanococcus jannaschii] | | | |
| 83 | 2 | 731 | 1549 | gi\|755152 | highly hydrophobic integral membrane | 61 | 41 | |
| | | | | | protein [Bacillus subtilis] | | | |
| | | | | | sp\|P42953\|TAGG_BACSU TEICHOIC ACID | | | |
| | | | | | TRANSLOCATION PERMEASE PROTEIN AGG. | | | |
| 87 | 2 | 2067 | 925 | gi\|1573129 | hypothetical [Haemophilus influenzae] | 61 | 46 | |
| 103 | 5 | 2689 | 3495 | gi\|1685111 | orf1091 [Streptococcus thermophilus] | 61 | 45 | |
| 110 | 13 | 11455 | 11820 | gi\|100182S5 transcriptional repressor SmtB | 61 | 42 | | |
| | | | | | [Synechocystis sp.] | | | |
| 110 | 15 | 14048 | 12588 | gi\|1573583 | H. influenzae predicted coding region | 61 | 38 | |
| | | | | | H10594 [Haemophilus influenzae] | | | |
| 111 | 3 | 1675 | 1055 | gnl\|PID\|e253280 | ORF YDL238c [Saccharomyces cerevisiae] | 61 | 34 | |
| 111 | 4 | 1838 | 2518 | gi\|1574513 | hypothetical [Haemophilus influenzae] | 61 | 50 | |
| 111 | 5 | 2535 | 3158 | gi\|537235 | Kenn Rudd identifies as gpmB [Escherichia | 61 | 40 | |
| | | | | | coli] | | | |
| 121 | 1 | 3 | 1397 | gi\|290643 | ATPase [Enterococcus hirae] | 61 | 50 | |
| 123 | 28 | 25608 | 27734 | gi\|143150 | levR [Bacillus subtilis] | 61 | 39 | |
| 125 | 5 | 3455 | 2589 | gi\|148921 | LicD protein [Haemophilus influenzae] | 61 | 47 | |
| 128 | 14 | 9382 | 9146 | gi\|575361 | protein kinase PkpA [Phycomyces | 61 | 38 | |
| | | | | | blakesleeanus] | | | |
| 138 | 32 | 23151 | 21628 | gi\|1184262 | GadC [Shigella flexneri] | 61 | 34 | |
| 144 | 8 | 6311 | 5325 | gi\|710422 | cmp-binding-factor 1 [Staphylococcus | 61 | 39 | |
| | | | | | aureus] | | | |
| 171 | 4 | 4601 | 5566 | gi\|41500 | ORF 3 (AA 1-352); 38 kD (put. ftsX) | 61 | 31 | |
| | | | | | [Escherichia coli] | | | |
| 172 | 3 | 2006 | 2848 | gi\|303560 | ORF271 [Escherichia coli] | 61 | 42 | |
| 173 | 7 | 5146 | 6228 | gi\|1256134 | YbbE [Bacillus subtilis] | 61 | 31 | |
| 197 | 8 | 9183 | 8182 | gi\|143803 | GerC3 [Bacillus subtilis] | 61 | 33 | |
| 217 | 5 | 3007 | 3462 | gi\|1749414 | unnamed protein product | 61 | 43 | |
| | | | | | [Schizosaccharomyces pombe] | | | |
| 217 | 8 | 6099 | 5464 | gi\|143456 | rpoE protein (ttg start codon) [Bacillus | 61 | 37 | |
| | | | | | subtilis] | | | |
| 222 | 6 | 3400 | 3927 | gnl\|PID\|e255118 | hypothetical protein [Bacillus subtilis] | 61 | 41 | |
| 225 | 3 | 1946 | 981 | gi\|1574660 | xylose operon regluatory protein (xylR) | 61 | 43 | |
| | | | | | [Haemophilus influenzae] | | | |
| 237 | 2 | 203 | 952 | gi\|1019108 | alternate start at bp 59; ORF | 61 | 52 | |
| | | | | | [Bacteriophage phi-80] | | | |
| 237 | 7 | 3058 | 3279 | gnl\|PID\|e246904 | ORF YPL169c [Saccharomyces cerevisiae] | 61 | 32 | |
| 262 | 1 | 20 | 913 | gnl\|PID\|e214719 | PlcR protein [Bacillus thuringiensis] | 61 | 35 | |
| 271 | 17 | 12725 | 13504 | gi\|143057 | ORF39 [Bacillus subtilis] | 61 | 31 | |
| 275 | 8 | 5370 | 3697 | gi\|1542975 | AbcB [Thermoanaerobacterium | 61 | 41 | |
| | | | | | thermosulfurigenes] | | | |
| 280 | 2 | 692 | 3079 | gi\|1001352 | ABC transporter [Synechocystis sp.] | 61 | 42 | |
| 294 | 7 | 2276 | 2767 | gi\|662792 | single-stranded DNA binding protein | 61 | 44 | |
| | | | | | [unidentified eubacterium] | | | |
| 301 | 12 | 9965 | 9519 | gi\|1303861 | YqgN [Bacillus subtilis] | 61 | 41 | |
| 308 | 1 | 1471 | 26 | gi\|1276882 | EpsI [Streptococcus thermophilus] | 61 | 36 | |
| 314 | 2 | 475 | 1662 | gi\|975351 | PatB [Bacillus subtilis] | 61 | 42 | |
| 321 | 9 | 3762 | 4193 | gi\|1732202 | PTS permease for mannose subunit IIIMan N | 61 | 40 | |
| | | | | | terminal domain [Vibrio furnissii] | | | |
| 323 | 5 | 5118 | 5537 | gi\|532540 | ORF7 [Enterococcus faecalis] | 61 | 28 | |
| 324 | 7 | 4800 | 5156 | gi\|146122 | H-protein [Escherichia coli] | 61 | 39 | |
| 338 | 3 | 1456 | 1989 | pir\|A47071\|A47071 | orfi immediately 5′ of nifS - Bacillus | 61 | 43 | |
| | | | | | subtilis | | | |
| 341 | 2 | 342 | 947 | gi\|1736577 | Octopine transport system permease protein | 61 | 41 | |
| | | | | | OccM. [Escherichia coli] | | | |
| 349 | 3 | 1788 | 1363 | pir\|G64143\|G64143 | hypothetical protein HI0143 - Haemophilus | 61 | 38 | |
| | | | | | influenzae (strain Rd KW20) | | | |
| 369 | 2 | 1261 | 587 | gi\|153744 | ORF X; putative [Streptococcus mutans] | 61 | 33 | |
| 371 | 2 | 1801 | 1562 | gi\|48836 | xylulokinase [Staphylococcus xylosus] | 61 | 40 | |
| 372 | 4 | 1575 | 2543 | gi\|149395 | lacC [Lactococcus lactis] | 61 | 43 | |
| 379 | 11 | 12683 | 11727 | gi\|887829 | D21141 uses 2nd start; frame determined by | 61 | 40 | |
| | | | | | Lac fusion [Escherichia oli] | | | |
| 383 | 5 | 5625 | 3820 | gi\|624072 | similar to Escherichia coli | 61 | 36 | |
| | | | | | glycerophosphoryl diester | | | |
| | | | | | hosphodiesterase, Swiss-Prot Accession | | | |
| | | | | | Number p10908 [Paramecium ursaria | | | |
| | | | | | Chlorella virus 1] | | | |
| 395 | 2 | 771 | 517 | gnl\|PID\|e276251 | T23G11.6 [Caenorhabditis elegans] | 61 | 42 | |
| 399 | 20 | 15621 | 15812 | gi\|472527 | protein phosphatase 1 [Schizosaccharomyces | 61 | 44 | |
| | | | | | pombe] | | | |
| 413 | 1 | 3 | 749 | gnl\|PID\|e289144 | ywpE [Bacillus subtilis] | 61 | 42 | |
| 427 | 1 | 1079 | 288 | gi\|403373 | glycerophosphoryl diester | 61 | 42 | |
| | | | | | phosphodiesterase [Bacillus subtilis] | | | |
| | | | | | pir\|S37251\|S37251 glycerophosphoryl | | | |
| | | | | | diester phosphodiesterase - acillus | | | |
| | | | | | subtilis | | | |
| 436 | 4 | 2045 | 1761 | gi\|48669 | pot. ORF B [Shigella sonnei] | 61 | 38 | |
| 437 | 1 | 1158 | 244 | gi\|580866 | ipa-12d gene product [Bacillus subtilis] | 61 | 47 | |
| 482 | 2 | 1676 | 1167 | bbs\|158786 | 4A11 antigen, sperm tail membrane | 61 | 42 | |
| | | | | | antigen=putative sucrose-specific | | | |
| | | | | | phosphotransferase enzyme II homolog | | | |
| | | | | | [mice, testis, Peptide Partial, 172 aa] | | | |
| | | | | | [Mus sp.] | | | |
| 490 | 3 | 1291 | 1094 | gnl\|PID\|e248473 | putative phosphate permease [Arabidopsis | 61 | 35 | |
| | | | | | thaliana] | | | |
| 514 | 1 | 687 | 142 | gi\|1742775 | msm operon regulatory protein. | 61 | 36 | |
| | | | | | [Escherichia coli] | | | |
| 541 | 1 | 758 | 3 | gi\|1591732 | cobalt transport ATP-binding protein 0 | 61 | 39 | |
| | | | | | [Methanococcus jannaschii] | | | |
| 551 | 3 | 2163 | 1600 | gi\|671632 | unknown [Staphylococcus aureus] | 61 | 38 | |
| 603 | 2 | 163 | 564 | gi\|1408587 | relaxase [Lactococcus lactis lactis] | 61 | 39 | |
| 637 | 8 | 4539 | 4769 | gi\|143559 | subtilin [Bacillus subtilis] | 61 | 38 | |
| 765 | 1 | 34 | 681 | gi\|408888 | orfA 5′ of intG [Lactobacillus | 61 | 40 | |
| | | | | | bacteriophage phi adh] pir\|PN0468\|PN0468 | | | |
| | | | | | hypothetical protein 106 - Lactobacillus | | | |
| | | | | | gasseri fragment) | | | |
| 773 | 1 | 53 | 1207 | gi\|143841 | xylose repressor [Bacillus subtilis] | 61 | 36 | |
| 798 | 1 | 175 | 381 | gi\|187572 | located at OATL1 [Homo sapiens] | 61 | 32 | |
| 5 | 2 | 303 | 998 | gi\|1783264 | homologous to DNA glycosylases; | 60 | 50 | |
| | | | | | hypothetical [Bacillus subtilis] | | | |
| 8 | 8 | 5891 | 6550 | gi\|1777939 | Pfs [Treponema pallidum] | 60 | 40 | |
| 11 | 7 | 4096 | 4935 | gi\|147404 | mannose permease subunit II-M-Man | 60 | 41 | |
| | | | | | [Escherichia coli] | | | |
| 11 | 8 | 4919 | 5254 | gi\|467125 | glmS; L-Glucosamine:D-fructose-6-Phosphate | 60 | 30 | |
| | | | | | aminotransferase; 229_C3_238 | | | |
| | | | | | [Mycobacterium leprae] | | | |
| 17 | 9 | 7736 | 8203 | gi\|496514 | orf zeta [Streptococcus pyogenes] | 60 | 42 | |
| 20 | 1 | 3 | 443 | gi\|861137 | chitin binding protein [Streptomyces | 60 | 40 | |
| | | | | | olivaceoviridis] pir\|S55001\|S55001 CHB1 | | | |
| | | | | | protein - Streptomyces olivaceoviridis | | | |
| | | | | | {SUB −30} | | | |
| 21 | 3 | 1970 | 684 | gi\|1778520 | hypothetical protein [Escherichia coli] | 60 | 43 | |
| 23 | 11 | 5357 | 5953 | gi\|619066 | NAST [Azotobacter vinelandii] | 60 | 31 | |
| 34 | 4 | 6662 | 3279 | gi\|153952 | polymerase III polymerase subunit (dnaE) | 60 | 37 | |
| | | | | | [Salmonella typhimurium] | pir\|A45915\|A45915 | | |
| | | | | | DNA-directed DNA polymerase (EC 2.7.7.7) | | | |
| | | | | | III lpha chain - Salmonella typhimurium | | | |
| 39 | 1 | 47 | 466 | gi\|1561567 | Unknown [Bacillus subtilis] | 60 | 35 | |
| 39 | 4 | 1855 | 1361 | gi\|298045 | Orf154 [Streptomyces ambofaciens] | 60 | 41 | |
| 48 | 4 | 2554 | 4128 | gi\|1255259 | o-succinylbenzoic acid (OSB) CoA ligase | 60 | 40 | |
| | | | | | [Staphylococcus aureus] | | | |
| 56 | 9 | 6682 | 5795 | gi\|413940 | ipa-16d gene product [Bacillus subtilis] | 60 | 40 | |
| 65 | 3 | 2105 | 2593 | gi\|1573061 | hypothetical [Haemophilus influenzae] | 60 | 34 | |
| 72 | 9 | 7854 | 8330 | gi\|606343 | CG Site No. 28964 [Escherichia coli] | 60 | 39 | |
| 81 | 3 | 2053 | 1406 | gi\|1574770 | phenylalanyl-tRNA synthetase beta-subunit | 60 | 46 | |
| | | | | | (pheT) [Haemophilus influenzae] | | | |
| 81 | 4 | 2987 | 2130 | gi\|147404 | mannose permease subunit II-M-Man | 60 | 34 | |
| | | | | | [Escherichia coli] | | | |
| 81 | 12 | 8280 | 7150 | gnl\|PID\|e254984 | hypothetical protein [Bacillus subtilis] | 60 | 44 | |
| 83 | 22 | 16887 | 16537 | gi\|509672 | repressor protein [Bacteriophage Tuc2009] | 60 | 33 | |
| 89 | 1 | 698 | 60 | gi\|840838 | hypothetical 21.7 kDa protein in ftsY 5′ | 60 | 36 | |
| | | | | | region [Pseudomonas eruginosa] | | | |
| 89 | 12 | 12641 | 11856 | gi\|1377843 | unknown [Bacillus subtilis] | 60 | 40 | |
| 89 | 17 | 18879 | 15844 | gi\|666069 | orf2 gene product [Lactobacillus | 60 | 37 | |
| | | | | | leichmannii] | | | |
| 94 | 6 | 2281 | 3384 | gi\|468760 | ORF334 [Rhizobium meliloti] | 60 | 36 | |
| 98 | 1 | 12 | 1970 | gi\|1652892 | ABC transporter [Synechocystis sp.] | 60 | 38 | |
| 99 | 3 | 978 | 1460 | gi\|473955 | DNA-binding protein [Lactobacillus sp.] | 60 | 31 | |
| 100 | 35 | 26818 | 26333 | gi\|347851 | junctional sarcoplasmic reticulum | 60 | 48 | |
| | | | | | glycoprotein [Oryctolagus uniculus] | | | |
| 100 | 45 | 30072 | 30449 | gi\|143547 | Sin regulatory protein (ttg start codon) | 60 | 43 | |
| | | | | | [Bacillus subtilis] gi\|1303886 SinR | | | |
| | | | | | [Baciilus subtilis] | | | |
| 102 | 8 | 5923 | 6561 | gi\|1633572 | Herpesvirus saimiri ORF73 homolog | 60 | 25 | |
| | | | | | [Kaposi's sarcoma-associated herpes-like | | | |
| | | | | | virus] | | | |
| 109 | 1 | 362 | 3 | pir\|S10655\|S10655 | hypothetical protein X - Pyrococcus woesei | 60 | 33 | |
| | | | | | (fragment) | | | |
| 110 | 16 | 14806 | 14087 | pir\|JH0364\|JH0364 | hypothetical protein 176 (SAGP 5′ region) | 60 | 35 | |
| | | | | | - Streptococcus pyogenes | | | |
| 110 | 20 | 18929 | 18414 | gi\|142450 | ahrC protein [Bacillus subtilis] | 60 | 39 | |
| 110 | 21 | 19124 | 19624 | gi\|142450 | ahrC protein [Bacillus subtilis] | 60 | 40 | |
| 111 | 1 | 289 | 2 | gi\|1256618 | transport protein [Bacillus subtilis] | 60 | 31 | |
| 122 | 7 | 5627 | 9589 | gi\|217191 | 5′-nucleotidase precursor [Vibrio | 60 | 39 | |
| | | | | | parahaemolyticus] | | | |
| 123 | 5 | 4390 | 3659 | gi\|1197667 | vitellogenin [Anolis pulchellus] | 60 | 27 | |
| 123 | 20 | 18102 | 18407 | gi\|1303705 | YrkF [Bacillus subtilis] | 60 | 34 | |
| 128 | 32 | 26229 | 25492 | gi\|1652485 | hypothetical protein [Synechocystis sp.] | 60 | 29 | |
| 129 | 5 | 4421 | 6259 | gi\|1303853 | YggF [Bacillus subtilis] | 60 | 36 | |
| 131 | 2 | 1112 | 2338 | gi\|699112 | ugpC gene product [Mycobacterium leprae] | 60 | 41 | |
| 131 | 4 | 3194 | 4036 | gi\|296356 | putative membrane transport protein | 60 | 32 | |
| | | | | | [Clostridium perfringens] | | | |
| | | | | | pir\|A56641\|A56641 probable membrane | | | |
| | | | | | transport protein - Clostridium erfringens | | | |
| 131 | 8 | 6669 | 7901 | gi\|537054 | 2′,3′-cyclic-nucleotide 2′- | 60 | 40 | |
| | | | | | phosphodiesterase [Escherichia coli] | | | |
| | | | | | pir\|S56438\|s56438 2′,3′-cyclic-nucleotide | | | |
| | | | | | 2-phosphodiesterase (EC .1.4.16) - | | | |
| | | | | | Escherichia coli | | | |
| 133 | 11 | 9854 | 10240 | gnl\|PID\|e249654 | YneR [Bacillus subtilis] | 60 | 37 | |
| 138 | 7 | 6793 | 6263 | gi\|1486247 | unknown [Bacillus subtilis] | 60 | 48 | |
| 146 | 4 | 2831 | 2328 | gi\|39979 | P18 [Bacillus subtilis] | 60 | 38 | |
| 149 | 6 | 3504 | 3316 | gi\|145173 | 35 kDa protein [Escherichia coli] | 60 | 47 | |
| 154 | 5 | 2599 | 3558 | gi\|1773109 | similar to S. typhimurium apbA | 60 | 41 | |
| | | | | | [Escherichia coli] | | | |
| 155 | 5 | 3061 | 4701 | gi\|388269 | traC [Plasmid pAD1] | 60 | 38 | |
| 155 | 11 | 8565 | 8927 | gi\|1197460 | MtfB [Escherichia coli] | 60 | 39 | |
| 158 | 10 | 11123 | 10032 | gi\|581809 | tmbC gene product [Treponema pallidum] | 60 | 39 | |
| 165 | 7 | 6131 | 5700 | gi\|1439527 | EIIA-man [Lactobacillus curvatus] | 60 | 35 | |
| 172 | 4 | 3169 | 3810 | gi\|1001342 | hypothetical protein [Synechocystis sp.] | 60 | 42 | |
| 174 | 2 | 1574 | 762 | gi\|1045808 | hypothetical protein (GB:U00021_19) | 60 | 35 | |
| | | | | | [Mycoplasma genitalium] | | | |
| 181 | 7 | 4975 | 4460 | gi\|683584 | shikimate kinase [Lactococcus lactis] | 60 | 33 | |
| 183 | 6 | 2719 | 2955 | gi\|1146198 | ferredoxin [Bacillus subtilis] | 60 | 37 | |
| 189 | 2 | 3528 | 2221 | gi\|396301 | matches PS00041: Bacterial regulatory | 60 | 35 | |
| | | | | | proteins, araC family ignature | | | |
| | | | | | [Escherichia coli] | | | |
| 193 | 5 | 3121 | 2600 | gi\|39788 | adaB [Bacillus subtilis] | 60 | 49 | |
| 195 | 11 | 4623 | 6569 | gnl\|PID\|e250887 | potential coding region [Clostridium | 60 | 39 | |
| | | | | | difficile] | | | |
| 202 | 2 | 1837 | 1607 | gi\|693939 | membrane ATPase [Haloferax volcanii] | 60 | 32 | |
| 206 | 7 | 4794 | 3754 | gi\|1574702 | hypothetical [Haemophilus influenzae] | 60 | 42 | |
| 209 | 2 | 1308 | 433 | pir\|A38587\|A38587 | collagen, corneal - chicken (fragment) | 60 | 51 | |
| 220 | 3 | 4263 | 1213 | gi\|437706 | alternative truncated translation product | 60 | 41 | |
| | | | | | from E.coli [Streptococcus neumoniae] | | | |
| 222 | 9 | 6019 | 6522 | gi\|882463 | protein-N(pi)-phosphohistidine-sugar | 60 | 47 | |
| | | | | | phosphotransferase [Escherichia oli] | | | |
| 222 | 12 | 8001 | 8336 | gi\|537035 | ORF_o101 [Escherichia coli] | 60 | 33 | |
| 233 | 2 | 1294 | 827 | gi\|145091 | flavodoxin [Desulfovibrio salexigens] | 60 | 39 | |
| 242 | 11 | 7370 | 7627 | gi\|1353404 | cytochrome oxidase subunit I [Metridium | 60 | 28 | |
| | | | | | senile] | | | |
| 249 | 3 | 1109 | 1768 | gi\|143156 | membrane bound protein [Bacillus subtilis] | 60 | 41 | |
| 251 | 3 | 4053 | 1933 | gi\|1235662 | RfbC [Myxococcus xanthus] | 60 | 42 | |
| 256 | 4 | 2614 | 3867 | gi\|532612 | ecotropic retrovirus receptor [Mus | 60 | 37 | |
| | | | | | musculus] | | | |
| 260 | 2 | 1539 | 802 | gi\|1208447 | metahloprotease transporter [Serratia | 60 | 35 | |
| | | | | | marcescens] | | | |
| 261 | 5 | 4528 | 3179 | gnl\|PID\|e246728 | histidine kinase [Streptococcus gordonii] | 60 | 25 | |
| 269 | 3 | 2723 | 1563 | gi\|1591618 | M. jannaschii predicted coding region | 60 | 39 | |
| | | | | | MJ0951 [Methanococcus jannaschii] | | | |
| 269 | 4 | 3541 | 2780 | gi\|1303794 | YgeM [Bacillus subtilis] | 60 | 36 | |
| 269 | 11 | 7164 | 6595 | gi\|1303787 | YgeG [Bacillus subtilis] | 60 | 38 | |
| 271 | 2 | 677 | 1651 | gnl\|PID\|e269877 | riboflavin kinase [Bacillus subtilis] | 60 | 43 | |
| 271 | 3 | 1639 | 2247 | gi\|537148 | ORF_f181 [Escherichia coli] | 60 | 41 | |
| 271 | 18 | 13502 | 13762 | pir\|S3934\|S39341 | grpE protein - Lactococcus lactis | 60 | 40 | |
| 277 | 2 | 1662 | 979 | gi\|1773109 | similar to S. typhimurium apbA | 60 | 41 | |
| | | | | | [Escherichia coli] | | | |
| 279 | 13 | 10627 | 9773 | gi\|290545 | f270 [Escherichia coli] | 60 | 41 | |
| 290 | 2 | 790 | 1695 | gi\|152886 | elongation factor Ts (tsf) [Spiroplasma | 60 | 38 | |
| | | | | | citri] | | | |
| 291 | 4 | 3571 | 2612 | gnl\|PID\|e257610 | sugar-binding transport protein | 60 | 40 | |
| | | | | | [Anaerocellum thermophilum] | | | |
| 295 | 3 | 1309 | 2094 | gi\|1000453 | TreR [Bacillus subtilis] | 60 | 37 | |
| 301 | 15 | 11063 | 11344 | gi\|535274 | ORF1 [Streptococcus thermophilus] | 60 | 36 | |
| 310 | 3 | 2903 | 1266 | gi\|809765 | aspartate aminotransferase (AA 1-402) | 60 | 44 | |
| | | | | | [Sulfolobus solfataricus] | | | |
| | | | | | pir\|S07088\|S07088 aspartate transaminase | | | |
| | | | | | (EC 2.6.1.1) - Sulfolobus olfataricus | | | |
| 316 | 2 | 319 | 119 | bbs\|115298 | polyprotein(coat protein) [raspberry | 60 | 28 | |
| | | | | | ringspot virus RRV, Peptide, 1107 aa] | | | |
| | | | | | [Raspberry ringspot virus] | | | |
| 320 | 4 | 3085 | 2483 | gi\|143002 | proton glutamate symnport protein [Bacillus | 60 | 26 | |
| | | | | | caldotenax] pir\|S26246\|S26246 | | | |
| | | | | | glutamate/aspartate transport protein - | | | |
| | | | | | Bacillus aldotenax | | | |
| 323 | 1 | 1 | 681 | gi\|1477486 | transposase [Burkholderia cepacia] | 60 | 44 | |
| 330 | 4 | 3361 | 4488 | gi\|1778517 | glycerol dehydrogenase homolog | 60 | 48 | |
| | | | | | [Escherichia coli] | | | |
| 356 | 3 | 2471 | 2205 | gi\|57633 | neuronal myosin heavy chain [Rattus | 60 | 40 | |
| | | | | | rattus] | | | |
| 362 | 5 | 2458 | 2925 | gnl\|PID\|e255090 | hypothetical protein [Bacillus subtilis] | 60 | 36 | |
| 364 | 4 | 4096 | 5349 | gi\|1657522 | hypothetical protein [Escherichia coli] | 60 | 41 | |
| 383 | 1 | 654 | 4 | gn\|PID\|e288399 | F56H6.k [Caenorhabditis elegans] | 60 | 39 | |
| 383 | 2 | 2208 | 853 | gi\|143536 | sigma factor 54 [Bacillus subtilis] | 60 | 37 | |
| 386 | 2 | 130 | 510 | gi\|1046053 | hypothetical protein (SP:P32049) | 60 | 42 | |
| | | | | | [Mycoplasma genitalium] | | | |
| 399 | 26 | 25892 | 27757 | gi\|895747 | putative cel operon regulator [Bacillus | 60 | 30 | |
| | | | | | subtilis] | | | |
| 399 | 27 | 27721 | 28239 | gi\|146281 | gut operon activator (gutM) [Escherichia | 60 | 35 | |
| | | | | | coli] | | | |
| 401 | 4 | 2081 | 3523 | gi\|142833 | ORF2 [Bacillus subtilis] | 60 | 36 | |
| 405 | 2 | 1353 | 763 | gi\|633113 | ORF3 [Streptococcus sobrinus] | 60 | 42 | |
| 407 | 7 | 4380 | 4589 | gi\|1674126 | (AE000043) Mycoplasma pneumoniae, MG280 | 60 | 39 | |
| | | | | | homolog, from M. genitalium [Mycoplasma | | | |
| | | | | | pneumoniae] | | | |
| 408 | 1 | 12 | 539 | gi\|455006 | orf6 [Rhodococcus fascians] | 60 | 42 | |
| 421 | 7 | 4113 | 3925 | gi\|60020 | ORF31 (AA1-868) [Human herpesvirus 3] | 60 | 43 | |
| 452 | 3 | 712 | 2223 | gi\|532554 | ORF21 [Enterococcus faecalis] | 60 | 38 | |
| 462 | 3 | 2066 | 1551 | gi\|1015903 | ORE YJR151c [Sacoharomyces cerevisiae] | 60 | 37 | |
| 480 | 1 | 12 | 272 | gi\|468715 | sss gene product [Pseudomonas aeruginosa] | 60 | 34 | |
| 487 | 1 | 1091 | 3 | gi\|388269 | traC [Plasmid pAD1] | 60 | 39 | |
| 490 | 5 | 2108 | 1479 | gi\|699379 | glvr-1 protein [Mycobacterium leprae] | 60 | 29 | |
| 507 | 1 | 221 | 751 | gi\|1303952 | YqjA [Bacillus subtilis] | 60 | 37 | |
| 511 | 1 | 449 | 63 | gi\|391610 | farnesyl diphosphate synthase [Bacillus | 60 | 42 | |
| | | | | | stearothermophilus] pir\|JX0257\|JX0257 | | | |
| | | | | | geranyltranstransferase (EC 2.5.1.10) - | | | |
| | | | | | Bacillus tearothermophilus | | | |
| 551 | 2 | 1521 | 604 | gi\|1256648 | putative [Bacillus subtilis] | 60 | 37 | |
| 552 | 1 | 887 | 63 | gi\|537235 | Kenn Rudd identifies as gpmB [Escherichia | 60 | 40 | |
| | | | | | coli] | | | |
| 610 | 1 | 1 | 792 | gi\|1321625 | exo-alpha-1, 4-glucosidase [Bacillus | 60 | 45 | |
| | | | | | stearothermophilus] | | | |
| 642 | 1 | 402 | 214 | gi\|992964 | thioredoxin [Arabidopsis thaliana] | 60 | 36 | |
| 646 | 1 | 642 | 265 | gi\|1041115 | TRAC [Plasmid pPD1] | 60 | 32 | |
| 661 | 2 | 305 | 943 | gi\|1651536 | 3-oxoacyl-[acyl-carrier-protein] reductase | 60 | 37 | |
| | | | | | [Escherichia coli] | | | |
| 678 | 1 | 536 | 3 | gi\|532554 | ORF21 [Enterococcus faecalis] | 60 | 39 | |
| 716 | 1 | 799 | 305 | gi\|886040 | ORFtxel [Clostridium difficile] | 60 | 38 | |
| 717 | 1 | 2 | 472 | gi\|1402529 | ORF8 [Enterococcus faecalis] | 60 | 31 | |
| 727 | 1 | 516 | 82 | gi\|471283 | ORF [Synechococcus PCC6301] | 60 | 41 | |
| 770 | 1 | 327 | 4 | gi\|467451 | unknown [Bacillus subtilis] | 60 | 33 | |
| 843 | 1 | 234 | 4 | gi\|2819 | transferase (GAL10) (AA 1 - 687) | 60 | 37 | |
| | | | | | [Kluyveromyces lactis] r\|S01407\|XUVKG | | | |
| | | | | | UDPglucose 4-epimerase (EC 5.1.3.2) - | | | |
| | | | | | yeast uyveromyces marxianus var. lactis) | | | |
| 21 | 1 | 341 | 3 | gi\|1778519 | hypothetical protein [Escherichia coli] | 59 | 47 | |
| 23 | 2 | 290 | 1303 | gi\|1407800 | ABC-type permease [Yersinia pestis] | 59 | 36 | |
| 23 | 13 | 6720 | 7388 | gi\|1652472 | ethylene response sensor protein | 59 | 37 | |
| | | | | | [Synechocystis sp.] | | | |
| 23 | 18 | 11892 | 12413 | gi\|825627 | malor carboxysome shell protein | 59 | 42 | |
| | | | | | [Thiobacillus neapolitanus] | | | |
| | | | | | pir\|S60136\|S60136 malor carboxysome shell | | | |
| | | | | | protein - Thiobacillus eapolitanus | | | |
| 29 | 4 | 1989 | 2852 | gi\|1742383 | ORF_D:o276#3; similar to [PIR Accession | 59 | 48 | |
| | | | | | Number S11432] [Escherichia coli] | | | |
| 32 | 8 | 4504 | 4064 | gi\|1046081 | hypothetical protein (GB:D26185_10) | 59 | 33 | |
| | | | | | [Mycoplasma genitalium] | | | |
| 37 | 9 | 6670 | 6284 | gi\|290561 | o188 [Escherichia coli] | 59 | 44 | |
| 47 | 1 | 2 | 2743 | gnl\|PID\|e248792 | unknown [Mycobacterium tuberculosis] | 59 | 46 | |
| 48 | 5 | 4017 | 5492 | gi\|1185288 | isochorismate synthase [Bacillus subtilis] | 59 | 40 | |
| 49 | 5 | 1797 | 2093 | gi\|496280 | structural protein [Bacteriophage Tuc2009] | 59 | 41 | |
| 59 | 8 | 3324 | 5057 | gi\|1486244 | unknown \|Bacillus subtilis] | 59 | 35 | |
| 72 | 14 | 13937 | 13434 | gi\|532540 | ORF7 [Enterococcus faecalis] | 59 | 25 | |
| 81 | 20 | 14659 | 14219 | gi\|39978 | P16 [Bacillus subtilis] | 59 | 38 | |
| 98 | 2 | 1961 | 2617 | gi\|41519 | P30 protein (AA 1-240) [Escherichia coli] | 59 | 39 | |
| 102 | 3 | 2542 | 3774 | gi\|1674376 | (AE000062) Mycoplasma pneumoniae, MG148 | 59 | 30 | |
| | | | | | homolog, from M. genitalium [Mycoplasma | | | |
| | | | | | pneumoniae] | | | |
| 116 | 2 | 907 | 1458 | gi\|1146225 | putative [Bacillus subtilis] | 59 | 37 | |
| 116 | 7 | 3532 | 4842 | gi\|1146238 | poly(A) polymerase [Bacillus subtilis] | 59 | 41 | |
| 128 | 20 | 15626 | 14310 | gi\|1001719 | ATP-dependent RNA helicase DeaD | 59 | 34 | |
| | | | | | [Synechocystis sp.] | | | |
| 134 | 4 | 3158 | 3850 | gi\|1477486 | transposase [Burkholderia cepacia] | 59 | 40 | |
| 137 | 1 | 1 | 999 | gi\|1065948 | similar to thymidine diphosphoglucose 4,6- | 59 | 40 | |
| | | | | | dehydratase [Caenorhabditis elegans] | | | |
| 138 | 8 | 7489 | 6827 | gnl\|PID\|e264435 | Putative orf YCLX8c, len:192 | 59 | 36 | |
| | | | | | [Saccharomyces cerevisiae] | | | |
| 140 | 1 | 3 | 656 | gnl\|PID\|e254943 | unknown [Mycobacterium tuberculosis] | 59 | 32 | |
| 165 | 13 | 10427 | 9849 | gi\|1732199 | PTS permease for mannose subunit IIIMan C | 59 | 37 | |
| | | | | | terminal domain [Vibrio furnissii] | | | |
| 167 | 1 | 2 | 1045 | gi\|1573128 | hypothetical [Haemophilus influenzae] | 59 | 38 | |
| 173 | 2 | 430 | 2160 | gi\|1486244 | unknown [Bacillus subtilis] | 59 | 31 | |
| 179 | 10 | 10432 | 11199 | gi\|288299 | ORF1 gene product [Bacillus megaterium] | 59 | 34 | |
| 179 | 12 | 12117 | 13148 | gi\|1045964 | hypothetical protein (GB:U14003_297) | 59 | 41 | |
| | | | | | [Mycoplasma genitalium] | | | |
| 181 | 11 | 9684 | 8575 | gi\|1653152 | 3-dehydroquinate synthase [Synechocystis | 59 | 41 | |
| | | | | | sp.] | | | |
| 223 | 24 | 20736 | 21974 | gi\|1573051 | succinyl-diaminopimelate desuccinylase | 59 | 48 | |
| | | | | | (dapE) [Haemophilus influenzae] | | | |
| 229 | 12 | 12818 | 11421 | gi\|1652035 | fmu and fmv protein [Synechocystis sp.] | 59 | 39 | |
| 244 | 3 | 2836 | 1565 | gi\|1303959 | YqjH [Bacillus subtilis] | 59 | 45 | |
| 265 | 9 | 4116 | 3868 | gi\|311100 | translational activator [Saccharomyces | 59 | 28 | |
| | | | | | cerevisiae] | | | |
| 272 | 1 | 1 | 546 | gi\|490320 | Y gene product [unidentified] | 59 | 41 | |
| 279 | 16 | 14774 | 14370 | gi\|1389549 | ORF3 [Bacillus subtilis] | 59 | 46 | |
| 283 | 8 | 3222 | 3401 | gi\|153047 | lysostaphin (ttg start codon) | 59 | 43 | |
| | | | | | [Staphylococcus simulans] | | | |
| | | | | | pir\|A25881\|A25881 lysostaphin precursor - | | | |
| | | | | | Staphylococcus simulans | | | |
| | | | | | sp\|P10547\|LSTP_STASI LYSOSTAPHIN PRECURSOR | | | |
| | | | | | (EC 3.5.1.-). | | | |
| 288 | 5 | 2617 | 3144 | gi\|1142714 | phosphoenolpyruvate:mannose | 59 | 45 | |
| | | | | | phosphotransferase element IIB | | | |
| | | | | | [Lactobacillus curvatus] | | | |
| 292 | 19 | 14837 | 16792 | gi\|495646 | ATPase [Transposon Tn5422] | 59 | 40 | |
| 295 | 1 | 49 | 495 | gi\|533098 | DnaD protein [Bacillus subtilis] | 59 | 39 | |
| 315 | 2 | 907 | 653 | gi\|1574802 | hypothetical [Haemophilus influenzae\| | 59 | 38 | |
| 318 | 6 | 4549 | 4058 | gi\|43941 | EIII-B Sor PTS [Klebsiella pneumoniae] | 59 | 35 | |
| 345 | 3 | 2707 | 3507 | gi\|895749 | putative cellobiose phosphotransferase | 59 | 38 | |
| | | | | | enzyme II″ [Bacillus ubtilis] | | | |
| 351 | 5 | 2646 | 2371 | gi\|1666506 | RfbC [Leptospira interrogans] | 59 | 30 | |
| 355 | 21 | 15237 | 17222 | gi\|515738 | ORF2; putative [Oenococcus oeni] | 59 | 35 | |
| 384 | 1 | 14 | 754 | gi\|1162959 | homologous to HI0365 in Haemophilus | 59 | 34 | |
| | | | | | influenzae; ORF1 [Pseudomonas aeruginosa] | | | |
| 385 | 1 | 3 | 533 | gi\|1146197 | utative [Bacillus subtilis] | 59 | 36 | |
| 394 | 13 | 13137 | 12160 | gnl\|PID\|e243582 | ORF YGR263c [Saccharomyces cerevisiae] | 59 | 36 | |
| 399 | 1 | 224 | 580 | gi\|580904 | homologous to E.coli rnpA [Bacillus | 59 | 38 | |
| | | | | | subtilis] | | | |
| 412 | 1 | 3 | 2927 | gi\|1620648 | surface protein Rib [Streptococcus | 59 | 43 | |
| | | | | | agalactiae] | | | |
| 412 | 2 | 2918 | 3559 | gi\|1620648 | surface protein Rib [Streptococcus | 59 | 43 | |
| | | | | | agalactiae] | | | |
| 416 | 6 | 5283 | 3940 | gi\|1100076 | PTS-dependent enzyme II [Clostridium | 59 | 38 | |
| | | | | | longisporum] | | | |
| 437 | 2 | 1561 | 1136 | gi\|580866 | ipa-12d gene product [Bacillus subtilis] | 59 | 44 | |
| 495 | 2 | 438 | 614 | gi\|1500472 | M. jannaschii predicted coding region | 59 | 45 | |
| | | | | | MJ1577 [Methanococcus jannaschii] | | | |
| 502 | 1 | 853 | 188 | gi\|1063248 | No homologous protein [Bacillus subtilis] | 59 | 25 | |
| 573 | 8 | 5092 | 4493 | gi\|1573226 | hypothetical [Haemophilus influenzae] | 59 | 39 | |
| 579 | 4 | 1716 | 2717 | gnl\|PID\|e280724 | unknown [Mycobacterium tuberculosis] | 59 | 41 | |
| 600 | 1 | 1 | 504 | gi\|49386 | internal region of the penicillin-binding | 59 | 40 | |
| | | | | | protein 2B gene treptococcus pneumoniae] | | | |
| 616 | 3 | 904 | 533 | gi\|289265 | [Bacillus sp. (KSM 64) endo-1,4-beta- | 59 | 44 | |
| | | | | | glucanase gene, complete cds.], ene | | | |
| | | | | | products [Bacillus sp.] | | | |
| 657 | 1 | 432 | 4 | gi\|1651338 | PnuC protein [Escherichia coli] | 59 | 37 | |
| 699 | 1 | 416 | 165 | gnl\|PID\|e199096 | PepR1 [Lactobacillus deibrueckii] | 59 | 23 | |
| 713 | 4 | 3709 | 2660 | gi\|515738 | ORF2; putative [Oenococcus oeni] | 59 | 37 | |
| 715 | 1 | 698 | 84 | gi\|1176399 | EpiF [Staphylococcus epidermidis] | 59 | 42 | |
| 737 | 2 | 660 | 199 | gi\|666000 | hypothetical protein [Bacillus subtilis] | 59 | 43 | |
| 744 | 1 | 395 | 3 | gi\|1732057 | MUC.CL-1 [Trypanosoma cruzi] | 59 | 45 | |
| 746 | 1 | 3 | 554 | gi\|141858 | replication-associated protein [Plasmid | 59 | 36 | |
| | | | | | pAD1] | | | |
| 869 | 1 | 2 | 250 | gi\|1432153 | cellobiose-specific PTS permease | 59 | 40 | |
| | | | | | [Klebsiella oxytoca] | | | |
| 4 | 8 | 6948 | 6067 | gi\|147516 | ribokinase [Escherichia coli] | 58 | 42 | |
| 11 | 6 | 3312 | 4121 | gi\|1732200 | PTS permease for mannose subunit IIPMan | 58 | 35 | |
| | | | | [Vibrio furnissii] | | | | |
| 16 | 9 | 7684 | 6932 | gnl\|PID\|e233879 | hypothetical protein [Bacillus subtilis] | 58 | 48 | |
| 23 | 14 | 7440 | 8903 | gi\|142940 | ftsA [Bacillus subtilis] | 58 | 39 | |
| 30 | 2 | 570 | 1283 | gi\|1644202 | unknown [Bacillus subtilis] | 58 | 37 | |
| 48 | 7 | 7186 | 8037 | gi\|1573247 | hypothetical [Haemophilus influenzae] | 58 | 35 | |
| 49 | 7 | 2395 | 2871 | gnl\|PID\|e210884 | c2 gene product [Bacteriophage B1] | 58 | 34 | |
| 54 | 1 | 1014 | 91 | gi\|46645 | ORF (rlx) [Staphylococcus aureus] | 58 | 46 | |
| 55 | 3 | 1221 | 511 | gi\|726443 | No definition line found [Caenorhabditis | 58 | 41 | |
| | | | | | elegans] | | | |
| 58 | 1 | 1904 | 696 | gi\|1591564 | molybdenum cofactor biosynthesis moeA | 58 | 39 | |
| | | | | | protein [Methanococcus jannaschii] | | | |
| 58 | 8 | 7238 | 6996 | gi\|1279769 | FdhC [Methanobacterium thermoformicicum] | 58 | 54 | |
| 72 | 12 | 12117 | 10897 | gi\|763052 | integrase [Bacteriophage T270] | 58 | 37 | |
| 77 | 2 | 1155 | 1910 | gi\|1245464 | YfeA [Yersinia pestis] | 58 | 34 | |
| 78 | 1 | 2589 | 49 | gi\|40663 | sialidase [Clostridium septicum] | 58 | 40 | |
| 88 | 9 | 5854 | 6528 | gi\|1619623 | hemin binding protein [Yersinia | 58 | 37 | |
| | | | | | enterocolitica] | | | |
| 93 | 6 | 2639 | 2863 | gi\|405133 | putative [Bacillus subtilis] | 58 | 33 | |
| 98 | 13 | 13523 | 12432 | gi\|147329 | transport protein [Escherichia coli] | 58 | 41 | |
| 100 | 12 | 8550 | 8224 | gi\|1736642 | Invasin. [Escherichia coli] | 58 | 47 | |
| 102 | 7 | 5688 | 5969 | gi\|808869 | human gcp372 [Homo sapiens] | 58 | 30 | |
| 105 | 5 | 3716 | 4501 | gi\|143729 | transcription activator [Bacillus | 58 | 40 | |
| | | | | | subtilis] | | | |
| 107 | 1 | 511 | 2 | gi\|1303827 | YqfI [Bacillus subtilis] | 58 | 34 | |
| 108 | 2 | 1040 | 1732 | gi\|1592142 | ABC transporter, probable ATP-binding | 58 | 37 | |
| | | | | | subunit [Methanococcus jannaschii] | | | |
| 114 | 6 | 7608 | 8444 | gi\|152719 | flavocytochrome c [Shewanella | 58 | 40 | |
| | | | | | putrefaciens] | | | |
| 117 | 14 | 11813 | 11115 | gi\|1575577 | DNA-binding response regulator [Thermotoga | 58 | 42 | |
| | | | | | maritima] | | | |
| 122 | 1 | 1 | 936 | gi\|393269 | adhesion protein [Streptococcus | 58 | 38 | |
| | | | | | pneumoniae] | | | |
| 123 | 23 | 20379 | 21617 | gi\|1653948 | hypothetical protein [Synechocystis sp.] | 58 | 38 | |
| 133 | 8 | 7362 | 8480 | gi\|143498 | degS protein [Bacillus subtilis] | 58 | 38 | |
| 133 | 9 | 8437 | 9087 | gi\|143089 | iep protein [Bacillus subtilis] | 58 | 31 | |
| 138 | 3 | 3551 | 2898 | gi\|216114 | DNA polymerase [Bacteriophage SPO1] | 58 | 41 | |
| 138 | 5 | 5819 | 5049 | gnl\|PID\|e289148 | highly similar to phosphotransferase | 58 | 38 | |
| | | | | | system regulator [Bacillus subtilis] | | | |
| 138 | 17 | 11419 | 10379 | gi\|1674137 | (A5000044) Mycoplasma pneumnoniae, lipoate | 58 | 37 | |
| | | | | | protein ligase; similar to Swiss-Prot | | | |
| | | | | | Accession Number P32099, from E. coli | | | |
| | | | | | [Mycoplasma pneumnoniae] | | | |
| 139 | 8 | 5002 | 4808 | gi\|153607 | dpnD gene product [Streptococcus | 58 | 43 | |
| | | | | | pneumoniae] | | | |
| 146 | 9 | 7817 | 6627 | gi\|606076 | ORF_o384 [Escherichia coli] | 58 | 43 | |
| 150 | 10 | 7529 | 7894 | gi\|141852 | sialidase [Actinomyces viscosus] | 58 | 28 | |
| 152 | 10 | 5717 | 6637 | gi\|296356 | putative membrane transport protein | 58 | 36 | |
| | | | | | [Clostridium perfringens] | | | |
| | | | | | pir\|A56641\|A56641 probable membrane | | | |
| | | | | | transport protein - Clostridium erfringens | | | |
| 162 | 10 | 11009 | 11185 | gi\|42655 | pi protein [Escherichia coli] | 58 | 37 | |
| 164 | 3 | 1793 | 1608 | gi\|881499 | parathion hydrolase (phosphotriesterase)- | 58 | 41 | |
| | | | | | related protein [Mus usculus] | | | |
| 165 | 6 | 5640 | 4975 | gi\|1146190 | 2-keto-3-deoxy-6-phosphogluconate aldolase | 58 | 39 | |
| | | | | | [Bacillus subtilis] | | | |
| 165 | 10 | 9038 | 8199 | gi\|606080 | ORF_290; Geneplot suggests frameshift | 58 | 35 | |
| | | | | | linking to o267, not found Escherichia | | | |
| | | | | | coli] | | | |
| 168 | 1 | 1 | 657 | gi\|413930 | ipa-6d gene product [Bacillus subtilis] | 58 | 41 | |
| 170 | 1 | 923 | 234 | gi\|1573505 | hypothetical [Haemophilus influenzae] | 58 | 30 | |
| 176 | 1 | 1 | 1101 | gi\|1652379 | cation-transporting P-ATPase | 58 | 30 | |
| | | | | | [Synechocystis sp.] | | | |
| 180 | 12 | 10237 | 10410 | gi\|408123 | V-ATPase 14kD subunit peptide [Drosophila | 58 | 33 | |
| | | | | | melanogaster]pir\|S38436\|S38436 H+- | | | |
| | | | | | transporting ATPase (EC 3.6.1.35) 14K | | | |
| | | | | | chain - ruit fly (Drosophila melanogaster) | | | |
| 193 | 3 | 2077 | 1388 | gi\|1256633 | putative [Bacillus subtilis] | 58 | 39 | |
| 193 | 4 | 2602 | 2075 | gi\|147920 | 3-methyladenine-DNA glycosylase I (tag) | 58 | 33 | |
| | | | | | [Escherichia coli] | | | |
| 194 | 9 | 6492 | 5500 | sp\|P09997\|YIDA_ECO | HYPOTHETICAL 29.7 KD PROTEIN IN IBPA-GYRB | 58 | 38 | |
| | | | | LI | INTERGENIC REGION. | | | |
| 201 | 5 | 5152 | 4466 | gi\|755152 | highly hydrophobic integral membrane | 58 | 28 | |
| | | | | | protein [Bacillus subtilis] | | | |
| | | | | | sp\|P42953\|TAGG_BACSU TEICHOIC ACID | | | |
| | | | | | TRANSLOCATION PERMEASE PROTEIN AGG. | | | |
| 210 | 9 | 6546 | 7265 | gi\|466520 | pocR [Salmonella typhimurium] | 58 | 36 | |
| 220 | 1 | 3 | 569 | gi\|467441 | expressed at the end of exponential growyh | 58 | 38 | |
| | | | | | under condtions in which he enzymes of the | | | |
| | | | | | TCA cycle are repressed [Bacillus | | | |
| | | | | | subtilis] sp\|P14194\|CTC_BACSU GENERAL | | | |
| | | | | | STRESS PROTEIN CTC. {SUB 2-204} gi\|40219 | | | |
| | | | | | partial ctc gene product (AA 1-186) | | | |
| | | | | | [Bacillus subtilis] | | | |
| 222 | 10 | 6520 | 7143 | gi\|1674024 | (AE000033) Mycoplasma pneumoniae, | 58 | 41 | |
| | | | | | hypothetical protein (yjfS) homolog; | | | |
| | | | | | similar to Swiss-Prot Accession Number | | | |
| | | | | | P39301, from E. coli [Mycoplasma | | | |
| | | | | | pneumoniae] | | | |
| 233 | 7 | 4984 | 3944 | gi\|147806 | selenium metabolism protein [Escherichia | 58 | 45 | |
| | | | | coli] | | | | |
| 238 | 14 | 12128 | 12910 | gi\|1736468 | Pectin degradation repressor protein KdgR. | 58 | 37 | |
| | | | | | [Escherichia coli] | | | |
| 244 | 11 | 8102 | 7809 | gi\|467418 | unknown [Bacillus subtilis] | 58 | 37 | |
| 246 | 1 | 1 | 276 | gi\|65291 | receptor tyrosine kiase preprotein | 58 | 32 | |
| | | | | | [Xiphophorus sp.] ir\|S06142\|S06142 kinase- | | | |
| | | | | | related transforming protein (Tu) (EC | | | |
| | | | | | 7.1.-) precursor - southern platyfish | | | |
| 255 | 4 | 2927 | 2559 | gi\|1652384 | ABC transporter [Synechocystis sp.] | 58 | 41 | |
| 258 | 9 | 8025 | 8966 | gi\|147402 | mannose permease subunit III-Man | 58 | 35 | |
| | | | | | [Escherichia coli] | | | |
| 259 | 2 | 1801 | 893 | gi\|1591564 | molybdenum cofactor biosynthesis moeA | 58 | 39 | |
| | | | | | protein [Methanococcus jannaschii] | | | |
| 260 | 3 | 1754 | 2254 | gi\|580841 | F1 [Bacillus subtilis] | 58 | 38 | |
| 271 | 4 | 2382 | 2738 | gi\|40067 | X gene product [Bacillus sphaericus] | 58 | 37 | |
| 279 | 8 | 6237 | 6536 | gi\|1783243 | homologous to jojc gene product (B. | 58 | 34 | |
| | | | | | subtilis; prf:2111327a); hypothetical | | | |
| | | | | | [Bacillus subtilis] | | | |
| 301 | 1 | 753 | 175 | gi\|499196 | ORF1 [Streptomyces lincolnensis] | 58 | 37 | |
| 304 | 1 | 100 | 849 | gi\|1653322 | hypothetical protein [Synechocystis sp.] | 58 | 41 | |
| 313 | 2 | 748 | 1650 | gi\|1658371 | cyclic beta-1,2-glucan modification | 58 | 36 | |
| | | | | | protein [Rhizobium meliloti] | | | |
| 321 | 11 | 6033 | 6533 | gi\|1573292 | hypothetical [Haemophilus influenzae] | 58 | 34 | |
| 322 | 6 | 3819 | 5069 | gi\|23897 | 5′-nucleotidase [Homo sapiens] | 58 | 34 | |
| 324 | 5 | 3259 | 4452 | gi\|1469784 | putative cell division protein ftsW | 58 | 37 | |
| | | | | | [Enterococcus hirae] | | | |
| 328 | 1 | 1 | 270 | gi\|882579 | CG Site No. 29739 [Escherichia coli] | 58 | 43 | |
| 330 | 8 | 6228 | 6758 | gi\|43941 | EIII-B Sor PTS [Klebsiella pneumoniae] | 58 | 37 | |
| 334 | 4 | 3634 | 3963 | gi\|1001306 | hypothetical protein [Synechocystis sp.] | 58 | 34 | |
| 345 | 17 18899 | 20044 | gi\|853809 | ORF3 [Clostridium perfringens] | 58 | 30 | | |
| 363 | 7 | 8475 | 9944 | gi\|348056 | trans-acting positive regulator [Bacillus | 58 | 33 | |
| | | | | | anthracis] | | | |
| 375 | 7 | 6472 | 5279 | gi\|1408501 | homologous to N-acyl-L-amino acid | 58 | 42 | |
| | | | | | amidohydrolase of Bacillus | | | |
| | | | | | stearothermophilus [Bacillus subtilis] | | | |
| 394 | 12 | 10689 | 12095 | gi\|537034 | ORF_o488 [Escherichia coli] | 58 | 32 | |
| 399 | 3 | 1383 | 2198 | gi\|580905 | B.subtilis genes rpmH, rnpA, 50kd, gidA | 58 | 36 | |
| | | | | | and gidB [Bacillus subtilis]gi\|580919 Jag | | | |
| | | | | | [Bacillus subtilis] | | | |
| 399 | 16 | 11544 | 12098 | gi\|1572965 | hypothetical [Haemophilus influenzae] | 58 | 39 | |
| 399 | 19 | 14776 | 15654 | gi\|1778530 | CitG homolog [Escherichia coli] | 58 | 40 | |
| 407 | 2 | 738 | 553 | gi\|170553 | pyruvate kinase [Trichoderma reesei] | 58 | 38 | |
| 416 | 5 | 4045 | 3389 | gi\|475112 | enzyme IIabc [Pediococcus pentosaceus] | 58 | 41 | |
| 449 | 4 | 1421 | 879 | gi\|928834 | integrase [Lactococcus lactis phage BK5-T] | 58 | 32 | |
| 497 | 1 | 3 | 458 | gi\|160628 | reticulocyte binding protein 2 [Plasmodium | 58 | 30 | |
| | | | | | vivax] | | | |
| 594 | 1 | 285 | 4 | gi\|1353874 | unknown [Rhodobacter capsulatus] | 58 | 39 | |
| 637 | 6 | 3451 | 2765 | pir\|D61615\|D61615 | sericin MG-1 - greater wax moth (fragment) | 58 | 52 | |
| 653 | 1 | 595 | 245 | gi\|1408585 | LtrD [Lactococcus lactis lactis] | 58 | 41 | |
| 656 | 4 | 3713 | 5209 | sp\|P13692\|P54_ENTF | P54 PROTEIN PRECURSOR. | 58 | 37 | |
| | | | | C | | | | |
| 656 | 6 | 5988 | 6467 | gi\|1017818 | phosphotyrosine protein phosphatase | 58 | 48 | |
| | | | | | [Streptomyces coelicolor] | | | |
| 667 | 1 | 88 | 1467 | bbs\|177441 | OsNramp1=Nramp1 homolog/Bcg product | 58 | 40 | |
| | | | | | homolog [Oryza sativa, indica, cv. IR 36, | | | |
| | | | | | etiolated shoots, Peptide, 517 aa] [Oryza | | | |
| | | | | | sativa] | | | |
| 686 | 1 | 892 | 233 | pir\|A24255\|A24255 | chorion class A protein L11 precursor - | 58 | 38 | |
| | | | | | silkworm | | | |
| 706 | 1 | 1002 | 607 | gi\|1001762 | hypothetical protein [Synechocystis sp.] | 58 | 32 | |
| 801 | 1 | 254 | 12 | gnl\|PID\|e243641 | unknown [Mycobacterium tuberculosis] | 58 | 29 | |
| 848 | 1 | 212 | 3 | gnl\|PID\|e254644 | membrane protein [Streptococcus | 58 | 37 | |
| | | | | | pneumoniae] | | | |
| 975 | 1 | 3 | 422 | gi\|290545 | f270 [Escherichia coli] | 58 | 35 | |
| 11 | 4 | 2345 | 2833 | gi\|1439527 | EIIA-man [Lactobacillus curvatis] | 57 | 46 | |
| 16 | 2 | 1426 | 365 | gi\|780550 | acetyl transferase [Rhizobium loti] | 57 | 35 | |
| 18 | 3 | 1593 | 925 | gnl\|PID\|e137594 | xerC recombinase [Lactobacillus | 57 | 36 | |
| | | | | | leichmannii] | | | |
| 19 | 15 | 8058 | 8267 | gi\|1590922 | cell division inhibitor [Methanococcus | 57 | 42 | |
| | | | | | jannaschii] | | | |
| 19 | 23 | 11938 | 12318 | gi\|1294760 | structural protein; orfL3; putative | 57 | 46 | |
| | | | | | [Bacteriophage phi-41] | | | |
| 25 | 9 | 7743 | 6958 | gnl\|PID\|e255000 | hypothetical protein [Bacillus subtilis] | 57 | 40 | |
| 47 | 3 | 3857 | 4462 | gi\|1353540 | ORF23 [Bacteriophage rlt] | 57 | 35 | |
| 65 | 10 | 7100 | 8919 | gi\|496254 | fibronectin/fibrinogen-binding protein | 57 | 40 | |
| | | | | | [Streptococcus pyogenes] | | | |
| 68 | 7 | 3923 | 3705 | gi\|336656 | ribosomal protein secY [Cyanophora | 57 | 28 | |
| | | | | | paradoxa] | | | |
| 70 | 4 | 2317 | 3645 | pir\|S11158\|YESAEE | erythromycin resistance protein - | 57 | 40 | |
| | | | | | Staphylococcus epidermidis plasmid pULSOSO | | | |
| 76 | 1 | 55 | 1095 | gi\|1353562 | Structural protein [Bacteriophage rlt] | 57 | 41 | |
| 91 | 11 | 9070 | 8849 | gi\|550321 | beta-fructofuranosidase [Chenopodium | 57 | 30 | |
| | | | | | rubrum] | | | |
| 94 | 4 | 1740 | 1495 | gif 47406 | penicillin-binding protein 1a | 57 | 30 | |
| | | | | | [Streptococcus pneumoniae] | | | |
| | | | | | ir\|S28031\|528031 penicillin-binding | | | |
| | | | | | protein 1a - Streptococcus eumoniae | | | |
| | | | | | (strain 456) (fragment) | | | |
| 98 | 6 | 7766 | 6849 | gi\|409286 | bmrU [Bacillus subtilis] | 57 | 31 | |
| 100 | 22 | 17294 | 15912 | gnl\|PID\|e289150 | member of the SNF2 helicase family | 57 | 30 | |
| | | | | | [Bacillus subtilis] | | | |
| 102 | 1 | 66 | 2465 | gi\|405564 | traE [Plasmid pSK41] | 57 | 28 | |
| 110 | 14 | 11757 | 12497 | gi\|854601 | unknown [Schizosaccharomyces pombe] | 57 | 38 | |
| 114 | 9 | 10291 | 11139 | gi\|853777 | product similar to E.coli PRFA2 protein | 57 | 38 | |
| | | | | | [Bacillus subtilis] pir\|555438\|S55438 ywkE | | | |
| | | | | | protein - Bacillus subtilis | | | |
| | | | | | sp\|P45873\|HEMK_BACSU POSSIBLE | | | |
| | | | | | PROTOPORPHYRINOGEN OXIDASE (EC .3.3.-). | | | |
| 115 | 3 | 955 | 1461 | gi\|396347 | alternate name yjaB [Escherichia coli] | 57 | 33 | |
| 123 | 3 | 1925 | 2932 | gi\|1001731 | low affinity sulfate transporter | 57 | 39 | |
| | | | | | [Synechocystis sp.] | | | |
| 124 | 7 | 6026 | 5118 | gi\|1674310 | (AE000058) Mycoplasma pneumoniae, MG085 | 57 | 30 | |
| | | | | | homolog, from M. genitalium [Mycoplasma | | | |
| | | | | | pneumoniae] | | | |
| 128 | 9 | 7530 | 6235 | gi\|413940 | ipa-16d gene product [Bacillus subtilis] | 57 | 36 | |
| 128 | 31 | 25487 | 25206 | gi\|1651915 | hypothetical protein [Synechocystis sp.] | 57 | 42 | |
| 128 | 33 | 26878 | 26150 | gi\|1001387 | hypothetical protein [Synechocystis sp.] | 57 | 30 | |
| 128 | 37 | 30730 | 29600 | gi\|406877 | DivIB protein [Bacillus licheniformis] | 57 | 35 | |
| 130 | 9 | 7408 | 8556 | gi\|343539 | NADH dehydrogenase subunit 4 [Trypanosoma | 57 | 27 | |
| | | | | | brucei] | | | |
| 144 | 1 | 1013 | 219 | gi\|1652518 | hypothetical protein [Synechocystis sp.] | 57 | 45 | |
| 144 | 6 | 4145 | 5254 | gi\|149581 | maturation protein [Lactobacillus | 57 | 38 | |
| | | | | | paracasei] | | | |
| 146 | 1 | 617 | 192 | gi\|147402 | mannose permease subunit III-Man | 57 | 33 | |
| | | | | | [Escherichia coli] | | | |
| 153 | 1 | 83 | 991 | gi\|147336 | transmembrane protein [Escherichia coli] | 57 | 33 | |
| 160 | 8 | 4718 | 4134 | gi\|305333 | zeta-crystallin [Cavia porcellus] | 57 | 39 | |
| 167 | 8 | 14891 | 14688 | gi\|206354 | protein kinase C, zeta subspecies [Rattus | 57 | 39 | |
| | | | | | norvegicus] pir\|A30314\|A30314 protein | | | |
| | | | | | kinase C (EC 2.7.1.-) zeta - rat | | | |
| | | | | | sp\|P09217\|KPCZ_RAT PROTEIN KINASE C, ZETA | | | |
| | | | | | TYPE (EC 2.7.1.-) NPKC-ZETA). | | | |
| 174 | 1 | 760 | 2 | gnl\|PID\|e191403 | ORFA gene product [Chloroflexus | 57 | 42 | |
| | | | | | aurantiacus] | | | |
| 176 | 4 | 3347 | 3568 | gi\|1236529 | cyclomaltodextrinase [Bacillus sp.] | 57 | 46 | |
| 194 | 8 | 4786 | 5457 | gi\|405516 | This ORF is homologous to nitroreductase | 57 | 26 | |
| | | | | | from Enterobacter cloacae, ccession Number | | | |
| | | | | | A38686, and Salmonella, Accession Number | | | |
| | | | | | P15888 Mycoplasma-like organism] | | | |
| 199 | 3 | 3207 | 3764 | gi\|216350 | ORF [Bacillus subtilis] | 57 | 38 | |
| 202 | 5 | 3356 | 3664 | gi\|1183841 | Holliday junction binding protein | 57 | 34 | |
| | | | | | [Pseudomonas aeruginosa] | | | |
| 202 | 12 | 10911 | 10192 | gi\|971338 | anaerobic regulatory protein [Bacillus | 57 | 27 | |
| | | | | | subtilis] | | | |
| 205 | 3 | 1022 | 468 | gi\|1783240 | hypothetical [Bacillus subtilis] | 57 | 38 | |
| 223 | 2 | 779 | 1501 | gi\|1208965 | hypothetical 23.3 kd protein [Escherichia | 57 | 32 | |
| | | | | | coli] | | | |
| 223 | 3 | 1499 | 2332 | gi\|303560 | ORF271 [Escherichia coli] | 57 | 35 | |
| 223 | 11 | 8404 | 12198 | gi\|158079 | period protein [Drosophila serrata] | 57 | 40 | |
| 237 | 9 | 3685 | 3906 | gi\|514919 | phosphofructokinase [Drosophila | 57 | 31 | |
| | | | | | melanogaster] | | | |
| 242 | 7 | 5760 | 5020 | gi\|1574596 | H. influenzae predicted coding region | 57 | 33 | |
| | | | | | HI1738 [Haemophilus influenzae] | | | |
| 250 | 2 | 1243 | 1485 | gnl\|PID\|e275819 | K08G2.8 [Caenorhabditis elegans] | 57 | 47 | |
| 276 | 28 | 16565 | 16332 | gi\|886375 | variant-specific surface protein | 57 | 47 | |
| | | | | | [Plasmodium falciparum] | | | |
| 288 | 6 | 3157 | 3363 | gi\|147403 | mannose permease subunit II-P-Man | 57 | 39 | |
| | | | | | [Escherichia coli] | | | |
| 289 | 1 | 141 | 818 | gi\|1742822 | Phosphoglycolate phosphatase (EC | 57 | 40 | |
| | | | | | 3.1.3.18). [Escherichia coli] | | | |
| 292 | 20 | 15930 | 15721 | gi\|854201 | putative polymerase [Infectious bursal | 57 | 47 | |
| | | | | | disease virus] | | | |
| 294 | 4 | 1454 | 2014 | gi\|454303 | LDJ2 gene product [Allium porrum] | 57 | 41 | |
| 295 | 4 | 2052 | 2342 | pir\|S48588\|S48588 | hypothetical protein - Mycoplasma | 57 | 39 | |
| | | | | | capricolum (SGC3) (fragment) | | | |
| 301 | 14 | 10921 | 10148 | gnl\|PID\|e262045 | putative orf [Bacillus subtilis] | 57 | 38 | |
| 306 | 1 | 2 | 793 | gi\|216715 | HpaI methyltransferase [Haemophilus | 57 | 36 | |
| | | | | | parainfluenzae] pir\|S28681\|S28681 site- | | | |
| | | | | | specific DNA-methyltransferase adenine- | | | |
| | | | | | specific) (EC 2.1.1.72) HpaI - Haemophilus | | | |
| | | | | | parainfluenzae sp\|P29538\|MTH1_HAEPA | | | |
| | | | | | MODIFICATION METHYLASE HPAI (EC 2.1.1.72) | | | |
| | | | | | ADENINE-SPECIFIC MET | | | |
| 306 | 8 | 5418 | 5663 | gi\|1591542 | M. jannaschii predicted coding region | 57 | 42 | |
| | | | | | MJ0857 [Methanococcus jannaschii] | | | |
| 308 | 2 | 1732 | 1487 | gi\|1518045 | FlbF protein [Borrelia burgdorferi] | 57 | 28 | |
| 321 | 2 | 1030 | 1458 | gi\|606080 | ORF_o290; Geneplot suggests frameshift | 57 | 30 | |
| | | | | | linking to o267, not found Escherichia | | | |
| | | | | | coli] | | | |
| 351 | 4 | 2342 | 1587 | gi\|1591853 | M. jannaschii predicted coding region | 57 | 37 | |
| | | | | | MJ1222 [Methanococcus jannaschii] | | | |
| 355 | 30 | 20619 | 20861 | gi\|1136394 | There are three putative hydrophobic | 57 | 42 | |
| | | | | | domains in the central region. [Homo | | | |
| | | | | | sapiens] | | | |
| 364 | 10 | 9415 | 8852 | gi\|38722 | precursor (aa −20 to 381) [Acinetobacter | 57 | 32 | |
| | | | | | calcoaceticus] ir\|29277\|A29277 aldose 1- | | | |
| | | | | | epimerase (EC 5.1.3.3) - Acinetobacter | | | |
| | | | | | lcoaceticus | | | |
| 365 | 3 | 4715 | 1812 | gi\|914990 | Similar to DEAD box family helicases | 57 | 35 | |
| | | | | | [Saccharomyces cerevisiae] | | | |
| | | | | | pir\|S59797\|S59797 hypothetical protein | | | |
| | | | | | P9798.1 - yeast Saccharomyces cerevisiae) | | | |
| 378 | 1 | 615 | 10 | gi\|1652989 | hypothetical protein [Synechocystis sp.] | 57 | 35 | |
| 379 | 1 | 1457 | 114 | gi\|1256618 | transport protein [Bacillus subtilis] | 57 | 36 | |
| 390 | 1 | 1426 | 2 | gi\|387880 | collagen adhesin [Staphylococcus aureus] | 57 | 37 | |
| 422 | 1 | 2 | 409 | gi\|1591837 | M. jannaschii predicted coding region | 57 | 37 | |
| | | | | | MJ1207 [Methanococcus jannaschii] | | | |
| 447 | 1 | 397 | 131 | gi\|214566 | keratin protein XK81 [Xenopus laevis] | 57 | 33 | |
| 454 | 2 | 1095 | 889 | gi\|1783256 | sigma factor [Bacillus subtilis] | 57 | 28 | |
| 504 | 2 | 641 | 1426 | gi\|42081 | nagD gene product (AA 1-250) [Escherichia | 57 | 32 | |
| | | | | | coli] | | | |
| 524 | 2 | 963 | 577 | gi\|143724 | putative [Bacillus subtilis] | 57 | 43 | |
| 535 | 4 | 4862 | 4305 | gi\|146549 | kdpC [Escherichia coli] | 57 | 40 | |
| 547 | 2 | 426 | 719 | gi\|533098 | DnaD protein [Bacillus subtilis] | 57 | 33 | |
| 548 | 1 | 316 | 717 | gi\|397973 | Mg2+ transport ATPase [Salmonella | 57 | 33 | |
| | | | | | typhimurium] | | | |
| 639 | 2 | 359 | 105 | gnl\|PID\|e247390 | P-type ATPase [Dictyostelium discoideum] | 57 | 31 | |
| 641 | 1 | 941 | 180 | gnl\|PID\|e261990 | putative orf [Bacillus subtilis] | 57 | 36 | |
| 686 | 3 | 1298 | 3259 | gi\|496506 | orf gamma [Streptococcus pyogenes] | 57 | 37 | |
| 686 | 6 | 2200 | 2847 | gi\|404800 | putative [Saccharopolyspora erythraea] | 57 | 47 | |
| 782 | 2 | 591 | 860 | gi\|1591270 | alanyl-tRNA synthetase [Methanococcus | 57 | 32 | |
| | | | | | jannaschii] | | | |
| 844 | 1 | 3 | 182 | gi\|849217 | Weak similarity to Streptococcus Protein | 57 | 34 | |
| | | | | | V, a type-II IgG receptor PIR accession | | | |
| | | | | | number S17354) and Giardia lamblia median | | | |
| | | | | | body rotein (PIR accession number S33821) | | | |
| | | | | | [Saccharomyces cerevisiae] | | | |
| | | | | | pir\|S61181\|S61181 hypothetical protein | | | |
| | | | | | D9740.10 - yeast Sacchar | | | |
| 859 | 1 | 174 | 4 | gi\|1762584 | polygalacturonase isoenzyme 1 beta subunit | 57 | 28 | |
| | | | | | homolog [Arabidopsis thaliana] | | | |
| 967 | 1 | 381 | 4 | gi\|309662 | pheromone binding protein [Plasmid pCF10] | 57 | 40 | |
| 11 | 5 | 2817 | 3314 | gi\|43941 | EIII-B Sor PTS [Klebsiella pneumoniae] | 56 | 30 | |
| 15 | 1 | 80 | 892 | gi\|1574803 | spermidine/putrescine-binding periplasmic | 56 | 32 | |
| | | | | | protein precursor (potD) [Haemophilus | | | |
| | | | | | influenzae] | | | |
| 37 | 8 | 6327 | 6088 | gi\|290561 | o188 [Escherichia coli] | 56 | 41 | |
| 44 | 2 | 1169 | 1360 | gi\|16096 | peroxidase [Armoracia rusticana] | 56 | 37 | |
| 56 | 3 | 1881 | 1363 | gi\|49272 | Asparaginase [Bacillus licheniformis] | 56 | 33 | |
| 65 | 1 | 102 | 887 | gi\|1377832 | unknown [Bacillus subtilis] | 56 | 41 | |
| 75 | 9 | 5817 | 4306 | gi\|1235712 | polyprotein [Infectious pancreatic | 56 | 30 | |
| | | | | | necrosis virus] | | | |
| 83 | 7 | 3260 | 4051 | gi\|1652645 | phosphoglycolate phosphatase | 56 | 30 | |
| | | | | | [Synechocystis sp.] | | | |
| 95 | 3 | 1793 | 2389 | pir\|C53610\|C53610 | ntpE protein - Enterococcus hirae | 56 | 28 | |
| 100 | 3 | 5076 | 1915 | gi\|1353559 | ORF42 [Bacteriophage rlt] | 56 | 35 | |
| 100 | 16 | 10581 | 10369 | gi\|868224 | No definition line found [Caenorhabditis | 56 | 35 | |
| | | | | | elegans] | | | |
| 100 | 48 | 31841 | 32770 | gi\|460025 | ORF2, putative [Streptococcus pneumoniae] | 56 | 38 | |
| 108 | 5 | 4007 | 3336 | gi\|288301 | ORF2 gene product [Bacillus megaterium] | 56 | 34 | |
| 109 | 2 | 1032 | 325 | gi\|413976 | ipa-52r gene product [Bacillus subtilis] | 56 | 36 | |
| 119 | 7 | 3958 | 5304 | gi\|498842 | VirS [Clostridium perfringens] | 56 | 35 | |
| 123 | 32 | 29479 | 30345 | gi\|39981 | [Bacillus subtilis] | 56 | 38 | |
| 126 | 1 | 521 | 3 | gi\|147403 | mannose permease subunit II-P-Man | 56 | 29 | |
| | | | | | [Escherichia coli] | | | |
| 130 | 6 | 4296 | 6104 | gi\|308854 | oligopeptide binding protein [Lactococcus | 56 | 33 | |
| | | | | | lactis] | | | |
| 131 | 7 | 5267 | 6613 | gi\|466589 | CG Site No. 39 [Escherichia coli] | 56 | 32 | |
| 133 | 5 | 4358 | 5758 | gi\|1573431 | ammnodeoxychonismate lyase (pabC) | 56 | 40 | |
| | | | | | [Haemophilus influenzae] | | | |
| 138 | 20 | 13680 | 12670 | gi\|1590951 | UDP-glucose 4-epimerase [Methanococcus | 56 | 40 | |
| | | | | | jannaschii] | | | |
| 138 | 29 | 19764 | 18823 | gi\|44864 | H.8 outer membrane protein (AA −17 to 71) | 56 | 33 | |
| | | | | | [Neisseria gonorrhoeae] ir\|S02720\|S02720 | | | |
| | | | | | outer membrane protein H.8 precursor - | | | |
| | | | | | Neisseria norrhoeae | | | |
| 145 | 7 | 5611 | 7179 | gi\|1652892 | ABC transporter [Synechocystis sp.] | 56 | 33 | |
| 146 | 10 | 8545 | 7811 | gi\|41519 | P30 protein (AA 1-240) | [Escherichia coli] | 56 | 28 |
| 150 | 4 | 2979 | 4637 | gi\|309662 | pheromone binding protein [Plasmid pCF10] | 56 | 32 | |
| 159 | 5 | 5362 | 5066 | gi\|576733 | apocytochrome b [Trypanoplasma borreli] | 56 | 43 | |
| 164 | 13 | 8864 | 15031 | gi\|1654116 | protein F2 [Streptococcus pyogenes] | 56 | 43 | |
| 179 | 7 | 7790 | 9118 | gi\|413926 | ipa-2r gene product [Bacillus subtilis] | 56 | 33 | |
| 187 | 4 | 2239 | 1667 | gi\|1573061 | hypothetical [Haemophilis influenzae] | 56 | 18 | |
| 200 | 19 | 11473 | 10724 | gi\|498817 | ORF8; homologous to small subunit of phage | 56 | 35 | |
| | | | | | terminases [Bacillus ubtilis] | | | |
| 206 | 6 | 3766 | 2759 | gi\|474837 | ORF1 [Thermoanaerobacterium | 56 | 34 | |
| | | | | | thermosulfurigenes] sp\|P3854\|YAMB_THETU | | | |
| | | | | | HYPOTHETICAL 35.6 KD PROTEIN IN AMYB | | | |
| | | | | | 5′REGION ORF1). | | | |
| 207 | 2 | 2091 | 1672 | gi\|1204258 | soluble protein [Escherichia coli] | 56 | 40 | |
| 217 | 9 | 6661 | 6158 | gi\|1017427 | elastic titin [Homo sapiens] | 56 | 28 | |
| 225 | 7 | 6007 | 5099 | gi\|1742675 | Phosphotransferase system enzyme II (EC | 56 | 46 | |
| | | | | | 2.7.1.69) MalX [Escherichia coli] | | | |
| 230 | 3 | 595 | 3153 | gi\|437706 | alternative truncated translation product | 56 | 34 | |
| | | | | | from E.coli [Streptococcus neumoniae] | | | |
| 236 | 2 | 1486 | 515 | gi\|415664 | catabolite control protein [Bacillus | 56 | 35 | |
| | | | | | megaterium] sp\|P46828\|CCPA_BACME GLUCOSE- | | | |
| | | | | | RESISTANCE AMYLASE REGULATOR CATABOLITE | | | |
| | | | | | CONTROL PROTEIN). | | | |
| 236 | 7 | 9255 | 8599 | gi\|343544 | ATPase 6 [Trypanosoma brucei] | 56 | 48 | |
| 238 | 15 | 13059 | 13718 | gi\|1146190 | 2-keto-3-deoxy-6-phosphogluconate aldolase | 56 | 37 | |
| | | | | | [Bacillus subtilis] | | | |
| 238 | 20 | 17734 | 18756 | gi\|1574060 | hypothetical [Haemophilus influenzae] | 56 | 32 | |
| 238 | 23 | 21613 | 20726 | gi\|151361 | member of the AraC/XylS family of | 56 | 36 | |
| | | | | | transcriptional regulators Pseudomonas | | | |
| | | | | | aeruginosa] | | | |
| 242 | 6 | 4103 | 4477 | gi\|886858 | nicotinic acetylcholine receptor | 56 | 35 | |
| | | | | | [Caenorhabditis elegans] pir\|S57648\|S57648 | | | |
| | | | | | nicotinic acetylcholine receptor - | | | |
| | | | | | Caenorhabditis legans | | | |
| 260 | 5 | 3170 | 3781 | gnl\|PID\|e58151 | F3 [Bacillus subtilis] | 56 | 43 | |
| 279 | 6 | 5140 | 2831 | gi\|581100 | gamma-glutamylcysteine synthetase (aa 1- | 56 | 42 | |
| | | | | | 518) [Escherichia coli] pir\|A24136\|SYECEC | | | |
| | | | | | glutamate--cysteine ligase (EC 6.3.2.2) - | | | |
| | | | | | scherichia coli | | | |
| 279 | 9 | 6434 | 7228 | gi\|1783243 | homologous to jojC gene product (B. | 56 | 29 | |
| | | | | | subtilis; prf:2111327a); hypothetical | | | |
| | | | | | [Bacillus subtilis] | | | |
| 292 | 14 | 10719 | 11504 | gi\|45738 | ORFC [Enterococcus faecalis] | 56 | 37 | |
| 313 | 3 | 3039 | 1831 | gi\|474915 | orf 337; translated orf similarity to SW: | 56 | 31 | |
| | | | | | BCR_ECOLI bicyclomycin esistance protein | | | |
| | | | | | of Escherichia coli [Coxiella burnetii] | | | |
| | | | | | pir\|S44207\|44207 hypothetical protein 337 | | | |
| | | | | | - Coxiella burnetti {SUB -338} | | | |
| 313 | 5 | 4233 | 3589 | gi\|405883 | yeiL [Escherichia coli] | 56 | 30 | |
| 322 | 5 | 1994 | 3715 | gi\|1377831 | unknown [Bacillus subtilis] | 56 | 34 | |
| 353 | 2 | 2353 | 1310 | gnl\|PID\|e254644 | membrane protein [Streptococcus | 56 | 26 | |
| | | | | | pneumoniae] | | | |
| 394 | 14 | 13289 | 14143 | gi\|142836 | repressor protein [Bacillus subtilis] | 56 | 30 | |
| 399 | 32 | 30208 | 30891 | gi\|396293 | similar to Bacillus subtilis hypoth. 20 | 56 | 38 | |
| | | | | | kDa protein, in tsr 3′ egion [Escherichia | | | |
| | | | | | coli] | | | |
| 402 | 2 | 1267 | 914 | gi\|170710 | alpha-type gliadin precursor protein | 56 | 45 | |
| | | | | | [Triticum aestivum] | | | |
| 408 | 4 | 2825 | 2220 | gnl\|PID\|e257696 | collagen binding protein [Lactobacillus | 56 | 36 | |
| | | | | | reuteri] | | | |
| 432 | 5 | 3105 | 3302 | gi\|11678 | atpE gene product [Marchantia polymorpha] | 56 | 33 | |
| 443 | 2 | 844 | 1089 | gi\|1256138 | YbbI [Bacillus subtilis] | 56 | 36 | |
| 499 | 2 | 875 | 1666 | gi\|1499876 | magnesium and cobalt transport protein | 56 | 30 | |
| | | | | | [Methanococcus jannaschii] | | | |
| 510 | 6 | 3864 | 4733 | gi\|147404 | mannose permease subunit II-M-Man | 56 | 34 | |
| | | | | | [Escherichia coli] | | | |
| 543 | 6 | 3706 | 3113 | gi\|563812 | XCAP-C [Xenopus laevis] | 56 | 32 | |
| 609 | 2 | 390 | 653 | gi\|48745 | principal sigma subunit (AA 1-442) | 56 | 37 | |
| | | | | | [Streptomyces coelicolor] ir\|S11712\|S11712 | | | |
| | | | | | translation initiation factor sigma hrdB - | | | |
| | | | | | reptomyces coelicolor | | | |
| 626 | 2 | 1124 | 2104 | gi\|950197 | unknown [Corynebacterium glutamicum] | 56 | 40 | |
| 787 | 1 | 2 | 634 | gnl\|PID\|e283826 | orf c04012 [Sulfolobus solfataricus] | 56 | 26 | |
| 820 | 1 | 1220 | 3 | gi\|44001 | galactose-1-P-uridyl transferase | 56 | 35 | |
| | | | | | [Lactobacillus helveticus] | | | |
| | | | | | ir\|B47032\|B47032 galactose-1-phosphate | | | |
| | | | | | uridyl transferase - ctobacillus | | | |
| | | | | | helveticus | | | |
| 875 | 1 | 1 | 144 | gi\|455178 | 16K protein [Escherichia coli] | 56 | 46 | |
| 906 | 2 | 307 | 846 | gi\|144858 | ORF A [Clostridium perfringens] | 56 | 34 | |
| 941 | 1 | 3 | 335 | gi\|160299 | glutamic acid-rich protein [Plasmodium | 56 | 23 | |
| | | | | | falciparum] pir\|A54514\|A54514 glutamnic | | | |
| | | | | | acid-rich protein precursor - Plasmodium | | | |
| | | | | | alciparum | | | |
| 5 | 5 | 2451 | 2951 | gi\|1303811 | YgeU [Bacillus subtilis] | 55 | 39 | |
| 8 | 10 | 8312 | 7947 | gi\|1196907 | daunorubicin resistance protein | 55 | 29 | |
| | | | | | [Streptomyces peucetius] | | | |
| 17 | 24 | 23626 | 24465 | gnl\|PID\|e285322 | RecX rotein [Mycobacterium smegmatis] | 55 | 28 | |
| 17 | 31 | 31027 | 30344 | gi″143830 | xpaC [Bacillus subtilis] | 55 | 22 | |
| 17 | 34 | 31991 | 32302 | gnl\|PID\|e229183 | C11G6.3 [Caenorhabditis elegans] | 55 | 34 | |
| 30 | 1 | 2 | 478 | pir\|S10655\|S10655 | hypothetical protein X - Pyrococcus woesei | 55 | 34 | |
| | | | | | (fragment) | | | |
| 49 | 14 | 9998 | 10411 | gi\|455154 | ORE D [Clostridium perfringens] | 55 | 36 | |
| 54 | 3 | 955 | 1332 | gnl\|PID\|e238660 | hypothetical protein [Bacillus subtilis] | 55 | 32 | |
| 54 | 10 | 3527 | 3231 | pir\|JQ0405\|JQ0405 | hypothetical 119.5K protein (uvrA region) | 55 | 45 | |
| | | | | | - Micrococcus luteus | | | |
| 67 | 4 | 2313 | 3044 | gi\|555750 | unknown [Neisseria gonorrhoeae] | 55 | 42 | |
| 69 | 4 | 2250 | 2020 | gnl\|PID\|e259955 | K04G11.5 [Caenorhabditis elegans] | 55 | 33 | |
| 77 | 5 | 3954 | 2938 | gi\|1001634 | hypothetical protein [Synechocystis sp.] | 55 | 34 | |
| 80 | 4 | 4806 | 2482 | gi\|466952 | B1620_F1_30 [Mycobacterium leprae] | 55 | 35 | |
| 81 | 6 | 4212 | 3730 | gi\|606073 | ORF_o169 [Escherichia coli] | 55 | 34 | |
| 83 | 1 | 66 | 737 | gi\|216064 | morphogenesis protein B [Bacteriophage | 55 | 36 | |
| | | | | | PZA] | | | |
| 89 | 10 | 9486 | 7714 | gi\|148221 | DNA-dependent ATPase, DNA helicase | 55 | 35 | |
| | | | | | [Escherichia coli] pir\|JS0137\|BVECRQ recQ | | | |
| | | | | | protein - Escherichia coli | | | |
| 91 | 5 | 2507 | 3289 | gi\|153015 | FemA protein [Staphylococcus aureus] | 55 | 35 | |
| 100 | 14 | 9974 | 9393 | gi\|558603 | synaptonemal complex protein 1 [Mus | 55 | 30 | |
| | | | | | musculus] | | | |
| 116 | 1 | 1 | 909 | gi\|473901 | ORF1 [Lactococcus lactis] | 55 | 33 | |
| 122 | 3 | 1801 | 2655 | gi\|1016216 | putative protein of 299 amino acids | 55 | 28 | |
| | | | | | [Cyanophora paradoxa] | | | |
| 123 | 30 | 28191 | 28721 | gi\|1142714 | phosphoenolpyruvate:mannose | 55 | 29 | |
| | | | | | phosphotransferase element IIB | | | |
| | | | | | [Lactobacillus curvatus] | | | |
| 128 | 22 | 16664 | 16029 | gi\|606025 | ORF_o221 [Escherichia coli] | 55 | 42 | |
| 150 | 7 | 5949 | 6521 | gi\|39573 | P20 (AA 1-178) [Bacillus licheniformis] | 55 | 32 | |
| 155 | 7 | 5767 | 6660 | gi\|1763974 | DPPA [Bacillus methanolicus] | 55 | 31 | |
| 157 | 1 | 867 | 70 | gi\|1067010 | M153.1 [Caenorhabditis elegans] | 55 | 34 | |
| 160 | 9 | 6090 | 4804 | gi\|1592141 | M. jannaschii predicted coding region | 55 | 31 | |
| | | | | | MJ1507 [Methanococcus jannaschii] | | | |
| 176 | 3 | 2060 | 3349 | gi\|153858 | wall-associated protein [Streptococcus | 55 | 37 | |
| | | | | | mutans] | | | |
| 201 | 2 | 3277 | 413 | gi\|1235662 | RfbC [Myxococcus xanthus] | 55 | 36 | |
| 202 | 9 | 6199 | 8001 | gi\|606018 | ORF_o783 [Escherichia coli] | 55 | 42 | |
| 222 | 7 | 4803 | 4021 | gnl\|PID\|e289148 | highly similar to phosphotransferase | 55 | 40 | |
| | | | | | system regulator [Bacillus subtilis] | | | |
| 238 | 12 | 11465 | 9942 | gnl\|PID\|e266573 | unknown [Mycobacterium tuberculosis] | 55 | 27 | |
| 238 | 13 | 11527 | 12027 | gi\|1129093 | unknown protein [Bacillus sp.] | 55 | 36 | |
| 240 | 4 | 1988 | 1215 | gnl\|PID\|e252616 | DcuC protein [Escherichia coli] | 55 | 34 | |
| 246 | 2 | 433 | 792 | gnl\|PID\|e233868 | hypothetical protein [Bacillus subtilis] | 55 | 25 | |
| 253 | 5 | 1827 | 1549 | gi\|142540 | aspartokinase II [Bacillus sp.] | 55 | 48 | |
| 259 | 1 | 895 | 74 | gi\|1006621 | molybdate-binding periplasmic protein | 55 | 37 | |
| | | | | | [Synechocystis sp.] | | | |
| 267 | 1 | 1183 | 2 | gi\|882672 | ORF_o313 [Escherichia coli] | 55 | 27 | |
| 292 | 16 | 12843 | 13325 | gi\|561746 | cyclin-dependent protein kinase [Mus | 55 | 26 | |
| | | | | | musculus] | | | |
| 294 | 9 | 3390 | 3752 | gi\|984582 | DinJ [Escherichia coli] | 55 | 26 | |
| 300 | 5 | 3914 | 3582 | gi\|1591957 | M. jannaschii predicted coding region | 55 | 38 | |
| | | | | | MJ1318 [Methanococcus jannaschii] | | | |
| 305 | 3 | 2769 | 3527 | gi\|606309 | ORF_o265; gtg start [Escherichia coli] | 55 | 36 | |
| 320 | 6 | 4479 | 3475 | gi\|1591732 | cobalt transport ATP-binding protein O | 55 | 32 | |
| | | | | | [Methanococcus jannaschii] | | | |
| 355 | 24 | 18149 | 18322 | gi\|344751 | MDV TK gene product [unidentified] | 55 | 40 | |
| 364 | 2 | 2083 | 386 | gi\|1573045 | hypothetical [Haemophilus influenzae] | 55 | 40 | |
| 364 | 9 | 8796 | 8575 | gnl\|PID\|e252108 | ORF YOR255w [Saccharomyces cerevisiae] | 55 | 27 | |
| 379 | 8 | 8248 | 6872 | gi\|1330236 | dihydropyrimidinase [Homo sapiens] | 55 | 37 | |
| 386 | 6 | 3847 | 4332 | gi\|976025 | HrsA [Escherichia coli] | 55 | 27 | |
| 441 | 2 | 939 | 1730 | gi\|144859 | ORF B [Clostridium perfringens] | 55 | 28 | |
| 482 | 6 | 3515 | 3156 | gi\|606162 | ORF_f229 [Escherichia coli] | 55 | 39 | |
| 497 | 9 | 4885 | 5937 | gi\|1041637 | replication initiator protein | 55 | 33 | |
| | | | | | [Staphylococcus xylosus] | | | |
| 546 | 1 | 1 | 1104 | gi\|467446 | similar to SpoVB [Bacillus subtilis] | 55 | 36 | |
| 634 | 4 | 2132 | 1524 | gi\|431950 | similar to a B.subtilis gene (GB: | 55 | 27 | |
| | | | | | BACHEMEHY_5) [Clostridium asteurianum] | | | |
| 660 | 2 | 249 | 401 | gnl\|PID\|e254995 | hypothetical protein [Bacillus subtilis] | 55 | 35 | |
| 671 | 1 | 288 | 58 | gi\|38722 | precursor (aa −20 to 381) [Acinetobacter | 55 | 33 | |
| | | | | | calcoaceticus] ir\|A29277\|A29277 aldose 1- | | | |
| | | | | | epimerase (EC 5.1.3.3) - Acinetobacter | | | |
| | | | | | lcoaceticus | | | |
| 686 | 2 | 245 | 1141 | gi\|1633572 | Herpesvirus saimiri ORF73 homolog | 55 | 36 | |
| | | | | | [Kaposi's sarcoma-associated herpes-like | | | |
| | | | | | virus] | | | |
| 713 | 3 | 2742 | 1438 | gnl\|PID\|e8901 | RESA NF7 Ag13 [Plasmodium falciparum] | 55 | 25 | |
| 815 | 1 | 2 | 226 | gi\|1113815 | histidine kinase [Borrelia burgdorferi] | 55 | 36 | |
| 857 | 1 | 2 | 520 | gi\|143024 | glucose-resistance amylase regulator | 55 | 31 | |
| | | | | | [Bacillus subtilis] pir\|515318\|S15318 ccpA | | | |
| | | | | | protein - Bacillus subtilis | | | |
| | | | | | sp\|P25144 CCPA_BACSU GLUCOSE-RESISTANCE | | | |
| | | | | | AMYLASE REGULATOR CATABOLITE CONTROL | | | |
| | | | | | PROTEIN). | | | |
| 931 | 1 | 3 | 557 | gi\|1098508 | putative spore germination apparatus | 55 | 32 | |
| | | | | | protein [Bacillus megaterium] | | | |
| 17 | 7 | 6379 | 7218 | gnl\|PID\|e250887 | potential coding region [Clostridium | 54 | 35 | |
| | | | | | difficile] | | | |
| 21 | 9 | 7265 | 6348 | gi\|13441 | NADH dehydrogenase subunit 4L [Phoca | 54 | 29 | |
| | | | | | vitulina] | | | |
| 28 | 2 | 2727 | 3425 | gi\|1001792 | hypothetical protein [Synechocystis sp.] | 54 | 29 | |
| 32 | 6 | 4044 | 3523 | gi\|1673660 | (AE000002) Mycoplasma pneumoniae, | 54 | 36 | |
| | | | | | hypothetical 28K protein; similar to | | | |
| | | | | | GenBank Accession Number JS0068, from M. | | | |
| | | | | | pneumoniae [Mycoplasma pneumoniae] | | | |
| 33 | 3 | 2274 | 3767 | gnl\|PID\|e245024 | unknown [Mycobacterium tuberculosis] | 54 | 36 | |
| 40 | 1 | 1 | 915 | gi\|773349 | BirA protein [Bacillus subtilis] | 54 | 32 | |
| 49 | 6 | 2120 | 2485 | gnl\|PID\|e139446 | a2 gene product [Bacteriophage Bi] | 54 | 38 | |
| 54 | 17 | 8969 | 8661 | gi\|334068 | ORF2 [Suid herpesvirus 1] | 54 | 51 | |
| 65 | 2 | 1311 | 2120 | gi\|537207 | ORF_277 [Escherichia coli] | 54 | 27 | |
| 72 | 20 | 21986 | 22435 | gi\|928848 | ORF70′; putative [Lactococcus lactis phage | 54 | 34 | |
| | | | | | BK5-T] | | | |
| 105 | 4 | 3039 | 3827 | gnl\|PID\|e205174 | orf2 gene product [Lactobacillus | 54 | 30 | |
| | | | | | helveticus] | | | |
| 127 | 1 | 884 | 150 | gi\|726443 | No definition line found [Caenorhabditis | 54 | 31 | |
| | | | | | elegans] | | | |
| 148 | 1 | 1204 | 62 | gi\|467456 | unknown [Bacillus subtilis] | 54 | 37 | |
| 156 | 4 | 4360 | 3167 | gi\|1032483 | unidentified ORF downstream of hydrogenase | 54 | 30 | |
| | | | | | cluster; ORF5 [Anabaena variabilis] | | | |
| 160 | 4 | 1523 | 2077 | gnl\|PID\|e255111 | hypothetical protein [Bacillus subtilis] | 54 | 27 | |
| 160 | 7 | 4260 | 3745 | gi\|1184121 | auxin-induced protein [Vigna radiata] | 54 | 30 | |
| 165 | 5 | 4996 | 3971 | gi\|1772652 | 2-keto-3-deoxygluconate kinase [Haloferax | 54 | 36 | |
| | | | | | alicantei] | | | |
| 176 | 2 | 1044 | 1937 | gi\|162201 | P-type ATPase [Trypanosoma brucei] | 54 | 38 | |
| 180 | 29 | 30833 | 29853 | gnl\|PID\|e254644 | membrane protein [Streptococcus | 54 | 29 | |
| | | | | | pneumoniae] | | | |
| 200 | 16 | 7933 | 6656 | gi\|1574238 | traN protein (traN) [Haemophilus | 54 | 31 | |
| | | | | | influenzae] | | | |
| 206 | 1 | 232 | 2 | gi\|1220501 | Rickettsia tsutsugamushi (strain Kp47) | 54 | 31 | |
| | | | | | gene, complete cds [Rickettsia | | | |
| | | | | | tsutsugamushi] | | | |
| 220 | 4 | 5235 | 4342 | gi\|606080 | ORF_o290; Geneplot suggests frameshift | 54 | 31 | |
| | | | | | linking to o267, not found Escherichia | | | |
| | | | | | coli] | | | |
| 220 | 5 | 5821 | 5135 | gi\|43942 | first subunit of EII-Sor [Klebsiella | 54 | 36 | |
| | | | | | pneumoniae] | | | |
| 223 | 20 | 17253 | 17747 | gi\|47932 | tonB protein [Salmonella typhimurium] | 54 | 38 | |
| 228 | 7 | 4866 | 4033 | gi\|1736828 | Thi4 protein [Escherichia coli] | 54 | 34 | |
| 229 | 4 | 5050 | 3371 | gi\|1046078 | M. genitalium predicted coding region | 54 | 42 | |
| | | | | | MG369 [Mycoplasma genitalium] | | | |
| 236 | 3 | 4777 | 1496 | gi\|152271 | 319-kDA protein [Rhizobium meliloti] | 54 | 28 | |
| 236 | 5 | 7822 | 6944 | gnl\|PID\|e285031 | Hyp1 protein [Hydra vulgaris] | 54 | 20 | |
| 238 | 30 | 27964 | 27746 | gnl\|PID\|e217586 | PlnM [Lactobacillus plantarum] | 54 | 42 | |
| 242 | 5 | 3508 | 4050 | gi\|149502 | beta-lactamase [Lactococcus lactis] | 54 | 35 | |
| 257 | 1 | 296 | 120 | gi\|1498064 | AtE1 [Arabidopsis thaliana] | 54 | 50 | |
| 257 | 6 | 6745 | 5633 | gi\|343949 | var1(40.0) [Saccharomyces cerevisiae] | 54 | 42 | |
| 258 | 8 | 7839 | 7114 | gi\|41519 | P30 protein (AA 1-240) [Escherichia coli] | 54 | 31 | |
| 276 | 20 | 13101 | 12880 | gi\|155322 | icsB gene product [Plasmid pWR100] | 54 | 37 | |
| 280 | 1 | 618 | 106 | gi\|467356 | unknown [Bacillus subtilis] | 54 | 21 | |
| 288 | 4 | 2183 | 2632 | gi\|39978 | P16 [Bacillus subtilis] | 54 | 39 | |
| 316 | 1 | 3 | 767 | gi\|143264 | membrane-associated protein [Bacillus | 54 | 34 | |
| | | | | | subtilis] | | | |
| 318 | 7 | 5035 | 4565 | gi\|606080 | ORF_o290; Geneplot suggests frameshift | 54 | 28 | |
| | | | | | linking to o267, not found Escherichia | | | |
| | | | | | coli] | | | |
| 319 | 3 | 1393 | 2163 | gi\|148327 | vancomycin response regulator | 54 | 34 | |
| | | | | | [Enterococcus faecium] | | | |
| 323 | 2 | 1256 | 2560 | gi\|413940 | ipa-16d gene product [Bacillus subtilis] | 54 | 26 | |
| 364 | 7 | 7335 | 7724 | gnl\|PID\|e250171 | F18C12.1 [Caenorhabditis elegans] | 54 | 31 | |
| 386 | 5 | 2399 | 3844 | gi\|155369 | PTS enzyme-II fructose [Xanthomonas | 54 | 37 | |
| | | | | | campestris] | | | |
| 392 | 3 | 2004 | 3353 | gi\|872306 | integral membrane protein [Streptomyces | 54 | 32 | |
| | | | | | pristinaespiralis] pir\|557509\|S57509 | | | |
| | | | | | integral membrane protein - Streptomyces | | | |
| | | | | | ristinaespiralis | | | |
| 424 | 5 | 1553 | 1371 | gi\|160316 | major merozoite surface antigen | 54 | 37 | |
| | | | | | [Plasmodium falciparum] | | | |
| | | | | sp\|P50495\|MSP1_PLAFP MEROZOITE SURFACE | | | | |
| | | | | | PROTEIN 1 PRECURSOR MEROZOITE SURFACE | | | |
| | | | | | ANTIGENS) (PMMSA) (GP195) | | | |
| 445 | 2 | 1897 | 1178 | gi\|1781503 | MigA [Pseudomonas aeruginosa] | 54 | 31 | |
| 452 | 5 | 2506 | 2805 | gi\|216292 | neopullulanase [Bacillus sp.] | 54 | 34 | |
| 457 | 2 | 2178 | 1024 | gi\|405570 | TraK protein shares sequence similarity | 54 | 35 | |
| | | | | | with a family of proteins ncoded on Gram- | | | |
| | | | | | negative gene transfer systems such as | | | |
| | | | | | TraD from the plasmid [Plasmid pSK41] | | | |
| 461 | 3 | 627 | 1418 | gi\|797332 | MocD [Agrobacterium tumefaciens] | 54 | 38 | |
| 466 | 5 | 5419 | 3770 | gi\|1652892 | ABC transporter [Synechocystis sp.] | 54 | 29 | |
| 475 | 3 | 2745 | 1990 | gi\|532546 | ORF13 [Enterococcus faecalis] | 54 | 35 | |
| 495 | 1 | 2 | 295 | gi\|304990 | ORF_o290 [Escherichia coli] | 54 | 21 | |
| 502 | 4 | 3518 | 3216 | gi\|1573270 | hemolysin (tlyC) [Haemophilus influenzae] | 54 | 33 | |
| 510 | 5 | 3089 | 3931 | gi\|1732200 | PTS permease for mannose subunit IIPMan | 54 | 29 | |
| | | | | | [Vibria furnissii] | | | |
| 570 | 1 | 1 | 930 | gi\|1001582 | penicillin-binding protein 1A | 54 | 31 | |
| | | | | | [Synechocystis sp.] | | | |
| 573 | 6 | 2763 | 3164 | gi\|416197 | homologous to plasmid R100 pemK gene | 54 | 35 | |
| | | | | | [Escherichia coli] | | | |
| 590 | 1 | 433 | 2 | gi\|532309 | 25 kDa protein [Escherichia coli] | 54 | 33 | |
| 643 | 2 | 1202 | 1477 | gnl\|PID\|e125689 | 256 kD golgin [Homo sapiens] | 54 | 29 | |
| 705 | 1 | 2 | 682 | gi\|148921 | LicD protein [Haemophilus influenzae] | 54 | 39 | |
| 730 | 1 | 370 | 167 | gnl\|PID\|e245531 | ORF YLR068w [Saccharornyces cerevisiae] | 54 | 29 | |
| 745 | 1 | 502 | 209 | gi\|581140 | NADH dehydrogenase [Escherichia coli] | 54 | 37 | |
| 749 | 1 | 413 | 3 | gi\|664840 | TagB [Dictyostelium discoideum] | 54 | 44 | |
| 932 | 1 | 3 | 320 | gi\|537207 | ORF_f277 [Escherichia coli] | 54 | 27 | |
| 4 | 6 | 5671 | 4748 | gi\|216267 | ORF2 [Bacillus megaterium] | 53 | 34 | |
| 16 | 8 | 6231 | 6806 | gi\|517105 | spermidine acetyltransferase [Escherichia | 53 | 35 | |
| | | | | | coli] | | | |
| 17 | 1 | 2 | 2497 | gi\|387880 | collagen adhesin [Staphylococcus aureus] | 53 | 35 | |
| 42 | 4 | 2942 | 3529 | gi\|1633572 | Herpesvirus saimiri ORF73 homolog | 53 | 20 | |
| | | | | | [Kaposi's sarcoma-associated herpes-like | | | |
| | | | | | virus] | | | |
| 69 | 6 | 3149 | 4879 | gi\|1486244 | unknown [Bacillus subtilis] | 53 | 30 | |
| 72 | 3 | 1455 | 2063 | gi\|1592197 | M. jannaschii predicted coding region | 53 | 32 | |
| | | | | | MJ1576 [Methanococcus jannaschii] | | | |
| 79 | 1 | 83 | 592 | gi\|633757 | pr2 [Mycoplasma hyopneumoniae] | 53 | 28 | |
| 83 | 8 | 5179 | 4412 | gi\|496100 | unknown function; putative [Bacteriophage | 53 | 39 | |
| | | | | | phi-LC3] | | | |
| 85 | 10 | 7180 | 6764 | gil 1303940 | YgiU [Bacillus subtilis] | 53 | 35 | |
| 92 | 2 | 789 | 986 | gi\|1372996 | Rho [Borrelia burgdorferi] | 53 | 28 | |
| 95 | 10 | 7546 | 7734 | gi\|162379 | variant surface glycoprotein [Trypanosoma | 53 | 28 | |
| | | | | | brucei] | | | |
| 99 | 4 | 1391 | 1861 | gi\|1499620 | M. jannaschii predicted coding region | 53 | 34 | |
| | | | | | MJ0798 [Methanococcus jannaschii] | | | |
| 100 | 44 | 29982 | 29749 | gi\|1590997 | M. jannaschii predicted coding region | 53 | 35 | |
| | | | | | MJ0272 [Methanococcus jannaschii] | | | |
| 102 | 5 | 4787 | 5089 | gi\|1399011 | immunogenic secreted protein precursor | 53 | 40 | |
| | | | | | [Streptococcus pyogenes] | | | |
| 113 | 1 | 825 | 4 | gnl\|PID\|e264148 | unknown [Mycobacterium tuberculosis] | 53 | 24 | |
| 114 | 4 | 6555 | 5113 | gi\|487282 | Na+ −ATPase subunit J [Enterococcus hirae] | 53 | 33 | |
| 119 | 6 | 3581 | 3994 | gi\|473707 | positive regulator for virulence factors | 53 | 31 | |
| | | | | | [Clostridium perfringens] | | | |
| 123 | 19 | 16463 | 18115 | gi\|1591361 | NADH oxidase [Methanococcus jannaschii] | 53 | 33 | |
| 136 | 1 | 381 | 4 | gi\|152744 | IpaD protein [Shigella flexneri] | 53 | 32 | |
| 138 | 9 | 8079 | 7594 | gi\|467371 | LACI family of transcriptional repreesor | 53 | 29 | |
| | | | | | (probable) [Bacillus ubtilis] | | | |
| 142 | 8 | 4594 | 4007 | gi\|755216 | N-acetylmuramidase [Lactococcus lactis] | 53 | 38 | |
| 162 | 12 | 12482 | 11937 | gi\|1063250 | low homology to P20 protein of Bacillus | 53 | 36 | |
| | | | | | lichiniformis and bleomycin | | | |
| | | | | | acetyltransferase of Streptomyces | | | |
| | | | | | verticillus [Bacillus subtilis] | | | |
| 163 | 1 | 546 | 31 | gi\|153767 | ORF [Streptococcus pneumoniae] | 53 | 34 | |
| 163 | 7 | 4973 | 3453 | gi\|29468 | beta-myosin heavy chain (1151 AA) [Homo | 53 | 36 | |
| | | | | | sapiens] | | | |
| 167 | 2 | 1038 | 2006 | gi\|413930 | ipa-6d gene product [Bacillus subtilis] | 53 | 27 | |
| 173 | 11 | 8865 | 7843 | gi\|1778569 | YaaF homolog [Escherichia coli] | 53 | 39 | |
| 190 | 8 | 6842 | 3549 | gi\|387880 | collagen adhesin [Staphylococcus aureus] | 53 | 38 | |
| 199 | 2 | 2725 | 950 | gi\|1652570 | nitrate transport protein NrtB | 53 | 32 | |
| | | | | | [Synechocystis sp.] | | | |
| 200 | 13 | 6184 | 5954 | gi\|1652679 | hypothetical protein [Synechocystis sp.] | 53 | 40 | |
| 200 | 17 | 9287 | 7890 | gi\|1574246 | H. influenzae predicted coding region | 53 | 35 | |
| | | | | | HI1409 [Haemophilus influenzae] | | | |
| 205 | 6 | 2048 | 3229 | gi\|148026 | topoisomerase III [Escherichia coli] | 53 | 32 | |
| 211 | 2 | 270 | 1052 | gi\|483940 | transcription regulator [Bacillus | 53 | 30 | |
| | | | | | subtilis] | | | |
| 221 | 10 | 5119 | 5994 | gi\|1353529 | ORF12 [Bacteriophage rlt] | 53 | 44 | |
| 232 | 7 | 4344 | 3925 | gi\|1665759 | Similar to Schistosoma mansoni amino acid | 53 | 35 | |
| | | | | | permease (L25068). [Homo sapiens] | | | |
| 238 | 21 | 18705 | 19247 | gi\|1574062 | hypothetical [Haemophilus influenzae] | 53 | 30 | |
| 239 | 1 | 2 | 1636 | gi\|433932 | activator of (R)-hydroxyglutaryl-CoA | 53 | 35 | |
| | | | | | dehydratase [Acidaminococcus ermentans] | | | |
| 250 | 1 | 1469 | 318 | gi\|987094 | membrane transport protein [Streptomyces | 53 | 22 | |
| | | | | | hygroscopicus] | | | |
| 253 | 4 | 1759 | 1028 | gi\|537245 | aspartokinase I-homoserine dehydrogenase I | 53 | 35 | |
| | | | | | [Escherichia coli] pir\|556629\|S56629 | | | |
| | | | | | aspartate kinase (EC 2.7.2.4)/homoserine | | | |
| | | | | | ehydrogenase (EC 1.1.1.3) - Escherichia | | | |
| | | | | | coli | | | |
| 271 | 8 | 4649 | 5800 | gi\|413966 | ipa-42d gene product [Bacillus subtilis] | 53 | 27 | |
| 276 | 26 | 15786 | 15112 | gi\|1699017 | ErpB2 [Borrelia burgdorferi] | 53 | 26 | |
| 279 | 11 | 8309 | 7797 | gi\|1651934 | hypothetical protein [Synechocystis sp.] | 53 | 35 | |
| 288 | 8 | 3997 | 4872 | gi\|43943 | second subunit of EII-Sor [Klebsiella | 53 | 32 | |
| | | | | | pneumoniae] | | | |
| 290 | 6 | 4391 | 5680 | gi\|466882 | pps1; B1496_C2_189 [Mycobacterium leprae] | 53 | 29 | |
| 294 | 3 | 1197 | 1481 | gi\|173004 | topoisomerase I [Saccharomyces cerevisiae] | 53 | 40 | |
| 330 | 3 | 2351 | 3367 | gi\|466691 | No definition line found [Escherichia | 53 | 34 | |
| | | | | | coli] | | | |
| 334 | 8 | 8172 | 9182 | gi\|1652483 | hypothetical protein [Synechocystis sp.] | 53 | 29 | |
| 368 | 1 | 620 | 102 | gi\|487273 | Na+ ATPase subunit I [Enterococcus hirae] | 53 | 29 | |
| 377 | 4 | 2424 | 2260 | gi\|221407 | FPS [Fowlpox virus] | 53 | 35 | |
| 382 | 1 | 257 | 36 | gi\|1592016 | M. jannaschii predicted coding region | 53 | 32 | |
| | | | | | MJ1371 [Methanococcus jannaschii] | | | |
| 387 | 1 | 2 | 460 | gi\|1574317 | repressor protein (GP:L22692_1) | 53 | 30 | |
| | | | | | [Haemophilus influenzae] | | | |
| 394 | 10 | 8379 | 10412 | gi\|882463 | protein-N(pi)-phosphohistidine-sugar | 53 | 34 | |
| | | | | | phosphotransferase [Escherichia oli] | | | |
| 399 | 4 | 2349 | 3098 | gi\|453287 | OmpR protein [Escherichia coli] | 53 | 27 | |
| 420 | 2 | 1378 | 719 | gi\|1437473 | nitrate transporter [Bacillus subtilis] | 53 | 28 | |
| 441 | 6 | 5361 | 7937 | gi\|1592205 | M. jannaschii predicted coding region | 53 | 38 | |
| | | | | | MJ1595 [Methanococcus jannaschii] | | | |
| 461 | 1 | 6 | 512 | gi\|1651800 | L-glutamine:D-fructose-6-P | 53 | 29 | |
| | | | | | amidotransferase [Synechocystis sp.] | | | |
| 497 | 3 | 1700 | 1960 | gi\|4328 | RIF1 gene product [Saccharomyces | 53 | 33 | |
| | | | | | cerevisiae] | | | |
| 503 | 1 | 669 | 4 | gnl\|PID\|e202290 | unknown [Lactobacillus sake] | 53 | 30 | |
| 538 | 2 | 1053 | 262 | gi\|1613769 | response regulator [Streptococcus | 53 | 30 | |
| | | | | | pneumoniae] | | | |
| 539 | 6 | 6172 | 5183 | gi\|567887 | putative repressor [Streptomyces | 53 | 32 | |
| | | | | | peucetius] | | | |
| 551 | 1 | 629 | 162 | gi\|1256649 | putative [Bacillus subtilis] | 53 | 26 | |
| 557 | 1 | 9 | 695 | gi\|143177 | putative [Bacillus subtilis] | 53 | 31 | |
| 569 | 2 | 418 | 1158 | gi\|1184684 | MucD [Pseudomonas aeruginosa] | 53 | 26 | |
| 614 | 1 | 99 | 581 | gi\|485280 | 28.2 kDa protein [Streptococcus | 53 | 32 | |
| | | | | | pneumoniae] | | | |
| 660 | 1 | 1 | 279 | gnl\|PID\|e288480 | R10E8.f [Caenorhabditis elegans] | 53 | 34 | |
| 776 | 1 | 3 | 635 | gi\|151352 | mandelate racemase (EC 5.1.2.2) | 53 | 33 | |
| | | | | | [Pseudomonas putida] | | | |
| 11 | 2 | 1117 | 1656 | gi\|143150 | levR [Bacillus subtilis] | 52 | 29 | |
| 17 | 6 | 5327 | 6559 | gnl\|PID\|e250887 | potential coding region [Clostridium | 52 | 37 | |
| | | | | | difficile] | | | |
| 19 | 31 | 17760 | 17978 | gi\|1079556 | dShc [Drosophila melanogaster] | 52 | 42 | |
| 19 | 38 | 20306 | 22627 | gn\|PID\|e139448 | host interacting protein [Bacteriophage | 52 | 32 | |
| | | | | | B1] | | | |
| 25 | 4 | 2662 | 2087 | gi\|1072067 | PepE [Rhodobacter sphaeroides] | 52 | 23 | |
| 25 | 6 | 5596 | 3407 | gi\|1303866 | YggS [Bacillus subtilis] | 52 | 34 | |
| 49 | 3 | 1135 | 1569 | gi\|496279 | putative [Bacteriophage Tuc2009] | 52 | 25 | |
| 53 | 1 | 850 | 2 | sp\|P52697\|YBHE_ECO | HYPOTHETICAL 30.2 KD PROTEIN IN MODC | 52 | 35 | |
| | | | | LI | 3′REGION. | | | |
| 54 | 9 | 10909 | 2687 | gi\|1633572 | Herpesvirus saimiri ORF73 homolog | 52 | 30 | |
| | | | | | [Kaposi's sarcoma-associated herpes-like | | | |
| | | | | | virus] | | | |
| 57 | 6 | 4779 | 8402 | gi\|142439 | ATP-dependent nuclease [Bacillus subtilis] | 52 | 31 | |
| 58 | 6 | 6446 | 5949 | gnl\|PID\|e255921 | F53F4.10 [Caenorhabditis elegans] | 52 | 31 | |
| 72 | 13 | 13446 | 13195 | gi\|532541 | ORF8 [Enterococcus faecalis] | 52 | 37 | |
| 81 | 17 | 13692 | 12520 | gi\|1732203 | GlcNAc 6-P deacetylase [Vibrio furnissii] | 52 | 35 | |
| 84 | 1 | 3 | 1355 | gi\|64288 | fast skeletal muscle Ca-ATPase [Rana | 52 | 34 | |
| | | | | | esculenta] | | | |
| 100 | 2 | 1917 | 1027 | gi\|1353560 | ORF43 [Bacteriophage rlt] | 52 | 34 | |
| 101 | 1 | 30 | 1862 | gi\|405957 | yeeF [Escherichia coli] | 52 | 24 | |
| 106 | 8 | 8517 | 7600 | gi\|454904 | rfbG gene product [Shigella flexneri] | 52 | 41 | |
| 108 | 1 | 1 | 1059 | gnl\|PID\|e255337 | unknown [Mycobacterium tuberculosis] | 52 | 29 | |
| 123 | 4 | 2899 | 3495 | gi\|1305720 | prs-associated putative membrane protein | 52 | 24 | |
| | | | | | [Escherichia coli] | | | |
| 128 | 23 | 17561 | 16740 | gi\|473805 | ‘regulatory protein sfs1 involved in | 52 | 32 | |
| | | | | | maltose metabolism’ Escherichia coli] | | | |
| 130 | 8 | 6693 | 7481 | gi\|1552775 | ATP-binding protein [Escherichia coli] | 52 | 30 | |
| 138 | 1 | 40 | 1359 | gi\|1045867 | oligoendopeptidase F [Mycoplasma | 52 | 31 | |
| | | | | | genitalium] | | | |
| 138 | 2 | 2757 | 1384 | gi\|1591425 | hypothetical protein (GP:X91006_2) | 52 | 26 | |
| | | | | | [Methanococcus jannaschii] | | | |
| 138 | 6 | 6317 | 5940 | gi\|1486247 | unknown [Bacillus subtilis] | 52 | 36 | |
| 142 | 10 | 7337 | 5466 | gi\|1151158 | repeat organellar protein [Plasmodium | 52 | 34 | |
| | | | | | chabaudi] | | | |
| 149 | 1 | 33 | 1133 | gi\|1762962 | FemA [Staphylococcus simulans] | 52 | 31 | |
| 161 | 1 | 3 | 245 | gi\|151276 | histidine utilization genes repressor | 52 | 35 | |
| | | | | | protein (hut) [Pseudomonas utida] | | | |
| 163 | 4 | 2048 | 1320 | gi\|1064810 | function unknown [Bacillus subtilis] | 52 | 27 | |
| 164 | 8 | 4882 | 5103 | gi\|57251 | precursor (AA −35 to 1766) [Rattus | 52 | 38 | |
| | | | | | norvegicus] | | | |
| 165 | 9 | 7247 | 7474 | gi\|1652671 | hypothetical protein [Synechocystis sp.] | 52 | 28 | |
| 178 | 5 | 1887 | 1681 | gi\|220704 | cAMP-dependent protein kinase catalytic | 52 | 36 | |
| | | | | | subunit-beta [Rattus sp.]gi\|191177 cAMP- | | | |
| | | | | | dependent protein kinase beta-catalytic | | | |
| | | | | | subunit Cricetulus sp.] | | | |
| 180 | 24 | 22536 | 23774 | gi\|581052 | cytosine deaminase [Escherichia coli] | 52 | 28 | |
| 190 | 9 | 8891 | 7056 | gi\|1592079 | M. jannaschii predicted coding region | 52 | 39 | |
| | | | | | MJ1429 [Methanococcus jannaschii] | | | |
| 195 | 8 | 2000 | 2272 | gi\|868024 | HIC-1 gene product [Homo sapiens] | 52 | 52 | |
| 202 | 11 | 9189 | 10145 | gi\|141861 | traA gene product [Plasmid pAD1] | 52 | 33 | |
| 204 | 4 | 1361 | 2011 | gi\|1184118 | mevalonate kinase [Methanobacterium | 52 | 33 | |
| | | | | | thermoautotrophicum] | | | |
| 204 | 8 | 4018 | 5142 | gnl\|PID\|e283860 | carotenoid biosynthetic gene ERWCRTS | 52 | 31 | |
| | | | | | homolog [Sulfolobus solfataricus] | | | |
| 208 | 2 | 1112 | 2296 | gi\|1408501 | homologous to N-acyl-L-amino acid | 52 | 35 | |
| | | | | | amidohydrolase of Bacillus | | | |
| | | | | | stearothermophilus [Bacillus subtilis] | | | |
| 215 | 1 | 772 | 2 | gi\|1480429 | putative transcriptional regulator | 52 | 26 | |
| | | | | | [Bacillus stearothemophilus] | | | |
| 218 | 4 | 4072 | 3425 | gi\|862630 | glyceraldehyde-3-Phosphate dehydrogenase | 52 | 35 | |
| | | | | | [Buchnera aphidicola] sp\|Q07234\|G3P_BUCAP | | | |
| | | | | | GLYCERALDEHYDE 3-PHOSPHATE DEHYDROGENASE | | | |
| | | | | | (EC .2.1.12) (GAPDH). | | | |
| 228 | 1 | 1 | 741 | gnl\|PID\|e264148 | unknown [Mycobacterium tuberculosis] | 52 | 29 | |
| 230 | 2 | 149 | 634 | gi\|437705 | hyaluronidase [Streptococcus pneumoniae] | 52 | 28 | |
| 233 | 8 | 6166 | 4982 | gi\|1001708 | NifS [Synechocystis sp.] | 52 | 31 | |
| 240 | 3 | 725 | 967 | gi\|399655 | Ca2+ regulatory protein [Saccharomyces | 52 | 21 | |
| | | | | | cerevisiae]sp ( P35206 I CSG2 YEAST CSG2 | | | |
| | | | | | PROTEIN PRECURSOR. | | | |
| 288 | 7 | 3171 | 4028 | gi\|147403 | mannose permease subunit II-P-Man | 52 | 27 | |
| | | | | | [Escherichia coli] | | | |
| 318 | 1 | 7 | 819 | gi\|1303849 | YggB [Bacillus subtilis] | 52 | 33 | |
| 330 | 1 | 1062 | 154 | gi\|144859 | ORF B [Clostridium perfringens] | 52 | 29 | |
| 330 | 9 | 6815 | 7213 | gi\|1439527 | EIIA-man [Lactobacillus curvatus] | 52 | 31 | |
| 345 | 9 | 8348 | 9397 | gi\|606292 | ORF_o696 [Escherichia coli] | 52 | 27 | |
| 398 | 3 | 2671 | 1877 | gi\|144859 | ORF B [Clostridium perfringens] | 52 | 29 | |
| 411 | 1 | 992 | 3 | gnl\|PID\|e283950 | daunorubicin resistance ATP-binding | 52 | 27 | |
| | | | | | protein DrrA [Sulfolobus solfataricus] | | | |
| 422 | 2 | 1292 | 585 | gi\|537214 | yjjG gene product [Escherichia coli] | 52 | 32 | |
| 436 | 2 | 1669 | 1205 | gi\|507323 | ORF1 [Bacillus stearothermophilus] | 52 | 29 | |
| 450 | 1 | 119 | 754 | gi\|1573916 | multidrug resistance protein (emrB) | 52 | 32 | |
| | | | | | [Haemophilus influenzae] | | | |
| 453 | 1 | 190 | 381 | gi\|182021 | elastin [Homo sapiens] | 52 | 40 | |
| 455 | 7 | 5767 | 4634 | gnl\|PID\|e155312 | integrase [Bacteriophage TP901-1] | 52 | 34 | |
| 479 | 1 | 138 | 758 | gi\|1742859 | ORF_ID:o327#7; similar to [SwissProt | 52 | 27 | |
| | | | | | Accession Number P54449] [Escherichia | | | |
| | | | | | coli] | | | |
| 517 | 1 | 763 | 2 | gi\|152780 | rhamnosyl transferase II [Shigella | 52 | 29 | |
| | | | | | dysenteriae] | | | |
| 518 | 3 | 1735 | 848 | gi\|153858 | wall-associated protein [Streptococcus | 52 | 20 | |
| | | | | | mutans] | | | |
| 526 | 3 | 2297 | 1848 | gi\|147402 | mannose permease subunit III-Man | 52 | 27 | |
| | | | | | [Escherichia coli] | | | |
| 617 | 1 | 1 | 462 | gi\|142863 | replication initiation protein [Bacillus | 52 | 35 | |
| | | | | | subtilis] | | | |
| 639 | 3 | 1068 | 259 | gi\|1591153 | hypothetical protein (SP:P46348) | 52 | 30 | |
| | | | | | [Methanococcus jannaschii] | | | |
| 703 | 1 | 773 | 81 | gi\|793910 | surface antigen [Homo sapiens] | 52 | 31 | |
| 737 | 1 | 235 | 2 | gi\|666000 | hypothetical protein [Bacillus subtilis] | 52 | 29 | |
| 791 | 4 | 1368 | 1802 | gnl\|PID\|e269549 | Unknown [Bacillus subtilis] | 52 | 28 | |
| 825 | 1 | 1 | 300 | gi\|732538 | No definition line found [Caenorhabditis | 52 | 28 | |
| | | | | | elegans] | | | |
| 981 | 1 | 226 | 2 | gi\|951100 | P45016a-ms1 [Mus spretus] | 52 | 36 | |
| 17 | 23 | 23542 | 22163 | gi\|1652483 | hypothetical protein [Synechocystis sp.] | 51 | 32 | |
| 65 | 6 | 4302 | 3691 | gi\|397498 | Membrane Ribose Binding Protein [Bacillus | 51 | 31 | |
| | | | | | subtilis] pir\|S42714\|S42714 membrane | | | |
| | | | | | ribose-binding protein - Bacillus ubtilis | | | |
| 69 | 5 | 2926 | 2537 | gi\|1773150 | hypothetical 14.8kd protein [Escherichia | 51 | 30 | |
| | | | | | coli] | | | |
| 92 | 1 | 973 | 44 | gnl\|PID\|e243523 | ORF YGR130c [Saccharomyces cerevisae] | 51 | 29 | |
| 103 | 6 | 5272 | 3593 | gi\|312940 | threonine kinase [Streptococcus | 51 | 32 | |
| | | | | | equisimilis] | | | |
| 111 | 7 | 4195 | 3317 | pir\|G64143\|G64143 | hypothetical protein HI0143 - Haemophilus | 51 | 29 | |
| | | | | | influenzae (strain Rd KW20) | | | |
| 115 | 7 | 4526 | 3414 | gi\|405879 | yeiH [Escherichia coli] | 51 | 27 | |
| 123 | 29 | 27788 | 28207 gi\|147402 | mannose permease subunit III-Man | 51 | 27 | | |
| | | | | | [Escherichia coli] | | | |
| 125 | 1 | 223 | 2 | gi\|4482 | SLY1 gene product [Saccharomyces | 51 | 37 | |
| | | | | | cerevisiae] | | | |
| 128 | 21 | 16156 | 15638 | gi\|606026 | ORF_o115 [Escherichia coli] | 51 | 27 | |
| 137 | 4 | 3207 | 5369 | gi\|1673692 | (AE000005) Mycoplasma pneumoniae, | 51 | 26 | |
| | | | | | C09_orf422 Protein [Mycoplasma pneumoniae] | | | |
| 138 | 28 | 18295 | 18771 | gi\|149647 | ORFZ [Listeria monocytogenes] | 51 | 31 | |
| 145 | 6 | 4054 | 5271 | gi\|1653860 | N-acyl-L-amino acid amidohydrolase | 51 | 41 | |
| | | | | | [Synechocystis sp.] | | | |
| 155 | 4 | 3019 | 2273 | gi\|1486242 | unknown [Bacillus subtilis] | 51 | 41 | |
| 180 | 8 | 7951 | 9189 | gi\|1657522 | hypothetical protein [Escherichia coli] | 51 | 32 | |
| 186 | 2 | 859 | 1620 | gi\|511497 | oleoyl-acyl carrier protein thioesterase | 51 | 29 | |
| | | | | | [Coriandrum sativum] | | | |
| 186 | 3 | 1644 | 2060 | sp\|P37348\|YECE_ECO | HYPOTHETICAL PROTEIN IN ASPS 5 REGION | 51 | 38 | |
| | | | | LI | (FRAGMENT). | | | |
| 194 | 3 | 1521 | 1276 | gi\|332697 | fusion protein [Human parainfluenza virus | 51 | 32 | |
| | | | | | 2] | | | |
| 195 | 7 | 1986 | 3767 | gi\|405570 | TraK protein shares sequence similarity | 51 | 28 | |
| | | | | | with a family of proteins ncoded on Gram- | | | |
| | | | | | negative gene transfer systems such as | | | |
| | | | | | TraD from the plasmid [Plasmid pSK41] | | | |
| 197 | 1 | 3 | 494 | gi\|1592234 | DNA topoisomerase I [Methanococcus | 51 | 32 | |
| | | | | | jannaschii] | | | |
| 198 | 2 | 1521 | 862 | gi\|1196483 | unknown protein [Lactobacillus casei] | 51 | 32 | |
| 238 | 16 | 13630 | 14730 | gi\|1772652 | 2-keto-3-deoxygluconate kinase [Haloferax | 51 | 36 | |
| | | | | | alicantei] | | | |
| 257 | 5 | 5646 | 4513 | pir\|S43367\|S43367 | metallothionein - Green crab, common shore | 51 | 38 | |
| | | | | | crab | | | |
| 261 | 6 | 4950 | 4519 | gi\|581545 | orf 4 [Staphylococcus aureus] | 51 | 26 | |
| 270 | 5 | 4480 | 4220 | gi\|1066975 | F49E2.5a [Caenorhabditis elegans] | 51 | 28 | |
| 306 | 10 | 5928 | 6905 | gi\|1752736 | gene required for phosphoylation of | 51 | 28 | |
| | | | | | oligosaccharides/ has high homology with | | | |
| | | | | | YJR061w [Saccharomyces cerevisiae] | | | |
| 324 | 3 | 1590 | 2405 | gi\|409925 | VirR positive regulator [Streptococcus | 51 | 25 | |
| | | | | | pyogenes] | | | |
| 328 | 2 | 632 | 309 | gi\|466475 | putative phospho-beta-glucosidase | 51 | 30 | |
| | | | | | [Bacillus stearothermophilus] | | | |
| | | | | | pir\|D49898\|D49898 cellobiose | | | |
| | | | | | phosphotransferase system celC - acillus | | | |
| | | | | | stearothermophilus | | | |
| 340 | 2 | 898 | 1152 | gi\|40046 | phosphoglucose isomerase A (AA 1-449) | 51 | 39 | |
| | | | | | [Bacillus stearothermophilus] | | | |
| | | | | | ir\|S15936\|NUBSSA glucose-6-phosphate | | | |
| | | | | | isomerase (EC 5.3.1.9) A - cillus | | | |
| | | | | | stearothermophilus | | | |
| 340 | 4 | 3617 | 2445 | gi\|763052 | integrase [Bacteriophage T270] | 51 | 33 | |
| 379 | 10 | 11742 | 11311 | gi\|887829 | D21141 uses 2nd start; frame determined by | 51 | 34 | |
| | | | | | Lac fusion [Escherichia oli] | | | |
| 380 | 1 | 2 | 1123 | gi\|309662 | pheromone binding protein [Plasmid pCF10] | 51 | 34 | |
| 395 | 1 | 526 | 95 | gi\|490986 | phi 105 repessor orf2 [unidentified] | 51 | 27 | |
| 424 | 4 | 2512 | 995 | gi\|1633572 | Herpesvirus saimiri ORF73 homolog | 51 | 31 | |
| | | | | | [Kaposi's sarcoma-associated herpes-like | | | |
| | | | | | virus] | | | |
| 444 | 1 | 737 | 483 | gi\|1245376 | cardiac ryanodine receptor [Oryctolagus | 51 | 34 | |
| | | | | | cuniculus] | | | |
| 483 | 1 | 1 | 642 | gi\|1303981 | YgkD [Bacillus subtilis] | 51 | 29 | |
| 500 | 1 | 2 | 550 | gi\|987094 | membrane transport protein [Streptomyces | 51 | 23 | |
| | | | | | hygroscopicus] | | | |
| 525 | 3 | 492 | 983 | pir\|A57438\|A57438 | tryptophan-rich sensory protein - | 51 | 38 | |
| | | | | | Rhodobacter sphaeroides (strain 2.4.1) | | | |
| 534 | 1 | 2 | 1165 | gi\|147516 | ribokinase [Escherichia coli] | 51 | 33 | |
| 547 | 1 | 1 | 387 | gi\|1353528 | ORF11 [Bacteriophage rlt] | 51 | 33 | |
| 553 | 2 | 1728 | 1330 | pir\|B55124\|B55124 | thioredoxin - Chlorobium sp. | 51 | 27 | |
| 574 | 1 | 2291 | 2476 | bbs\|129435 | RprX=inner membrane signal-transducing | 51 | 36 | |
| | | | | | protein [Bacteroides fragilis, Peptide, | | | |
| | | | | | 519 aa] [Bacteroides fragilis] | | | |
| 574 | 2 | 3145 | 3420 | gi\|1732202 | PTS permease for mannose subunit IIIMan N | 51 | 29 | |
| | | | | | terminal domain [Vibrio furnissii] | | | |
| 594 | 2 | 530 | 225 | gi\|1657696 | tryptophan hydroxylase [Gallus gallus] | 51 | 40 | |
| 605 | 3 | 1220 | 1936 | gnl\|PID\|e289149 | similar to B. subtilis YcsE hypothetical | 51 | 32 | |
| | | | | | protein [Bacillus subtilis] | | | |
| 609 | 1 | 1027 | 74 | gi\|1226279 | strong similarity to Schistosoma amino | 51 | 26 | |
| | | | | | acid permease (GB:L25068) [Caenorhabditis | | | |
| | | | | | elegans] | | | |
| 656 | 2 | 2033 | 2950 | gi\|143213 | putative [Bacillus subtilis] | 51 | 26 | |
| 670 | 1 | 1508 | 369 | gi\|1652222 | hypothetical protein [Synechocystis sp.] | 51 | 25 | |
| 673 | 1 | 2 | 1135 | gi\|532553 | ORF20 [Enterococcus faecalis] | 51 | 27 | |
| 674 | 2 | 1158 | 778 | gi\|467451 | unknown [Bacillus subtilis] | 51 | 26 | |
| 735 | 2 | 477 | 725 | gi\|757791 | aromatic amino acid permease | 51 | 38 | |
| | | | | | [Corynebacterium glutamicum] | | | |
| | | | | | pir\|S52754\|S52754 aromatic amino acid | | | |
| | | | | | permease - Corynebacterium lutamicum | | | |
| 924 | 1 | 794 | 3 | gi\|40663 | sialidase [Clostridium septicum] | 51 | 35 | |
| 4 | 5 | 3811 | 4728 | gi\|413948 | ipa-24d gene product [Bacillus subtilis] | 50 | 29 | |
| 8 | 3 | 3310 | 2180 | gi\|1592205 | M. jannaschii predicted coding region | 50 | 28 | |
| | | | | | MJ1595 [Methanococcus jannaschii] | | | |
| 11 | 9 | 5269 | 5520 | gi\|1651800 | L-glutamine:D-fructose-6-P | 50 | 25 | |
| | | | | | amidotransferase [Synechocystis sp.] | | | |
| 12 | 6 | 9045 | 8662 | gnl\|PID\|e254943 | unknown [Mycobacterium tuberculosis] | 50 | 23 | |
| 15 | 4 | 2911 | 4269 | gi\|1592173 | N-ethylammeline chlorohydrolase | 50 | 28 | |
| | | | | | [Methanococcus jannaschii] | | | |
| 19 | 10 | 4934 | 5530 | gi\|825569 | unknown [Saccharomyces cerevisiae] | 50 | 20 | |
| 28 | 5 | 7515 | 7057 | gi\|1230586 | orf10; Method: conceptual translation | 50 | 38 | |
| | | | | | supplied by author [Vibrio cholerae O139] | | | |
| 45 | 9 | 4279 | 5019 | gi\|1591029 | thioredoxin/glutaredoxin [Methanococcus | 50 | 32 | |
| | | | | | jannaschii] | | | |
| 54 | 16 | 7739 | 7590 | gi\|1589837 | cuticle preprocollagen [Meloidogyne | 50 | 46 | |
| | | | | | incognita] | | | |
| 59 | 5 | 1551 | 2345 | gi\|144297 | acetyl esterase (XynC) [Caldocellum | 50 | 34 | |
| | | | | | saccharolyticum] pir\|B37202\|B37202 | | | |
| | | | | | acetylesterase (EC 3.1.1.6) (XynC) - | | | |
| | | | | | Caldocellum accharolyticum | | | |
| 62 | 3 | 1650 | 1360 | gnl\|PID\|e205266 | LEA76 homologue type2 [Arabidopsis | 50 | 31 | |
| | | | | | thaliana] | | | |
| 91 | 10 | 8858 | 7521 | gi\|758229 | integrase [Bacteriophage phi-13] | 50 | 31 | |
| 112 | 5 | 3548 | 2133 | gi\|1184262 | GadC [Shigella flexneri] | 50 | 25 | |
| 123 | 13 | 13099 | 14319 | gi\|178273 | alanine:glyoxylate aminotransferase [Homo | 50 | 31 | |
| | | | | | sapiens] | | | |
| 123 | 15 | 14395 | 15675 | gi\|467342 | unknown [Bacillus subtilis] | 50 | 28 | |
| 123 | 31 | 28700 | 29494 | gi\|43942 | first subunit of EII-Sor [Klebsiella | 50 | 27 | |
| | | | | | pneumoniae] | | | |
| 124 | 2 | 1666 | 1061 | gi\|556016 | similar to plant water stress proteins; | 50 | 34 | |
| | | | | | ORF2 [Bacillus subtilis gi\|556016 similar | | | |
| | | | | | to plant water stress proteins; ORF2 | | | |
| | | | | | [Bacillus ubtilis] | | | |
| 128 | 39 | 32767 | 31829 | gi\|39993 | UDP-N-acetylmuramoylalanine--D-glutamate | 50 | 33 | |
| | | | | | ligase [Bacillus subtilis] | | | |
| 135 | 11 | 8803 | 7694 | gi\|895747 | putative cel operon regulator [Bacillus | 50 | 26 | |
| | | | | | subtilis] | | | |
| 138 | 21 | 14648 | 13653 | gi\|1591472 | malic acid transport protein | 50 | 26 | |
| | | | | | [Methanococcus jannaschii] | | | |
| 146 | 3 | 2338 | 1415 | gi\|1732200 | PTS permease for mannose subunit IIPMan | 50 | 27 | |
| | | | | | [Vibrio furnissii] | | | |
| 160 | 2 | 724 | 1302 | gnl\|PID\|e264218 | F54F3.4 [Caenorhabditis elegans] | 50 | 30 | |
| 164 | 15 | 15432 | 16364 | gi\|409286 | bmrU Bacillus subtilis] | 50 | 27 | |
| 167 | 9 | 17082 | 15394 | gi\|143156 | membrane bound protein [Bacillus subtilis] | 50 | 30 | |
| 179 | 3 | 2350 | 4485 | gi\|1408485 | yxdM gene product [Bacillus subtilis] | 50 | 24 | |
| 180 | 30 | 31056 | 30643 | gnl\|PID\|e254644 | membrane protein [Streptococcus | 50 | 27 | |
| | | | | | pneumoniae] | | | |
| 184 | 1 | 2 | 1015 | gi\|854232 | cymE gene product [Klebsiella oxytoca] | 50 | 24 | |
| 194 | 7 | 4335 | 4817 | gi\|1256652 | 25% identity to the E.coli regulatory | 50 | 30 | |
| | | | | | protein MprA; putative [Bacillus subtilis] | | | |
| 195 | 29 | 11712 | 12422 | gi\|662263 | ORF5 [Plasmid pIP501] | 50 | 25 | |
| 204 | 1 | 2 | 166 | gi\|328656 | envelope polyprotein [Human | 50 | 45 | |
| | | | | | immunodeficiency virus type 1] | | | |
| 205 | 7 | 3118 | 3861 | gi\|437697 | traE [Plasmid RP4] | 50 | 31 | |
| 216 | 11 | 7181 | 7750 | gnl\|PID\|e254644 | membrane protein [Streptococcus | 50 | 30 | |
| | | | | | pneumoniae] | | | |
| 223 | 10 | 7036 | 8082 | gi\|606423 | T09B9.1 [Caenorhabditis elegans] | 50 | 30 | |
| 223 | 22 | 19257 | 19799 | gi\|1256141 | YbbL [Bacillus subtilis] | 50 | 29 | |
| 233 | 4 | 3102 | 2320 | gi\|887826 | GUG start [Escherichia coli] | 50 | 32 | |
| 238 | 6 | 5102 | 3906 | gi\|1161219 | hoinolgous to D-amino acid dehydrogenase | 50 | 29 | |
| | | | | | enzyme [Pseudomonas aeruginosa] | | | |
| 239 | 3 | 4449 | 5159 | gi\|41519 | P30 protein (AA 1-240) [Escherichia coli] | 50 | 31 | |
| 242 | 2 | 147 | 2210 | gi\|160299 | glutamic acid-rich protein [Plasmodium | 50 | 30 | |
| | | | | | falciparum] pir\|A54514\|A54514 glutamic | | | |
| | | | | | acid-rich protein precursor - Plasmodium | | | |
| | | | | | alciparum | | | |
| 248 | 2 | 263 | 712 | gi\|143725 | putative [Bacillus subtilis] | 50 | 32 | |
| 256 | 8 | 8531 | 7395 | gnl\|PID\|e250452 | C44H9.4 [Caenorhabditis elegans] | 50 | 38 | |
| 265 | 3 | 1150 | 893 | gi\|1402527 | ORF6 [Enterococcus faecalis] | 50 | 39 | |
| 276 | 24 | 14203 | 14000 | gi\|1591019 | M. jannaschii predicted coding region | 50 | 33 | |
| | | | | | MJ0297 [Methanococcus jannaschii] | | | |
| 276 | 32 | 20601 | 19924 | gi\|1334905 | BXLF2 late reading frame, encodes gp85; | 50 | 29 | |
| | | | | | homologous to RF 37 VZV and glycoprotein H | | | |
| | | | | | of HSV (gpIII of VZV) [Human herpesvirus | | | |
| | | | | | 4] | | | |
| 286 | 1 | 1 | 747 | gnl\|PID\|e257895 | homology with truncated ORF2 of pepF2 | 50 | 32 | |
| | | | | | [Lactococcus lactis] | | | |
| 301 | 17 | 11706 | 13313 | gi\|562039 | NADH dehydrogenase, subunit 2 | 50 | 26 | |
| | | | | | [Acanthamoeba castellanii] | | | |
| | | | | | pir\|S53835\|S53835 NADH dehydrogenase chain | | | |
| | | | | | 2 - Acanthamoeba astellanii mitochondrion | | | |
| | | | | | (SGC6) | | | |
| 338 | 5 | 2206 | 3729 | gi\|829194 | bacterial cell wall hydrolase | 50 | 34 | |
| | | | | | [Enterococcus faecalis] pir\|A38109\|A38109 | | | |
| | | | | | autolysin - Enterococcus faecalis | | | |
| | | | | | sp\|P37710\|ALYS_ENTFA AUTOLYSIN (EC | | | |
| | | | | | 3.5.1.28) N-ACETYLMURAMOYL-L-ALANINE | | | |
| | | | | | AMIDASE). | | | |
| 345 | 12 | 11781 | 13379 | gnl\|PID\|e235181 | unknown [Mycobacterium tuberculosis] | 50 | 32 | |
| 360 | 2 | 2879 | 408 | gi\|40782 | bps2 gene product [Desulfurolobus | 50 | 25 | |
| | | | | | ambivalens] | | | |
| 372 | 1 | 6 | 440 | gi\|1552733 | similar to voltage-gated chloride channel | 50 | 31 | |
| | | | | | protein [Escherichia coli] | | | |
| 372 | 2 | 391 | 738 | gi\|1591749 | TRK system potassium uptake protein A | 50 | 23 | |
| | | | | | [Methanococcus jannaschii] | | | |
| 377 | 3 | 2262 | 1846 | gi\|52797 | kinesin heavy chain [Mus musculus] | 50 | 22 | |
| 392 | 1 | 433 | 2 | gi\|147213 | phnP protein [Escherichia coli] | 50 | 33 | |
| 399 | 31 | 29803 | 30186 | gi\|146288 | PTS enzyme III glucitol [Escherichia coli] | 50 | 30 | |
| 518 | 4 | 2885 | 2040 | gi\|475107 | regulatory protein [Pediococcus | 50 | 29 | |
| | | | | | pentosaceus] | | | |
| 528 | 1 | 3 | 665 | gi\|215098 | excisionase [Bacteriophage 154a] | 50 | 38 | |
| 562 | 1 | 631 | 107 | gi\|1592205 | M. jannaschii predicted coding region | 50 | 28 | |
| | | | | | MJ1595 [Methanococcus jannaschii] | | | |
| 596 | 1 | 227 | 1153 | gi\|963039 | orf gene product [Enterococcus hirae] | 50 | 26 | |
| 680 | 1 | 2 | 1090 | gi\|1050297 | product p150Glued [Neurospora crassa] | 50 | 27 | |
| 755 | 1 | 2 | 430 | gi\|1736469 | Tetracenomycin C resistance and export | 50 | 33 | |
| | | | | | protein. [Escherichia coli] | | | |
| 838 | 1 | 428 | 3 | gi\|530424 | 50S ribosomal protein [Mycoplasma | 50 | 30 | |
| | | | | | capricolum] | | | |
| 14 | 2 | 3453 | 538 | gi\|47049 | asa1 gene product (AA 1-1296) | 49 | 25 | |
| | | | | | [Enterococcus faecalis] ir\|S10223\|HMSO1F | | | |
| | | | | | aggregation protein asa1 - Enterococcus | | | |
| | | | | | faecalis asmid pAD1 | | | |
| 56 | 7 | 5367 | 4822 | gi\|924754 | glycine reductase complex selenoprotein B | 49 | 31 | |
| | | | | | [Clostridium litorale] | | | |
| 68 | 9 | 4741 | 7389 | gi\|1591494 | M. jannaschii predicted coding region | 49 | 21 | |
| | | | | | MJ0797 [Methanococcus jannaschii] | | | |
| 94 | 10 | 9425 | 6633 | gi\|1146243 | 22.4% identity with Escherichia coli DNA- | 49 | 30 | |
| | | | | | damage inducible protein . . . ; putative | | | |
| | | | | | [Bacillus subtilis] | | | |
| 98 | 12 | 12306 | 11701 | gi\|1303784 | YqeD [Bacillus subtilis] | 49 | 26 | |
| 117 | 7 | 4789 | 6228 | gi\|435493 | orf4 gene product [Lactococcus lactis] | 49 | 26 | |
| 123 | 21 | 18576 | 19745 | gi\|298032 | EF [Streptococcus suis] | 49 | 29 | |
| 125 | 4 | 2358 | 1594 | gnl\|PID\|e237295 | unknown [Saccharomyces cerevisiae] | 49 | 27 | |
| 125 | 6 | 4235 | 3453 | gi\|1573885 | glycosyl transferase (lgtD) [Haemophilus | 49 | 32 | |
| | | | | | influenzae] | | | |
| 144 | 5 | 3715 | 4062 | gi\|507130 | emm64 gene product [Streptococcus | 49 | 30 | |
| | | | | | pyogenes] | | | |
| 162 | 8 | 10472 | 9120 | gi\|47045 | NADH oxidase [Enterococcus faecalis] | 49 | 34 | |
| 179 | 18 | 18426 | 17848 | gi\|40060 | DNA polymerase III (AA 1-1437) [Bacillus | 49 | 27 | |
| | | | | | subtilis] p\|P13267\|DP3A_BACSU DNA | | | |
| | | | | | POLYMEPASE III, ALPHA CHAIN (EC 2.7.7.7). | | | |
| 180 | 19 | 18727 | 19917 | gi\|143000 | proton glutamate symport protein [Bacillus | 49 | 31 | |
| | | | | | stearothermophilus] pir\|S26247\|S26247 | | | |
| | | | | | glutamate/aspartate transport protein - | | | |
| | | | | | Bacillus tearothermophilus | | | |
| 224 | 1 | 145 | 1371 | gi\|1103862 | TolA [Pseudomonas aeruginosa] | 49 | 32 | |
| 236 | 8 | 10955 | 9249 | gi\|431272 | lysis protein [Bacillus subtilis] | 49 | 28 | |
| 278 | 1 | 757 | 2 | gi\|467478 | unknown [Bacillus subtilis] | 49 | 29 | |
| 290 | 8 | 6860 | 7366 | gi\|466875 | nifU; B1496_C1_157 [Mycobacterium leprae] | 49 | 35 | |
| 318 | 5 | 4065 | 3190 | gi\|144859 | ORF B [Clostridium perfringens] | 49 | 25 | |
| 318 | 8 | 6052 | 5033 | gi\|1439528 | EIIC-man [Lactobacillus curvatus] | 49 | 30 | |
| 335 | 1 | 534 | 40 | gi\|216861 | 24K membrane protein [Pseudomonas | 49 | 24 | |
| | | | | | aeruginosa] | | | |
| 338 | 4 | 2861 | 2169 | gnl\|PID\|e288536 | F37H8.a [Caenorhabditis elegans] | 49 | 30 | |
| 346 | 4 | 1257 | 2273 | gi\|536970 | ORF_f543 [Escherichia coli] | 49 | 25 | |
| 355 | 20 | 12902 | 15262 | gi\|292836 | trichohyalin [Homo sapiens] | 49 | 20 | |
| 366 | 1 | 1 | 1437 | gi\|405857 | yehU [Escherichia coli] | 49 | 26 | |
| 375 | 8 | 7663 | 6470 | gi\|1573546 | H. influenzae predicted coding region | 49 | 30 | |
| | | | | | H10561 [Haemophilus influenzae] | | | |
| 377 | 2 | 1624 | 392 | gi\|532553 | ORF20 [Enterococcus faecalis] | 49 | 27 | |
| 399 | 5 | 3960 | 3142 | gi\|1742362 | nta operon transcriptional regulator. | 49 | 29 | |
| | | | | | [Escherichia coli] | | | |
| 456 | 1 | 1070 | 342 | gi\|290533 | similar to E. coli ORF adjacent to suc | 49 | 27 | |
| | | | | | operon; similar to gntR class f regulatory | | | |
| | | | | | proteins [Escherichia coli] | | | |
| 619 | 1 | 2 | 232 | gi\|665956 | ribosomal protein S20 homolog [Aeromonas | 49 | 41 | |
| | | | | | sobria] sp\|P45786\|RS20_AERHY 30S RIBOSOMAL | | | |
| | | | | | PROTEIN S20 (FRAGMENT). | | | |
| | | | | | sp\|P45788\|RS20_AERSO 30S RIBOSOMAL PROTEIN | | | |
| | | | | | S20 (FRAGMENT). | | | |
| 621 | 1 | 319 | 942 | gi\|149456 | nisin-resistance protein [Lactococcus | 49 | 29 | |
| | | | | | lactis] | | | |
| 630 | 1 | 3 | 1190 | gi\|537145 | ORF_f437 [Escherichia coli] | 49 | 34 | |
| 736 | 1 | 859 | 2 | gi\|1592020 | hypothetical protein (SP:P37555) | 49 | 27 | |
| | | | | | [Methanococcus jannaschii[ | | | |
| 849 | 1 | 232 | 11 | gi\|145514 | cyclopropane fatty acid synthase | 49 | 35 | |
| | | | | | [Escherichia coli] | | | |
| 47 | 11 | 14140 | 13307 | gi\|1045937 | M. genitalium predicted coding region | 48 | 34 | |
| | | | | | MG246 [Mycoplasma genitalium] | | | |
| 103 | 4 | 2492 | 1605 | gi\|1591514 | membrane protein [Methanococcus | 48 | 19 | |
| | | | | | jannaschii] | | | |
| 127 | 7 | 6836 | 5736 | gi\|1573128 | hypothetical [Haemophilus influenzae] | 48 | 24 | |
| 138 | 22 | 14742 | 15590 | gi\|580884 | ipa-89d gene product [Bacillus subtilis] | 48 | 33 | |
| 160 | 6 | 3048 | 3665 | gi\|1652295 | serine esterase [Synechocystis sp.] | 48 | 28 | |
| 162 | 3 | 3048 | 2491 | gn\|143830 | xpaC [Bacillus subtilis] | 48 | 13 | |
| 193 | 2 | 1257 | 310 | gi\|1591153 | hypothetical protein (SP:P46348) | 48 | 24 | |
| | | | | | [Methanococcus jannaschii] | | | |
| 219 | 1 | 61 | 573 | gnl\|PID\|e257628 | ORF [Lactococcus lactis] | 48 | 32 | |
| 221 | 11 | 5952 | 6428 | gi\|1303733 | YgaN [Bacillus subtilis] | 48 | 31 | |
| 232 | 4 | 2776 | 1712 | gi\|142707 | comG2 gene product [Bacillus subtilis] | 48 | 24 | |
| 236 | 6 | 8618 | 7689 | gi\|550075 | cephalosporin-C deacetylase [Bacillus | 48 | 26 | |
| | | | | | subtilis] | | | |
| 238 | 28 | 25896 | 26825 | gi\|47906 | rha regulatory protein [Salmonella | 48 | 31 | |
| | | | | | typhimurium] | | | |
| 251 | 2 | 1935 | 640 | gi\|1143026 | ORF10 [Spiroplasma virus] | 48 | 30 | |
| 252 | 1 | 2036 | 3 | gnl\|PID\|e228699 | homologous to yqb0 of the skin element | 48 | 37 | |
| | | | | | [Bacillus subtilis] | | | |
| 269 | 1 | 481 | 2 | gi\|1045975 | sensory rhodopsin II transducer | 48 | 28 | |
| | | | | | [Mycoplasma genitalium] | | | |
| 315 | 5 | 4604 | 2649 | gi\|396400 | similar to eukaryotic Na+/H+ exchangers | 48 | 30 | |
| | | | | | [Escherichia coli] sp\|P32703\|YJCE_ECOLI | | | |
| | | | | | HYPOTHETICAL 60.5 KD PROTEIN IN SOXR-ACS | | | |
| | | | | | NTERGENIC REGION (O549). | | | |
| 327 | 1 | 128 | 916 | gi\|216314 | esterase [Bacillus stearothermophilus] | 48 | 30 | |
| 330 | 6 | 4486 | 5337 | gi\|43942 | first subunit of EII-Sor [Klebsiella | 48 | 21 | |
| | | | | | pneumoniae] | | | |
| 330 | 7 | 5325 | 6230 | gi\|147404 | mannose permease subunit II-M-Man | 48 | 33 | |
| | | | | | [Escherichia coli] | | | |
| 345 | 10 | 9571 | 10521 | gi\|1736789 | Collagenase precursor (EC 3.4.-.-). | 48 | 26 | |
| | | | | | [Escherichia coli] | | | |
| 509 | 1 | 1 | 444 | gi\|606376 | ORF_o162 [Escherichia coli] | 48 | 33 | |
| 531 | 1 | 624 | 109 | sp\|P50848\|YPWA_BAC | HYPOTHETICAL 58.2 KD PROTEIN IN KDGT-XPT | 48 | 33 | |
| | | | | SU | INTERGENIC REGION. | | | |
| 549 | 3 | 962 | 369 | gi\|1001212 | molybdenum cofactor biosynthesis protein C | 48 | 32 | |
| | | | | | [Synechocystis sp.] | | | |
| 725 | 1 | 3 | 500 | gi\|1151158 | repeat organellar protein [Plasmodium | 48 | 25 | |
| | | | | | chabaudi] | | | |
| 789 | 1 | 133 | 717 | gi\|42724 | rhaS (AA 1-278) [Escherichia coli] | 48 | 39 | |
| 936 | 1 | 32 | 316 | gi\|532549 | ORF16 [Enterococcus faecalis] | 48 | 45 | |
| 2 | 2 | 2662 | 449 | gi\|929878 | J1027 gene product [Saccharomyces | 47 | 20 | |
| | | | | | cerevisiae] | | | |
| 4 | 2 | 1002 | 2192 | gi\|763052 | integrase [Bacteriophage T270] | 47 | 29 | |
| 21 | 8 | 6350 | 5355 | gi\|1066343 | mu-crystallin [Homo sapiens] | 47 | 29 | |
| 25 | 3 | 915 | 2048 | gi\|1064813 | homologous to sp:PHOR_BACSU [Bacillus | 47 | 21 | |
| | | | | | subtilis] | | | |
| 59 | 2 | 953 | 1378 | gi\|872306 | integral membrane protein [Streptomyces | 47 | 26 | |
| | | | | | pristinaespiralis] pir\|S57509\|S57509 | | | |
| | | | | | integral membrane protein - Streptomyces | | | |
| | | | | | ristinaespiralis | | | |
| 81 | 7 | 4970 | 4206 | gi\|1591754 | hypothetical protein (SP:P39364) | 47 | 22 | |
| | | | | | [Methanococcus jannaschii] | | | |
| 82 | 3 | 1534 | 866 | gi\|397526 | clumping factor [Staphylococcus aureus] | 47 | 21 | |
| 110 | 5 | 2313 | 3767 | gil 151928 | 48 kDa protein [Rhodobacter sphaeroides] | 47 | 26 | |
| 150 | 11 | 7839 | 9107 | gnl\|PID\|e275490 | C30H6.k [Caenorhabditis elegans] | 47 | 16 | |
| 161 | 2 | 116 | 1450 | gnl\|PID\|e283830 | aminotransferase [Sulfolobus solfataricus] | 47 | 23 | |
| 165 | 8 | 8081 | 6129 | gi\|924925 | heparinase III protein [Cytophaga | 47 | 29 | |
| | | | | | heparina] | | | |
| 180 | 31 | 31515 | 31054 | gi\|1591753 | N-acetylglucosamine-1-phosphate | 47 | 29 | |
| | | | | | transferase [Methanococcus jannaschii] | | | |
| 194 | 11 | 8247 | 9236 | gi\|1480429 | putative transcriptional regulator | 47 | 26 | |
| | | | | | [Bacillus stearothermophilus] | | | |
| 225 | 2 | 1039 | 701 | gi\|212992 | Protein sequence and annotation available | 47 | 33 | |
| | | | | | soon via Swiss-Prot; available at present | | | |
| | | | | | via e-mail from LABEIT@EMBL-Heidelberg.DE | | | |
| | | | | | [Homo sapiens] | | | |
| 232 | 1 | 196 | 969 | gi\|293033 | integrase [Bacteriophage phi-LC3] | 47 | 30 | |
| 232 | 6 | 3687 | 3340 | gi\|142706 | comGl gene product [Bacillus subtilis] | 47 | 28 | |
| 233 | 10 | 8424 | 6739 | gi\|887816 | possible start 13 codons upstream, for | 47 | 35 | |
| | | | | | o765 [Escherichia coli] | | | |
| 346 | 2 | 706 | 1083 | gi\|536970 | ORF_f543 [Escherichia coli] | 47 | 27 | |
| 352 | 1 | 112 | 843 | gi\|1591857 | H+-transporting ATPase [Methanococcus | 47 | 28 | |
| | | | | | jannaschii] | | | |
| 410 | 1 | 3 | 980 | gi\|1652869 | NADH dehydrogenase [Synechocystis sp.] | 47 | 30 | |
| 465 | 2 | 1976 | 1749 | gi\|211659 | p68 protein; c-rel proto-oncogene [Gallus | 47 | 30 | |
| | | | | | gallus] | | | |
| 491 | 3 | 3752 | 2466 | gi\|881434 | ORFP [Bacillus subtilis] | 47 | 24 | |
| 501 | 1 | 48 | 809 | gi\|467429 | unknown [Bacillus subtilis] | 47 | 33 | |
| 532 | 1 | 3 | 287 | gi\|755724 | alpha-toxin [Clostridium novyi] | 47 | 32 | |
| 578 | 1 | 707 | 81 | gi\|532547 | ORF14 [Enterococcus faecalis] | 47 | 30 | |
| 605 | 4 | 2051 | 2470 | gi\|1783233 | hypothetical [Bacillus subtilis] | 47 | 22 | |
| 626 | 3 | 2459 | 2169 | gi\|1573573 | 2′,3′-cyclic-nucleotide 2′- | 47 | 44 | |
| | | | | | phosphodiesterase (cpdB) [Haemophilus | | | |
| | | | | | influenzae] | | | |
| 650 | 1 | 1042 | 341 | gi\|404802 | integrase [Saccharopolyspora erythraea] | 47 | 26 | |
| 665 | 1 | 714 | 1175 | gi\|143655 | sporulation protein [Bacillus subtilis] | 47 | 22 | |
| 754 | 2 | 1086 | 736 | gi\|143835 | PBSX repressor [Bacillus subtilis] | 47 | 27 | |
| 845 | 1 | 2 | 241 | gi\|1303952 | YqjA [Bacillus subtilis] | 47 | 26 | |
| 911 | 1 | 1 | 456 | gi\|1019640 | ORFX (a homolog to the prgX gene of the | 47 | 26 | |
| | | | | | pheromone response plasmid pCF10); | | | |
| | | | | | putative [Plasmid pHKK701] | | | |
| 933 | 1 | 16 | 303 | gi\|331002 | first methionine codon in the ECLF1 ORF | 47 | 29 | |
| | | | | | [Saimiriine herpesvirus 2] gi\|60394 ORF | | | |
| | | | | | 73; ECLF1 [Saimiriine herpesvirus 2] | | | |
| 17 | 17 | 13073 | 13675 | gi\|1304597 | abortive phage resistance protein | 46 | 27 | |
| | | | | | [Lactococcus lactis] | | | |
| 19 | 11 | 5515 | 6393 | gi\|1353529 | ORF12 [Bacteriophage rlt] | 46 | 28 | |
| 42 | 3 | 2460 | 3011 | gi\|1064814 | homologous to sp:PHOP_BACEUB [Bacillus | 46 | 33 | |
| | | | | | subtilis] | | | |
| 49 | 9 | 4042 | 5793 | gnl\|PID\|e59644 | predicted 86.4kd protein; S2Kd observed | 46 | 22 | |
| | | | | | [Mycobacteriophage 15] | | | |
| 74 | 6 | 4039 | 3434 | gi\|143542 | PNA polymerase sigma-30 factor [Bacillus | 46 | 27 | |
| | | | | | licheniformis] pir\|B28625\|SZBSSL | | | |
| | | | | | transcription initiation factor sigma H - | | | |
| | | | | | acillus licheniformis | | | |
| 89 | 14 | 14259 | 12967 | gi\|1499089 | M. jannaschii predicted coding region | 46 | 32 | |
| | | | | | MJ0305 [Methanococcus jannaschii] | | | |
| 89 | 15 | 15737 | 14427 | gi\|1653339 | hypothetical protein [Synechocystis sp.] | 46 | 22 | |
| 94 | 13 | 12634 | 11132 | gi\|1402515 | membrane-spanning transporter protein | 46 | 23 | |
| | | | | | [Clostnidium perfringens] | | | |
| 100 | 18 | 13493 | 11958 | gi\|15470 | portal protein [Bacteriophage SPP1] | 46 | 31 | |
| 144 | 2 | 2364 | 1126 | gnl\|PID\|e183450 | hypothetical EcsB protein [Bacillus | 46 | 25 | |
| | | | | | subtilis] | | | |
| 144 | 9 | 8977 | 6236 | gi\|710421 | unknown [Staphylococcus aureus] | 46 | 24 | |
| 152 | 7 | 3397 | 4557 | gnl\|PID\|e254991 | hypothetical protein [Bacillus subtilis] | 46 | 25 | |
| 158 | 7 | 7144 | 5993 | gi\|1045800 | ribose transport system permease protein | 46 | 28 | |
| | | | | | [Mycoplasma genitalium] | | | |
| 180 | 11 | 10882 | 10055 | gi\|303953 | esterase [Acinetobacter calcoaceticus] | 46 | 23 | |
| 181 | 3 | 1173 | 976 | gi\|1591638 | M. jannaschii predicted coding region | 46 | 36 | |
| | | | | | MJ0975 [Methanococcus jannaschii] | | | |
| 240 | 1 | 715 | 221 | gi\|1766062 | Ats1 [Schizosaccharomyces pombe] | 46 | 28 | |
| 254 | 2 | 499 | 2 | gi\|153661 | translational initiation factor IF2 | 46 | 32 | |
| | | | | | [Enterococcus faecium] sp\|P18311\|IF2_ENTFC | | | |
| | | | | | INITIATION FACTOR IF-2. | | | |
| 262 | 4 | 5276 | 4431 | pir\|A45605\|A45605 | mature-parasite-infected erythrocyte | 46 | 20 | |
| | | | | | surface antigen MESA - Plasmodium | | | |
| | | | | | falciparum | | | |
| 309 | 1 | 2 | 673 | gi\|1651714 | type 4 prepilin peptidase [Synechocystis | 46 | 40 | |
| | | | | | sp.] | | | |
| 312 | 1 | 18 | 872 | gi\|580884 | ipa-89d gene product [Bacillus subtilis] | 46 | 32 | |
| 324 | 6 | 4450 | 4836 | gi\|1061418 | ArsC [Plasmid R46] | 46 | 28 | |
| 345 | 1 | 2241 | 1333 | gi\|144859 | ORF B [Clostridium perfringens] | 46 | 24 | |
| 386 | 4 | 1438 | 2421 | gi\|405894 | 1-phosphofructokinase [Escherichia coli] | 46 | 31 | |
| 395 | 8 | 3584 | 3853 | gnl\|PID\|e120267 | sucrose-phosphate synthase [Beta vulgaris] | 46 | 25 | |
| 491 | 2 | 2527 | 1169 | gnl\|PID\|e267595 | Unknown, similar to peptidases [Bacillus | 46 | 29 | |
| | | | | | subtilis] | | | |
| 495 | 3 | 612 | 869 | gi\|406286 | triose phosphate/phosphate translocator | 46 | 27 | |
| | | | | | [Flaveria pringlei] pir\|537553\|S37553 | | | |
| | | | | | triose phosphate/3- | | | |
| | | | | | phosphoglycerate/phosphate ranslocator - | | | |
| | | | | | Flaveria pringlei | | | |
| 513 | 1 | 2 | 946 | gi\|143024 | glucose-resistance amylase regulator | 46 | 26 | |
| | | | | | [Bacillus subtilis] pir\|S15318\|515318 ccpA | | | |
| | | | | | protein - Bacillus subtilis | | | |
| | | | | | sp\|P25144\|CCPA_BACSU GLUCOSE-RESISTANCE | | | |
| | | | | | AMYLASE REGULATOR CATABOLITE CONTROL | | | |
| | | | | | PROTEIN). | | | |
| 520 | 3 | 914 | 2674 | gi\|1163086 | microfilarial sheath protein SHP3 [Brugia | 46 | 27 | |
| | | | | | malayi] | | | |
| 554 | 1 | 3 | 788 | gi\|413972 | ipa-48r gene product [Bacillus subtilis] | 46 | 27 | |
| 568 | 1 | 1574 | 3 | gi\|532549 | ORF16 [Enterococcus faecalis] | 46 | 28 | |
| 809 | 1 | 506 | 135 | gi\|49021 | surface exclusion protein (SEA1) | 46 | 28 | |
| | | | | | [Enterococcus faecalis] ir\|522452\|S22452 | | | |
| | | | | | surface exclusion protein sea1 precursor - | | | |
| | | | | | terococcus faecalis plasmid pAD1 | | | |
| 813 | 1 | 2 | 1090 | gi\|150556 | surface protein [Plasmid pCF10] | 46 | 34 | |
| 78 | 2 | 4915 | 2516 | gi\|577295 | The ha1225 gene product is related to | 45 | 20 | |
| | | | | | human alpha-glucosidase. [Homo apiens] | | | |
| 81 | 9 | 6123 | 5386 | gi\|147200 | phnF protein [Escherichia coli] | 45 | 28 | |
| 85 | 1 | 120 | 761 | gi\|457514 | gltC [Bacillus subtilis] | 45 | 19 | |
| 94 | 11 | 10681 | 9668 | gi\|289753 | homology with nucleolin protein; putative | 45 | 23 | |
| | | | | | [Caenorhabditis elegans] pir\|S44897\|S44897 | | | |
| | | | | | ZK1236.2 protein - Caenorhabditis elegans | | | |
| | | | | | sp\|P34618\|Y082_CAEEL HYPOTHETICAL 33.8 KD | | | |
| | | | | | PROTEIN ZK1236.2 IN HROMOSOME III. | | | |
| 108 | 3 | 2427 | 1789 | gnl\|PID\|e263931 | OrfD [Streptococcus pneumoniae] | 45 | 27 | |
| 108 | 4 | 3338 | 2352 | gi\|606150 | ORF_f309 [Escherichia coli] | 45 | 25 | |
| 131 | 6 | 3981 | 5309 | gi\|1590845 | hypothetical protein (PIR:551413) | 45 | 36 | |
| | | | | | [Methanococcus jannaschii] | | | |
| 144 | 11 | 10215 | 8944 | gi\|1001554 | hypothetical protein [Synechocystis sp.] | 45 | 30 | |
| 164 | 11 | 8247 | 6736 | gi\|409925 | VirR positive regulator [Streptococcus | 45 | 22 | |
| | | | | | pyogenes] | | | |
| 192 | 1 | 1598 | 591 | gi\|1736826 | Lysozyme M1 precursor (EC 3.2.1.17) (1,4- | 45 | 27 | |
| | | | | | b-N-acetylmuramidase M1). [Escherichia | | | |
| | | | | | coli] | | | |
| 223 | 16 | 14409 | 15212 | gi\|1651958 | hypothetical protein [Synechocystis sp.] | 45 | 32 | |
| 279 | 7 | 5236 | 5772 | gi\|1736514 | Isochorismatase (EC 3.3.2.1) (2,3 dihydro- | 45 | 29 | |
| | | | | | 2,3 dihydroxybenzoate synthase). | | | |
| | | | | | [Escherichia coli] | | | |
| 364 | 3 | 2419 | 4098 | gi\|309662 | pheromone binding protein [Plasmid pCF10] | 45 | 26 | |
| 459 | 1 | 2 | 307 | gi\|1679640 | ORFA [Mycoplasma mycoides mycoides SC] | 45 | 27 | |
| 491 | 1 | 1022 | 135 | sp\|P27434\|YFGA_ECO | HYPOTHETICAL 36.2 KD PROTEIN IN NDK-GCPE | 45 | 20 | |
| | | | | LI | INTERGENIC REGION. | | | |
| 496 | 1 | 847 | 2 | gi\|1208489 | serum resistance locus BrkB [Synechocystis | 45 | 19 | |
| | | | | | sp.] | | | |
| 542 | 2 | 1169 | 804 | gi\|1064811 | function unknown [Bacillus subtilis] | 45 | 28 | |
| 63 | 3 | 1047 | 1919 | gi\|39848 | U3 [Bacillus subtilis] | 44 | 26 | |
| 93 | 3 | 1108 | 1374 | sp\|Q4747\|SRF2_SAC | SURFACTIN SYNTHETASE SUBUNIT 2. | 44 | 27 | |
| | | | | SU | | | | |
| 155 | 10 | 8354 | 7620 | sp\|P35136\|SERA_BAC | D-3-PHOSPHOGLYCERATE DEHYDROGENASE (EC | 44 | 29 | |
| | | | | SU | 1.1.1.95) (PGDH). | | | |
| 215 | 2 | 2192 | 1134 | gi\|468760 | ORF334 [Rhizobium meliloti] | 44 | 31 | |
| 303 | 1 | 466 | 2 | gi\|431950 | similar to a B.subtilis gene (GB: | 44 | 22 | |
| | | | | | BACHEMEHY_5) [Clostridium asteurianum] | | | |
| 310 | 1 | 284 | 39 | pir\|S01294\|S01294 | intermediate filament protein B - Roman | 44 | 26 | |
| | | | | | snail | | | |
| 311 | 1 | 122 | 2668 | gi\|532549 | ORF16 [Enterococcus faecalis] | 44 | 27 | |
| 320 | 1 | 709 | 2 | gi\|290801 | member of super-family of ABC proteins | 44 | 23 | |
| | | | | | [Francisella tularensis (var. ovicida)] | | | |
| 341 | 14 | 13882 | 12998 | gi\|142863 | replication initiation protein [Bacillus | 44 | 16 | |
| | | | | | subtilis] | | | |
| 345 | 15 | 16445 | 18001 | gi\|151282 | DL-hydantoinase [Pseudomonas sp.] | 44 | 34 | |
| 386 | 3 | 1340 | 570 | sp\|P46117\|YARA_PRO | HYPOTHETICAL 31.5 KD PROTEIN IN AARA | 44 | 19 | |
| | | | | ST | 3′REGION. | | | |
| 862 | 1 | 483 | 4 | gi\|929796 | precursor of the major merozoite surface | 44 | 26 | |
| | | | | | antigens [Plasmodium alciparum] | | | |
| 19 | 3 | 1695 | 1372 | gi\|603263 | Ye1055p [Saccharomyces cerevisiae] | 43 | 31 | |
| 45 | 17 | 14045 | 14995 | gnl\|PID\|e233895 | hypothetical protein [Bacillus subtilis] | 43 | 32 | |
| 57 | 1 | 667 | 317 | gi\|664840 | TagB [Dictyostelium discoideum] | 43 | 22 | |
| 71 | 2 | 1537 | 2568 | gi\|1303981 | YgkD [Bacillus subtilis] | 43 | 36 | |
| 72 | 18 | 20511 | 20164 | gi\|349045 | merozoite surface antigen 2 [Plasmodium | 43 | 36 | |
| | | | | | falciparum] | | | |
| 94 | 9 | 6581 | 6039 | gi\|1146245 | putative [Bacillus subtilis] | 43 | 28 | |
| 180 | 17 | 16391 | 17656 | gi\|290540 | f445 [Escherichia coli] | 43 | 24 | |
| 252 | 2 | 2407 | 1829 | gi\|154381 | chemoreceptor [Salmonella typhimurium] | 43 | 19 | |
| 276 | 30 | 19091 | 18480 | gi\|15470 | portal protein [Bacteriophage SPP1] | 43 | 23 | |
| 311 | 2 | 2666 | 4639 | gi\|160299 | glutamic acid-rich protein [Plasmodium | 43 | 28 | |
| | | | | | falciparum] pir\|A54514\|A54514 glutamic | | | |
| | | | | | acid-rich protein precursor - Plasmodium | | | |
| | | | | | alciparum | | | |
| 631 | 2 | 1126 | 2328 | gi\|1519696 | coded for by C. elegans cDNA yk126f9.5; | 43 | 27 | |
| | | | | | coded for by C. elegans cDNA yk159h6.3; | | | |
| | | | | | coded for by C. elegans cDNA yk126f9.3; | | | |
| | | | | | coded for by C. elegans cDNA yk159h6.5 | | | |
| | | | | | [Caenorhabditis elegans] | | | |
| 11 | 3 | 1509 | 2342 | gi\|143150 | levR [Bacillus subtilis] | 42 | 21 | |
| 45 | 14 | 10730 | 12028 | gi\|666069 | orf2 gene product [Lactobacillus | 42 | 23 | |
| | | | | | leichmannii] | | | |
| 72 | 19 | 21070 | 21981 | gnl\|PID\|e236595 | orf7 gene product [Enterococcus faecalis] | 42 | 23 | |
| 123 | 35 | 32205 | 32768 | gi\|1772652 | 2-keto-3-deoxygluconate kinase [Haloferax | 42 | 27 | |
| | | | | | alicantei] | | | |
| 136 | 5 | 2737 | 2375 | gi\|153858 | wall-associated protein [Streptococcus | 42 | 27 | |
| | | | | | mutans | | | |
| 167 | 4 | 2701 | 6540 | gi\|1519696 | coded for by C. elegans cDNA yk126f9.5; | 42 | 27 | |
| | | | | | coded for by C. elegans cDNA yk159h6.3; | | | |
| | | | | | coded for by C. elegans cDNA yk126f9.3; | | | |
| | | | | | coded for by C. elegans cDNA yk159h6.5 | | | |
| | | | | | [Caenorhabditis elegans] | | | |
| 195 | 31 | 12430 | 13155 | pir\|S33124\|S33124 | tpr protein - human | 42 | 24 | |
| 211 | 1 | 187 | 2 | gi\|1653346 | GDP-mannose pyrophosphorylase | 42 | 33 | |
| | | | | | [Synechocystis sp.] | | | |
| 242 | 13 | 8089 | 12447 | gi\|951460 | FIM-C.1 gene product [Xenopus laevis] | 42 | 31 | |
| 305 | 5 | 4354 | 5340 | gi\|1408485 | yxdM gene product [Bacillus subtilis] | 42 | 25 | |
| 355 | 18 | 9964 | 12549 | gi\|532549 | ORF16 [Enterococcus faecalis] | 42 | 30 | |
| 446 | 4 | 4428 | 5261 | gi\|47528 | glucosyltransferase S [Streptococcus | 42 | 25 | |
| | | | | | salivarius] | | | |
| 656 | 3 | 2866 | 3456 | gi\|142857 | MreD protein [Bacillus subtilis] | 42 | 25 | |
| 686 | 11 | 3646 | 3921 | pir\|A44805\|A44805 | eggshell protein - fluke (Schistosoma | 42 | 42 | |
| | | | | | haematobium) (subelone SH.E 2-1) | | | |
| 920 | 1 | 41 | 316 | gi\|532549 | ORF16 [Enterococcus faecalis] | 42 | 40 | |
| 23 | 3 | 729 | 487 | gi\|414525 | meiotin-1 [Lilium longiflorum] | 41 | 41 | |
| 456 | 5 | 3511 | 2324 | gi\|1591610 | probable ATP-dependent helicase | 41 | 21 | |
| | | | | | [Methanococcus jannaschii] | | | |
| 98 | 17 | 16843 | 16274 | gi\|1742129 | Immunity repressor protein. [Escherichia | 41 | 23 | |
| | | | | | coli] | | | |
| 167 | 6 | 6734 | 9811 | gnl\|PID\|e249616 | F56H9.1 [Caenorhabditis elegans] | 41 | 37 | |
| 171 | 13 | 10879 | 11871 | gi\|331002 | first methionine codon in the ECLF1 ORE | 41 | 23 | |
| | | | | | [Saimiriine herpesvirus 2] gi\|60394 ORF | | | |
| | | | | | 73; ECLF1 [Saimiriine herpesvirus 2] | | | |
| 181 | 2 | 1012 | 500 | gi\|455315 | ORF 4 [Plasmid pIP404] | 41 | 24 | |
| 230 | 4 | 3664 | 3224 | gi\|498251 | glutamate/aspartate transporter II [Homo | 41 | 22 | |
| | | | | | sapiens] | | | |
| 718 | 1 | 2 | 613 | gi\|984656 | ORF3 [Salmonella typhimurium] | 41 | 22 | |
| 219 | 30 | 16391 | 17770 | gi\|806704 | Upf2p [Saccharomyces cerevisiae] | 40 | 21 | |
| 164 | 16 | 16440 | 17951 | gi\|348056 | trans-acting positive regulator [Bacillus | 40 | 22 | |
| | | | | | anthracis] | | | |
| 200 | 12 | 5956 | 4841 | gi\|1574243 | H. influenzae predicted coding region | 40 | 24 | |
| | | | | | HI1405 [Haemophilus influenzae] | | | |
| 216 | 10 | 6799 | 7194 | gi\|146279 | glucitol-specific enzyme III (gutB) | 40 | 27 | |
| | | | | | [Escherichia coli] | | | |
| 292 | 13 | 8633 | 10741 | gi\|1008233 | ORF YJL076w [Saccharomyces cerevisiae] | 40 | 18 | |
| 345 | 13 | 14050 | 15333 | gi\|581051 | cytosine permease [Escherichia coli] | 40 | 25 | |
| 521 | 1 | 177 | 1466 | gi\|289614 | homology with glucose induced repressor, | 40 | 18 | |
| | | | | | GRR1; putative Caenorhabditis elegans] | | | |
| 64 | 3 | 2646 | 1855 | gi\|154924 | spectinomycin adenyltransferase | 39 | 27 | |
| | | | | | [Transposon Tn554] | | | |
| 100 | 17 | 12037 | 10565 | gi\|1052806 | product required for head morphogenesis | 39 | 24 | |
| | | | | | [Bacteriophage SPP1] | | | |
| 529 | 1 | 326 | 4939 | gi\|295671 | selected as a weak suppressor of a mutant | 39 | 19 | |
| | | | | | of the subunit AC40 of DNA ependant RNA | | | |
| | | | | | polymerase I and III [Saccharomyces | | | |
| | | | | | cerevisiae] | | | |
| 49 | 2 | 518 | 931 | gi\|166162 | Bacteriophage phi-11 int gene activator | 38 | 19 | |
| | | | | | [Staphylococcus acteriophage phi 11] | | | |
| 54 | 19 | 11264 | 10854 | gi\|160186 | circumsporozoite protein [Plasmodium | 38 | 31 | |
| | | | | | vivax] | | | |
| 164 | 21 | 22793 | 23587 | gi\|603857 | secreted acid phosphatase 2 (SAP2) | 38 | 18 | |
| | | | | | [Leishniania mexicana] | | | |
| 167 | 3 | 2322 | 2756 | gi\|435039 | proline-rich cell wall protein [Gossypium | 38 | 36 | |
| | | | | | hirsutum] | | | |
| 204 | 2 | 133 | 798 | gi\|396401 | No definition line found [Escherichia | 38 | 25 | |
| | | | | | coli] | | | |
| 475 | 2 | 761 | 1792 | gi\|1574532 | H. influenzae predicted coding region | 38 | 27 | |
| | | | | | HI1680 [Haemophilus influenzae] | | | |
| 164 | 19 | 20738 | 21385 | gi\|165704 | [Rabbit smooth muscle myosin light chain | 37 | 20 | |
| | | | | | kinase mRNA, complete DS.], gene product | | | |
| | | | | | [Oryctolagus cuniculus] | | | |
| 394 | 6 | 5649 | 6395 | gi\|603857 | secreted acid phosphatase 2 (SAP2) | 36 | 16 | |
| | | | | | [Leishmania mexicana] | | | |
| 958 | 1 | 1 | 459 | gi\|951460 | FITA-C.1 gene product [Xenopus laevis] | 36 | 28 | |
| 399 | 21 | 16383 | 21359 | gi\|1707247 | partial CDS [Caenorhabditis elegans] | 34 | 13 | |
| 150 | 12 | 9056 | 11740 | gi\|1015903 | ORF YJR151c [Saccharomyces cerevisiae] | 33 | 19 | |
| 195 | 34 | 13017 | 15512 | gi\|632549 | NF-180 [Petromyzon marinus] | 33 | 18 | |

TABLE 3
E. faecalis-Putative coding regions of novel proteins not similar
to known proteins

| Contig ID | ORF ID | Start (nt) | Stop (nt) |
|---|---|---|---|
| 2 | 1 | 458 | 3 |
| 2 | 3 | 2208 | 2624 |
| 5 | 3 | 928 | 1440 |
| 8 | 6 | 4792 | 5877 |
| 8 | 7 | 5480 | 5262 |
| 12 | 1 | 2 | 832 |
| 12 | 2 | 771 | 4622 |
| 13 | 1 | 2 | 1684 |
| 14 | 1 | 531 | 130 |
| 15 | 2 | 862 | 1197 |
| 16 | 1 | 51 | 200 |
| 17 | 4 | 3309 | 3665 |
| 17 | 13 | 10079 | 10261 |
| 17 | 18 | 14431 | 13682 |
| 17 | 22 | 21525 | 21956 |
| 17 | 27 | 27055 | 27567 |
| 18 | 4 | 2172 | 1591 |
| 18 | 5 | 2524 | 2249 |
| 18 | 7 | 3467 | 3715 |
| 18 | 8 | 4082 | 3555 |
| 18 | 9 | 4333 | 4055 |
| 18 | 10 | 4395 | 4204 |
| 18 | 11 | 4498 | 4677 |
| 18 | 12 | 4656 | 5393 |
| 18 | 13 | 5878 | 5492 |
| 18 | 15 | 6296 | 6931 |
| 19 | 1 | 1047 | 676 |
| 19 | 2 | 1068 | 1247 |
| 19 | 4 | 1747 | 2031 |
| 19 | 5 | 2244 | 2612 |
| 19 | 7 | 2797 | 2943 |
| 19 | 9 | 3873 | 4730 |
| 19 | 13 | 6884 | 7420 |
| 19 | 14 | 7428 | 8042 |
| 19 | 16 | 9246 | 8425 |
| 19 | 17 | 9412 | 9615 |
| 19 | 19 | 9733 | 9918 |
| 19 | 20 | 10032 | 10334 |
| 19 | 21 | 10422 | 11009 |
| 19 | 22 | 11516 | 11944 |
| 19 | 24 | 12423 | 12881 |
| 19 | 26 | 14606 | 15427 |
| 19 | 27 | 15414 | 15848 |
| 19 | 28 | 15802 | 16134 |
| 19 | 29 | 16064 | 16393 |
| 19 | 32 | 17846 | 18052 |
| 19 | 33 | 18021 | 18356 |
| 19 | 34 | 18334 | 18684 |
| 19 | 35 | 18659 | 19036 |
| 19 | 36 | 18991 | 19677 |
| 19 | 37 | 19671 | 20132 |
| 19 | 39 | 22603 | 23337 |
| 19 | 40 | 23319 | 25580 |
| 21 | 2 | 762 | 262 |
| 21 | 5 | 3440 | 2925 |
| 21 | 10 | 7684 | 7241 |
| 23 | 5 | 2098 | 2652 |
| 23 | 8 | 4912 | 4709 |
| 23 | 9 | 4911 | 5246 |
| 23 | 10 | 5087 | 5353 |
| 23 | 22 | 14318 | 14926 |
| 23 | 23 | 14924 | 15565 |
| 23 | 24 | 15559 | 16083 |
| 23 | 29 | 17567 | 18022 |
| 25 | 2 | 553 | 1005 |
| 25 | 5 | 3363 | 2653 |
| 26 | 2 | 1220 | 1654 |
| 27 | 1 | 297 | 4 |
| 28 | 1 | 239 | 2833 |
| 29 | 5 | 3244 | 2822 |
| 29 | 6 | 4014 | 3301 |
| 29 | 7 | 4168 | 4557 |
| 29 | 8 | 5620 | 4595 |
| 32 | 3 | 2646 | 1375 |
| 32 | 4 | 2573 | 3010 |
| 39 | 9 | 4636 | 4986 |
| 40 | 2 | 1346 | 981 |
| 43 | 1 | 120 | 620 |
| 43 | 4 | 1972 | 2280 |
| 45 | 3 | 1557 | 1961 |
| 45 | 4 | 2012 | 2230 |
| 45 | 5 | 2218 | 2553 |
| 45 | 11 | 7226 | 5670 |
| 45 | 12 | 7270 | 10113 |
| 45 | 13 | 10013 | 10732 |
| 46 | 1 | 42 | 872 |
| 46 | 2 | 886 | 1125 |
| 46 | 4 | 2807 | 3100 |
| 47 | 4 | 5101 | 5625 |
| 47 | 10 | 13239 | 12847 |
| 49 | 1 | 106 | 504 |
| 49 | 8 | 2858 | 4132 |
| 49 | 10 | 5777 | 6193 |
| 49 | 11 | 6166 | 6720 |
| 52 | 5 | 3505 | 3110 |
| 52 | 7 | 5160 | 5603 |
| 52 | 8 | 5662 | 5459 |
| 54 | 2 | 400 | 729 |
| 54 | 4 | 1326 | 1610 |
| 54 | 5 | 2354 | 1335 |
| 54 | 6 | 1676 | 2080 |
| 54 | 7 | 2151 | 2576 |
| 54 | 12 | 4181 | 3954 |
| 54 | 13 | 5975 | 6289 |
| 54 | 14 | 6869 | 7144 |
| 54 | 15 | 7433 | 7107 |
| 54 | 18 | 9764 | 11086 |
| 55 | 2 | 252 | 440 |
| 56 | 2 | 1344 | 658 |
| 57 | 9 | 12450 | 12605 |
| 58 | 7 | 7066 | 6425 |
| 59 | 3 | 1350 | 952 |
| 59 | 4 | 1225 | 1515 |
| 59 | 7 | 2958 | 3200 |
| 62 | 6 | 4116 | 3007 |
| 63 | 1 | 77 | 364 |
| 63 | 2 | 455 | 1060 |
| 63 | 7 | 5422 | 5910 |
| 63 | 8 | 5870 | 6751 |
| 63 | 9 | 6688 | 7296 |
| 64 | 2 | 1849 | 1523 |
| 64 | 4 | 3183 | 2644 |
| 64 | 5 | 3422 | 3213 |
| 65 | 5 | 3787 | 3389 |
| 65 | 7 | 5043 | 4300 |
| 65 | 8 | 5354 | 4959 |
| 65 | 9 | 7005 | 6328 |
| 67 | 6 | 3719 | 4060 |
| 68 | 2 | 569 | 348 |
| 68 | 5 | 3234 | 2821 |
| 68 | 6 | 3808 | 3221 |
| 68 | 10 | 7495 | 8106 |
| 70 | 2 | 2102 | 1614 |
| 70 | 3 | 2019 | 2231 |
| 71 | 3 | 3362 | 3787 |
| 72 | 21 | 22464 | 22709 |
| 72 | 22 | 22690 | 23019 |
| 72 | 23 | 23013 | 23834 |
| 73 | 1 | 154 | 2 |
| 74 | 1 | 61 | 486 |
| 74 | 3 | 1334 | 1981 |
| 75 | 4 | 3227 | 2136 |
| 75 | 5 | 3994 | 3251 |
| 75 | 6 | 3348 | 3632 |
| 75 | 7 | 4519 | 4043 |
| 75 | 8 | 4296 | 4529 |
| 75 | 10 | 6518 | 5769 |
| 76 | 2 | 1079 | 1897 |
| 76 | 4 | 2113 | 2436 |
| 76 | 6 | 4737 | 4105 |
| 77 | 3 | 1874 | 2704 |
| 77 | 4 | 2665 | 2459 |
| 78 | 3 | 5814 | 5398 |
| 79 | 3 | 848 | 1645 |
| 79 | 4 | 2121 | 1642 |
| 81 | 8 | 5392 | 4961 |
| 81 | 13 | 8428 | 8874 |
| 81 | 21 | 15746 | 14802 |
| 82 | 1 | 858 | 4 |
| 82 | 2 | 198 | 383 |
| 83 | 3 | 2194 | 2604 |
| 83 | 4 | 2728 | 2405 |
| 83 | 6 | 2855 | 3172 |
| 83 | 10 | 7188 | 6184 |
| 83 | 11 | 7415 | 7065 |
| 83 | 17 | 12259 | 12561 |
| 83 | 21 | 15890 | 16456 |
| 83 | 23 | 16946 | 17251 |
| 84 | 5 | 7071 | 7949 |
| 85 | 7 | 6518 | 6174 |
| 89 | 2 | 1012 | 599 |
| 89 | 3 | 1382 | 939 |
| 89 | 4 | 2350 | 1370 |
| 89 | 5 | 2523 | 2314 |
| 89 | 9 | 7505 | 7182 |
| 89 | 16 | 15846 | 15673 |
| 89 | 19 | 20070 | 19045 |
| 90 | 1 | 3 | 689 |
| 91 | 7 | 3834 | 4127 |
| 91 | 8 | 4288 | 5268 |
| 91 | 9 | 7259 | 5748 |
| 91 | 12 | 9737 | 8973 |
| 91 | 13 | 10162 | 9731 |
| 92 | 3 | 1458 | 958 |
| 92 | 4 | 1934 | 1287 |
| 93 | 2 | 479 | 949 |
| 93 | 4 | 1344 | 1727 |
| 94 | 1 | 770 | 45 |
| 94 | 3 | 1460 | 1618 |
| 94 | 5 | 2279 | 1734 |
| 94 | 12 | 11000 | 10641 |
| 95 | 11 | 7674 | 7907 |
| 95 | 12 | 8604 | 8056 |
| 95 | 13 | 8725 | 8546 |
| 96 | 1 | 758 | 1018 |
| 96 | 2 | 1038 | 1469 |
| 98 | 5 | 6809 | 5994 |
| 98 | 10 | 10338 | 10652 |
| 98 | 11 | 10650 | 11558 |
| 99 | 2 | 232 | 513 |
| 100 | 4 | 3728 | 4048 |
| 100 | 6 | 5866 | 5378 |
| 100 | 7 | 6574 | 5921 |
| 100 | 8 | 6923 | 6534 |
| 100 | 9 | 7355 | 6921 |
| 100 | 10 | 7698 | 7339 |
| 100 | 11 | 8226 | 7744 |
| 100 | 13 | 9395 | 8514 |
| 100 | 15 | 10368 | 10102 |
| 100 | 19 | 14770 | 13505 |
| 100 | 20 | 15300 | 14758 |
| 100 | 21 | 15783 | 15298 |
| 100 | 23 | 17699 | 17292 |
| 100 | 25 | 20933 | 20625 |
| 100 | 26 | 21200 | 20946 |
| 100 | 28 | 23713 | 23156 |
| 100 | 29 | 23948 | 23691 |
| 100 | 30 | 24312 | 23965 |
| 100 | 31 | 24550 | 24287 |
| 100 | 32 | 24912 | 24565 |
| 100 | 33 | 25173 | 24910 |
| 100 | 34 | 26339 | 25158 |
| 100 | 36 | 27251 | 26994 |
| 100 | 37 | 27945 | 27232 |
| 100 | 39 | 28442 | 28227 |
| 100 | 40 | 28657 | 28403 |
| 100 | 46 | 30439 | 31146 |
| 100 | 47 | 31158 | 31712 |
| 101 | 2 | 850 | 464 |
| 101 | 3 | 2453 | 1899 |
| 102 | 6 | 5023 | 5616 |
| 102 | 9 | 6704 | 7111 |
| 103 | 7 | 5454 | 5296 |
| 105 | 2 | 1244 | 1828 |
| 106 | 4 | 5114 | 3294 |
| 106 | 6 | 7622 | 6168 |
| 106 | 7 | 6577 | 6867 |
| 108 | 6 | 5192 | 4158 |
| 110 | 1 | 2 | 454 |
| 110 | 6 | 3689 | 4207 |
| 110 | 9 | 9374 | 8553 |
| 110 | 10 | 9903 | 9361 |
| 110 | 11 | 10175 | 9843 |
| 111 | 6 | 3118 | 3267 |
| 112 | 4 | 2170 | 1043 |
| 114 | 2 | 1347 | 1135 |
| 116 | 8 | 4782 | 5147 |
| 117 | 4 | 2437 | 2670 |
| 117 | 6 | 3876 | 4640 |
| 117 | 8 | 5643 | 5927 |
| 117 | 9 | 6195 | 6488 |
| 117 | 12 | 9655 | 9837 |
| 119 | 1 | 3 | 500 |
| 119 | 2 | 670 | 1158 |
| 119 | 4 | 2730 | 2284 |
| 121 | 3 | 2276 | 3670 |
| 123 | 14 | 14304 | 14555 |
| 123 | 16 | 15305 | 15147 |
| 123 | 24 | 21896 | 22663 |
| 123 | 34 | 31458 | 32207 |
| 125 | 3 | 1581 | 1300 |
| 125 | 7 | 4516 | 4346 |
| 126 | 2 | 85 | 312 |
| 127 | 2 | 1047 | 787 |
| 127 | 3 | 2006 | 1299 |
| 127 | 4 | 3432 | 1924 |
| 128 | 4 | 3094 | 2747 |
| 128 | 5 | 3466 | 3305 |
| 128 | 6 | 4625 | 3507 |
| 128 | 7 | 4726 | 4550 |
| 128 | 13 | 8947 | 8522 |
| 128 | 15 | 9325 | 9582 |
| 128 | 17 | 10126 | 10380 |
| 128 | 24 | 17649 | 18038 |
| 129 | 1 | 276 | 1769 |
| 130 | 7 | 6478 | 6702 |
| 130 | 11 | 9386 | 9769 |
| 133 | 7 | 6622 | 7380 |
| 135 | 2 | 2289 | 1153 |
| 135 | 3 | 3380 | 2271 |
| 135 | 5 | 3778 | 3930 |
| 135 | 6 | 5835 | 5137 |
| 135 | 7 | 6649 | 5852 |
| 135 | 8 | 7021 | 6647 |
| 135 | 9 | 7420 | 7034 |
| 136 | 2 | 963 | 379 |
| 136 | 3 | 2009 | 939 |
| 136 | 4 | 2344 | 1973 |
| 138 | 4 | 5051 | 3636 |
| 138 | 11 | 8499 | 8753 |
| 138 | 12 | 8682 | 8536 |
| 138 | 13 | 8923 | 9270 |
| 138 | 14 | 9333 | 9887 |
| 138 | 15 | 9628 | 10308 |
| 138 | 16 | 10422 | 10216 |
| 138 | 23 | 15980 | 15678 |
| 138 | 24 | 16437 | 16063 |
| 138 | 30 | 19388 | 19828 |
| 139 | 3 | 1068 | 1466 |
| 139 | 4 | 3338 | 1983 |
| 139 | 5 | 3769 | 3317 |
| 139 | 6 | 4114 | 3818 |
| 139 | 7 | 4838 | 4236 |
| 139 | 10 | 5639 | 5175 |
| 142 | 1 | 369 | 106 |
| 142 | 2 | 1005 | 367 |
| 142 | 3 | 2140 | 980 |
| 142 | 4 | 2504 | 2127 |
| 142 | 5 | 2821 | 2474 |
| 142 | 6 | 3294 | 2806 |
| 142 | 7 | 4000 | 3635 |
| 143 | 1 | 650 | 3 |
| 143 | 3 | 1090 | 173 |
| 143 | 4 | 1044 | 433 |
| 144 | 10 | 7570 | 8403 |
| 144 | 12 | 10727 | 10335 |
| 145 | 1 | 188 | 30 |
| 145 | 2 | 775 | 978 |
| 150 | 9 | 6876 | 7166 |
| 150 | 13 | 11538 | 11242 |
| 152 | 1 | 35 | 445 |
| 152 | 2 | 405 | 914 |
| 152 | 3 | 912 | 1430 |
| 152 | 4 | 1349 | 2212 |
| 152 | 5 | 2210 | 2896 |
| 152 | 6 | 2739 | 3368 |
| 152 | 8 | 4479 | 4694 |
| 152 | 11 | 6647 | 7321 |
| 154 | 7 | 4557 | 4195 |
| 155 | 3 | 1227 | 2180 |
| 155 | 12 | 8726 | 9022 |
| 156 | 3 | 3179 | 2664 |
| 158 | 11 | 10876 | 11220 |
| 160 | 1 | 545 | 3 |
| 162 | 1 | 228 | 1349 |
| 162 | 2 | 2513 | 1653 |
| 162 | 7 | 9163 | 7664 |
| 162 | 9 | 10619 | 10990 |
| 162 | 11 | 11891 | 11427 |
| 163 | 3 | 1043 | 1234 |
| 163 | 5 | 3217 | 2021 |
| 163 | 6 | 3455 | 3198 |
| 163 | 8 | 5611 | 4931 |
| 163 | 9 | 5969 | 5580 |
| 163 | 10 | 6144 | 5926 |
| 164 | 2 | 1100 | 1687 |
| 164 | 9 | 5729 | 5259 |
| 164 | 10 | 6778 | 5639 |
| 164 | 12 | 8277 | 8450 |
| 164 | 17 | 18224 | 18526 |
| 164 | 24 | 24751 | 24536 |
| 164 | 27 | 25764 | 26369 |
| 165 | 1 | 17 | 481 |
| 165 | 2 | 2213 | 1389 |
| 165 | 12 | 9871 | 9689 |
| 165 | 14 | 11416 | 10367 |
| 166 | 3 | 1250 | 1669 |
| 167 | 5 | 3774 | 3439 |
| 167 | 7 | 10479 | 14498 |
| 167 | 10 | 17476 | 18768 |
| 168 | 2 | 665 | 393 |
| 172 | 9 | 7018 | 6701 |
| 172 | 10 | 7097 | 7930 |
| 173 | 1 | 2 | 412 |
| 173 | 3 | 2341 | 2024 |
| 173 | 6 | 4234 | 5055 |
| 173 | 9 | 7882 | 7295 |
| 173 | 10 | 7413 | 7571 |
| 173 | 14 | 12308 | 11748 |
| 174 | 4 | 2350 | 3021 |
| 174 | 5 | 3082 | 3498 |
| 178 | 3 | 866 | 1105 |
| 179 | 8 | 8115 | 7816 |
| 179 | 17 | 17407 | 17135 |
| 180 | 4 | 3524 | 4537 |
| 180 | 5 | 4686 | 5687 |
| 180 | 6 | 5897 | 6949 |
| 180 | 9 | 9721 | 9299 |
| 180 | 10 | 9996 | 9715 |
| 180 | 20 | 19805 | 19954 |
| 180 | 23 | 21808 | 21509 |
| 180 | 25 | 24127 | 26460 |
| 180 | 27 | 27977 | 27474 |
| 181 | 1 | 381 | 82 |
| 183 | 1 | 190 | 2 |
| 183 | 4 | 1849 | 2211 |
| 183 | 5 | 2350 | 2568 |
| 183 | 7 | 3592 | 2978 |
| 183 | 8 | 4176 | 3571 |
| 185 | 2 | 1260 | 1424 |
| 185 | 3 | 2722 | 1301 |
| 185 | 4 | 3612 | 2671 |
| 187 | 2 | 727 | 1302 |
| 187 | 3 | 1293 | 1745 |
| 187 | 5 | 2592 | 2173 |
| 189 | 1 | 18 | 2180 |
| 190 | 1 | 466 | 68 |
| 190 | 2 | 896 | 411 |
| 190 | 4 | 1878 | 2165 |
| 190 | 5 | 2740 | 2384 |
| 190 | 10 | 10281 | 8875 |
| 191 | 2 | 861 | 658 |
| 191 | 3 | 1096 | 827 |
| 192 | 2 | 1881 | 1564 |
| 193 | 1 | 316 | 2 |
| 193 | 7 | 4667 | 3813 |
| 194 | 1 | 30 | 641 |
| 194 | 2 | 608 | 1582 |
| 195 | 1 | 2 | 433 |
| 195 | 2 | 431 | 943 |
| 195 | 3 | 1055 | 465 |
| 195 | 4 | 972 | 1487 |
| 195 | 5 | 1507 | 1995 |
| 195 | 6 | 3314 | 1851 |
| 195 | 9 | 3089 | 3529 |
| 195 | 10 | 3521 | 3312 |
| 195 | 12 | 6604 | 6837 |
| 195 | 13 | 7049 | 6786 |
| 195 | 14 | 6825 | 7700 |
| 195 | 15 | 7682 | 7047 |
| 195 | 16 | 7202 | 7417 |
| 195 | 18 | 8278 | 9036 |
| 195 | 20 | 8583 | 8837 |
| 195 | 21 | 8871 | 9602 |
| 195 | 22 | 9251 | 9403 |
| 195 | 23 | 9600 | 10022 |
| 195 | 25 | 10020 | 10226 |
| 195 | 26 | 11229 | 10024 |
| 195 | 27 | 10659 | 10946 |
| 195 | 28 | 10944 | 11318 |
| 195 | 30 | 12449 | 12246 |
| 195 | 32 | 13212 | 12505 |
| 195 | 33 | 12558 | 12773 |
| 195 | 35 | 13673 | 14011 |
| 195 | 36 | 14811 | 14143 |
| 195 | 38 | 16061 | 16363 |
| 195 | 39 | 16320 | 16799 |
| 195 | 40 | 16515 | 16333 |
| 196 | 1 | 608 | 1411 |
| 197 | 9 | 9269 | 9553 |
| 200 | 2 | 1103 | 249 |
| 200 | 3 | 1335 | 1033 |
| 200 | 4 | 1769 | 1284 |
| 200 | 5 | 2124 | 1747 |
| 200 | 6 | 2792 | 2106 |
| 200 | 7 | 3073 | 2708 |
| 200 | 8 | 3510 | 3061 |
| 200 | 9 | 4126 | 3467 |
| 200 | 10 | 4350 | 4042 |
| 200 | 11 | 4847 | 4368 |
| 200 | 14 | 6487 | 6182 |
| 200 | 15 | 6681 | 6499 |
| 200 | 18 | 10749 | 9307 |
| 200 | 20 | 11787 | 11464 |
| 200 | 22 | 12859 | 12410 |
| 201 | 1 | 509 | 105 |
| 201 | 3 | 3704 | 3237 |
| 202 | 7 | 5296 | 4817 |
| 205 | 2 | 117 | 323 |
| 205 | 5 | 1669 | 2148 |
| 206 | 2 | 546 | 196 |
| 206 | 3 | 841 | 632 |
| 206 | 4 | 1622 | 777 |
| 206 | 9 | 5466 | 5035 |
| 209 | 1 | 472 | 86 |
| 209 | 3 | 1510 | 1280 |
| 210 | 3 | 3175 | 2363 |
| 210 | 6 | 5281 | 4868 |
| 210 | 8 | 5619 | 6002 |
| 211 | 4 | 1708 | 3756 |
| 212 | 1 | 919 | 2 |
| 213 | 2 | 1107 | 1826 |
| 214 | 2 | 2106 | 1237 |
| 214 | 4 | 3677 | 3132 |
| 217 | 6 | 3548 | 3162 |
| 218 | 1 | 1 | 1218 |
| 218 | 3 | 2731 | 3378 |
| 218 | 5 | 4188 | 4667 |
| 219 | 3 | 1386 | 910 |
| 219 | 4 | 1595 | 1344 |
| 220 | 2 | 794 | 1144 |
| 221 | 1 | 110 | 295 |
| 221 | 2 | 326 | 880 |
| 221 | 4 | 1496 | 1825 |
| 221 | 5 | 1907 | 2200 |
| 221 | 6 | 2169 | 2555 |
| 221 | 8 | 3425 | 4246 |
| 221 | 9 | 4233 | 5111 |
| 221 | 12 | 6419 | 6757 |
| 221 | 13 | 6751 | 6987 |
| 221 | 14 | 6911 | 7120 |
| 221 | 16 | 7400 | 7909 |
| 221 | 17 | 7963 | 8199 |
| 221 | 19 | 8597 | 9079 |
| 222 | 17 | 11376 | 11597 |
| 223 | 6 | 5328 | 5008 |
| 223 | 12 | 12189 | 13307 |
| 223 | 13 | 13291 | 13716 |
| 223 | 14 | 13601 | 13434 |
| 223 | 17 | 15331 | 15068 |
| 223 | 19 | 15940 | 17160 |
| 223 | 21 | 17710 | 19089 |
| 223 | 23 | 19800 | 20708 |
| 223 | 25 | 22857 | 22027 |
| 223 | 26 | 22757 | 23365 |
| 225 | 1 | 756 | 394 |
| 225 | 5 | 3793 | 2945 |
| 226 | 1 | 141 | 536 |
| 226 | 2 | 521 | 871 |
| 228 | 8 | 5473 | 4835 |
| 229 | 7 | 6749 | 6057 |
| 232 | 2 | 1461 | 910 |
| 233 | 5 | 3359 | 3063 |
| 233 | 11 | 7226 | 7456 |
| 236 | 1 | 3 | 482 |
| 237 | 1 | 1 | 219 |
| 237 | 3 | 1197 | 991 |
| 237 | 5 | 2009 | 2329 |
| 237 | 6 | 2319 | 3056 |
| 237 | 8 | 3261 | 3701 |
| 237 | 10 | 3900 | 4763 |
| 237 | 11 | 4730 | 4963 |
| 238 | 11 | 9966 | 9238 |
| 238 | 19 | 16613 | 17728 |
| 238 | 29 | 26812 | 27663 |
| 239 | 2 | 1576 | 4245 |
| 239 | 5 | 6393 | 6956 |
| 239 | 6 | 6902 | 7237 |
| 240 | 5 | 1537 | 1809 |
| 241 | 1 | 228 | 1040 |
| 242 | 9 | 6581 | 7015 |
| 242 | 10 | 6988 | 7368 |
| 242 | 12 | 7488 | 7928 |
| 245 | 2 | 1670 | 1251 |
| 247 | 2 | 1558 | 1812 |
| 250 | 4 | 3210 | 2998 |
| 251 | 1 | 622 | 2 |
| 252 | 3 | 2598 | 2383 |
| 252 | 4 | 2911 | 2564 |
| 253 | 1 | 1 | 345 |
| 253 | 2 | 359 | 898 |
| 254 | 1 | 2 | 307 |
| 254 | 3 | 318 | 4 |
| 256 | 5 | 3768 | 4040 |
| 256 | 7 | 7292 | 6639 |
| 256 | 9 | 9589 | 8465 |
| 257 | 2 | 992 | 294 |
| 257 | 4 | 4528 | 3596 |
| 257 | 7 | 6894 | 6718 |
| 257 | 8 | 7252 | 6884 |
| 257 | 9 | 7986 | 7231 |
| 258 | 2 | 544 | 804 |
| 258 | 3 | 1224 | 2921 |
| 258 | 4 | 2964 | 2728 |
| 258 | 5 | 2919 | 3752 |
| 258 | 6 | 4120 | 5298 |
| 261 | 1 | 3 | 362 |
| 264 | 1 | 582 | 361 |
| 264 | 2 | 881 | 561 |
| 264 | 3 | 1367 | 879 |
| 264 | 4 | 1966 | 1361 |
| 264 | 5 | 2316 | 1945 |
| 264 | 6 | 2636 | 2295 |
| 264 | 7 | 3194 | 2634 |
| 264 | 8 | 3531 | 3055 |
| 265 | 2 | 398 | 817 |
| 265 | 4 | 1583 | 1071 |
| 265 | 6 | 3293 | 3009 |
| 265 | 7 | 3186 | 3046 |
| 266 | 1 | 451 | 2 |
| 266 | 4 | 1983 | 2225 |
| 266 | 7 | 2540 | 2325 |
| 268 | 1 | 798 | 1223 |
| 268 | 2 | 1912 | 1265 |
| 270 | 4 | 3977 | 4186 |
| 270 | 6 | 4397 | 4573 |
| 271 | 5 | 2719 | 3066 |
| 271 | 6 | 3041 | 3352 |
| 271 | 9 | 6278 | 5862 |
| 271 | 10 | 6550 | 5993 |
| 271 | 14 | 10291 | 10004 |
| 272 | 3 | 1870 | 1199 |
| 272 | 4 | 3378 | 1831 |
| 276 | 5 | 2350 | 1994 |
| 276 | 8 | 3702 | 3103 |
| 276 | 9 | 4441 | 3692 |
| 276 | 10 | 4595 | 4416 |
| 276 | 12 | 8173 | 7382 |
| 276 | 14 | 10001 | 9762 |
| 276 | 15 | 11065 | 9890 |
| 276 | 17 | 11642 | 11250 |
| 276 | 19 | 12892 | 12503 |
| 276 | 21 | 13302 | 13099 |
| 276 | 22 | 13663 | 13271 |
| 276 | 23 | 13995 | 13642 |
| 276 | 25 | 15065 | 14211 |
| 276 | 27 | 16293 | 15955 |
| 276 | 29 | 18482 | 16563 |
| 276 | 31 | 19951 | 19016 |
| 279 | 3 | 1469 | 1675 |
| 279 | 4 | 1600 | 1923 |
| 279 | 5 | 2269 | 2105 |
| 279 | 10 | 7698 | 7279 |
| 280 | 3 | 3138 | 2968 |
| 281 | 4 | 2055 | 2552 |
| 282 | 1 | 316 | 2 |
| 282 | 2 | 456 | 1232 |
| 282 | 3 | 1957 | 1346 |
| 283 | 1 | 1 | 450 |
| 283 | 3 | 1098 | 1556 |
| 283 | 5 | 2062 | 2238 |
| 283 | 7 | 3127 | 3312 |
| 286 | 3 | 2883 | 2698 |
| 287 | 4 | 2359 | 2180 |
| 290 | 10 | 8820 | 9074 |
| 290 | 11 | 9008 | 9172 |
| 291 | 2 | 1103 | 855 |
| 291 | 3 | 2622 | 1123 |
| 292 | 1 | 2 | 283 |
| 292 | 2 | 701 | 330 |
| 292 | 5 | 2459 | 2866 |
| 292 | 7 | 4252 | 4995 |
| 292 | 9 | 6704 | 7096 |
| 292 | 10 | 7066 | 7827 |
| 292 | 12 | 8377 | 8622 |
| 292 | 15 | 11502 | 12674 |
| 292 | 17 | 13326 | 13727 |
| 292 | 18 | 13738 | 14778 |
| 294 | 1 | 117 | 623 |
| 294 | 2 | 905 | 723 |
| 294 | 6 | 2496 | 2272 |
| 295 | 7 | 4274 | 4510 |
| 300 | 4 | 3525 | 3337 |
| 301 | 6 | 6714 | 4852 |
| 301 | 13 | 10150 | 9914 |
| 301 | 16 | 11316 | 11657 |
| 301 | 18 | 13199 | 14398 |
| 301 | 19 | 15724 | 14657 |
| 306 | 3 | 1135 | 2727 |
| 306 | 4 | 2742 | 4025 |
| 306 | 5 | 4004 | 4552 |
| 306 | 6 | 4527 | 5117 |
| 306 | 7 | 5131 | 5466 |
| 306 | 9 | 5642 | 5968 |
| 306 | 11 | 7000 | 8013 |
| 306 | 12 | 7926 | 8138 |
| 306 | 13 | 8180 | 8908 |
| 306 | 14 | 8899 | 9120 |
| 306 | 15 | 9118 | 9510 |
| 306 | 16 | 9508 | 9963 |
| 306 | 17 | 9964 | 11313 |
| 306 | 18 | 11319 | 11570 |
| 306 | 19 | 11540 | 11707 |
| 306 | 20 | 11626 | 11856 |
| 310 | 2 | 1126 | 176 |
| 310 | 5 | 4215 | 3556 |
| 311 | 4 | 5671 | 6006 |
| 311 | 5 | 6173 | 6778 |
| 311 | 6 | 6833 | 7225 |
| 311 | 7 | 7236 | 7520 |
| 311 | 8 | 7492 | 7926 |
| 312 | 2 | 859 | 1506 |
| 312 | 3 | 1449 | 1808 |
| 312 | 4 | 2043 | 2306 |
| 313 | 4 | 3568 | 3122 |
| 319 | 1 | 3 | 881 |
| 319 | 2 | 832 | 1185 |
| 321 | 1 | 638 | 898 |
| 321 | 4 | 1862 | 2131 |
| 321 | 5 | 2168 | 2548 |
| 321 | 6 | 2470 | 3159 |
| 321 | 7 | 3069 | 3395 |
| 321 | 8 | 3461 | 3733 |
| 324 | 1 | 3 | 692 |
| 324 | 2 | 867 | 1592 |
| 324 | 4 | 2392 | 3021 |
| 327 | 6 | 5052 | 5213 |
| 330 | 5 | 3745 | 3464 |
| 333 | 2 | 998 | 717 |
| 333 | 3 | 947 | 1534 |
| 335 | 2 | 1024 | 521 |
| 338 | 11 | 8869 | 8591 |
| 340 | 5 | 3931 | 3608 |
| 341 | 6 | 3484 | 3155 |
| 341 | 7 | 4348 | 3482 |
| 341 | 8 | 6419 | 4332 |
| 341 | 10 | 9264 | 7672 |
| 341 | 11 | 10777 | 9245 |
| 341 | 12 | 12026 | 10779 |
| 343 | 1 | 459 | 262 |
| 343 | 4 | 3905 | 2661 |
| 345 | 4 | 3467 | 3201 |
| 345 | 14 | 15320 | 16447 |
| 345 | 16 | 18409 | 18927 |
| 345 | 18 | 19974 | 20465 |
| 347 | 1 | 763 | 1155 |
| 350 | 5 | 3273 | 2980 |
| 351 | 1 | 693 | 280 |
| 351 | 2 | 1268 | 654 |
| 351 | 3 | 1716 | 1222 |
| 353 | 4 | 2749 | 2546 |
| 354 | 1 | 2 | 298 |
| 355 | 16 | 8911 | 9399 |
| 355 | 19 | 12476 | 12904 |
| 355 | 22 | 15766 | 15608 |
| 355 | 23 | 17165 | 17461 |
| 355 | 25 | 18313 | 19104 |
| 355 | 26 | 19092 | 19598 |
| 355 | 27 | 19692 | 19495 |
| 355 | 28 | 19734 | 20198 |
| 355 | 29 | 20196 | 20471 |
| 356 | 2 | 2204 | 1536 |
| 356 | 4 | 2887 | 2537 |
| 356 | 5 | 3167 | 2859 |
| 357 | 1 | 381 | 4 |
| 360 | 3 | 3167 | 2877 |
| 361 | 1 | 7 | 909 |
| 363 | 1 | 1405 | 167 |
| 363 | 6 | 7178 | 8404 |
| 364 | 1 | 41 | 331 |
| 366 | 2 | 1386 | 1598 |
| 367 | 19 | 8690 | 8941 |
| 368 | 4 | 1786 | 1947 |
| 369 | 4 | 1652 | 1428 |
| 372 | 6 | 5262 | 4534 |
| 376 | 2 | 625 | 293 |
| 377 | 1 | 331 | 2 |
| 379 | 4 | 2975 | 3142 |
| 382 | 3 | 2951 | 3277 |
| 382 | 4 | 4183 | 3320 |
| 383 | 6 | 6158 | 5637 |
| 386 | 9 | 5725 | 6027 |
| 387 | 2 | 486 | 980 |
| 390 | 2 | 1668 | 2057 |
| 390 | 3 | 3499 | 2867 |
| 391 | 1 | 2 | 154 |
| 392 | 5 | 5163 | 5387 |
| 394 | 1 | 1 | 375 |
| 394 | 8 | 6437 | 7585 |
| 394 | 9 | 7542 | 7967 |
| 394 | 11 | 10354 | 10713 |
| 395 | 5 | 1957 | 2229 |
| 395 | 9 | 3869 | 4216 |
| 395 | 11 | 4571 | 4960 |
| 398 | 1 | 395 | 1180 |
| 399 | 7 | 5691 | 6134 |
| 399 | 10 | 7662 | 7820 |
| 399 | 14 | 10111 | 9845 |
| 399 | 22 | 16699 | 16481 |
| 399 | 29 | 28519 | 28244 |
| 401 | 1 | 189 | 4 |
| 401 | 2 | 178 | 1044 |
| 401 | 3 | 1038 | 2141 |
| 401 | 5 | 3517 | 3939 |
| 402 | 3 | 919 | 1269 |
| 404 | 1 | 578 | 12 |
| 405 | 1 | 293 | 643 |
| 405 | 3 | 1926 | 1501 |
| 407 | 1 | 80 | 406 |
| 407 | 4 | 3188 | 3670 |
| 408 | 5 | 3037 | 2681 |
| 408 | 6 | 3786 | 3475 |
| 410 | 2 | 811 | 1092 |
| 413 | 2 | 742 | 1314 |
| 413 | 3 | 1275 | 1532 |
| 414 | 2 | 908 | 678 |
| 414 | 3 | 1137 | 1889 |
| 414 | 4 | 2738 | 1959 |
| 416 | 3 | 1945 | 1709 |
| 418 | 1 | 3 | 350 |
| 418 | 2 | 331 | 930 |
| 419 | 2 | 619 | 296 |
| 419 | 4 | 937 | 773 |
| 419 | 5 | 1305 | 910 |
| 419 | 6 | 1183 | 1521 |
| 419 | 7 | 1859 | 1299 |
| 419 | 8 | 2170 | 1850 |
| 419 | 9 | 2483 | 2160 |
| 419 | 10 | 3399 | 2470 |
| 419 | 11 | 3708 | 3397 |
| 420 | 3 | 1649 | 1452 |
| 421 | 6 | 3983 | 3510 |
| 424 | 1 | 797 | 3 |
| 424 | 2 | 513 | 851 |
| 424 | 3 | 1029 | 733 |
| 424 | 6 | 1859 | 1551 |
| 424 | 7 | 3076 | 2780 |
| 425 | 1 | 52 | 384 |
| 425 | 2 | 1031 | 777 |
| 425 | 3 | 1127 | 1936 |
| 427 | 2 | 1488 | 1114 |
| 427 | 3 | 2114 | 1464 |
| 430 | 2 | 1334 | 1489 |
| 431 | 1 | 420 | 196 |
| 431 | 2 | 634 | 269 |
| 432 | 2 | 1133 | 1372 |
| 432 | 3 | 2014 | 1439 |
| 432 | 6 | 3869 | 3378 |
| 433 | 1 | 292 | 2007 |
| 435 | 1 | 706 | 131 |
| 435 | 2 | 1730 | 1047 |
| 439 | 1 | 1 | 627 |
| 441 | 1 | 1 | 513 |
| 441 | 7 | 10592 | 7974 |
| 443 | 1 | 31 | 744 |
| 447 | 2 | 744 | 322 |
| 449 | 1 | 3 | 212 |
| 449 | 2 | 471 | 286 |
| 449 | 3 | 551 | 393 |
| 451 | 1 | 823 | 314 |
| 452 | 2 | 322 | 714 |
| 452 | 6 | 2806 | 3342 |
| 452 | 7 | 3358 | 3792 |
| 454 | 1 | 1033 | 2 |
| 455 | 3 | 3214 | 3837 |
| 455 | 5 | 4078 | 4488 |
| 455 | 6 | 4965 | 4117 |
| 455 | 8 | 5123 | 5473 |
| 457 | 1 | 940 | 35 |
| 461 | 2 | 476 | 691 |
| 461 | 4 | 1548 | 1991 |
| 461 | 5 | 2322 | 1948 |
| 461 | 6 | 2664 | 2449 |
| 462 | 5 | 2810 | 2064 |
| 464 | 2 | 2162 | 1530 |
| 465 | 1 | 1762 | 38 |
| 465 | 3 | 2373 | 2050 |
| 467 | 2 | 652 | 1260 |
| 467 | 3 | 1149 | 1442 |
| 469 | 2 | 922 | 1101 |
| 470 | 2 | 971 | 1768 |
| 473 | 2 | 450 | 220 |
| 475 | 1 | 1 | 969 |
| 477 | 2 | 1064 | 843 |
| 482 | 1 | 1 | 534 |
| 484 | 1 | 130 | 543 |
| 484 | 2 | 1320 | 1159 |
| 487 | 2 | 1258 | 1929 |
| 488 | 2 | 509 | 162 |
| 488 | 4 | 2247 | 1945 |
| 489 | 1 | 1 | 396 |
| 489 | 2 | 560 | 255 |
| 490 | 2 | 1096 | 458 |
| 491 | 5 | 5167 | 4433 |
| 491 | 6 | 5975 | 5247 |
| 491 | 7 | 6811 | 6041 |
| 494 | 1 | 650 | 3 |
| 497 | 5 | 3351 | 3536 |
| 497 | 8 | 4757 | 4308 |
| 497 | 10 | 5229 | 5086 |
| 497 | 11 | 5967 | 5671 |
| 499 | 1 | 663 | 247 |
| 502 | 2 | 1324 | 851 |
| 504 | 1 | 3 | 650 |
| 507 | 2 | 727 | 906 |
| 507 | 3 | 840 | 1010 |
| 510 | 3 | 2056 | 2574 |
| 512 | 2 | 854 | 300 |
| 514 | 2 | 1067 | 669 |
| 518 | 5 | 3119 | 2970 |
| 520 | 1 | 3 | 467 |
| 520 | 2 | 452 | 231 |
| 520 | 4 | 2218 | 1859 |
| 521 | 2 | 988 | 821 |
| 522 | 1 | 409 | 885 |
| 524 | 1 | 579 | 4 |
| 525 | 1 | 1 | 144 |
| 525 | 2 | 86 | 352 |
| 529 | 2 | 5731 | 6147 |
| 533 | 1 | 1044 | 157 |
| 536 | 3 | 587 | 1462 |
| 539 | 7 | 6180 | 6662 |
| 540 | 1 | 198 | 476 |
| 543 | 3 | 2179 | 1835 |
| 543 | 4 | 2404 | 2177 |
| 543 | 7 | 3924 | 3700 |
| 544 | 2 | 1004 | 870 |
| 546 | 2 | 497 | 324 |
| 547 | 3 | 717 | 965 |
| 549 | 2 | 371 | 135 |
| 550 | 1 | 527 | 3 |
| 550 | 2 | 864 | 709 |
| 550 | 3 | 1540 | 1277 |
| 550 | 4 | 2039 | 1509 |
| 552 | 5 | 4681 | 5073 |
| 552 | 8 | 8390 | 8223 |
| 555 | 1 | 470 | 267 |
| 560 | 1 | 635 | 210 |
| 560 | 2 | 834 | 514 |
| 563 | 2 | 1215 | 1469 |
| 564 | 1 | 8 | 511 |
| 564 | 2 | 1019 | 555 |
| 564 | 3 | 577 | 744 |
| 565 | 1 | 321 | 4 |
| 565 | 5 | 1266 | 1619 |
| 567 | 2 | 1055 | 531 |
| 571 | 3 | 1149 | 886 |
| 573 | 1 | 208 | 666 |
| 573 | 2 | 651 | 1148 |
| 573 | 5 | 2558 | 2809 |
| 575 | 1 | 262 | 2 |
| 584 | 1 | 268 | 110 |
| 584 | 4 | 1310 | 795 |
| 584 | 5 | 1329 | 1574 |
| 586 | 1 | 771 | 4 |
| 588 | 1 | 346 | 56 |
| 588 | 2 | 1078 | 434 |
| 589 | 1 | 1 | 555 |
| 591 | 1 | 217 | 2 |
| 592 | 2 | 674 | 868 |
| 593 | 1 | 190 | 2 |
| 593 | 3 | 1035 | 1268 |
| 601 | 1 | 77 | 274 |
| 601 | 2 | 172 | 576 |
| 602 | 2 | 759 | 415 |
| 604 | 6 | 2868 | 2416 |
| 606 | 1 | 271 | 798 |
| 607 | 2 | 633 | 797 |
| 613 | 1 | 420 | 82 |
| 616 | 2 | 593 | 435 |
| 616 | 4 | 975 | 730 |
| 619 | 3 | 641 | 817 |
| 620 | 1 | 863 | 3 |
| 621 | 2 | 1493 | 2014 |
| 627 | 1 | 113 | 763 |
| 628 | 1 | 2 | 163 |
| 631 | 1 | 1 | 516 |
| 631 | 3 | 1715 | 1521 |
| 633 | 1 | 280 | 2 |
| 634 | 3 | 1139 | 1387 |
| 637 | 2 | 1613 | 738 |
| 637 | 3 | 1597 | 2208 |
| 637 | 4 | 2242 | 2694 |
| 637 | 7 | 3550 | 4545 |
| 637 | 9 | 4767 | 5171 |
| 639 | 1 | 175 | 2 |
| 640 | 2 | 468 | 689 |
| 643 | 1 | 496 | 320 |
| 645 | 1 | 1 | 537 |
| 645 | 2 | 539 | 1024 |
| 647 | 1 | 64 | 855 |
| 647 | 2 | 1419 | 895 |
| 649 | 1 | 2 | 364 |
| 651 | 1 | 539 | 3 |
| 653 | 2 | 738 | 550 |
| 656 | 8 | 7784 | 8587 |
| 657 | 2 | 1356 | 967 |
| 657 | 3 | 1708 | 1376 |
| 661 | 1 | 2 | 244 |
| 664 | 3 | 1149 | 820 |
| 672 | 1 | 546 | 10 |
| 673 | 2 | 1207 | 1827 |
| 676 | 1 | 443 | 790 |
| 679 | 1 | 998 | 219 |
| 682 | 3 | 749 | 1171 |
| 685 | 1 | 176 | 511 |
| 685 | 2 | 498 | 199 |
| 685 | 3 | 480 | 947 |
| 685 | 4 | 1000 | 1443 |
| 686 | 4 | 1567 | 2001 |
| 686 | 5 | 3238 | 1712 |
| 686 | 7 | 2965 | 3435 |
| 686 | 8 | 3441 | 3067 |
| 686 | 9 | 3752 | 3339 |
| 686 | 10 | 3530 | 3826 |
| 688 | 2 | 628 | 894 |
| 689 | 2 | 582 | 331 |
| 690 | 1 | 275 | 90 |
| 690 | 2 | 487 | 248 |
| 696 | 1 | 239 | 9 |
| 696 | 2 | 1237 | 233 |
| 696 | 3 | 1424 | 1200 |
| 697 | 1 | 20 | 520 |
| 698 | 1 | 29 | 313 |
| 698 | 2 | 217 | 483 |
| 701 | 5 | 1061 | 1534 |
| 707 | 2 | 855 | 538 |
| 709 | 1 | 1 | 675 |
| 710 | 1 | 3 | 416 |
| 712 | 1 | 674 | 96 |
| 713 | 1 | 933 | 139 |
| 713 | 2 | 1125 | 1436 |
| 716 | 2 | 1226 | 765 |
| 721 | 1 | 3 | 371 |
| 726 | 1 | 543 | 94 |
| 729 | 1 | 19 | 210 |
| 731 | 1 | 532 | 2 |
| 736 | 2 | 309 | 644 |
| 738 | 1 | 561 | 4 |
| 740 | 1 | 488 | 3 |
| 749 | 2 | 20 | 475 |
| 751 | 1 | 1 | 456 |
| 751 | 2 | 454 | 774 |
| 753 | 1 | 76 | 729 |
| 754 | 1 | 761 | 21 |
| 755 | 2 | 345 | 539 |
| 756 | 1 | 1 | 375 |
| 764 | 2 | 528 | 1088 |
| 772 | 1 | 1 | 558 |
| 772 | 2 | 432 | 866 |
| 775 | 1 | 706 | 2 |
| 778 | 2 | 992 | 834 |
| 780 | 1 | 52 | 351 |
| 782 | 1 | 3 | 557 |
| 783 | 1 | 28 | 609 |
| 791 | 1 | 1 | 582 |
| 791 | 2 | 859 | 641 |
| 791 | 3 | 1235 | 711 |
| 797 | 1 | 2 | 289 |
| 797 | 2 | 287 | 3 |
| 801 | 2 | 598 | 191 |
| 805 | 1 | 1 | 414 |
| 806 | 1 | 392 | 3 |
| 810 | 1 | 3 | 317 |
| 810 | 2 | 407 | 3 |
| 815 | 2 | 443 | 282 |
| 819 | 1 | 39 | 668 |
| 830 | 1 | 291 | 4 |
| 830 | 2 | 476 | 162 |
| 834 | 1 | 561 | 46 |
| 834 | 2 | 953 | 453 |
| 837 | 1 | 3 | 317 |
| 837 | 2 | 320 | 589 |
| 839 | 1 | 1 | 753 |
| 841 | 1 | 1 | 489 |
| 855 | 1 | 308 | 3 |
| 861 | 1 | 1 | 330 |
| 863 | 1 | 451 | 221 |
| 870 | 1 | 21 | 503 |
| 890 | 2 | 1548 | 1255 |
| 895 | 1 | 3 | 140 |
| 896 | 1 | 2 | 400 |
| 897 | 2 | 244 | 498 |
| 902 | 1 | 1 | 300 |
| 904 | 1 | 294 | 4 |
| 910 | 1 | 143 | 3 |
| 917 | 1 | 36 | 518 |
| 918 | 1 | 3 | 167 |
| 918 | 2 | 116 | 373 |
| 920 | 2 | 243 | 515 |
| 922 | 1 | 669 | 259 |
| 926 | 1 | 2 | 394 |
| 927 | 1 | 119 | 556 |
| 928 | 1 | 493 | 179 |
| 930 | 1 | 526 | 344 |
| 933 | 2 | 257 | 418 |
| 936 | 2 | 243 | 683 |
| 937 | 1 | 341 | 3 |
| 942 | 1 | 58 | 228 |
| 945 | 1 | 318 | 4 |
| 953 | 1 | 254 | 48 |
| 959 | 1 | 1198 | 164 |
| 959 | 2 | 1740 | 1123 |
| 963 | 2 | 462 | 232 |
| 965 | 1 | 403 | 2 |
| 969 | 1 | 360 | 4 |
| 970 | 3 | 673 | 314 |
| 972 | 1 | 3 | 470 |
| 973 | 1 | 2 | 700 |
| 974 | 1 | 2 | 235 |
| 974 | 3 | 270 | 467 |
| 981 | 2 | 154 | 405 |
| 984 | 3 | 164 | 337 |

### SEQUENCE LISTING PLACE INDICATOR

PAGES 280 TO 2076, WHICH ARE THE COMPLETE SEQUENCE LISTINGS FOR THIS APPLICATION, ARE LOCATED IN THE FOUR (4) ATTACHED REDWELDS IDENTIFIED BY THE FOLLOWING INFORMATION ON THE INDIVIDUAL VOLUME COVER SHEETS:

Applicants: Kunsch et al.

Serial No.: Unassigned

Filed: Concurrently herewith

For: Enterococcos faecalis Polynucleotides and Polypeptides

Attorney Docket No. PB369

What is claimed is:

1. Computer readable medium having recorded thereon the nucleotide sequence depicted in SEQ ID NOS: 1-982, a representative fragment thereof or a nucleotide sequence at least 95% identical to a nucleotide sequence depicted in SEQ ID NOS:1-982.

2. The computer readable medium of claim 1 having recorded thereon any one of the fragments of SEQ ID NOS:1-982 depicted in Tables 2 and 3 or a degenerate variant thereof.

3. The computer readable medium of claim 1, wherein said medium is selected from the group consisting of a floppy disc, a hard disc, random access memory (RAM), read only memory (ROM), and CD-ROM.

4. The computer readable medium of claim 3, wherein said medium is selected from the group consisting of a floppy disc, a hard disc, random access memory (RAM), read only memory (ROM), and CD-ROM.

5. A computer-based system for identifying fragments of the Enterococcus faecalis genome of commercial importance comprising the following elements:
a) a data storage means comprising the nucleotide sequence of SEQ ID NOS:1-982, a representative fragment thereof, or a nucleotide sequence at least 95% identical to a nucleotide sequence of SEQ ID NOS:1-982;
b) search means for comparing a target sequence to the nucleotide sequence of the data storage means of step (a) to identify homologous sequence(s), and
c) retrieval means for obtaining said homologous sequence(s) of step (b).

6. A method for identifying commercially important nucleic acid fragments of the Enterococcus faecalis genome comprising the step of comparing a database comprising the nucleotide sequences depicted in SEQ ID NOS:1-982, a representative fragment thereof, or a nucleotide sequence at least 95% identical to a nucleotide sequence of SEQ ID NOS:1-982 with a target sequence to obtain a nucleic acid molecule comprised of a complementary nucleotide sequence to said target sequence, wherein said target sequence is not randomly selected.

7. A method for identifying an expression modulating fragment of Enterococcus faecalis genome comprising the step of comparing a database comprising the nucleotide sequences depicted in SEQ ID NOS: 1-982, a representative fragment thereof, or a nucleotide sequence at least 95% identical to the nucleotide sequence of SEQ ID NOS:1-982 with a target sequence to obtain a nucleic acid molecule comprised of a complementary nucleotide sequence to said target sequence, wherein said target sequence comprises sequences known to regulate gene expression.

8. An isolated protein-encoding nucleic acid fragment of the Enterococcus faecalis genome, wherein said fragment consists of the nucleotide sequence of any one of the fragments of SEQ ID NOS:1-982 depicted in Tables 2 and 3, or a degenerate variant thereof.

9. A vector comprising any one of the fragments of the Enterococcus faecalis genome of claim 8.

10. An isolated fragment of the Enterococcus faecalis genome, wherein said fragment modulates the expression of an operably linked open reading frame, wherein said fragment consists of the nucleotide sequence from about 10 to 200 bases in length which is 5′ to any one of the open reading of claim 8.

11. A vector comprising any one of the fragments of the Enterococcus faecalis genome of claim 8.

12. An organism which has been altered to contain any one of the fragments of the Enterococcus faecalis genome of claim 8.

13. An organism which has been altered to contain any one of the fragments of the Enterococcus faecalis genome of claim 10.

14. A method for regulating the expression of a nucleic acid molecule comprising the step of covalently attaching to said nucleic acid molecule to a a nucleic acid molecule of claim 10.

15. An isolated polypeptide encoded by any of the fragments of the Enterococcus faecalis genome of claim 8.

16. An isolated polynucleotide molecule encoding any one of the polypeptides of claim 15.

17. An antibody which selectively binds to any one of the polypeptides of claim 15.

18. A method for producing a polypeptide in a host cell comprising the steps of:
a) incubating a host containing a heterologous nucleic acid molecule whose nucleotide sequence consists of any one of the fragments of the Enterococcus faecalis genome of claim 8, under conditions where said heterologous nucleic acid molecule is expressed to produce said protein, and
b) isolating said protein.
